(19) United States
(12) Patent Application Publication
(10) Pub. No.: US 2026/0009051 A1
(43) Pub. Date: Jan. 8, 2026

(54) COMPOSITIONS AND METHODS FOR ARTIFICIAL PROTOSPACER ADJACENT MOTIF (PAM) GENERATION

(71) Applicant: VOR BIOPHARMA INC., Cambridge, MA (US)

(72) Inventors: John Lydeard, Cambridge, MA (US); Tirtha Chakraborty, Cambridge, MA (US); Alejandra Falla, Cambridge, MA (US); Julian Scherer, Cambridge, MA (US); Abhinav Dhall, Cambridge, MA (US)

(73) Assignee: VOR BIOPHARMA INC., Cambridge, MA (US)

(21) Appl. No.: 18/881,462

(22) PCT Filed: Jul. 13, 2023
(86) PCT No.: PCT/US2023/070156

§ 371 Date: Jan. 6, 2025

Related U.S. Application Data
(60) Provisional application No. 63/388,970, filed on Jul. 13, 2022.

(51) Int. Cl. C12N 15/90; A61K 35/28; C07K 14/52; C07K 14/705; C07K 14/715; C12N 9/22; C12N 9/78; C12N 15/11

(52) U.S. Cl. CPC C12N 15/907; A61K 35/28; C07K 14/521; C07K 14/70503; C07K 14/70596; C07K 14/7155; C12N 9/226; C12N 9/78; C12N 15/11; C12Y 305/04005; C07K 2319/00; C12N 2310/20

(57) ABSTRACT

This disclosure provides, inter alia, genetically engineered cells having an artificial protospacer-adjacent motif (PAM) introduced via a CRISPR/Cas-mediated gene editing, and optionally, one or more modifications in a target gene. The disclosure also provides methods and compositions, including gRNAs, that can be used to make such modifications.

### RELATED APPLICATIONS

The application claims the benefit under 35 U.S.C. 119 (e) of U.S. Provisional Application No. 63/388,970 filed Jul. 13, 2022, the contents of which is incorporated by reference in its entirety.

### REFERENCE TO AN ELECTRONIC SEQUENCE LISTING

The contents of the electronic sequence listing (V029170045WO00-SEQ-CEW.xml; Size: 132,171 bytes; and Date of Creation: Jul. 13, 2023) is herein incorporated by reference in its entirety.

### BACKGROUND

Conventional CRISPR/Cas systems are associated with certain limitations. For example, targeting a CRISPR/Cas nuclease to a specific nucleic acid target domain, e.g., a target sequence within a genome of a cell, typically requires the CRISPR/Cas nuclease to complex with a suitable guide RNA (gRNA) comprising a sequence that is complementary to a sequence of the target domain, as well as the presence of a short sequence flanking the target domain referred to as a protospacer-adjacent motif (PAM). A sequence lacking a suitable PAM sequence can thus not efficiently be targeted using conventional CRISPR/Cas systems. See, e.g., Collias et al., Nat Commun. 12 (1): 555, 2021. Accordingly, targetable sequences for a given CRISPR/Cas nuclease are limited by the requirement of conventional CRISPR/Cas nucleases for a suitable PAM sequence to be present in proximity to the target cut site.

There is a need for safe and effective methods to achieve gene editing of target domains which lack a suitable PAM in cells for therapeutic applications.

### SUMMARY

The present disclosure is based, at least in part, on the surprising discovery that nucleic acid sequences lacking a suitable PAM sequence can efficiently be targeted by CRISPR/Cas systems using the strategies, compositions, methods, and modalities provided herein. In some aspects, the present disclosure provides gene editing strategies, compositions, methods, and modalities that relate to genetic modification, or editing, of target nucleic acid sequences lacking a suitable PAM sequence for efficient editing by a CRISPR/Cas nuclease. In some embodiments, strategies, compositions, methods, and modalities provided herein relate to the introducing a mutation and generation of a suitable artificial PAM sequence (also referred to herein as an “artificial PAM” or “new PAM”) in proximity of a target sequence (i.e., target domain) lacking such a suitable PAM sequence (i.e., at a second or subsequent target site/domain), which thus renders the target sequence suitable for efficient targeting by a CRISPR/Cas system.

In some embodiments, the generation of an artificial PAM is effected using various CRISPR/Cas systems, e.g., base editing technology or homology directed repair (HDR)-mediated gene editing technology, and utilizing an existing PAM sequence, or a plurality of existing PAM sequences, in proximity to a first site, i.e., not suitable for directly editing or for effecting a desired mutation in the target sequence at the second or subsequent site. In some embodiments, the existing PAM sequence used to generate the new PAM (i.e., artificial PAM, also referred to herein as an artificial PAM sequence) to the target sequence at the second or subsequent site.

In some embodiments, the existing PAM sequence used for introducing a mutation and generating the artificial PAM proximal to the second or subsequent site is itself not suitable for effecting a desired mutation within the second or subsequent target sequence.

Some aspects of this disclosure provide genetically engineered cells (e.g., hematopoietic stem cells (HSCs) and/or hematopoietic progenitor cells (HPCs)) comprising a modification in a genomic DNA molecule encoding a protein, e.g., a modification of an epitope of the protein encoded by the respective gene, that diminishes the binding of a therapeutic agent to the protein. The disclosure also provides methods and compositions, including gRNAs, that can be used to make such modifications. In exemplary embodiments, such genetically engineered cells may be resistant to an anti-cancer therapy, and can therefore repopulate the hematopoietic system during or after anti-cancer therapy.

Some aspects of this disclosure provide genetically engineered cells, comprising: a modified genomic DNA molecule, wherein the modified genomic DNA molecule comprises a mutation in a nucleotide sequence at a first target domain, wherein the modified genomic DNA molecule comprises an artificial protospacer adjacent motif (PAM) sequence comprising the mutation at the first target domain, or a part of it, and wherein an unmodified genomic DNA molecule of the same type does not comprise the artificial PAM at the first target domain.

Some aspects of this disclosure provide cell populations, comprising a plurality of the genetically engineered cells described herein.

Some aspects of this disclosure provide cell populations comprising a plurality of genetically engineered cells, wherein at least a portion of the cells comprise: (a) a mutation within a first target domain in a genomic DNA molecule which generates an artificial PAM; (b) an artificial PAM in proximity of a second or subsequent target domain; and (c) an edit within the second or subsequent target domain.

Aspects of this disclosure are directed to the modification of DNA in a cell using one or more guide RNAs (gRNAs) to direct a CRISPR/Cas system to a target location on the DNA wherein the CRISPR/Cas system introduces a mutation. In exemplary embodiments, modification of a genomic DNA molecule in a cell uses one or more guide RNAs (gRNAs) to direct an RNA-guided CRISPR/Cas nuclease fused to a deaminase that targets and deaminates a specific nucleobase, e.g., a cytosine or adenosine nucleobase of a C or A nucleotide, which, via cellular mismatch repair mechanisms, results in a change from a C to a T nucleotide, or a change from an A to a G nucleotide, to a target location on the DNA wherein the base editor introduces a mutation. Such gene editing (i.e., modification of the genomic DNA) may result in the generation of a PAM. Alternatively, such gene editing may result in the introduction of a deletion, a substitution, an insertion, or an inversion of one or more amino acid residues, e.g., modification of an epitope of the protein encoded by the respective gene, that diminishes the binding of an agent to the protein.

Some aspects of this disclosure provide genetically engineered cells, e.g., HSCs or HPCs, having a modification or a plurality of modifications including an artificial PAM or a plurality of artificial PAMs and a mutation in one or more target genes (e.g., a plurality of mutations in one or more lineage-specific cell-surface antigens, such as in the endogenous CD33, CD20, CLL-1, CD123, CD38, CD19, CD117, EMR2, and/or CD5 gene(s)). In some embodiments, a genomic DNA molecule is genetically modified simultaneously or sequentially to include an artificial PAM or a plurality of artificial PAMs and a modification in one or more target genes (e.g., a plurality of modifications in one or more lineage-specific cell-surface antigens).

Some aspects of this disclosure provide methods, comprising administering to a subject in need thereof: (i) a population of the genetically engineered cells described herein. Aspects of this disclosure provide methods comprising: genetically modifying a cell to generate an artificial PAM in proximity of a target domain, comprising contacting a genomic DNA molecule with: (a) a first CRISPR/Cas system comprising a Cas nuclease and a first gRNA configured to direct the first CRISPR/Cas system to a first target domain resulting in the generation of an artificial PAM comprising introducing a mutation at the first target domain of the genomic DNA molecule; (b) a second CRISPR/Cas system comprising a Cas nuclease and a second gRNA configured to direct the second CRISPR/Cas system to a second or subsequent target domain comprising introducing a mutation at the second or subsequent target domain, or a part of it; wherein the second CRISPR/Cas system recognizes the artificial PAM, and wherein the second or subsequent target domain is in proximity to the artificial PAM.

Some aspects of this disclosure provide mixtures comprising an mRNA encoding one, two, three, or all of: (a) a first guide RNA (gRNA) configured to direct a first CRISPR/Cas system to a first target domain to provide an edit to generate an artificial PAM in a genomic DNA molecule; (b) a second gRNA configured to direct a second CRISPR/Cas system to a second or subsequent target domain to provide an edit within a target gene; (c) a CRISPR/Cas system that binds the first gRNA; and (d) a CRISPR/Cas system that binds the second gRNA. In some embodiments, (a)-(b) are encoding by the same mRNA or separate mRNA.

Some aspects of this disclosure provide kits or compositions comprising: (a) a first guide RNA (gRNA) configured to direct a first CRISPR/Cas system to a first target domain to provide an edit to generate an artificial PAM in a genomic DNA molecule; and (b) a second gRNA configured to direct a second CRISPR/Cas system to a second or subsequent target domain to provide an edit within a target gene.

Some aspects of this disclosure provide kits or compositions comprising: (a) a first guide RNA (gRNA) configured to direct a first CRISPR/Cas system to a first target domain to provide an edit to generate an artificial PAM in a genomic DNA molecule; (b) a second gRNA configured to direct a second CRISPR/Cas system to a second or subsequent target domain to provide an edit within a target gene; (c) a CRISPR/Cas system that binds the first gRNA; and (d) a CRISPR/Cas system that binds the second gRNA.

Some aspects of this disclosure also provide methods for genetically modifying a cell that can be used to make such genetically engineered cells. Some aspects of this disclosure provide methods of using the genetically engineered cells provided herein.

Particular aspects of this disclosure provide methods of using the compositions provided herein, e.g., methods of using certain gRNAs and/or CRISPR/Cas systems provided to create genetically engineered cells, e.g., cells having an artificial PAM and a modification in a protein of interest or an antigen of a protein of interest.

Some aspects of this disclosure provide methods of administering genetically engineered cells provided herein, e.g., cells having an artificial PAM and a modification in a cell-surface protein, such as in the CD33, CD20, CLL-1, CD123, CD38, CD19, CD117, EMR2, and/or CD5 gene(s), to a subject in need thereof. In some embodiments, the subject has, or has been diagnosed with, a cancer or a premalignant condition. In some embodiments, the cancer is a hematologic malignancy. In some embodiments, the pre-malignant condition is myelodysplastic syndrome. In some embodiments, the cancer or the pre-malignant condition is characterized by expression of the lineage-specific cell-surface protein on the surface of malignant cells in the subject. In some embodiments, the cancer or the pre-malignant condition is characterized by expression of a CD33, CD20, CLL-1, CD123, CD38, CD19, CD117, EMR2, and/or CD5 protein on the surface of malignant cells in the subject. In some embodiments, the cancer or the pre-malignant condition is characterized by expression of a CD33, CD38, CD123, and/or CD20 protein on the surface of malignant cells in the subject. In some embodiments, the cancer or the pre-malignant condition is characterized by expression of a CD33 protein on the surface of malignant cells in the subject. In some embodiments, the cancer or the pre-malignant condition is characterized by expression of a CD20 protein on the surface of malignant cells in the subject. In some embodiments, the cancer or the pre-malignant condition is characterized by expression of a CLL-1 protein on the surface of malignant cells in the subject. In some embodiments, the cancer or the pre-malignant condition is characterized by expression of a CD123 protein on the surface of malignant cells in the subject. In some embodiments, the cancer or the pre-malignant condition is characterized by expression of a CD38 protein on the surface of malignant cells in the subject. In some embodiments, the cancer or the pre-malignant condition is characterized by expression of a CD19 protein on the surface of malignant cells in the subject. In some embodiments, the cancer or the pre-malignant condition is characterized by expression of a CD117 protein on the surface of malignant cells in the subject. In some embodiments, the cancer or the pre-malignant condition is characterized by expression of a EMR2 protein on the surface of malignant cells in the subject. In some embodiments, the cancer or the pre-malignant condition is characterized by expression of a CD5 protein on the surface of malignant cells in the subject.

Some aspects of this disclosure provide strategies, compositions, methods, and treatment modalities for the treatment of patients having a malignancy or cancer and receiving or in need of receiving an anti-cancer therapy, (e.g., an anti-CD33 therapy). In some embodiments, the subject has, or has been diagnosed with, a cancer, malignancy or a premalignant condition. In some embodiments, the cancer is a hematologic malignancy. In some embodiments, the pre-malignant condition is myelodysplastic syndrome. In some embodiments, the cancer or the pre-malignant condition is characterized by expression of a cell-surface protein on the surface of malignant cells in the subject. In some embodiments, the cancer or the pre-malignant condition is characterized by expression of a CD33, CD20, CLL-1, CD123, CD38, CD19, CD117, EMR2, and/or CD5 protein on the surface of malignant cells in the subject. In some embodiments, the cancer or the pre-malignant condition is characterized by expression of a CD33 protein on the surface of malignant cells in the subject. In some embodiments, the cancer or the pre-malignant condition is characterized by expression of a CD20 protein on the surface of malignant cells in the subject. In some embodiments, the cancer or the pre-malignant condition is characterized by expression of a CLL-1 protein on the surface of malignant cells in the subject. In some embodiments, the cancer or the pre-malignant condition is characterized by expression of a CD123 protein on the surface of malignant cells in the subject. In some embodiments, the cancer or the pre-malignant condition is characterized by expression of a CD38 protein on the surface of malignant cells in the subject. In some embodiments, the cancer or the pre-malignant condition is characterized by expression of a CD19 protein on the surface of malignant cells in the subject. In some embodiments, the cancer or the pre-malignant condition is characterized by expression of a CD117 protein on the surface of malignant cells in the subject. In some embodiments, the cancer or the pre-malignant condition is characterized by expression of a EMR2 protein on the surface of malignant cells in the subject. In some embodiments, the cancer or the pre-malignant condition is characterized by expression of a CD5 protein on the surface of malignant cells in the subject.

Some aspects of this disclosure provide methods for treating a cancer, the method comprising administering to a subject in need thereof (a) an effective amount of an agent targeting a lineage-specific cell-surface antigen, wherein the agent comprises an antigen-binding fragment that binds the lineage-specific cell-surface antigen; and (b) a population of hematopoietic cells that (i) contain an artificial protospacer-adjacent motif (PAM) introduced via a gene mutation and (ii) express a variant lineage-specific cell-surface antigen comprising a modification in an epitope to which the agent binds. In some embodiments, the agent can be an immune cell (e.g., a T cell) expressing a chimeric receptor that comprises the antigen-binding fragment that binds the lineage-specific cell-surface antigen. In some embodiments, the immune cells, the hematopoietic cells, or both, are allogeneic or autologous. In some embodiments, the hematopoietic cells are hematopoietic stem cells (HSCs) and/or hematopoietic progenitor cells (HPCs). In some embodiments, the hematopoietic cells are CD33+, CD20+, CLL-1+, CD123+, CD38+, CD19+, CD117+, EMR2+, and/or CD5+ HSCs and/or HPCs. In some embodiments, the hematopoietic cells are CD33+ HSCs and/or HPCs. In some embodiments, the hematopoietic cells are CD20+ HSCs and/or HPCs. In some embodiments, the hematopoietic cells are CLL-1+ HSCs and/or HPCs. In some embodiments, the hematopoietic cells are CD123+ HSCs and/or HPCs. In some embodiments, the hematopoietic cells are CD38+ HSCs and/or HPCs. In some embodiments, the hematopoietic cells are CD19+ HSCs and/or HPCs. In some embodiments, the hematopoietic cells are CD117+ HSCs and/or HPCs. In some embodiments, the hematopoietic cells are EMR2+ HSCs and/or HPCs. In some embodiments, the hematopoietic cells are CD5+ HSCs and/or HPCs. In some embodiments, the hematopoietic stem cells can be obtained from bone marrow cells or peripheral blood mononuclear cells (PBMCs). In some embodiments, the antigen-binding fragment binds a lineage-specific cell-surface antigen selected from the group consisting of CD33, CD20, CLL-1, CD123, CD38, CD19, CD117, EMR2, and CD5. In some embodiments, the antigen-binding fragment binds a lineage-specific cell-surface antigen selected from the group consisting of CD33, CD38, CD123, and CD20. In some embodiments, the antigen-binding fragment binds CD33. In some embodiments, the antigen-binding fragment binds CD20. In some embodiments, the antigen-binding fragment binds CLL-1. In some embodiments, the antigen-binding fragment binds CD123. In some embodiments, the antigen-binding fragment binds CD38. In some embodiments, the antigen-binding fragment binds CD19. In some embodiments, the antigen-binding fragment binds CD117. In some embodiments, the antigen-binding fragment binds EMR2. In some embodiments, the antigen-binding fragment binds CD5.

Other features, objects, and advantages of the invention will be apparent from the description and drawings, and from the claims and the Enumerated Embodiments below.

### Enumerated Embodiments

1. A genetically engineered cell, comprising:
a modified genomic DNA molecule,
   wherein the modified genomic DNA molecule comprises a mutation in a nucleotide sequence at a first target domain,
   wherein the modified genomic DNA molecule comprises an artificial protospacer adjacent motif (PAM) comprising the mutation at the first target domain, or a part of it, and
   wherein an unmodified genomic DNA molecule of the same type does not comprise a PAM sequence at the first target domain.
      2. The genetically engineered cell of embodiment 1, wherein the mutation at the first target domain comprises a nucleotide substitution, insertion, or deletion, as compared to the nucleotide sequence of an unmodified genomic DNA molecule of the same type.
      3. The genetically engineered cell of embodiment 1 or 2, wherein the mutation at the first target domain comprises a nucleotide substitution as compared to the nucleotide sequence of an unmodified genomic DNA molecule of the same type.
      4. The genetically engineered cell of any one of embodiments 1-3, wherein the mutation at the first target domain comprises a nucleotide substitution of 1, 2, 3, 4, 5, 6, 7, 8, 9, or 10 nucleotides as compared to the nucleotide sequence of an unmodified genomic DNA molecule of the same type.
      5. The genetically engineered cell of any one of embodiments 1-4, wherein the first target domain is within proximity of a PAM which is capable of targeting a Clustered Regularly Interspaced Short Palindromic Repeats (CRISPR)-CRISPR associated (Cas) (CRISPR/Cas) system comprising a Cas nuclease and a guide RNA (gRNA) to the first target domain.
      6. The genetically engineered cell of any one of embodiments 1-5, wherein the first target domain is within about 1, 2, 3, 4, 5, 6, 7, 8, 9, 10, 11, 12, 13, 14, 15, 16, 17, 18, 19, 20, 21, 22, 23, 24, or 25 nucleotides of a naturally occurring PAM which is capable of targeting a CRISPR/Cas system to the first target domain.
      7. The genetically engineered cell of any one of embodiments 1-6, wherein the unmodified genomic DNA molecule lacks a PAM in proximity of a second or subsequent target domain which is capable of targeting a CRISPR/Cas system to the second or subsequent target domain.
      8. The genetically engineered cell of any one of embodiments 1-7, wherein the unmodified genomic DNA molecule lacks a PAM within about 1, 2, 3, 4, 5, 6, 7, 8, 9, 10, 11, 12, 13, 14, 15, 16, 17, 18, 19, 20, 21, 22, 23, 24, or 25 nucleotides of a second or subsequent target domain which is capable of targeting a CRISPR/Cas system to the second or subsequent target domain.
      9. The genetically engineered cell of any one of embodiments 1-8, wherein the PAM which is capable of targeting a CRISPR/Cas system to the first target domain and the artificial PAM are separated by about 1, 2, 3, 4, 5, 6, 7, 8, 9, 10, 11, 12, 13, 14, 15, 16, 17, 18, 19, 20, 21, 22, 23, 24, or 25 nucleotides.
      10. The genetically engineered cell of any one of embodiments 1-9, wherein the modified genomic DNA molecule further comprises a mutation at a second or subsequent target domain as compared to the nucleotide sequence of an unmodified genomic DNA molecule of the same type, wherein the second or subsequent target domain is in proximity to the artificial PAM.
      11. The genetically engineered cell of embodiment 10, wherein the mutation at the second or subsequent target domain comprises a mutation within 8, 9, 10, 11, 12, 13, 14, 15, 16, 17, 18, 19, 20, 21, or 22 nucleotides from the artificial PAM.
      12. The genetically engineered cell of embodiment 10, wherein the mutation at the second or subsequent target domain comprises a mutation within 8, 9, 10, 11, 12, 13, 14, 15, 16, 17, 18, 19, 20, 21, or 22 nucleotides upstream of the 5′-terminal nucleotide of the artificial PAM.
      13. The genetically engineered cell of embodiment 10, wherein the mutation at the second or subsequent target domain comprises a mutation within 16-18 nucleotides upstream of the 5′-terminal nucleotide of the artificial PAM.
      14. The genetically engineered cell of embodiment 10, wherein the mutation at the second or subsequent target domain comprises a mutation at least 8, 9, 10, 11, or 12 nucleotides upstream of the 5′-terminal nucleotide of the artificial PAM.
      15. The genetically engineered cell of any one of embodiments 1-14, wherein the mutation at the first target domain and/or the mutation at the second or subsequent target domain comprises a C-to-T substitution and/or an A-to-G nucleotide substitution.
      16. The genetically engineered cell of any one of embodiments 1-15, wherein the mutation at the first target domain comprises an A-to-G nucleotide substitution, and the mutation at the second or subsequent target domain comprises a C-to-T and/or an A-to-G nucleotide substitution.
      17. The genetically engineered cell of any one of embodiments 1-16, wherein the mutation at the first target domain comprises a C-to-T nucleotide substitution, and the mutation at the second or subsequent target domain comprises a C-to-T and/or an A-to-G nucleotide substitution.
      18. The genetically engineered cell of any one of embodiments 1-17, wherein the mutation in the nucleotide sequence at the second or subsequent target domain is in a target gene.
      19. The genetically engineered cell of any one of embodiments 1-18, wherein the target gene encodes a cell-surface protein.
      20. The genetically engineered cell of any one of embodiments 1-19, wherein the target gene encodes a lineage-specific cell-surface protein.
      21. The genetically engineered cell of any one of embodiments 1-20, wherein the target gene encodes a cell-surface protein epitope.
      22. The genetically engineered cell of any one of embodiments 1-21, wherein the mutation in the nucleotide sequence at the second or subsequent target domain alters the amino acid sequence of an epitope in the cell-surface protein epitope, resulting in a modified epitope.
      23. The genetically engineered cell of embodiment 22, wherein the mutation in the nucleotide sequence at the second or subsequent target domain results in a deletion, a substitution, an insertion, or an inversion of one or more amino acid residues, or a combination thereof.
      24. The genetically engineered cell of any one of embodiments 22 or 23, wherein the mutation in the nucleotide sequence at the second or subsequent target domain results in an amino acid substitution in the cell-surface protein epitope.
      25. The genetically engineered cell of any one of embodiments 23 or 24, wherein the amino acid substitution is a non-conservative amino acid substitution.
      26. The genetically engineered cell of any one of embodiments 22-25, wherein the cell-surface protein epitope is bound by an agent, and wherein the modified epitope is characterized by a reduction or an abolishment of binding activity of the agent.
      27. The genetically engineered cell of embodiment 26, wherein the binding activity of the agent to the modified epitope is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, or more, as compared to an unmodified epitope.
      28. The genetically engineered cell of embodiment 26 or 27, wherein the reduction in binding activity comprises an increase in K_{D}, IC₅₀, and/or EC₅₀.
      29. The genetically engineered cell of embodiment 27, wherein:
(i) the K_{D}, IC₅₀, and/or EC₅₀ is increased by the amino acid substitution by at least 5%, 10%, 15%, 20%, 25%, 30%, 35%, 40%, 45%, 50%, 55%, 60%, 65%, 70%, 75%, 80%, 85%, 90%, 95%, or 99% or more as compared to the unmodified epitope; or
(ii) the K_{D}, IC₅₀, and/or EC₅₀ is increased by the amino acid substitution by at least 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold as compared to the unmodified epitope.
   30. The genetically engineered cell of anyone of embodiments 20-29, wherein the lineage-specific cell-surface protein is CD19; CD123; C-type lectin-like molecule-1 (CLL-1) CD38; KIT (CD117); CD20; or EGF-like module-containing mucin-like hormone receptor-like 2 (EMR2).
   31. The genetically engineered cell of anyone of embodiments 20-29, wherein the lineage-specific cell-surface protein is CD5, CD19, CD20, CD38, CD117, or CD123.
   32. The genetically engineered cell of anyone of embodiments 20-29, wherein the lineage-specific cell-surface protein is CD19, CD20, CD38, C-type lectin-like molecule-1 (CLL-1) CD123, or CD33.
   33. The genetically engineered cell of anyone of embodiments 20-29, wherein the cell-surface lineage-specific protein is CD33.
   34. The genetically engineered cell of anyone of embodiments 20-29, wherein the cell-surface lineage-specific protein is CD20.
   35. The genetically engineered cell of anyone of embodiments 20-29, wherein the cell-surface lineage-specific protein is CLL-1.
   36. The genetically engineered cell of anyone of embodiments 20-29, wherein the cell-surface lineage-specific protein is CD123.
   37. The genetically engineered cell of anyone of embodiments 20-29, wherein the cell-surface lineage-specific protein is CD38.
   38. The genetically engineered cell of anyone of embodiments 20-29, wherein the cell-surface lineage-specific protein is CD19.
   39. The genetically engineered cell of anyone of embodiments 20-29, wherein the cell-surface lineage-specific protein is CD117.
   40. The genetically engineered cell of anyone of embodiments 20-29, wherein the cell-surface lineage-specific protein is EMR2.
   41. The genetically engineered cell of anyone of embodiments 20-29, wherein the cell-surface lineage-specific protein is CD5.
   42. The genetically engineered cell of any one of embodiments 1-41, wherein the target gene, the cell-surface protein, and/or the cell-surface lineage-specific protein expression is associated with a cancer.
   43. The genetically engineered cell of any one of embodiments 1-42, wherein the target gene, the cell-surface protein, and/or the cell-surface lineage-specific protein expression is associated with a hematopoietic malignancy.
   44. The method of embodiment 43, wherein the hematopoietic malignancy is Hodgkin's lymphoma, non-Hodgkin's lymphoma, leukemia, multiple myeloma (MM), myelodysplastic syndrome (MDS), or blastic plasmacytoid dendritic cell neoplasm (BPDCN).
   45. The method of embodiment 43 or 44, wherein the hematopoietic malignancy is acute myeloid leukemia, B-cell acute lymphoblastic leukemia (B-ALL), chronic myelogenous leukemia, acute lymphoblastic leukemia, or chronic lymphoblastic leukemia.
   46. The genetically engineered cell of any one of embodiments 1-45, wherein the PAM and/or the artificial PAM is a Cas nuclease PAM.
   47. The genetically engineered cell of any one of embodiments 1-46, wherein the PAM and/or the artificial PAM is a Cas9 PAM.
   48. The genetically engineered cell of any one of embodiments 1-47, wherein the PAM and/or the artificial PAM is a Cas12a PAM 49. The genetically engineered cell of any one of embodiments 1-48 wherein the PAM and/or the artificial PAM is a SpyCas9 PAM, a SpGCas9 PAM, a SpRYCas9 PAM, a Sth1Cas9 PAM, a SauCas9 PAM, a NmeCas9 PAM, a RspCas9 PAM, a Cca1Cas9 PAM, a PspCas9 PAM, a OrhCas9 PAM, a ScCas9 PAM, a SmacCas9 PAM, a TdeCas9 PAM, a Nme2Cas9 PAM, a CjeCas9 PAM, a SmuCas9 PAM, a Smu2Cas9 PAM, a PmuCas9 PAM, a SpaCas9 PAM, a NciCas9 PAM, a ClaCas9 PAM, a PlaCas9 PAM, a CdCas9 PAM, a IgnaviCas9 PAM, a ThermoCas9 PAM, a GeoCas9 PAM, a G. LC300 Cas9 PAM, a AceCas9 PAM, a Sth3Cas9 PAM, a BlatCas9 PAM, a FnCas9 PAM, a LfeCas9 PAM, a LpnCas9 PAM, a KhuCas9 PAM, a AinCas9 PAM, a CglCas9 PAM, a Esp1Cas9 PAM, a Esp2Cas9 PAM, a FmaCas9 PAM, a LceCas9 PAM, a LrhCas9 PAM, a Lsp1Cas9 PAM, a Lsp2Cas9 PAM, a PacCas9 PAM, a TbaCas9 PAM, a TpuCas9 PAM, a VpaCas9 PAM, a EfaCas9 PAM, a EitCas9 PAM, a LanCas9 PAM, a LmoCas9 PAM, a Sag1Cas9 PAM, a Sag2Cas9 PAM, a SdyCas9 PAM, a Seq1Cas9 PAM, a Seq2Cas9 PAM, a SgaCas9 PAM, a Smu3Cas9 PAM, a SraCas9 PAM, a BniCas9 PAM, a EceCas9 PAM, a EdoCas9 PAM, a FhoCas9 PAM, a MgaCas9 PAM, a MseCas9 PAM, a SgoCas9 PAM, a SmaCas9 PAM, a SsaCas9 PAM, a SsiCas9 PAM, a SsuCas9 PAM, a Sth1ACas9 PAM, a TspCas9 PAM, a BokCas9 PAM, a CcoCas9 PAM, a CpeCas9 PAM, a DdeCas9 PAM, a Ghc2Cas9 PAM, a Ghy3Cas9 PAM, a Ghy4Cas9 PAM, a GspCas9 PAM, a KkiCas9 PAM, a NspCas9 PAM, a TmoCas9 PAM, a NsaCas9 PAM, a JpaCas9 PAM, a BboCas9 PAM, a Cca2Cas9 PAM, a Cme2Cas9 PAM, a Cme3Cas9 PAM, a Cme4Cas9 PAM, a CsaCas9 PAM, a Ghc1Cas9 PAM, a GheCas9 PAM, a Ghh1Cas9 PAM, a Ghh2Cas9 PAM, a Ghy1Cas9 PAM, a MscCas9 PAM, a SdoCas9 PAM, a SpacCas9 PAM, a CgaCas9 PAM, a CmelCas9 PAM, a FfrCas9 PAM, a Ghy2Cas9 PAM, a PhiCas9 PAM, a WviCas9 PAM, a CcCas9 PAM, a FnCas12a PAM, a AsCas12a PAM, a HkCas12a PAM, a PiCas12a PAM, a PdCas12a PAM, a LbCas12a PAM, a Lb2Cas12a or Lb5Cas12a PAM, a CMtCas12a PAM, a MbCas12a PAM, a TsCas12a PAM, a Pb2Cas12a PAM, a MICas12a PAM, a Mb2Cas12a PAM, a Mb3Cas12a PAM, a CMaCas12a PAM, a BsCas12a PAM, a BfCas12a PAM, a BoCas12a PAM, a Adurb193Cas12a PAM, a Adurb336Cas12a PAM, a Fn3Cas12a PAM, a Lb6Cas12a PAM, a EcCas12a PAM, a PsCas12a PAM, a McCas12a PAM, a AacCas12b PAM, a BthCas12b PAM, a AkCas12b PAM, a EbCas12b PAM, a BvCas12b PAM, a BhCas12b PAM, a LsCas12b PAM, a BrCas12b PAM, a Cas12c1 PAM, a Cas12c2 PAM, a OspCas12c PAM, a Cas12d.15 PAM, a Cas12d.1 (CasY.1) PAM, a DpbCas12e (DpbCasX) PAM, a PlmCas12e (PlmCasX) PAM, a MilCas12f2 PAM, a Un1Cas12f1 PAM, a Un2Cas12f1 PAM, a Mi2Cas12f2 PAM, a AuCas12f2 PAM, a PtCas12f1 PAM, a AsCas12f1 PAM, a RuCas12f1 PAM, a SpCas12f1 PAM, a CnCas12f1 PAM, a Cas12h1 PAM, a Cas12i1 PAM, a Cas1212 PAM, a Cas12j-1 (CasPhi-1) PAM, a Cas12j-2 (CasPhi-2) PAM, a Cas12j-3 (CasPhi-3) PAM, a ShCas12k PAM, or a AcCas12k PAM.
   50. The genetically engineered cell of any one of embodiments 1-49, wherein the PAM, and/or the artificial PAM comprises, and/or consists of a nucleotide sequence of NGG; NNAGAAW; NNGRRT; NNNNGATT; NVNDCCY; BRTTTTT; NR (A or G) TTTT; NNAAAR (G or A); N (N or A) G; NAAN; NAAAAY; NHDTCCA; NNNVRYM; NNNNRYAC; NAA; GNNNNCNNA; NNGTGA; NNNNGTA; NNGGG; NNNCAT; NNRHHHY; NRRNAT; NNNNCNAA; NNNNCMCA; NNNNCRAA; NNNNGMAA; NNNCC; NGGNG; NNNNCNDD; NYAAA; NRGNN; N (C or D) GGN (T or A or G or C) NN; NRTAW; N (C or K or A) AARC; NAAAG; NV (A or G or C) R (A or G) ACCN; NNGAC; NATGNT; N (T or V) NTAAW (A or T); NNGW (A or T) AY (T or C); NCAA (H (Y or A) B (Y or G); NH (T or C or A) AAAA; NNNATTT; NATAWN (A or T or S); NATARCH; B (T or G or C) GGD (A or T or G) TNN; N (G or T or M) GGAH (T or A or C) N (A or C or K) N; NRG; N (B or A) GG; NGGD (A or K) W (T or A); N (T or C or R) AGAN (A or K or C) NN; NGGD (A or T or G) H (T or M); NGGDT; NGGD (A or T or G) GNN; NNGTAM (A or C) Y; NNGH (W or C) AAA; NTGAR (G or A) N (A or Y or G) N (Y or R); NNGAAAN; NNGAD; NHARMC; NNAAAG; NHGYNAN (A or B); NNAGAAA; NHAAAAA; NH (T or M) AAAAA; NHGYRAA; NNAAACN; NN (H or G) D (A or K) GGDN (A or B); NNNNCTA; NNNNCVGAA; NNNNGYAA; NNNNATN (W or S) ANN; NNWHR (G or A) TA (not G) AA; YHHNGTH; NNNNCDAANN; NNNNCTAA; N (C or D) NNTCCN; NNNNCCAA; NAGRGN (T or V) N (T or C); NNAH (T or M) ACN; CN (C or W or G) AV (A or S) GAC; NAR (G or A) H (W or C) H (A or T or C) GN (C or T or R); NAGNGC; NATCCTN; NGTGANN; HGCNGCR; NAR (A or G) W (T or A) AC; N (C or D) M (A or C) RN (A or B) AY (C or T); NNNCAC; BGGGTCD; NNRRCC; NRRNTT; KARDAT; BRRTTTW; NARNCCN; NAR (A or G) TC; NAAN (A or T or S) RCN; HHAAATD; NNNNGNA; TTV; TTTV; YYV; KKYV; TTTM; TTYV; TTTN; TTTTA; TTN; BTTV; YTV; YTN; NYTV; DTTD; ATTN; RTTNT; HATT; ATTW; RTTN; TVT; TG; TN; TR; TA; TTCN; TTAT; TTTR; TTR; YTTR; YTTN; CTT; TTC; CCD; RTR; VTTR; TBN; VTTN; NGTT; CGTT; AGG; CGG; GTT, or RGTG, wherein “N” is any nucleotide or base, “W” is adenine (A) or thymine (T), “R” is A or guanine (G), “V” is A, cytosine (C), or G, “Y” is C or T, and “H” is A, C, or T.
   51. The genetically engineered cell of any one of embodiments 1-50, wherein the PAM, and/or the artificial PAM comprises, and/or consists of a nucleotide sequence of NGG.
   52. The genetically engineered cell of any one of embodiments 1-51, wherein the PAM, and/or the artificial PAM comprises, and/or consists of a nucleotide sequence of TTN.
   53. The genetically engineered cell of any one of embodiments 1-52, wherein the PAM, and/or the artificial PAM comprises, and/or consists of a nucleotide sequence of NGG, wherein “N” is any nucleotide or base.
   54. The genetically engineered cell of any one of embodiments 1-53, wherein the PAM, and/or the artificial PAM comprises, and/or consists of a nucleotide sequence of AGG.
   55. The genetically engineered cell of any one of embodiments 1-54, wherein the PAM, and/or the artificial PAM comprises, and/or consists of a nucleotide sequence of CGG.
   56. The genetically engineered cell of any one of embodiments 1-55, wherein the PAM, and/or the artificial PAM, comprises and/or consists of a nucleotide sequence of GTT.
   57. The genetically engineered cell of any one of embodiments 1-56, wherein the modified genomic DNA molecule encodes CD33, wherein the modified genomic DNA molecule comprises a nucleotide sequence comprising

(SEQ ID NO: 1)
ATGGATCCAAATTTCTGGCTGCA₃A₄GTGCAGGAGTCAGTGACGGTACA
GGAG;
(SEQ ID NO: 3)
ATGGA₃TCCA₇A₈A₉TTTCTGGCTGCAGGTGCAGGAGTCAGTGACGGTA
CAGGAG;
or
(SEQ ID NO: 4)
ATGGATCCAGATTTCTGGCTGCAGGTGCAGGAGTCAGTGACGGTACAG
GAG,

wherein each of A₃, A₄, A₇, A₈, and A₉ independently comprise either A or G.
58. The genetically engineered cell of any one of embodiments 1-57, wherein the modified genomic DNA molecule encodes a modified CD33 protein, wherein the modified CD33 protein comprises, e.g., a mutation dEpi, characterized by the deletion of MDPNFWLQVQE (SEQ ID NO: 2) at amino acid residue positions 17-27; NFW, characterized by the substitution of NEW with AAA at residue positions 20-22;
LQV, characterized by the substitution of LQV with AAA at residue positions 23-25;
N20D, characterized by the substitution of N with D at residue position 20;
F21Y, characterized by the substitution of F with Y at residue position 21;
L23I, characterized by the substitution of L with I at residue position 23; and/or
Q24E, characterized by the substitution of Q with E at residue position 24.
   59. The genetically engineered cell of any one of embodiments 1-58, wherein the modified genomic DNA molecule encodes a modified CD33 protein, wherein the modified CD33 protein comprises an amino acid sequence comprising

(SEQ ID NO: 8)
MPLLLLLPLLWAGALA-----------SVTVQEGLCVLVPCTFFHPIPY
YDKSNP (dEpi);
(SEQ ID NO: 9)
MPLLLLLPLLWAGLALMDPAAALQVQESVTVQEGLCVLVPCTFFHPIPY
YDKNSP (NFW);
(SEQ ID NO: 10)
MPLLLLLPLLWAGALAMDPNFWAAAQESVTVQEGLCVLVPCTFFHPIPY
YDKNSP (LQV);
(SEQ ID NO: 11)
MPLLLLLPLLWAGALAMDPDFWLQVQESVTVQEGLCVLVPCTFFHPIPY
YDKNSP (N20D);
(SEQ ID NO: 12)
MPLLLLLPLLWAGALAMDPNYWLQVQESVTVQEGLCVLVPCTFFHPIPY
YDKNSP;
(SEQ ID NO: 13)
MPLLLLLPLLWAGALAMDPNFWIQVQESVTVQEGLCVLVPCTFFHPIPY
YDKSNP (L23I);
or
(SEQ ID NO: 14)
MPLLLLLPLLWAGALAMDPNFWLEVQESVTVQEGLCVLVPCTFFHPIPY
YDKSNP.

60. The genetically engineered cell of any one of embodiments 1-59, wherein the modified genomic DNA molecule encodes CD20, wherein the modified genomic DNA molecule comprises a nucleotide sequence comprising

(SEQ ID NO: 64)
CGAGTGTGTGGTATATAATTGTA₁₀TA₈TGTTGA₂CACTTGGTCGATTAG
GGAGACTCTTTTTGAGGGGTAGATGGGTTATGACAATG;
(SEQ ID NO: 65)
CGAGTGTGTGGTATATAATTGTGTGTGTTGGCACTTGGTCGA₁₁TTA₈G
GGA₄GA₂CTCTTTTTGAGGGGTAGATGGGTTATGACAATG;
or
(SEQ ID NO: 66)
CGAGTGTGTGGTATATAATTGTGTGTGTTGGCACTTGGTCGGTTGGGG
GGGCTCTTTTTGAGGGGTAGATGGGTTATGACAATG,

wherein each of A₂, A₄, A₈, A₁₀, A₁₁ independently comprise either A or G.
61. The genetically engineered cell of any one of embodiments 1-60, wherein the modified genomic DNA molecule encodes a modified CD20 protein, wherein the modified CD20 protein comprises, e.g., a mutation
(i) characterized by the substitution of I with T at residue position 8;
(ii) characterized by the substitution of Y with H at residue position 9;
(iii) characterized by the substitution of C with A at residue position 11;
(iv) characterized by the substitution of N with L at residue position 15; and/or
(v) characterized by the substitution of S with P at residue position 17.
   62. The genetically engineered cell of any one of embodiments 1-61, wherein the modified genomic DNA molecule encodes a modified CD20 protein, wherein the modified CD20 protein comprises an amino acid sequence comprising

(SEQ ID NO: 24)
AHTPYINTHNAEPANPSEKNSPSTQYCY;
or
(SEQ ID NO: 67)
AHTPYINTHNAEPALPPEKNSPSTQYCY.

63. The genetically engineered cell of any one of embodiments 1-62, wherein:
(i) the first gRNA comprises a targeting domain which binds a site in a CD33 gene, optionally, wherein the site comprises the nucleic acid sequence of GCAAGTGCAGGAGTCAGTGA (SEQ ID NO: 68), or a portion thereof; or
(ii) the first gRNA comprises a targeting domain which binds a site in a CD20 gene, optionally, wherein the site comprises the nucleic acid sequence of ATATAATTGTATATGTTGAC (SEQ ID NO: 69), or a portion thereof
   64 The genetically engineered cell of any one of embodiments 1-63, wherein:
(i) the second gRNA comprises a targeting domain which binds a site in a CD33 gene, optionally, wherein the site comprises the nucleic acid sequence of GGATCCAAATTTCTGGCTGC (SEQ ID NO: 70), or a portion thereof; or
(ii) the second gRNA comprises a targeting domain which binds a site in a CD20 gene, optionally, wherein the site comprises the nucleic acid sequence of ACTTGGTCGATTAGGGAGAC (SEQ ID NO: 71), or a portion thereof.
   65 The genetically engineered cell of any one of embodiments 1-64, wherein the modified genomic DNA molecule encodes CD38, wherein the modified genomic DNA molecule comprises a nucleotide sequence comprising

(SEQ ID NO: 72)
CGCAGGGTAAGTACCAAGTGGTGAAATTCTAGAGCTTTGGAGA;
(SEQ ID NO: 73)
CGCAGGGTAAGTACCAAGTAGTGA₁A₂A₃TTCTAGAGCTTTGGAGA;
(SEQ ID NO: 74)
CGCGGGGTAAGTACCAAGTGGTGAAATTCTAGAGCTTTGGAGA;
or
(SEQ ID NO: 75)
CGCAGGGTA4A5GTACCAAGTAGTGA₁A₂A₃TTCTAGAGCTTTGGAGA,

wherein each of A₁, A₂, A₃, A₄, and A₅ independently comprise either A or G, wherein at least one of A₁, A₂, A₃, A₄, and A₅ comprise a G.
   66. The genetically engineered cell of any one of embodiments 1-65, wherein the modified genomic DNA molecule encodes a modified CD38 protein, wherein the modified CD38 protein comprises R195G, characterized by the substitution of R with G at residue position 195.
   67. The genetically engineered cell of any one of embodiments 1-66, wherein the modified genomic DNA molecule encodes a modified CD38 protein, wherein the modified CD38 protein comprises an amino acid sequence KINYQSCPDWRKDCSNNPVSVFWKTVSRG (SEQ ID NO: 76) corresponding to residue positions 167-195 of CD38.
   68. The genetically engineered cell of any one of embodiments 1-67, wherein the modified genomic DNA molecule encodes CD123, wherein the modified genomic DNA molecule comprises a nucleotide sequence comprising

(SEQ ID NO: 77)
TACCAGGAGGAAACCGAGTGCGGCGAGGACTAGCGGGACGGGACAGAG
GACGTTTGCTTCCTT;
or
(SEQ ID NO: 78)
TACCAGGAGGAAACCGAGTGCGGCGAGGACTAGCGGGGGGGGACAGAG
GACGTTTGCTTCCTT.

69. The genetically engineered cell of any one of embodiments 1-68, wherein the modified genomic DNA molecule encodes a modified CD123 protein, wherein the modified CD123 protein comprises
L8P, characterized by the substitution of L with P at residue position 8; or
L8P and L13P, characterized by the substitutions of L with P at residue position 8 and L with P at residue position 13.
   70. The genetically engineered cell of any one of embodiments 1-69, wherein the modified genomic DNA molecule encodes a modified CD123 protein, wherein the modified CD123 protein comprises an amino acid sequence comprising

(SEQ ID NO: 79)
MVLLWLTPLLIALPCLLQTKE;
or
(SEQ ID NO: 80)
MVLLWLTPLLIAPPCLLQTKE,

corresponding to residue positions 1-21 of CD123.
   71. The genetically engineered cell of any one of embodiments 1-70, wherein:
(i) the first gRNA comprises a targeting domain which binds a site in a CD33 gene, optionally, wherein the site comprises the nucleic acid sequence of GCAAGTGCAGGAGTCAGTGA (SEQ ID NO: 68), or a portion thereof;
(ii) the first gRNA comprises a targeting domain which binds a site in a CD20 gene, optionally, wherein the site comprises the nucleic acid sequence of ATATAATTGTATATGTTGAC (SEQ ID NO: 69), or a portion thereof;
(iii) the first gRNA comprises a targeting domain which binds a site in a CD38 gene, optionally, wherein the site comprises the nucleic acid sequence of GTAGTGAAATTCTAGAGCTT (SEQ ID NO: 81), or a portion thereof; or
(iv) the first gRNA comprises a targeting domain which binds a site in a CD123 gene, optionally, wherein the site comprises the nucleic acid sequence of CCTTTGGCTCACGCTGCTCC (SEQ ID NO: 82), or a portion thereof.
   72. The genetically engineered cell of any one of embodiments 1-71, wherein:
(i) the second gRNA comprises a targeting domain which binds a site in a CD33 gene, optionally, wherein the site comprises the nucleic acid sequence of GGATCCAAATTTCTGGCTGC (SEQ ID NO: 70), or a portion thereof;
(ii) the second gRNA comprises a targeting domain which binds a site in a CD20 gene, optionally, wherein the site comprises the nucleic acid sequence of ACTTGGTCGATTAGGGAGAC (SEQ ID NO: 71), or a portion thereof;
(iii) the second gRNA comprises a targeting domain which binds a site in a CD38 gene, optionally, wherein the site comprises the nucleic acid sequence of CCCGCAGGGTAAGTACCAAG (SEQ ID NO: 83) or GCAGGGTAAGTACCAAGTAG (SEQ ID NO: 84), or a portion thereof; or
(iv) the second gRNA comprises a targeting domain which binds a site in a CD123 gene, optionally, wherein the site comprises the nucleic acid sequence of CTCCTGATCGCCCTGCCTG (SEQ ID NO: 85), or a portion thereof.
   73. The genetically engineered cell of any one of embodiments 1-72, wherein the Cas nuclease is Cas9, Cas12a or Cas12b.
   74. The genetically engineered cell of any one of embodiments 1-73, wherein the Cas nuclease comprises a catalytically inactive Cas molecule.
   75. The genetically engineered cell of any one of embodiments 1-74, wherein the Cas nuclease comprises a dead Cas (dCas).
   76. The genetically engineered cell of any one of embodiments 1-75, wherein the Cas nuclease comprises a dead Cas9 (dCas9).
   77. The genetically engineered cell of any one of embodiments 1-76, wherein the Cas nuclease comprises a nickase (nCas).
   78. The genetically engineered cell of any one of embodiments 1-77, wherein the Cas nuclease comprises a nCas9.
   79. The genetically engineered cell of any one of embodiments 1-78, wherein the Cas nuclease comprises a dCas or a nCas fused to one or more uracil glycosylase inhibitor (UGI) domains.
   80. The genetically engineered cell of any one of embodiments 1-79, wherein the Cas nuclease comprises a dCas or a nCas fused to an adenine base editor (ABE).
   81. The genetically engineered cell of embodiment 80, wherein the ABE comprises an adenine deaminase enzyme.
   82. The genetically engineered cell of any one of embodiments 1-79, wherein the Cas nuclease comprises a dCas or a nCas fused to a cytosine base editor (CBE).
   83. The genetically engineered cell of embodiment 82, wherein the CBE comprises a cytidine deaminase enzyme.
   84. The genetically engineered cell of any one of embodiments 1-83, wherein the cell is a hematopoietic cell, a progenitor cell, or a descendant thereof.
   85. The genetically engineered cell of embodiment 84, wherein the hematopoietic cell is a hematopoietic stem cell (HSC).
   86. The genetically engineered cell of any one of embodiments 84 or 85, wherein the hematopoietic cell is a CD34+ cell.
   87. The genetically engineered cell of any one of embodiments 84-86, wherein the hematopoietic cell is obtained from bone marrow, blood, umbilical cord, or peripheral blood mononuclear cells (PBMCs).
   88. The genetically engineered cell of any one of embodiments 84-87, wherein the hematopoietic cell is a human cell.
   89. A cell population, comprising a plurality of the genetically engineered cells of any one of embodiments 1-88.
   90. A cell population comprising a plurality of genetically engineered cells, wherein at least a portion of the cells comprise:
(a) a mutation within a first target domain in a genomic DNA molecule which generates an artificial PAM;
(b) an artificial PAM in proximity of a second or subsequent target domain; and
(c) an edit within the second or subsequent target domain.
   91. A method, comprising administering to a subject in need thereof:
(i) a population of the genetically engineered cells of any one of embodiments 1-90.
   92. The method of embodiment 91, further comprising administering (ii) an effective amount of the agent that specifically binds the cell-surface protein epitope.
   93. The method of either one of embodiments 91 or 92, wherein the subject has a hematopoietic malignancy.
   94. The method of embodiment 92 or 93, wherein the agent is a single-chain antibody fragment (scFv).
   95. The method of any one of embodiments 92 or 93, wherein the agent is an antibody or an antibody-drug conjugate (ADC).
   96. The method of embodiment 92, wherein the agent is an immune cell expressing a chimeric antigen receptor that comprises the antigen-binding fragment.
   97. The method of embodiment 96, wherein the immune cells are T cells.
   98. The method of embodiment 97, wherein the T cells express CD3, CD4, and/or CD8.
   99. The method of any one of embodiments 96-98, wherein the chimeric antigen receptor further comprises:
(a) a hinge domain
(b) a transmembrane domain,
(c) at least one co-stimulatory domain,
(d) a cytoplasmic signaling domain, or
(e) a combination thereof.
   100. The method of any one of embodiments 96-99, wherein the agent comprises: a CD33 antibody, a CD20 antibody, a CLL-1 antibody, a CD123 antibody, a CD38 antibody, a CD19 antibody, CD117 antibody, EMR2 antibody, or a CD5 antibody.
   101. The method of any one of 93-100, wherein the hematopoietic malignancy is Hodgkin's lymphoma, non-Hodgkin's lymphoma, leukemia, multiple myeloma (MM), myelodysplastic syndrome (MDS), or blastic plasmacytoid dendritic cell neoplasm (BPDCN).
   102. The method of any one of 93-101, wherein the hematopoietic malignancy is acute myeloid leukemia, B-cell acute lymphoblastic leukemia (B-ALL), chronic myelogenous leukemia, acute lymphoblastic leukemia, or chronic lymphoblastic leukemia.
   103. The method of any one of 93-102, wherein the hematopoietic malignancy is B-cell acute lymphoblastic leukemia (B-ALL).
   104. The method of any one of 93-102, wherein the hematopoietic malignancy is acute myeloid leukemia (AML).
   105. The method of any one of 93-102, wherein the hematopoietic malignancy is multiple myeloma (MM).
   106. The method of any one of 93-102, wherein the hematopoietic malignancy is myelodysplastic syndrome (MDS).
   107. A method comprising:
genetically modifying a cell to generate an artificial PAM in proximity of a target domain, comprising contacting a genomic DNA molecule with:
   (a) a first CRISPR/Cas system comprising a Cas nuclease and a first gRNA configured to direct the first CRISPR/Cas system to a first target domain resulting in the generation of an artificial PAM comprising introducing a mutation at the first target domain of the genomic DNA molecule;
   (b) a second CRISPR/Cas system comprising a Cas nuclease and a second gRNA configured to direct the second CRISPR/Cas system to a second or subsequent target domain comprising introducing a mutation at the second or subsequent target domain, or a part of it;
   wherein the second CRISPR/Cas system recognizes the artificial PAM, and wherein the second or subsequent target domain is in proximity to the artificial PAM.
      108. The method of embodiment 107, wherein the genomic DNA molecule is in a cell.
      109. The method of embodiment 107 or 108, wherein the cell is a genetically engineered cell of any one of embodiments 1-68.
      110. The method of any one embodiments 107-109, wherein the second or subsequent target domain is in a target gene.
      111. The method of embodiment 110, wherein the target gene encodes a cell-surface protein.
      112. The method of embodiment 110 or 111, wherein the target gene encodes a cell-surface protein epitope.
      113. The method of embodiment 112, wherein the mutation at the second or subsequent target domain results in an amino acid substitution in the cell-surface protein epitope.
      114. The method of embodiment 113, wherein the amino acid substitution is a non-conservative amino acid substitution.
      115. The method of embodiment 113 or 114, wherein the cell-surface protein epitope is bound by an agent, and wherein the amino acid substitution results in a reduction of the binding activity of the agent to the epitope comprising the amino acid substitution as compared to the unmodified epitope.
      116. The method of embodiment 115, wherein:
(i) the binding activity of the agent to the epitope is reduced by the amino acid substitution by at least about 5%, 10%, 15%, 20%, 25%, 30%, 35%, 40%, 45%, 50%, 55%, 60%, 65%, 70%, 75%, 80%, 85%, 90%, 95%, or 99% or more as compared to the unmodified epitope; and/or
(ii) the binding activity of the agent to the epitope is reduced by the amino acid substitution by at least about or at least about 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold as compared to the unmodified epitope.
   117. The method of embodiment 115 or 116, wherein the reduction in binding activity comprises an increase in K_{D}, IC₅₀, and/or EC₅₀.
   118. The method of embodiment 117, wherein:
(i) the K_{D}, IC₅₀, and/or EC₅₀ is increased by the amino acid substitution by at least about 5%, 10%, 15%, 20%, 25%, 30%, 35%, 40%, 45%, 50%, 55%, 60%, 65%, 70%, 75%, 80%, 85%, 90%, 95%, or 99% or more as compared to the unmodified epitope; or
(ii) the K_{D}, IC₅₀, and/or EC₅₀ is increased by the amino acid substitution by at least about or at least about 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold as compared to the unmodified epitope.
   119. The method of any one of embodiments 107-118, wherein the target gene encodes a cell-surface lineage-specific protein.
   120. The method of embodiment 119, wherein the cell-surface lineage-specific protein is CD19; CD123; CD38; KIT (CD117); CD20; or EGF-like module-containing mucin-like hormone receptor-like 2 (EMR2).
   121. The method of embodiment 119, wherein the cell-surface lineage-specific protein is CD5, CD19, CD20, CD38, CD117, or CD123.
   122. The method of embodiment 119, wherein the cell-surface lineage-specific protein is CD19, CD20, CD38, C-type lectin-like molecule-1 (CLL-1), CD123, or CD33.
   123. The method of embodiment 119, wherein the cell-surface lineage-specific protein is CD33.
   124. The method of embodiment 119, wherein the cell-surface lineage-specific protein is CD20.
   125. The method of embodiment 119, wherein the cell-surface lineage-specific protein is CLL-1.
   126. The method of embodiment 119, wherein the cell-surface lineage-specific protein is CD123.
   127. The method of embodiment 119, wherein the cell-surface lineage-specific protein is CD38.
   128. The method of embodiment 119, wherein the cell-surface lineage-specific protein is CD19.
   129. The method of embodiment 119, wherein the cell-surface lineage-specific protein is CD117.
   130. The method of embodiment 119, wherein the cell-surface lineage-specific protein is EMR2.
   131. The method of embodiment 119, wherein the cell-surface lineage-specific protein is CD5.
   132. The method of any one of embodiments 107-131, wherein the target gene, the cell-surface protein, and/or the cell-surface lineage-specific protein expression is associated with a cancer.
   133. The method of any one of embodiments 107-131, wherein the target gene, the cell-surface protein, and/or the cell-surface lineage-specific protein expression is associated with a hematopoietic malignancy.
   134. The method of embodiment 133, wherein the hematopoietic malignancy is Hodgkin's lymphoma, non-Hodgkin's lymphoma, leukemia, multiple myeloma (MM), myelodysplastic syndrome (MDS), or blastic plasmacytoid dendritic cell neoplasm (BPDCN).
   135. The method of embodiment 133 or 134, wherein the hematopoietic malignancy is acute myeloid leukemia, B-cell acute lymphoblastic leukemia (B-ALL), chronic myelogenous leukemia, acute lymphoblastic leukemia, or chronic lymphoblastic leukemia.
   136. The method of any one of embodiments 107-135, wherein the PAM and/or the artificial PAM is a Cas nuclease PAM.
   137. The method of any one of embodiments 107-135, wherein the PAM and/or the artificial PAM is a Cas9 PAM.
   138. The method of any one of embodiments 107-135, wherein the PAM and/or the artificial PAM is a Cas12a PAM.
   139. The method of any one of embodiments 107-135, wherein the PAM and/or the artificial PAM is a SpyCas9 PAM, a SpGCas9 PAM, a SpRYCas9 PAM, a Sth1Cas9 PAM, a SauCas9 PAM, a NmeCas9 PAM, a RspCas9 PAM, a Cca1Cas9 PAM, a PspCas9 PAM, a OrhCas9 PAM, a ScCas9 PAM, a SmacCas9 PAM, a TdeCas9 PAM, a Nme2Cas9 PAM, a CjeCas9 PAM, a SmuCas9 PAM, a Smu2Cas9 PAM, a PmuCas9 PAM, a SpaCas9 PAM, a NciCas9 PAM, a ClaCas9 PAM, a PlaCas9 PAM, a CdCas9 PAM, a IgnaviCas9 PAM, a ThermoCas9 PAM, a GeoCas9 PAM, a G. LC300 Cas9 PAM, a AceCas9 PAM, a Sth3Cas9 PAM, a BlatCas9 PAM, a FnCas9 PAM, a LfeCas9 PAM, a LpnCas9 PAM, a KhuCas9 PAM, a AinCas9 PAM, a CglCas9 PAM, a Esp1Cas9 PAM, a Esp2Cas9 PAM, a FmaCas9 PAM, a LceCas9 PAM, a LrhCas9 PAM, a Lsp1Cas9 PAM, a Lsp2Cas9 PAM, a PacCas9 PAM, a TbaCas9 PAM, a TpuCas9 PAM, a VpaCas9 PAM, a EfaCas9 PAM, a EitCas9 PAM, a LanCas9 PAM, a LmoCas9 PAM, a Sag1Cas9 PAM, a Sag2Cas9 PAM, a SdyCas9 PAM, a Seq1Cas9 PAM, a Seq2Cas9 PAM, a SgaCas9 PAM, a Smu3Cas9 PAM, a SraCas9 PAM, a BniCas9 PAM, a EceCas9 PAM, a EdoCas9 PAM, a FhoCas9 PAM, a MgaCas9 PAM, a MseCas9 PAM, a SgoCas9 PAM, a SmaCas9 PAM, a SsaCas9 PAM, a SsiCas9 PAM, a SsuCas9 PAM, a Sth1ACas9 PAM, a TspCas9 PAM, a BokCas9 PAM, a CcoCas9 PAM, a CpeCas9 PAM, a DdeCas9 PAM, a Ghc2Cas9 PAM, a Ghy3Cas9 PAM, a Ghy4Cas9 PAM, a GspCas9 PAM, a KkiCas9 PAM, a NspCas9 PAM, a TmoCas9 PAM, a NsaCas9 PAM, a JpaCas9 PAM, a BboCas9 PAM, a Cca2Cas9 PAM, a Cme2Cas9 PAM, a Cme3Cas9 PAM, a Cme4Cas9 PAM, a CsaCas9 PAM, a Ghc1Cas9 PAM, a GheCas9 PAM, a Ghh1Cas9 PAM, a Ghh2Cas9 PAM, a Ghy1Cas9 PAM, a MscCas9 PAM, a SdoCas9 PAM, a SpacCas9 PAM, a CgaCas9 PAM, a CmelCas9 PAM, a FfrCas9 PAM, a Ghy2Cas9 PAM, a PhiCas9 PAM, a WviCas9 PAM, a CcCas9 PAM, a FnCas12a PAM, a AsCas12a PAM, a HkCas12a PAM, a PiCas12a PAM, a PdCas12a PAM, a LbCas12a PAM, a Lb2Cas12a or Lb5Cas12a PAm, a CMtCas12a PAM, a MbCas12a PAM, a TsCas12a PAM, a Pb2Cas12a PAM, a MICas12a PAM, a Mb2Cas12a PAM, a Mb3Cas12a PAm, a CMaCas12a PAM, a BsCas12a PAM, a BfCas12a PAM, a BoCas12a PAM, a Adurb193Cas12a PAM, a Adurb336Cas12a PAM, a Fn3Cas12a PAM, a Lb6Cas12a PAM, a EcCas12a PAM, a PsCas12a PAM, a McCas12a PAM, a AacCas12b PAM, a BthCas12b PAM, a AkCas12b PAM, a EbCas12b PaM, a BvCas12b PAM, a BhCas12b PAM, a LsCas12b PAM, a BrCas12b PAM, a Cas12c1 PAM, a Cas12c2 PAM, a OspCas12c PAM, a Cas12d.15 PAM, a Cas12d.1 (CasY.1) PAM, a DpbCas12e (DpbCasX) PAM, a PlmCas12e (PlmCasX) PAM, a MilCas12f2 PAM, a Un1Cas12f1 PAM, a Un2Cas12f1 PAM, a Mi2Cas12f2 PAM, a AuCas12f2 PAM, a PtCas12f1 PAM, a AsCas12f1 PAM, a RuCas12f1 PAM, a SpCas12f1 PAM, a CnCas12f1 PAM, a Cas12h1 PAM, a Cas12i1 PAM, a Cas1212 PAM, a Cas12j-1 (CasPhi-1) PAM, a Cas12j-2 (CasPhi-2) PAM, a Cas12j-3 (CasPhi-3) PAM, a ShCas12k PAM, or a AcCas12k PAM.
   140. The method of any one of embodiments 107-139, wherein the PAM, and/or the artificial PAM comprises, and/or consists of a nucleotide sequence selected from: NGG; NNAGAAW; NNGRRT; NNNNGATT; NVNDCCY; BRTTTTT; NR (A or G) TTTT; NNAAAR (G or A); N (N or A) G; NAAN; NAAAAY; NHDTCCA; NNNVRYM; NNNNRYAC; NAA; GNNNNCNNA; NNGTGA; NNNNGTA; NNGGG; NNNCAT; NNRHHHY; NRRNAT; NNNNCNAA; NNNNCMCA; NNNNCRAA; NNNNGMAA; NNNCC; NGGNG; NNNNCNDD; NYAAA; NRGNN; N (C or D) GGN (T or A or G or C) NN; NRTAW; N (C or K or A) AARC; NAAAG; NV (A or G or C) R (A or G) ACCN; NNGAC; NATGNT; N (T or V) NTAAW (A or T); NNGW (A or T) AY (T or C); NCAA (H (Y or A) B (Y or G); NH (T or C or A) AAAA; NNNATTT; NATAWN (A or T or S); NATARCH; B (T or G or C) GGD (A or T or G) TNN; N (G or T or M) GGAH (T or A or C) N (A or C or K) N; NRG; N (B or A) GG; NGGD (A or K) W (T or A); N (T or C or R) AGAN (A or K or C) NN; NGGD (A or T or G) H (T or M); NGGDT; NGGD (A or T or G) GNN; NNGTAM (A or C) Y; NNGH (W or C) AAA; NTGAR (G or A) N (A or Y or G) N (Y or R); NNGAAAN; NNGAD; NHARMC; NNAAAG; NHGYNAN (A or B); NNAGAAA; NHAAAAA; NH (T or M) AAAAA; NHGYRAA; NNAAACN; NN (H or G) D (A or K) GGDN (A or B); NNNNCTA; NNNNCVGAA; NNNNGYAA; NNNNATN (W or S) ANN; NNWHR (G or A) TA (not G) AA; YHHNGTH; NNNNCDAANN; NNNNCTAA; N (C or D) NNTCCN; NNNNCCAA; NAGRGN (T or V) N (T or C); NNAH (T or M) ACN; CN (C or W or G) AV (A or S) GAC; NAR (G or A) H (W or C) H (A or T or C) GN (C or T or R); NAGNGC; NATCCTN; NGTGANN; HGCNGCR; NAR (A or G) W (T or A) AC; N (C or D) M (A or C) RN (A or B) AY (C or T); NNNCAC; BGGGTCD; NNRRCC; NRRNTT; KARDAT; BRRTTTW; NARNCCN; NAR (A or G) TC; NAAN (A or T or S) RCN; HHAAATD; NNNNGNA; TTV; TTTV; YYV; KKYV; TTTM; TTYV; TTTN; TTTTA; TTN; BTTV; YTV; YTN; NYTV; DTTD; ATTN; RTTNT; HATT; ATTW; RTTN; TVT; TG; TN; TR; TA; TTCN; TTAT; TTTR; TTR; YTTR; YTTN; CTT; TTC; CCD; RTR; VTTR; TBN; VTTN; NGTT; CGTT; AGG; CGG; GTT, or RGTG,
wherein “N” is any nucleotide or base, “W” is adenine (A) or thymine (T), “R” is A or guanine (G), “V” is A, cytosine (C), or G, “Y” is C or T, and “H” is A, C, or T.
   141. The method of any one of embodiments 107-140, wherein the PAM, and/or the artificial PAM comprises, and/or consists of a nucleotide sequence of NGG, wherein “N” is any nucleotide or base.
   142. The method of any one of embodiments 107-140, wherein the PAM, and/or the artificial PAM comprises, and/or consists of a nucleotide sequence of AGG.
   143. The method of any one of embodiments 107-140, wherein the PAM, and/or the artificial PAM comprises, and/or consists of a nucleotide sequence of CGG.
   144. The method of any one of embodiments 107-140, wherein the PAM, and/or the artificial PAM comprises, and/or consists of a nucleotide sequence of GTT.
   145. The method of any one of embodiments 107-144, wherein the first target domain is within about 1, 2, 3, 4, 5, 6, 7, 8, 9, 10, 11, 12, 13, 14, 15, 16, 17, 18, 19, 20, 21, 22, 23, 24, or 25 nucleotides of a PAM which is capable of targeting a CRISPR/Cas system to the first target domain.
   146. The method of any one of embodiments 107-145, wherein the genomic DNA molecule lacks a PAM in proximity of the second or subsequent target domain which is capable of targeting a CRISPR/Cas system to the second or subsequent target domain in order to provide a mutation within the second or subsequent target domain.
   147. The method of embodiment 146, wherein the genomic DNA molecule lacks a suitable PAM within about 1, 2, 3, 4, 5, 6, 7, 8, 9, 10, 11, 12, 13, 14, 15, 16, 17, 18, 19, 20, 21, 22, 23, 24, or 25 nucleotides of the second or subsequent target domain which is capable of targeting a CRISPR/Cas system to the second or subsequent target domain in order to provide a mutation within the second or subsequent target domain.
   148. The method of any one of embodiments 107-147, wherein the PAM which is capable of targeting a CRISPR/Cas system to the first target domain and the artificial PAM are separated by a predefined number of nucleotides.
   149. The method of embodiment 148, wherein the PAM which is capable of targeting a CRISPR/Cas system to the first target domain and the artificial PAM are separated by about 1, 2, 3, 4, 5, 6, 7, 8, 9, 10, 11, 12, 13, 14, 15, 16, 17, 18, 19, 20, 21, 22, 23, 24, or 25 nucleotides.
   150. The method of embodiment 149, wherein the artificial PAM is within about 1, 2, 3, 4, 5, 6, 7, 8, 9, 10, 11, 12, 13, 14, 15, 16, 17, 18, 19, 20, 21, 22, 23, 24, or 25 nucleotides of the second or subsequent target domain.
   151. The method of any one of embodiments 107-150, wherein the mutation within the first target domain is within a predefined number of nucleotides of the PAM which is capable of targeting a CRISPR/Cas system to the first target domain.
   152. The method of embodiment 151, wherein the mutation within the first target domain is within about 1, 2, 3, 4, 5, 6, 7, 8, 9, 10, 11, 12, 13, 14, 15, 16, 17, 18, 19, 20, 21, 22, 23, 24, or 25 nucleotides of the PAM which is capable of targeting a CRISPR/Cas system to the first target domain.
   153. The method of embodiment 152, wherein the mutation within the second or subsequent target domain is within about 1, 2, 3, 4, 5, 6, 7, 8, 9, 10, 11, 12, 13, 14, 15, 16, 17, 18, 19, 20, 21, 22, 23, 24, or 25 nucleotides of the artificial PAM.
   154. The method of any one of embodiments 148-153, wherein the predefined number of nucleotides comprises between about 1 to about 25 nucleotides, optionally, about 1, 2, 3, 4, 5, 6, 7, 8, 9, 10, 11, 12, 13, 14, 15, 16, 17, 18, 19, 20, 21, 22, 23, 24, or 25 nucleotides.
   155. The method of any one of embodiments 107-154, wherein:
(i) the first gRNA comprising a targeting domain which binds a site in a CD33 gene, optionally, wherein the site comprises the nucleic acid sequence of GCAAGTGCAGGAGTCAGTGA (SEQ ID NO: 68), or a portion thereof; or
(ii) the first gRNA comprising a targeting domain which binds a site in a CD20 gene, optionally, wherein the site comprises the nucleic acid sequence of ATATAATTGTATATGTTGAC (SEQ ID NO: 69), or a portion thereof.
   156. The method of any one of embodiments 107-155, wherein:
(i) the second gRNA comprising a targeting domain which binds a site in a CD33 gene, optionally, wherein the site comprises the nucleic acid sequence of GGATCCAAATTTCTGGCTGC (SEQ ID NO: 70), or a portion thereof; or
(ii) the second gRNA comprising a targeting domain which binds a site in a CD20 gene, optionally, wherein the site comprises the nucleic acid sequence of ACTTGGTCGATTAGGGAGAC (SEQ ID NO: 71), or a portion thereof.
   157. The method of any one embodiments 107-155, wherein:
(i) the first gRNA comprising a targeting domain which binds a site in a CD33 gene, optionally, wherein the site comprises the nucleic acid sequence of GCAAGTGCAGGAGTCAGTGA (SEQ ID NO: 68), or a portion thereof;
(ii) the first gRNA comprising a targeting domain which binds a site in a CD20 gene, optionally, wherein the site comprises the nucleic acid sequence of ATATAATTGTATATGTTGAC (SEQ ID NO: 69), or a portion thereof;
(iii) the first gRNA comprising a targeting domain which binds a site in a CD38 gene, optionally, wherein the site comprises the nucleic acid sequence of GTAGTGAAATTCTAGAGCTT (SEQ ID NO: 81), or a portion thereof; or
(iv) the first gRNA comprising a targeting domain which binds a site in a CD123 gene, optionally, wherein the site comprises the nucleic acid sequence of CCTTTGGCTCACGCTGCTCC (SEQ ID NO: 82), or a portion thereof.
   158. The method of any one of embodiments 107-157, wherein:
(i) the second gRNA comprising a targeting domain which binds a site in a CD33 gene, optionally, wherein the site comprises the nucleic acid sequence of GGATCCAAATTTCTGGCTGC (SEQ ID NO: 70), or a portion thereof;
(ii) the second gRNA comprising a targeting domain which binds a site in a CD20 gene, optionally, wherein the site comprises the nucleic acid sequence of ACTTGGTCGATTAGGGAGAC (SEQ ID NO: 71), or a portion thereof;
(iii) the second gRNA comprising a targeting domain which binds a site in a CD38 gene, optionally, wherein the site comprises the nucleic acid sequence of CCCGCAGGGTAAGTACCAAG (SEQ ID NO: 83) or GCAGGGTAAGTACCAAGTAG (SEQ ID NO: 84), or a portion thereof; or
(iv) the second gRNA comprising a targeting domain which binds a site in a CD123 gene, optionally, wherein the site comprises the nucleic acid sequence of CTCCTGATCGCCCTGCCTG (SEQ ID NO: 85), or a portion thereof.
   159. The method of any one of embodiments 107-158, wherein the Cas nuclease is Cas9, Cas12a, or Cas12b.
   160. The method of any one of embodiments 107-159, wherein the Cas nuclease comprises a catalytically inactive Cas molecule.
   161. The method of any one of embodiments 107-160, wherein the Cas nuclease comprises a dead Cas (dCas).
   162. The method of any one of embodiments 107-161, wherein the Cas nuclease comprises a dead Cas9 (dCas9).
   163. The method of any one of embodiments 107-162, wherein the Cas nuclease comprises a nickase (nCas).
   164. The method of any one of embodiments 107-163, wherein the Cas nuclease comprises a nCas9.
   165. The method of any one of embodiments 107-164, wherein the Cas nuclease comprises a dCas or a nCas fused to one or more uracil glycosylase inhibitor (UGI) domains.
   166. The method of any one of embodiments 107-165, wherein the Cas nuclease comprises a dCas or a nCas fused to an adenine base editor (ABE).
   167. The method of embodiment 166, wherein the ABE comprises an adenine deaminase enzyme.
   168. The method of any one of embodiments 107-167, wherein the Cas nuclease comprises a dCas or a nCas fused to a cytosine base editor (CBE).
   169. The method of embodiment 168, wherein the CBE comprises a cytidine deaminase enzyme.
   170. The method of any one of embodiments 107-169, wherein the contacting comprises introducing the CRISPR/Cas system into the cell in the form of a pre-formed ribonucleoprotein (RNP) complex.
   171. The method of any one of embodiments 107-170, wherein the ribonucleoprotein complex is introduced into the cell via electroporation.
   172. The method of any one of embodiments 107-171, in which (a) and (b) are introduced into the cell at the simultaneously.
   173. The method of any one of embodiments 107-171, in which in which (a) and (b) are introduced into the cell sequentially.
   174. The method of any one of embodiments 107-173, wherein the mutation at the first target domain comprises:
(i) a base (BE) editing mutation, optionally, wherein the base editing mutation comprises a modification which converts C to T, A to G, T to C, or G to A;
(ii) a high-fidelity homology directed repair (HDR)-based mutation, optionally, wherein the HDR-based mutation comprises a single nucleotide change and/or a insertion of a PAM sequence;
(iii) a ssODN mutation;
(iv) a prime editing (PE) mutation;
(v) a nucleobase transition, optionally, wherein nucleobase transition comprises the interchange of purine nucleobases A and G, or the interchange of pyrimidine nucleobases C and T; or
(vi) a nucleobase transversion, optionally, wherein the nucleobase transversion comprises the interchange of purine nucleobases A and G and pyrimidine nucleobases C and T.
   175. The method of any one of embodiments 107-174, wherein the mutation at the second or subsequent target domain comprises:
(i) a base (BE) editing mutation, optionally, wherein the base editing mutation comprises a modification which converts C to T, A to G, T to C, or G to A;
(ii) a high-fidelity homology directed repair (HDR)-based mutation, optionally, wherein the HDR-based mutation comprises a single nucleotide change and/or a insertion of a PAM sequence;
(iii) a ssODN mutation;
(iv) a prime editing (PE) mutation;
(v) a nucleobase transition, optionally, wherein nucleobase transition comprises the interchange of purine nucleobases A and G, or the interchange of pyrimidine nucleobases C and T;
(vi) a nucleobase transversion, optionally, wherein the nucleobase transversion comprises the interchange of purine nucleobases A and G and pyrimidine nucleobases C and T; or
(vii) a non-homologous end joining (NHEJ)-based mutation, optionally, wherein the NHEJ-based mutation comprises an indel that results in an amino acid deletion, insertion, or frameshift mutation, and/or wherein the NHEJ-based mutation results in a premature stop codon within the open reading frame (ORF) of the target gene.
   176. The method of any one of embodiments 107-175, wherein the method further comprises introducing into the cell:
(a) two or more gRNA configured to direct two or more CRISPR/Cas systems to two or more sites to provide two or more mutations to generate two or more artificial PAMs in the genomic DNA molecule; and, optionally,
(b) two or more CRISPR/Cas systems that binds the two or more gRNA of (a).
   177. The method of any one of embodiments 107-176, wherein the method further comprises introducing into the cell:
(a) two or more gRNA configured to direct two or more CRISPR/Cas systems to two or more sites to provide two or more mutations in two or more target genes; and, optionally,
(b) two or more CRISPR/Cas systems that binds the two or more gRNA of (a).
   178. The method of any one of embodiments 107-177, wherein the method results in three or more mutations, optionally, wherein the method results in a plurality of mutations such that the mutation generates an artificial PAM in the genomic DNA molecule and the last mutation generates a desired modification or effect on a target gene and/or on a protein encoded by the target gene.
   179. The method of embodiment 178, wherein the desired modification or effect on the target gene and/or the protein encoded by the target gene comprises healing a mutation, knocking out gene and/or protein expression, generating a target epitope that is not bound by a specific binding agent, optionally, wherein the binding agent is a ligand or an antibody.
   180. The method of any one of embodiments 107-179, wherein the method results in four or more mutations, five or more mutations, six or more mutations, seven or more mutations, eight or more mutations, nine or more mutations, or ten or more mutations.
   181. The method of any one of embodiments 178-180, wherein the mutation comprises the deamination of a cytosine.
   182. The method of embodiment 181, wherein the mutation comprises the deamination of an adenine.
   183. The method of embodiment 182, wherein the mutation comprises a nucleobase transition.
   184. The method of embodiment 182, wherein the mutation comprises a nucleobase transversion.
   185. The method of embodiment 184, wherein the mutation comprises converting a cytosine-guanine (C-G) base pair into a thymine-adenine (T-A) base pair within the target nucleic acid molecule.
   186. The method of embodiment 182, wherein the mutation comprises converting a thymine-adenine (T-A) base pair into a cytosine-guanine (C-G) base pair within the target nucleic acid molecule.
   187. The method of embodiment 182, wherein the mutation comprises introducing a premature STOP codon within the target nucleic acid molecule.
   188. The method of embodiment 182, wherein the mutation comprises introducing a splice site within the target nucleic acid molecule.
   189. The method of embodiment 182, wherein the mutation comprises disrupting a splice site within the target nucleic acid molecule.
   190. The method of embodiment 189, wherein the mutation comprises disrupting a splice donor.
   191. The method of embodiment 189, wherein the mutation comprises disrupting a splice acceptor.
   192. The method of embodiment 189, wherein the mutation comprises disrupting a splice enhancer.
   193. The method of embodiment 192, wherein the mutation comprises disrupting a exonic splice enhancer (ESE).
   194. The method of any one of embodiments 107-193, wherein the mutation reduces the activity of and/or expression of a target gene in a cell.
   195. A mixture comprising an mRNA encoding one, two, three, or all of:
(a) a first guide RNA (gRNA) configured to direct a first CRISPR/Cas system to a first target domain to provide an edit to generate an artificial PAM in a genomic DNA molecule;
(b) a second gRNA configured to direct a second CRISPR/Cas system to a second or subsequent target domain to provide an edit within a target gene;
(c) a CRISPR/Cas system that binds the first gRNA; and
(d) a CRISPR/Cas system that binds the second gRNA.
   196. A mixture of embodiment 195, wherein (a)-(b) are encoding by the same mRNA or separate mRNA.
   197. A kit or composition comprising:
(a) a first guide RNA (gRNA) configured to direct a first CRISPR/Cas system to a first target domain to provide an edit to generate an artificial PAM in a genomic DNA molecule; and
(b) a second gRNA configured to direct a second CRISPR/Cas system to a second or subsequent target domain to provide an edit within a target gene.
   198. A kit or composition comprising:
(a) a first guide RNA (gRNA) configured to direct a first CRISPR/Cas system to a first target domain to provide an edit to generate an artificial PAM in a genomic DNA molecule;
(b) a second gRNA configured to direct a second CRISPR/Cas system to a second or subsequent target domain to provide an edit within a target gene;
(c) a CRISPR/Cas system that binds the first gRNA; and
(d) a CRISPR/Cas system that binds the second gRNA.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The accompanying drawings are not intended to be drawn to scale. For purposes of clarity, the drawings are illustrative only and are not required for enablement of the disclosure. Not every component may be labeled in every drawing. In the drawings:

FIG. 1 is a schematic showing an exemplary approach for adenine base editing within exon 2 of the CD33 gene to generate an artificial protospacer adjacent motif (PAM). From top to bottom, the SEQ ID NOs are 1-5. The schematic shows a first target domain having the nucleic acid sequence GCAAGTGCAGGAGTCAGTGA (SEQ ID NO: 68) (or GCAAGTGCAGGAGTCAGTG (SEQ ID NO: 86)) in proximity to a CGG PAM which is suitable to introduce a mutation at a first site to generate an artificial PAM having the nucleic acid sequence of AGG in the double-stranded DNA (e.g., genomic) molecule in proximity to a second or subsequent target domain having the nucleic acid sequence of GGATCCAAATTTCTGGCTGC (SEQ ID NO: 70) (or GGATCCAAATTTCTGGCTG (SEQ ID NO: 87)) that lacks a PAM suitable to introduce a mutation at the second site. The schematic further shows the introduction of a mutation (i.e., amino acid substitution) at the second site that results in the amino acid substitution of N20D in the CD33 epitope recognized by lintuzumab, an anti-CD33 humanized monoclonal antibody used in the treatment of cancer. The amino acid substitution results in a reduction of the binding activity of lintuzumab to the epitope as compared to the unmodified epitope, as shown in FIG. 5.

FIG. 2 is a schematic showing an exemplary experimental procedure using adenine base editor sequential delivery in CD34+ cells to generate an artificial protospacer adjacent motif (PAM) in proximity of a target domain which lacks a suitable PAM. At Day 0, CD34+ cells were thawed for culture. At Day 2, a first CRISPR/Cas system comprising a first guide RNA (gRNA) and an adenine base editor 8 (ABE8), referred to as a “PAM generator,” configured to direct a first CRISPR/Cas system to a first target domain in proximity to an existing PAM and to introduce a first base edit (i.e., mutation) to generate an artificial PAM in a genomic DNA molecule, was introduced into the CD34+ cells via electroporation (EP). At Day 4, genomic DNA (gDNA) was obtained from the modified CD34+ cell populations to analyze genomic editing, e.g., via DNA sequencing. Additionally, at Day 4, a second CRISPR/Cas system comprising a second gRNA and an ABE8, referred to herein as a “targeting guide,” configured to direct a second CRISPR/Cas system to a second or subsequent target domain in proximity to the artificial PAM and to introduce a second base edit (i.e., mutation) at a second site, was introduced into the CD34+ cells via a subsequent EP. At Day 6, genomic DNA (gDNA) was obtained from the sequentially modified CD34+ cell populations to analyze genomic editing, e.g., via DNA sequencing.

FIG. 3 is a schematic showing an exemplary experimental procedure using adenine base editor simultaneous delivery in CD34+ cells to generate an artificial protospacer adjacent motif (PAM) in proximity of a target domain which lacks a suitable PAM. At Day 0, CD34+ cells were thawed for culture. At Day 2, a first CRISPR/Cas system comprising a first guide RNA (gRNA) and an adenine base editor 8 (ABE8), referred to as a “PAM generator,” configured to direct a first CRISPR/Cas system to a first target domain in proximity to an existing PAM and to introduce a first base edit (i.e., mutation) to generate an artificial PAM in a genomic DNA molecule; and a second CRISPR/Cas system comprising a second gRNA and an ABE8, also referred to as a “targeting guide,” configured to direct a second CRISPR/Cas system to a second or subsequent target domain in proximity to the artificial PAM and to introduce a second base edit (i.e., mutation) at a second site, were introduced into CD34+ cells simultaneously by electroporation. At Days 4 and 6, gDNA was obtained from the simultaneously modified CD34+ cell populations to analyze genomic editing, e.g., via DNA sequencing.

FIG. 4 shows histograms of MOLM-13 wildtype (WT) and CD33 knockout (CD33KO) cells stained with lintuzumab (hM195) (left panel), sequence alignments of various CD33 N-terminus mutants having one or more amino acid substitutions in the lintuzumab epitope, which includes the amino acid sequence MDPNFWLQVQE (SEQ ID NO: 2) (right panel). The CD33 N-terminus (amino acid residues 1-55) includes the wildtype amino acid sequence of MPLLLLLPLLWAGALAMDPNFWLQVQESVTVQEGLCVLVPCTFFHPIPYYDKNSP (SEQ ID NO: 6). The CD33 N-terminus mutants (from top to bottom, SEQ ID NOs: 8-14) included: dEpi, characterized by the deletion of MDPNFWLQVQE (SEQ ID NO: 2) at amino acid residue positions 17-27 and comprising the sequence of SEQ ID NO: 8 at its N-terminus; NFW, characterized by the substitution of NFW with AAA at residue positions 20-22 and comprising the sequence of SEQ ID NO: 9 at its N-terminus; LQV, characterized by the substitution of LQV with AAA at residue positions 23-25 and comprising the sequence of SEQ ID NO: 10 at its N-terminus; N20D, characterized by the substitution of N with D at residue position 20 and comprising the sequence of SEQ ID NO: 11 at its N-terminus; F21Y, characterized by the substitution of F with Y at residue position 21 and comprising the sequence of SEQ ID NO: 12 at its N-terminus; L23I, characterized by the substitution of L with I at residue position 23 and comprising the sequence of SEQ ID NO: 13 at its N-terminus; and Q24E, characterized by the substitution of Q with E at residue position 24 and comprising the sequence of SEQ ID NO: 14 at its N-terminus.

FIG. 5 shows histograms comparing recombinant CD33 expression in 293 FT cells stained with secondary antibody (negative control), P67.6 (positive control), or lintuzumab. Expression of CD33 WT, CD33 delta-epitope (dEpi), and CD33 N20D was evaluated. CD33 WT was bound by both P67.6 and lintuzumab; CD33 delta-epitope (dEpi) displayed no surface expression or shared an epitope between P67.6 and lintuzumab; and CD33 N20D displayed no reactivity with lintuzumab, while P67.6 binding confirmed surface expression.

FIG. 6 shows the sequences and table summarizing lintuzumab binding activity to the various CD33 N-terminus mutants. From top to bottom, SEQ ID NOs are 6 (“WT”) and 8-14 (corresponding to “dEpi”, “NEW”, “LQV”, “N20D”, “F21Y”, “L23I”, “Q24E”, respectively). Lintuzumab binding was disrupted by the N20D and Q24E mutants. GFP was included as a negative binding control.

FIGS. 7A-7C show that the binding site on CD20 is different among CD20 monoclonal antibodies (mAbs). FIG. 7A shows epitope mapping with CD20 wild type (WT) and CD20 mutants (XDO=T159K/N163D/N1660, AxP=A170S/P1725). HDC293F cells were transferred with plasmids and binding of monoclonal antibodies (mAbs; 5 μg/ml) to CD20 was measured by fluorescence-activated cell sorting. Binding was compared to Type I CD20 mAbs (m708 and mRTX) and Type II mAbs (B1 and m1188). FIG. 7B shows determination of residues crucial for CD20 mAb binding. Binding of CD20 mAbs (5 μg/ml) to CD20 mutants single mutant library spanning the 168EPANPSEKN5177 sequence (SEQ ID NO: 88) transiently expressed on HEK2939P cells was measured by FACS. Data are represented as % of best binder. Binding compared to best binder is shown by color (grey: 0-20%=loss of binding; light grey: 21-70%=intermediate binding; dark grey: 71-100% full binding). Results in A and B are representative of 2 separate experiments. SEQ ID NO: 15 is shown. FIG. 7C shows epitope mapping using cyclic variants of the CD20 amino acid sequence of YNCEPANPSEKNSPSTQYCYS (SEQ ID NO: 16; binding properties of amino acid residue positions within SEQ ID NO: 16 are show along the X-axis) which resulted in identification of the epitope of ml (left) but only marginally of m2 (right). Binding of 1 μg/ml mAB to the peptide and mutants with each amino acid replaced with all other available (positional scan; excluding cysteine) was determined by enzyme-linked immunosorbent assay. Horizontal line and shaded area represents binding to WT peptide±SEM. Results are displayed with Tukey-whiskers.

FIG. 8 shows CD20 sequence analysis. There are no suitable Cas9 PAMs (NGG) close to the target ANPSE (SEQ ID NO: 15) epitope that would allow base editing of the asparagine residue at position 171 (N171). The closest NGG PAMs are (on lower strand, shaded GGT on the right which is also located 5′ to the second set of underlined nucleotides encoding the ANPSE (SEQ ID NO: 15) epitope; and, on the upperstrand, shaded TGG of the terminal five nucleotides of the 3′ end) are shaded in grey. The upstream existing GGT PAM on the complementary strand cannot be used to directly edit the ANPSE (SEQ ID NO: 15) motif but could be used to generate a second (artificial) PAM. From top to bottom, SEQ ID NOs: 17-19 and 15 are shown.

FIG. 9 shows an exemplary approach for editing the CD20 ANPSE (SEQ ID NO: 15) epitope. The schematic shows a first target domain having the nucleic acid sequence ATATAATTGTATATGTTGAC (SEQ ID NO: 69) (indicated in underline in the top schematic) in proximity to an existing GGT PAM sequence (indicated as “PAM1”) which is suitable to provide a mutation to generate an artificial PAM having the nucleic acid sequence of GGC (indicated as “PAM2” in the middle schematic) in the genomic DNA molecule in proximity to a second or subsequent target domain having the nucleic acid sequence of ACTTGGTCGATTAGGGAGAC (SEQ ID NO: 71) (indicated in underline in the middle schematic) that lacks a PAM suitable to provide mutations at the second site. The schematic further shows a second or subsequent mutation that results in the modification of the CD20 epitope ANPSE (SEQ ID NO: 15) to ALPPE (SEQ ID NO: 90). The amino acid substitutions are likely to result in a reduction of the binding activity of an anti-CD20 antibody to the epitope as compared to the unmodified epitope. From top to bottom, SEQ ID NOs: 20-25 are shown.

FIG. 10 shows an exemplary CD38 gene sequence and its corresponding amino acid sequence and a structural model of CD38 wherein an epitope comprising arginine at amino acid residue position 195 (R195) is predicted to interact with the anti-CD38 antibody “isatuximab” via non-covalent, polar interactions (dashed lines). Arrows indicate amino acid side chain structures of CD38 R195 and isatuximab. From top to bottom, SEQ ID NOs: 26-29 are shown.

FIGS. 11A-11D show a non-limiting example of a strategy for modification of a CD38 epitope comprising R195. “R195G” indicates substitution of an arginine residue for a glycine residue at amino acid residue position 195 of CD38. “ABE” indicates adenosine base editor. “Starting PAM” indicates a naturally occurring distal PAM in the endogenous CD38 gene. “1^{st} ABE Editing Window” indicates first target domain. “CD38 gRNA-A” indicates first guide RNA comprising a targeting domain that hybridizes with the first target domain. Left “2^{nd} ABE Editing Window indicates second target domain for editing to encode CD38 R195G (left side “Final Result”). “CD38 gRNA-B” indicates the second guide RNA comprising a targeting domain that hybridizes with the 2^{nd} ABE Window target domain in the left schematic. The “2^{nd} ABE Editing Window in the right schematic indicates a second target domain for editing to generate the mutant CD38 R195G or disrupt the splice donor site sequence GTAA resulting in CD38 knockout (“KO”) (right side “Final Result” possibilities). “CD38 gRNA-C” indicates the second guide RNA comprising a targeting domain that hybridizes with the 2^{nd} ABE Editing Window target domain in the right schematic. Stars indicate positions of target adenine “A” nucleotides that may be converted to guanine “G” nucleotides using ABEs (“A>G”). FIG. 11A shows two CD38 modification methods comprising editing of an endogenous CD38 gene to produce either an edited endogenous CD38 gene encoding CD38 R195G, or CD38 knockout. SEQ ID NOs: 26, 28, 30, 32, 33, 41, 42, and 44 indicate genomic nucleotide positions within CD38 and SEQ ID NOs: 27, 40, and 42 indicate CD38 amino acids corresponding to coding sequences within the shown CD38 sequences. FIG. 11B shows representative DNA sequencing analysis of genomic DNA obtained from CD34+ hematopoietic stem or progenitor cells (“HSPCs”) that were electroporated (“EP”) with mRNA encoding ABE and CD38 gRNA-A, CD38 gRNA-B, or both CD38 gRNA-A and CD38 gRNA-B simultaneously. SEQ ID NOs: 45 and 46 indicate unmodified CD38 sequences. In the bottom left panel, SEQ ID NOs: 33 and 41 correspond to the top and bottom strands of unmodified CD38 sequences, wherein SEQ ID NO: 40 corresponds to CD38 amino acids encoded in the coding regions in SEQ ID NOs: 33 and 41. SEQ ID NOs: 47-49 indicate sequenced nucleotide positions within CD38. FIG. 11C shows representative DNA sequencing analysis of genomic DNA obtained from Jurkat cells that were electroporated (“EP”) with mRNA encoding ABE (“Mock EP;” top panel) or Jurkat cells (middle panel) and Daudi cells (bottom panel) that were electroporated with mRNA encoding ABE and CD38 gRNA-A. SEQ ID NOs: 45 and 46 indicate unmodified CD38 sequences. In the bottom left panel, SEQ ID NOs: 33 and 41 correspond to the top and bottom strands of unmodified CD38 sequences, wherein SEQ ID NO: 40 corresponds to CD38 amino acids encoded in the coding regions in SEQ ID NOs: 33 and 41. SEQ ID NOS: 50-52 indicates sequenced nucleotide positions within CD38. FIG. 11D shows representative DNA sequencing analysis of genomic DNA obtained from Jurkat cells that were electroporated (“EP”) with mRNA encoding ABE (“Mock EP”) or mRNA encoding ABE and CD38 gRNA-A, or CD38 gRNA-A followed by CD38 gRNA-B. SEQ ID NOs: 45 and 46 indicate unmodified CD38 sequences. In the bottom left panel, SEQ ID NOs: 33 and 41 correspond to the top and bottom strands of unmodified CD38 sequences, wherein SEQ ID NO: 40 corresponds to CD38 amino acids encoded in the coding regions in SEQ ID NOs: 33 and 41. SEQ ID NOs: 54 and 56 indicate sequenced nucleotide positions within CD38.

FIG. 12 shows representative flow cytometry analyses of Jurkat cell clones expressing CD38 comprising the R195G modification (“CD38 R195G”) generated by sequential electroporation (“EP”) with CD38 gRNA-A followed by CD38 gRNA-B. “HB-7” (left panel) and “Isatuximab” (middle panel) indicates anti-CD38 antibody clones. “Approx. EE %” indicates percentage of editing efficiency. “Mock EP” indicates cells that underwent electroporation with mRNA encoding ABE only. The right side panel shows genomic DNA sequencing analysis of select Jurkat cell clones expressing an edited endogenous CD38 gene encoding the CD38 R195G mutant. From top to bottom, SEQ ID NOs are 57-58 and indicate sequenced nucleotide positions in CD38 in genomic DNA samples obtained from the indicated clones.

FIG. 13 shows a non-limiting example of a strategy for modification of an epitope of CD123 (also referred to as IL3RA) within the predicted signal peptide region having leucine residues at amino acid positions 8 and 13. “Starting PAM” indicates a naturally occurring (existing) distal PAM in an endogenous CD123 gene. “ABE” indicates adenosine base editor. “1^{st} ABE Editing Window” indicates a first target domain for editing to encode L8P (substitution of leucine for proline at amino acid residue position 8 of CD123). “CD123 gRNA-A” indicates a first guide RNA comprising a targeting domain that hybridizes with the 1^{st} ABE Editing Window target domain. “2^{nd} ABE Editing Window” indicates a second target domain for editing to generate the L13P mutation (substitution of leucine for proline at amino acid residue position 13 of CD123). “CD123 gRNA-B” indicates the second guide RNA comprising a targeting domain that hybridizes with the 2^{nd} ABE Editing Window target domain. Stars indicate positions of target adenine “A” nucleotides that may be converted using ABEs to guanine “G” nucleotides (“A>G”). The CD123 epitope modification strategy comprising editing of an endogenous CD123 gene, wherein a distal PAM in a first target domain hybridizes with CD123 gRNA-A to induce an ABE-catalyzed A>G edit, thereby generating an artificial PAM. The artificial PAM is subsequently used by CD123 gRNA-B to induce an ABE-catalyzed A>G edit, thereby producing an edited endogenous CD123 gene encoding the modified signal peptide region comprising L8P and L13P (substitution of leucine for proline at amino acid residue positions 8 and 13 of CD123). From top to bottom, SEQ ID NOs are 59-62, 60, 63, 62, 60, 63, wherein SEQ ID NOs: 59, 61, 62, and indicate nucleotide positions within CD123 and SEQ ID NO: 60 indicates CD123 amino acids corresponding to coding sequences within the shown CD123 sequences.

FIG. 14 shows representative flow cytometry analyses of CD34+ hematopoietic stem cells (“HSPCs”; Donor 1 and Donor 2) expressing CD123 comprising the L8P mutant or both L8P and L13P mutations following electroporation (“EP”) with mRNA encoding ABE and CD123 gRNA-A, CD123 gRNA-B, or both CD123 gRNA-A and CD123 gRNA-B simultaneously. “Mock EP” indicates cells that underwent electroporation with mRNA encoding ABE only.

### DETAILED DESCRIPTION

### Definitions

The term “binds”, as used herein with reference to a gRNA interaction with a target domain (e.g., a first target domain and/or a second or subsequent target domain), refers to the gRNA molecule and the target domain forming a complex. The complex may comprise two strands forming a duplex structure, or three or more strands forming a multi-stranded complex. The binding may constitute a step in a more extensive process, such as the cleavage of the target domain by a Cas nuclease. In some embodiments, the gRNA binds to the target domain with perfect complementarity, and in other embodiments, the gRNA binds to the target domain with partial complementarity, e.g., with one or more mismatches. In some embodiments, when a gRNA binds to a target domain, the full targeting domain of the gRNA base pairs with the targeting domain. In other embodiments, only a portion of the target domain and/or only a portion of the targeting domain base pairs with the other. In an embodiment, the interaction is sufficient to mediate a target domain-mediated cleavage event.

As used herein, the terms “binds,” “specifically binds,” “specifically recognizes” and analogous terms as used herein with reference to a protein (e.g., a cell-surface protein) and an agent (e.g., a ligand or an antibody) interaction refer to the specific binding or association between the an agent (e.g., a ligand or an antibody) and protein (e.g., a cell-surface protein). Agents that specifically bind a cell-surface protein are known in the art and/or can be identified, for example, by immunoassays, BIAcore™, or other techniques known to those of skill in the art.

The term “reduced binding,” as used herein with reference to the binding activity of an agent (e.g., a ligand or an antibody) to a cell-surface protein epitope, refers to binding that is reduced by at least about 5%. The level of binding may refer to the amount of binding of the agent (e.g., a ligand or an antibody) to a cell, such as a hematopoietic cell or descendant thereof, or to the amount of binding of the agent (e.g., a ligand or an antibody) to the cell-surface protein. The level of binding of a cell, such as a hematopoietic cell or descendant thereof, that has been modified, for example, to include an amino acid substitution in a cell-surface protein epitope, may be relative to the level of binding of the agent (e.g., a ligand or an antibody) to a cell that has not been modified as determined by the same assay under the same conditions. In exemplary embodiments, the level of binding of a cell-surface protein that includes an amino acid substitution in a cell-surface protein epitope to an agent (e.g., a ligand or an antibody) may be relative to the level of binding of the agent to a cell-surface protein that does not include the amino acid substitution in the cell-surface protein epitope (e.g., a wild-type protein or a variant thereof) as determined by the same assay under the same conditions. Alternatively, the level of binding of a cell-surface protein that lacks an epitope to an agent (e.g., a ligand or an antibody) may be relative to the level of binding of the agent to a cell-surface protein that contains the unmodified epitope (e.g., a wild-type protein) as determined by the same assay under the same conditions.

In some embodiments, the binding is reduced by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 100% or more, e.g., as compared to the unmodified epitope (e.g., a wild-type protein). In some embodiments, the binding is reduced by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, or at least about 100-fold, e.g., as compared to the unmodified epitope (e.g., a wild-type protein). In some embodiments, the binding is reduced such that there is substantially no detectable binding in a conventional assay.

The binding affinity or binding specificity for an epitope or protein can be determined by a variety of methods including equilibrium dialysis, equilibrium binding, gel filtration, ELISA, surface plasmon resonance, or spectroscopy.

As used herein, “no binding” refers to substantially no binding, e.g., no detectable binding or only baseline binding as determined in a conventional binding assay. In some embodiments, there is no binding between the cells, e.g., hematopoietic cells or descendants thereof, that have been modified and the agent. In some embodiments, there is no detectable binding between the cells, e.g., hematopoietic cells or descendants thereof, that have been modified and the agent. In some embodiments, no binding of the cells, e.g., hematopoietic cells or descendant thereof, to the agent refers to a baseline level of binding, as shown using any conventional binding assay known in the art. In some embodiments, the level of binding of the cells, e.g., hematopoietic cells or descendants thereof, that have been modified and the agent is not biologically significant. The term “no binding” is not intended to require the absolute absence of binding. Without wishing to be bound by theory, a subject can be administered the rescue cells (e.g., hematopoietic stem cells (HSCs) and/or hematopoietic progenitor cells (HPCs)) described herein comprising a modification in a cell-surface antigen gene, e.g., a genetic edit (i.e., mutation) that results in the rescue cells having reduced or eliminated expression of the respective gene, or a modification of an epitope of the protein encoded by the respective gene that diminishes the binding of the therapeutic agent to the protein. These genetically engineered (modified) cells can thus be resistant to an agent, such as an anti-cancer therapy, and can therefore repopulate the hematopoietic system during or after anti-cancer therapy.

As used herein, the terms “protein,” “peptide,” and “polypeptide” may be used interchangeably and refer to a polymer of amino acid residues linked together by peptide bonds. In general, a protein may be naturally occurring, recombinant, synthetic, or any combination of these. Also within the scope of the term are variant proteins, which comprise a mutation (e.g., substitution, insertion, or deletion) of one or more amino acid residues relative to the unmodified, e.g., wild-type, counterpart.

As used herein, the term “cell-surface protein” refers to a protein, at least a portion of which is present on the extracellular surface of a cell. In some embodiments, a “cell-surface protein” may be a lineage-specific cell-surface protein. A cell-surface protein may be displayed on the surface of a cell such that it is capable of being bound by another agent, such as a ligand or an antibody.

As used herein, the terms “lineage-specific cell-surface protein” and “cell-surface lineage-specific protein” may be used interchangeably and refer to any protein that is sufficiently present on the surface of a cell and is associated with one or more populations of cell lineage(s). For example, the protein may be present on one or more populations of cell lineage(s) and absent (or at reduced levels) on the cell-surface of other cell populations. In some embodiments, the terms lineage-specific cell-surface antigen” and “cell-surface lineage-specific antigen” may be used interchangeably and refer to any antigen of a lineage-specific cell-surface protein. The compositions and methods described herein may be used to achieve editing of a cell-surface protein, which may result in making a variant lineage-specific cell-surface protein. Such editing may result in an amino acid substitution in an epitope of interest, such as a cell-surface protein epitope.

In some instances, a variant, which may include a protein comprising an amino acid substitution obtained or obtainable by any one of the editing methods described herein, may contain one or more amino acid substitutions (e.g., 2, 3, 4, 5, or more) within the epitope of interest such that the agent does not bind or has reduced binding to the mutated epitope. Such a variant may have substantially reduced binding affinity to the agent (e.g., having a binding affinity that is at least about 5%, about 10%, about 15%, about 20%, about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 99% or more lower as compared to the unmodified epitope (e.g., its wild-type counterpart). In some examples, such a variant may have abolished binding activity to the agent. In other instances, the variant contains a deletion of a region that comprises the epitope of interest. Such a region may be encoded by an exon. In some embodiments, the region is a domain of the protein of interest that encodes the epitope. In one example, the variant has just the epitope deleted. The length of the deleted region may range from about 1 to about 100 amino acids, e.g., 1-25, 1-50, 25-75, 50-75, 50-100, 3-60, 5-50, 5-40, 10-30, 10-20, etc.

As used herein, the term “epitope” refers to an amino acid sequence (linear or conformational) of a protein (e.g., a cell-surface antigen) that is bound by, e.g., a ligand or the complementarity determining regions (CDRs), e.g., CDR1, CDR2, and CDR3, and framework regions (FRs), of an antibody. The term “cell-surface antigen” or “cell-surface epitope” refers to an antigen or epitope on the surface of a cell that is extracellularly accessible. The antigen or epitope on the surface of the cell may be extracellularly accessible at any cell cycle stage of the cell, including antigens or epitopes that are predominantly or only extracellularly accessible during cell division. “Extracellularly accessible” in this context may refer to an antigen or epitope that can be bound by a ligand or an antibody provided outside the cell without need for permeabilization of the cell membrane.

In some embodiments, at least one epitope of a protein (e.g., cell-surface protein) has been modified, preventing at least one agent (e.g., a ligand or an antibody) from being targeted to the at least one epitope. In some embodiments, two or more (e.g., 2, 3, 4, 5 or more) epitopes of a protein (e.g., a cell-surface protein) have been modified, preventing two or more (e.g., 2, 3, 4, 5 or more) different agents (e.g., two ligands or two or more antibodies) from being targeted to the two or more epitopes. In some embodiments, the agent (e.g., a ligand or an antibody) binds to one or more (e.g., at least 2, 3, 4, 5 or more) epitopes of a protein (e.g., a cell-surface protein). In some embodiments, the agent binds to more than one epitope of the cell-surface protein and the cells, for example, hematopoietic cells, are modified such that each of the epitopes is absent and/or unavailable for binding by the agent.

In some embodiments, the genetically engineered cells described herein have one or more modified genes of proteins, for example, cell-surface proteins, such that the modified genes express mutated cell-surface proteins with mutations in one or more epitopes. In certain embodiments, a modified cell, such as a modified hematopoietic cell described herein, comprising a deletion or mutation of an epitope of a cell-surface protein is able to proliferate and/or undergo erythropoietic differentiation to a similar level as hematopoietic cells that contain the unmodified epitope (e.g., a wild-type cell-surface protein). In certain embodiments, the genetically engineered HSCs described herein comprise one or more artificial protospacer adjacent motifs (PAMs). In certain embodiments, a modified cell, such as a modified hematopoietic cell described herein, comprises two or more (e.g., 2, 3, 4, 5 or more) artificial PAMs. The artificial PAM may be about 1, 2, 3, 4, 5, 6, 7, 8, 9, 10, 11, 12, 13, 14, 15, 16, 17, 18, 19, 20, 21, 22, 23, 24, or 25 or more nucleotides from a mutation in the nucleic acid sequence encoding an epitope of a cell-surface protein.

In certain embodiments, as used herein, an “agent” or “agent” can refer to an active agent, including, without limitation, a protein-agent that permits modulation of activity of proteins or disrupts interactions of proteins and other biomolecules, such as but not limited to disrupting protein-protein interaction, ligand-receptor interaction, antibody-protein, or protein-nucleic acid interaction. Agents may include a fragment, a derivative, and an analog of an active agent. The terms “fragment,” “derivative,” and “analog” when referring to polypeptides as used herein refers to polypeptides which either retain substantially the same biological function or activity as such polypeptides. Such agents include, but are not limited to, antibodies (“antibodies” includes antigen-binding portions of antibodies such as epitope- or antigen-binding peptides, paratopes, functional CDRs; recombinant antibodies; chimeric antibodies; humanized antibodies; NANOBODIES® (single domain antibodies such as VH and VHH domains); tribodies; midibodies; or antigen binding derivatives, analogs, variants, portions, or fragments thereof), protein-agents, nucleic acid molecules, small molecules, recombinant protein, peptides, aptamers, avimers and protein-binding derivatives, portions or fragments thereof. Generally, the term “agent” as used herein, may also refer to any therapeutic or biologically active agent, including an agent associated with gene editing technologies, such as CRISPR/Cas systems.

In some embodiments, the term “active agent” refers to any agent that possesses therapeutic, prophylactic, or diagnostic properties in vivo, for example when administered to a subject. Examples of active agents include, but are not limited to, adjuvants, antibodies, antibody chains, antibody fragments, antigens, bi-specific molecules, cells, cellular therapeutic agents, chemokines, cytokines, enzymes, growth factors, immunogens, immunoglobulins, interleukins, nucleic acids, oligonucleotides, peptides, proteins, small molecules, sugars, vaccines, or a combination of two or more thereof. The terms “chemotherapeutic agent” or “anti-cancer agent” are used herein to refer to all agents that are effective in treating cancer regardless of mechanism of action. The term “immunotherapeutic agent” refers to an agent that comprises or consists of one or more adjuvants, antibodies, antibody chains, antibody fragments, antigens, bi-specific molecules, cells, cellular therapeutic agents, chemokines, cytokines, enzymes, growth factors, immunogens, immunoglobulins, interleukins, nucleic acids, oligonucleotides, peptides, proteins, small molecules, sugars, vaccines, or a combination of two or more thereof, for use in a therapeutic or prophylactic treatment, of a disease or disorder intended to and/or producing an immune response (e.g., an active or passive immune response).

A “Cas nuclease” as that term is used herein, refers to a CRISPR/Cas nuclease (e.g., Cas9) that can interact with a gRNA and, in concert with the gRNA, home or localize to a site which comprises a target domain. Cas nucleases include naturally occurring Cas nucleases and engineered, altered, or modified Cas nucleases that differ, e.g., by at least one amino acid residue, from a naturally occurring Cas nuclease.

The term, a “mutation at a first target domain,” is used herein to refer to a first mutation to generate an artificial protospacer adjacent motif (PAM) in a double-stranded (genomic) DNA molecule. The genomic DNA molecule may lack a suitable PAM in proximity of a second or subsequent target domain which is capable of targeting a CRISPR/Cas system to the second or subsequent target domain in order to provide an mutation within the second or subsequent target domain. Accordingly, the first mutation can introduce an artificial PAM in proximity of the second or subsequent target domain which can allow for an mutation within the second or subsequent target domain. For example, the methods provided herein may be used to edit a genomic DNA molecule to generate an artificial PAM in proximity of a target domain in a gene of interest. The method may comprise providing a cell comprising a genomic DNA molecule, and introducing into the cell a first guide RNA (gRNA) configured to direct a first CRISPR/Cas system to a first target domain proximal to a naturally occurring PAM (i.e., non-artificial or existing PAM) to provide a first mutation to generate an artificial PAM in a genomic DNA molecule. The mutation may also result in the introduction of a base editing mutation, a homology-directed repair (HDR)-based mutation, a single-strand oligodeoxynucleotide (ssODN), a prime editing mutation, a nucleobase transition, a nucleobase transversion, an HDR-based substitution, or an insertion of an artificial PAM.

The term, a “mutation at a second or subsequent target domain” is used herein to refer to a second or subsequent mutation to modify a target domain, e.g., within a gene of interest, within proximity of an artificial PAM. The target gene may encode a cell-surface protein epitope, and the second mutation may result in an amino acid substitution or deletion in a cell-surface protein epitope. Such an amino acid substitution may be a non-conservative amino acid substitution. The cell-surface protein epitope may be bound by an agent (e.g., a ligand or an antibody), and the amino acid substitution may result in a reduction of the binding activity of the agent (e.g., a ligand or an antibody) to the epitope comprising the amino acid substitution as compared to the unmodified epitope.

The terms “gRNA” and “guide RNA” are used interchangeably throughout and refer to a nucleic acid that promotes the specific targeting or homing of a CRISPR/Cas system to a target nucleic acid sequence. A gRNA can be unimolecular (having a single RNA molecule), sometimes referred to herein as sgRNAs, or modular (comprising more than one, and typically two, separate RNA molecules). A gRNA may bind to a target domain in the genome of a host cell. The gRNA may comprise a targeting domain that may be partially or completely complementary to the target domain. The gRNA may also comprise a “scaffold sequence,” (e.g., a tracrRNA sequence), that recruits a Cas nuclease to a target domain bound to a gRNA sequence (e.g., by the targeting domain of the gRNA sequence). The scaffold sequence may comprise at least one stem loop structure and recruits an endonuclease. Exemplary scaffold sequences can be found, for example, in Jinek, et al. Science (2012) 337 (6096): 816-821, Ran, et al. Nature Protocols (2013) 8:2281-2308, International Publication No. WO2014/093694, and International Publication No. WO2013/176772.

As used herein, the terms “proximity,” “in proximity,” or “within proximity,” refers to a first, second or subsequent target site within distance of a PAM or artificial PAM that can be bound by a CRISPR/Cas system, according to any method described herein. In various embodiments, the mutation of the genomic DNA sequence at the first target site is within 1, 2, 3, 4, 5, 6, 7, 8, 9, 10, 11, 12, 13, 14, 15, 16, 17, 18, 19, 20, 21, 22, 23, 24, or 25 nucleotides from a PAM sequence. In various embodiments, the mutation of the genomic DNA sequence at the second or subsequent target site is within 1, 2, 3, 4, 5, 6, 7, 8, 9, 10, 11, 12, 13, 14, 15, 16, 17, 18, 19, 20, 21, 22, 23, 24, or 25 nucleotides from an artificial PAM.

As used herein, the term “sgRNA,” refers to single guide RNA used in conjunction with CRISPR associated systems (Cas). sgRNAs are a fusion of crRNA and tracrRNA and contain nucleotides of sequence complementary to the desired target site (Jinek et al., Science, 343 (6176): 1247997, 2014). Watson-Crick pairing of the sgRNA with the target site permits R-loop formation, which in conjunction with a functional PAM permits DNA cleavage or in the case of nuclease-deficient Cas9 allows binding to the DNA at that locus. As used herein, the term “protospacer adjacent motif” or “PAM” refers to a DNA sequence that may be required for a nuclease and a gRNA (e.g., Cas9/sgRNA) to form an R-loop to interrogate a specific DNA sequence through Watson-Crick pairing of its guide RNA with the genome. The PAM specificity may be a function of the DNA-binding specificity of the nuclease, e.g., Cas9 protein, e.g., a “protospacer adjacent motif recognition domain” at the C-terminus of Cas9. In some embodiments, the PAM comprises a 2 to 6 base pair DNA sequence immediately downstream or upstream of the target site in the genome, which may be recognized directly by an Cas nuclease, e.g., a Cas9, to promote cleavage of the target site by the Cas nuclease, or in the case of nuclease-deficient Cas9 allows binding to the DNA at that locus. In some embodiments, the PAM can be a 5′ PAM (i.e., located upstream of the 5′ end of the protospacer). In other embodiments, the PAM can be a 3′ PAM (i.e., located downstream of the 5′ end of the protospacer).

As used herein, the term “Cas nuclease PAM” refers to a DNA sequence that may be required to home or localize a CRISPR/Cas nuclease (e.g., Cas9 or other Cas nucleases, such as Cas12a (Cpf1) or Cas12b) interacting with a gRNA to sequence comprising a target domain. Non-limiting examples of CRISPR/Cas nucleases and their corresponding consensus PAM are described herein.

For example, the Cas9 endonuclease from Streptococcus pyogenes (SpyCas9) can recognize a NGG consensus PAM (e.g., a 5′-NGG on the 3′ end of the gRNA sequence) (N=any base). SpyCas9 can also weakly recognize NGA, NNGG, and a selection of other sequences. The SpGCas9 (a variant of SpyCas9) can recognize a NGN (N=any base) consensus PAM. The SpRYCas9 (a variant of SpGCas9) can recognize a NRN (N=any base; R=A,G) consensus PAM. SpRYCas9 can also weakly some NYN (N=any base; Y=C or T) sequences. The Cas9 nuclease from the CRISPR1 locus (Sth1Cas9) in the model lactic-acid bacterium Streptococcus thermophilus can recognize a NNATAAW (W=A, T; N=any base) consensus PAM. The Cas9 nuclease from the pathogen Staphylococcus aureus (SauCas9) can recognize an NNGRRT consensus PAM (R=A, G; N=any base). The Cas9 nuclease from the pathogen Neisseria meningitidis (NmeCas9) can recognize a NNNNGATT consensus PAM (N=any base). The MAD7 endonuclease from Eubacterium rectale can recognize 5′-TTTV on the 5′ end of the gRNA sequence, and to some extent 5′-YTTV and YTTN. The Cpf1 endonuclease from Acidaminococcus sp. And Lachnospiraceae sp. Recognize 5′-TTTN and the Cpf1 endonuclease from Francisella novicide can recognize 5′-TTN-3′ on the 5′ end of the gRNA.

In certain embodiments, the PAM is a SpyCas9 PAM, a SpGCas9 PAM, a SpRYCas9 PAM, a Sth1Cas9 PAM, a SauCas9 PAM, a NmeCas9 PAM, a RspCas9 PAM, a Cca1Cas9 PAM, a PspCas9 PAM, a OrhCas9 PAM, a ScCas9 PAM, a SmacCas9 PAM, a TdeCas9 PAM, a Nme2Cas9 PAM, a CjeCas9 PAM, a SmuCas9 PAM, a Smu2Cas9 PAM, a PmuCas9 PAM, a SpaCas9 PAM, a NciCas9 PAM, a ClaCas9 PAM, a PlaCas9 PAM, a CdCas9 PAM, a IgnaviCas9 PAM, a ThermoCas9 PAM, a GeoCas9 PAM, a G. LC300 Cas9 PAM, a AceCas9 PAM, a Sth3Cas9 PAM, a BlatCas9 PAM, a FnCas9 PAM, a LfeCas9 PAM, a LpnCas9 PAM, a KhuCas9 PAM, a AinCas9 PAM, a CglCas9 PAM, a Esp1Cas9 PAM, a Esp2Cas9 PAM, a FmaCas9 PAM, a LceCas9 PAM, a LrhCas9 PAM, a Lsp1Cas9 PAM, a Lsp2Cas9 PAM, a PacCas9 PAM, a TbaCas9 PAM, a TpuCas9 PAM, a VpaCas9 PAM, a EfaCas9 PAM, a EitCas9 PAM, a LanCas9 PAM, a LmoCas9 PAM, a Sag1Cas9 PAM, a Sag2Cas9 PAM, a SdyCas9 PAM, a Seq1Cas9 PAM, a Seq2Cas9 PAM, a SgaCas9 PAM, a Smu3Cas9 PAM, a SraCas9 PAM, a BniCas9 PAM, a EceCas9 PAM, a EdoCas9 PAM, a FhoCas9 PAM, a MgaCas9 PAM, a MseCas9 PAM, a SgoCas9 PAM, a SmaCas9 PAM, a SsaCas9 PAM, a SsiCas9 PAM, a SsuCas9 PAM, a Sth1ACas9 PAM, a TspCas9 PAM, a BokCas9 PAM, a CcoCas9 PAM, a CpeCas9 PAM, a DdeCas9 PAM, a Ghc2Cas9 PAM, a Ghy3Cas9 PAM, a Ghy4Cas9 PAM, a GspCas9 PAM, a KkiCas9 PAM, a NspCas9 PAM, a TmoCas9 PAM, a NsaCas9 PAM, a JpaCas9 PAM, a BboCas9 PAM, a Cca2Cas9 PAM, a Cme2Cas9 PAM, a Cme3Cas9 PAM, a Cme4Cas9 PAM, a CsaCas9 PAM, a Ghc1Cas9 PAM, a GheCas9 PAM, a Ghh1Cas9 PAM, a Ghh2Cas9 PAM, a Ghy1Cas9 PAM, a MscCas9 PAM, a SdoCas9 PAM, a SpacCas9 PAM, a CgaCas9 PAM, a CmelCas9 PAM, a FfrCas9 PAM, a Ghy2Cas9 PAM, a PhiCas9 PAM, a WviCas9 PAM, a CcCas9 PAM, a FnCas12a PAM, a AsCas12a PAM, a HkCas12a PAM, a PiCas12a PAM, a PdCas12a PAM, a LbCas12a PAM, a Lb2Cas12a or Lb5Cas12a PAM, a CMtCas12a PAM, a MbCas12a PAM, a TsCas12a PAM, a Pb2Cas12a PAM, a MICas12a PAM, a Mb2Cas12a PAM, a Mb3Cas12a PAM, a CMaCas12a PAM, a BsCas12a PAM, a BfCas12a PAM, a BoCas12a PAM, a Adurb193Cas12a PAM, a Adurb336Cas12a PAM, a Fn3Cas12a PAM, a Lb6Cas12a PAM, a EcCas12a PAM, a PsCas12a PAM, a McCas12a PAM, a AacCas12b PAM, a BthCas12b PAM, a AkCas12b PAM, a EbCas12b PAM, a BvCas12b PAM, a BhCas12b PAM, a LsCas12b PAM, a BrCas12b PAM, a Cas12c1 PAM, a Cas12c2 PAM, a OspCas12c PAM, a Cas12d.15 PAM, a Cas12d.1 (CasY.1) PAM, a DpbCas12e (DpbCasX) PAM, a PlmCas12e (PlmCasX) PAM, a MilCas12f2 PAM, a Un1Cas12f1 PAM, a Un2Cas12f1 PAM, a Mi2Cas12f2 PAM, a AuCas12f2 PAM, a PtCas12f1 PAM, a AsCas12f1 PAM, a RuCas12f1 PAM, a SpCas12f1 PAM, a CnCas12f1 PAM, a Cas12h1 PAM, a Cas12i1 PAM, a Cas1212 PAM, a Cas12j-1 (CasPhi-1) PAM, a Cas12j-2 (CasPhi-2) PAM, a Cas12j-3 (CasPhi-3) PAM, a ShCas12k PAM, or a AcCas12k PAM.

In certain embodiments, the PAM comprises and/or consists of a nucleotide sequence selected from: NGG; NNAGAAW; NNGRRT; NNNNGATT; NVNDCCY; BRTTTTT; NR (A or G) TTTT; NNAAAR (G or A); N (N or A) G; NAAN; NAAAAY; NHDTCCA; NNNVRYM; NNNNRYAC; NAA; GNNNNCNNA; NNGTGA; NNNNGTA; NNGGG; NNNCAT; NNRHHHY; NRRNAT; NNNNCNAA; NNNNCMCA; NNNNCRAA; NNNNGMAA; NNNCC; NGGNG; NNNNCNDD; NYAAA; NRGNN; N (C or D) GGN (T or A or G or C) NN; NRTAW; N (C or K or A) AARC; NAAAG; NV (A or G or C) R (A or G) ACCN; NNGAC; NATGNT; N (T or V) NTAAW (A or T); NNGW (A or T) AY (T or C); NCAA (H (Y or A) B (Y or G); NH (T or C or A) AAAA; NNNATTT; NATAWN (A or T or S); NATARCH; B (T or G or C) GGD (A or T or G) TNN; N (G or T or M) GGAH (T or A or C) N (A or C or K) N; NRG; N (B or A) GG; NGGD (A or K) W (T or A); N (T or C or R) AGAN (A or K or C) NN; NGGD (A or T or G) H (T or M); NGGDT; NGGD (A or T or G) GNN; NNGTAM (A or C) Y; NNGH (W or C) AAA; NTGAR (G or A) N (A or Y or G) N (Y or R); NNGAAAN; NNGAD; NHARMC; NNAAAG; NHGYNAN (A or B); NNAGAAA; NHAAAAA; NH (T or M) AAAAA; NHGYRAA; NNAAACN; NN (H or G) D (A or K) GGDN (A or B); NNNNCTA; NNNNCVGAA; NNNNGYAA; NNNNATN (W or S) ANN; NNWHR (G or A) TA (not G) AA; YHHNGTH; NNNNCDAANN; NNNNCTAA; N (C or D) NNTCCN; NNNNCCAA; NAGRGN (T or V) N (T or C); NNAH (T or M) ACN; CN (C or W or G) AV (A or S) GAC; NAR (G or A) H (W or C) H (A or T or C) GN (C or T or R); NAGNGC; NATCCTN; NGTGANN; HGCNGCR; NAR (A or G) W (T or A) AC; N (C or D) M (A or C) RN (A or B) AY (C or T); NNNCAC; BGGGTCD; NNRRCC; NRRNTT; KARDAT; BRRTTTW; NARNCCN; NAR (A or G) TC; NAAN (A or T or S) RCN; HHAAATD; NNNNGNA; TTV; TTTV; YYV; KKYV; TTTM; TTYV; TTTN; TTTTA; TTN; BTTV; YTV; YTN; NYTV; DTTD; ATTN; RTTNT; HATT; ATTW; RTTN; TVT; TG; TN; TR; TA; TTCN; TTAT; TTTR; TTR; YTTR; YTTN; CTT; TTC; CCD; RTR; VTTR; TBN; VTTN; NGTT; CGTT; AGG; CGG; GTT; or RGTG, wherein “N” is any nucleotide or base, “W” is adenine (A) or thymine (T), “R” is A or guanine (G), “V” is A, cytosine (C), or G, “Y” is C or T, and “H” is A, C, or T.

For an overview of other PAM sequences, see, for example, Shah et al., Protospacer recognition motifs. RNA Biol. 10 (5): 891-899, 2013; and Collias et al., CRISPR technologies and the search for the PAM-free nuclease. Nat Commun. 12 (1): 555, 2021. The term “mutation” is used herein to refer to a genetic change (e.g., insertion, deletion, inversion, or substitution) in a nucleic acid compared to a reference sequence, e.g., the corresponding sequence of a cell not having such a mutation or corresponding wild-type nucleic acid sequence. In some embodiments provided herein, a mutation in a gene encoding a cell-surface protein, such as a cell-surface protein, results in an amino acid substitution in the cell-surface protein epitope. In some embodiments, the amino acid substitution is a non-conservative amino acid substitution. In some embodiments, the cell-surface protein epitope is bound by an agent (e.g., a ligand or an antibody), and the amino acid substitution results in a reduction of the binding activity of the agent (e.g., a ligand or an antibody) to the epitope comprising the amino acid substitution as compared to the unmodified epitope.

The binding activity of the agent (e.g., a ligand or an antibody) to the epitope may be reduced by the amino acid substitution by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 100% or more, e.g., as compared to the unmodified epitope (e.g., a wild-type protein). In some embodiments, the binding activity of the agent (e.g., a ligand or an antibody) to the epitope may be reduced by the amino acid substitution by at least about at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, or at least about 100-fold, e.g., as compared to the unmodified epitope (e.g., a wild-type protein). In some embodiments, the binding is reduced such that there is substantially no detectable binding in a conventional assay. The binding affinity or binding specificity for an epitope or protein can be determined by a variety of methods including equilibrium dialysis, equilibrium binding, gel filtration, ELISA, surface plasmon resonance, or spectroscopy.

In some embodiments, the reduction in binding activity may include an increase in K_{D}, IC₅₀, and/or EC₅₀. For example, the K_{D}, IC₅₀, and/or EC₅₀ may be increased by the amino acid substitution by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 100% or more, e.g., as compared to the unmodified epitope (e.g., a wild-type protein). In some embodiments, the K_{D}, IC₅₀, and/or EC₅₀ may be increased by the amino acid substitution by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, or at least about 100-fold, e.g., as compared to the unmodified epitope (e.g., a wild-type protein).

As used herein, the term “IC₅₀” refers to the concentration of an agent (e.g., an inhibitor, such as a ligand or an antibody) that produces 50% of the maximal inhibition of activity or expression measurable using the same assay in the absence of the binding partner. The IC₅₀ can be as measured in vitro or in vivo. The IC₅₀ can be determined by measuring activity using a conventional in vitro assay (e.g., a protein activity assay and/or a gene expression assay).

As used herein, the term “EC₅₀,” refers to the concentration of a binding partner (e.g., an activator, such as a ligand or an antibody) that produces 50% of maximal activation of measurable activity or expression using the same assay in the absence of the agent. Stated differently, the “EC₅₀” is the concentration of agent that gives 50% activation, when 100% activation is set at the amount of activity that does not increase with the addition of more agent. The EC₅₀ may refer to the half maximal effective concentration, which includes the concentration of an antibody which induces a response halfway between the baseline and maximum after a specified exposure time. The EC₅₀ may represent the concentration of an antibody where 50% of its maximal effect is observed. In certain embodiments, the EC₅₀ value may equal the concentration of an agent (e.g., a ligand or an antibody) that gives half-maximal binding to cells expressing a cell-surface protein, such as a cell-surface protein, as determined by, e.g. a FACS binding assay. Thus, reduced or weaker binding is observed with an increased EC₅₀ value, or half maximal effective concentration value such that 500 nM EC₅₀ is indicative of a weaker binding affinity than 50 nM EC₅₀. The EC₅₀ can be as measured in vitro or in vivo.

The term “K_{D}” refers to the dissociation equilibrium constant of a particular agent-protein interaction, such as an antibody-antigen interaction or a ligand-receptor interaction, or the dissociation rate constant of an antibody or antibody-binding fragment. There is an inverse relationship between K_{D} and binding affinity, therefore the smaller the K_{D} value, the higher, i.e. stronger, the affinity. Thus, the terms “higher affinity” or “stronger affinity” relate to a higher ability to form an interaction and therefore a smaller K_{D} value, and conversely the terms “lower affinity” or “weaker affinity” relate to a lower ability to form an interaction and therefore a larger K_{D} value. In some circumstances, a higher binding affinity (or K_{D}) of a particular molecule (e.g., antibody) to its interactive partner molecule (e.g., antigen X) compared to the binding affinity of the molecule (e.g., antibody) to another interactive partner molecule (e.g. antigen Y) may be expressed as a binding ratio determined by dividing the larger K_{D} value (lower, or weaker, affinity) by the smaller K_{D} (higher, or stronger, affinity), for example expressed as 5-fold or 10-fold greater binding affinity, as the case may be.

In some embodiments, “low affinity” refers to less strong binding interaction. In some embodiments, the low binding affinity corresponds to greater than about 1 nM K_{D}, greater than about 1 nM, about 2 nM, about 3 nM, about 4 nM, about 5 nM, about 6 nM, about 7 nM, about 8 nM, about 9 nM, about 10 nM, about 11 nM, about 12 nM, about 13 nM, about 14 nM, about 15 nM, about 16 nM, about 17 nM, about 18 nM, about 19 nM, about 20 nM, about 21 nM, about 22 nM, about 23 nM, about 24 nM, about 25 nM, about 26 nM, about 27 nM, about 28 nM, about 29 nM, about 30 nM, about 31 nM, about 32 nM, about 33 nM, about 34 nM, about 35 nM, about 36 nM, about 37 nM, about 38 nM, about 39 nM, or about 40 nM K_{D}, wherein such K_{D} binding affinity value is measured, e.g., in an in vitro surface plasmon resonance binding assay, or equivalent biomolecular interaction sensing assay. In some embodiments, the low binding affinity corresponds to greater than about 10 nM, about 11 nM, about 12 nM, about 13 nM, about 14 nM, about 15 nM, about 16 nM, about 17 nM, about 18 nM, about 19 nM, about 20 nM, about 21 nM, about 22 nM, about 23 nM, about 24 nM, about 25 nM, about 26 nM, about 27 nM, about 28 nM, about 29 nM, about 30 nM, about 31 nM, about 32 nM, about 33 nM, about 34 nM, about 35 nM, about 36 nM, about 37 nM, about 38 nM, about 39 nM, or about 40 nM EC₅₀, wherein such EC₅₀ binding affinity value is measured, e.g., in an in vitro FACS binding assay, or equivalent cell-based binding assay.

In some embodiments, “weak affinity” refers to weak binding interaction. In some embodiments, the weak binding affinity corresponds to greater than about 100 nM K_{D} or EC₅₀, greater than about 200, 300, or greater than about 500 nM K_{D} or EC₅₀, wherein such K_{D} binding affinity value is measured, e.g., in an in vitro surface plasmon resonance binding assay, or equivalent biomolecular interaction sensing assay, and such EC₅₀ binding affinity value is measured, e.g., in an in vitro FACS binding assay, or equivalent cell-based interaction detecting assay to detect monovalent biding. No detectable binding means that the affinity between the two biomolecules, for example, especially between the monovalent antibody binding arm and its target antigen, is beyond the detection limit of the assay being used.

In some embodiments provided herein, a mutation in a gene encoding a cell-surface antigen (e.g., CD33, CD20, CLL-1, CD123, CD38, CD19, CD117, EMR2, and/or CD5) results in a loss of expression of the cell-surface antigen in a cell harboring the mutation/modification. In some embodiments, a mutation to a gene detargetizes the protein produced by the gene. In some embodiments, a detargetized cell-surface antigen protein is not bound by, or is bound at a lower level by, an agent that targets the cell-surface antigen. In some embodiments, a mutation in a gene encoding a cell-surface antigen (e.g., CD33, CD20, CLL-1, CD123, CD38, CD19, CD117, EMR2, and/or CD5) results in the expression of a variant form of the cell-surface antigen that is not bound by an agent, such as an immunotherapeutic agent, targeting the cell-surface antigen, or bound at a significantly lower level than the non-mutated or unmodified cell-surface antigen form encoded by the gene. In some embodiments, a cell harboring a genomic mutation in the cell-surface antigen (e.g., CD33, CD20, CLL-1, CD123, CD38, CD19, CD117, EMR2, and/or CD5) gene as provided herein is not bound by, or is bound at a significantly lower level by an agent, such as an immunotherapeutic agent, that targets the cell-surface antigen, e.g., an anti-CD33 antibody or chimeric antigen receptor (CAR), an anti-CD20 antibody or CAR, an anti-CLL-1 antibody or CAR, an anti-CD123 antibody or CAR, an anti-CD38 antibody or CAR, an anti-CD19 antibody or CAR, an anti-CD117 antibody, an anti-EMR2 antibody, or an anti-CD5 antibody or CAR.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The “targeting domain” of the gRNA is complementary to the “target domain” on the target nucleic acid. The strand of the target nucleic acid comprising the nucleotide sequence complementary to the core domain of the gRNA is referred to herein as the “complementary strand” of the target nucleic acid. The targeting domain mediates targeting of the gRNA-bound Cas nuclease to a target site. Guidance on the selection of targeting domains can be found, e.g., in Fu Y et al, Nat Biotechnol 2014 (doi: 10.1038/nbt.2808) and Sternberg S H et al., Nature 2014 (doi: 10.1038/naturel3011).

The term “base editing” refers to a genome editing technology which includes the use of a base editor, e.g., a nuclease-impaired or partially nuclease impaired Cas enzyme (e.g., RNA-guided CRISPR/Cas nuclease, such as dCas9 or nCas9) fused to a deaminase that targets and deaminates a specific nucleobase, e.g., a cytosine or adenosine nucleobase of a C or A nucleotide, which, via cellular mismatch repair mechanisms, results in a change from a C to a T nucleotide, or a change from an A to a G nucleotide. See, e.g., Komor et al. Nature (2016) 533:420-424; Rees et al. Nat. Rev. Genet. (2018) 19 (12): 770-788; Anzalone et al. Nat. Biotechnol. (2020) 38:824-844.

The term “target domain,” “target site,” or “target sequence” refers to a sequence within a nucleic acid molecule (e.g., a DNA molecule) that is mutated (e.g., deaminated) by a CRISPR/Cas system (e.g. base editor) as described herein. In some embodiments, the target sequence is a polynucleotide (e.g., a genomic DNA molecule), wherein the polynucleotide comprises a coding strand and a complementary strand. In some embodiments, a modified genomic DNA molecule comprises a mutation in a nucleotide sequence at a first target domain. In some embodiments, a modified genomic DNA molecule comprises a mutation in a nucleotide sequence at a second or subsequent target domain. The meaning of a “coding strand” and “complementary strand” is the common meaning of the terms in the art. In some embodiments, the target sequence is a sequence in the genome of a mammal. In some embodiments, the target sequence is a sequence in the genome of a human. The term “target codon” refers to the amino acid codon that is modified by the base editor and converted to a different codon via deamination of a nucleobase. In some embodiments, the target codon is modified in the coding strand. In some embodiments, the target codon is modified in the complementary strand.

### Introduction

The present disclosure is based, at least in part, on the surprising discovery that nucleic acid sequences lacking a suitable PAM sequence can efficiently be targeted by CRISPR/Cas systems using the strategies, compositions, methods, and modalities provided herein. In some aspects, the present disclosure provides gene editing strategies, compositions, methods, and modalities that relate to genetic modification, or editing, of target nucleic acid sequences lacking a suitable PAM sequence for efficient editing by a CRISPR/Cas system (e.g., a base editor). In some embodiments, strategies, compositions, methods, and modalities provided herein relate to the generation of a suitable PAM sequence (also referred to herein as an “artificial PAM” or “artificial PAM sequence”) in proximity of a target sequence lacking such a suitable PAM sequence, which thus renders the target sequence suitable for efficient targeting by a CRISPR/Cas system.

In some embodiments, the generation of such an artificial PAM is effected using CRISPR/Cas systems, e.g., base editing technology or HDR-mediated gene editing technology, and utilizing an existing PAM sequence, or a plurality of existing PAM sequences, in proximity to the target sequence, i.e., not suitable for directly editing the target sequence or introducing a mutation in the target sequence. In some embodiments, the existing PAM sequence used for the gene editing and generating the artificial PAM proximal to the target sequence is itself not suitable for editing the target sequence. In some embodiments, the existing PAM sequence used for the mutation generating the artificial PAM proximal to the target sequence is itself not suitable for effecting a desired mutation within the target sequence.

In some embodiments, the gene mutation generating the artificial PAM comprises a single nucleotide substitution. In some embodiments, the gene mutation generating the artificial PAM consists of a single nucleotide substitution. In some embodiments, the gene mutation generating the artificial PAM comprises a deamination of a nucleobase. In some embodiments, the gene mutation generating the artificial PAM comprises an HDR-meditated repair mechanism. In some embodiments, the gene mutation generating the artificial PAM comprises a single nucleotide substitution. In some embodiments, the gene mutation generates the artificial PAM.

The PAM-generating strategies, modalities, compositions, and methods provided herein are useful for targeting nucleic acid sequences using CRISPR/Cas nucleases that are not directly targetable by conventional CRISPR technology. The PAM-generating strategies, modalities, compositions, and methods provided herein are widely applicable to gene editing approaches, where targeting a sequence lacking a suitable PAM sequence is desirable. In some embodiments, such gene editing approaches relate to therapeutic gene editing, e.g., in the context of the correction of a genetic variant associated with a disease or disorder, or in the context of genetically engineering cells for therapeutic uses. While some such approaches and strategies are described in more detail and exemplified herein, additional approaches and strategies will be apparent to the skilled artisan based on the present disclosure, and the disclosure is not limited in this respect.

In the context of CRISPR-based genome editing, the requirement for a CRISPR/Cas nuclease to recognize a short flanking sequence, referred to as a protospacer-adjacent motif (PAM), reduces targeting by CRISPR/Cas-based technologies and leaves many target sites, e.g., many target sites within the human genome, within the genome of non-human mammals, and within other genomes inaccessible to editing. Some aspects of the present disclosure advantageously provide strategies, modalities, compositions, and methods for editing such inaccessible genome sites via the generation of an artificial PAM. In some embodiments, an artificial PAM is generated by CRISPR/Cas systems within proximity of a previously inaccessible target site.

Some aspects of the present disclosure provide strategies, modalities, compositions, and methods related to the simultaneous or sequential modification of a genomic DNA molecule to generate an artificial protospacer-adjacent motif (PAM). The artificial PAM may be used to modify a target site that lacks a suitable PAM prior to such modification.

Accordingly, some aspects of the present disclosure provide nucleotides, e.g., genomic nucleic acid molecules, comprising an artificial protospacer-adjacent motif (PAM). Methods for generating an artificial PAM are also provided. In addition, some aspects of the present disclosure provide cells, e.g., human or non-human mammalian cells, comprising a nucleic acid molecule, e.g., a genomic nucleic acid molecule, that comprises an artificial PAM.

In some aspects, this disclosure provides genetically engineered cells having an artificial protospacer-adjacent motif (PAM) introduced into their genome via a gene mutation, such as a CRISPR-mediated gene mutation, and optionally, one or more modifications in a target gene. In some aspects, the genetically engineered cells having an artificial protospacer-adjacent motif (PAM) comprise an artificial PAM in a genomic sequence, wherein the artificial PAM comprises a single nucleotide substitution (e.g., a substitution of an adenine (A), a guanine (G), a cytosine (C), or a thymine (T), e.g., a substitution that changes a purine nucleotide to another purine (A↔G), or a pyrimidine nucleotide to another pyrimidine (C↔T), e.g., a substitution in which a single (two ring) purine (A or G) is changed for a (one ring) pyrimidine (T or C), or vice versa) as compared to a homologous, naturally occurring genomic sequence, or as compared to the genomic sequence prior to a gene mutation creating the artificial PAM. In some aspects, the genetically engineered cells having an artificial protospacer-adjacent motif (PAM) comprise an artificial PAM in a genomic sequence, wherein the artificial PAM comprises a single nucleotide substitution at an adenine (A), a guanine (G), a cytosine (C), or a thymine (T) that changes the nucleotide to an adenine (A), a guanine (G), a cytosine (C), or a thymine (T) as compared to a homologous, naturally occurring genomic sequence, or as compared to the genomic sequence prior to a gene mutation creating the artificial PAM. In some aspects, the genetically engineered cells having an artificial protospacer-adjacent motif (PAM) comprise an artificial PAM in a genomic sequence, wherein the artificial PAM comprises a single nucleotide substitution at adenine (A), a cytosine (C), or a thymine (T) that changes the nucleotide to a guanine (T) as compared to a homologous, naturally occurring genomic sequence, or as compared to the genomic sequence prior to a gene mutation creating the artificial PAM. In some embodiments, the artificial PAM is located in a non-coding region of a genomic sequence. In other embodiments, the artificial PAM is located in a protein-coding region of a genomic sequence. In some such embodiments, the single nucleotide substitution that created the PAM does not alter the amino acid sequence of the protein encoded by the protein-coding region of the genomic sequence. In some embodiments, the artificial PAM is introduced into the genome of a cell via a gene mutation, such as a CRISPR-mediated gene mutation.

In some embodiments, genetically engineered cells comprising an artificial PAM are human cells. In some embodiments, genetically engineered cells comprising an artificial PAM are non-human primate cells. In some embodiments, genetically engineered cells comprising an artificial PAM are mammalian cells. In some embodiments, genetically engineered cells comprising an artificial PAM are rodent cells. In some embodiments, genetically engineered cells comprising an artificial PAM are insect cells. In some embodiments, genetically engineered cells comprising an artificial PAM are chordate cells. In some embodiments, genetically engineered cells comprising an artificial PAM are plant cells.

In some embodiments, genetically engineered human cells comprising an artificial PAM are human cells that are useful for further editing via a CRISPR/Cas-based gene mutation utilizing the artificial PAM. In some embodiments, the genetically engineered human cells are useful for gene editing a target sequence utilizing the artificial PAM, wherein the target sequence is of clinical significance, e.g., in that the non-modified form of the target sequence is associated with an undesirable clinical phenomenon or effect, for example, with a disease or disorder, or with a side effect of a clinical intervention, such as, for example, a chemotherapeutic or immunotherapeutic agent. In some embodiments, the a target sequence in proximity of the artificial PAM and comprised in the genome of a cell is associated with a disease or disorder, for example, with a malignancy or a pre-malignancy, or with a hereditary disease or disorder, e.g., a monogenetic disorder.

In some embodiments, the target sequence can be modified utilizing the artificial PAM to inactivate a gene product, e.g., a protein, encoded by the target genomic sequence. In some embodiments, the target sequence can be modified utilizing the artificial PAM to modify a gene product, e.g., a protein, encoded by the target genomic sequence, for example, to modify an epitope of such an encoded protein that is recognized by a binding partner, ligand, or antibody. In some embodiments, the target sequence can be modified utilizing the artificial PAM to modify a gene product, e.g., a protein, encoded by the target genomic sequence, for example, to modify an epitope of such an encoded protein that is recognized by a binding partner, ligand, or antibody, wherein such editing results in a single amino acid substitution, and wherein such editing does not affect the remainder of the amino acid sequence of the encoded protein.

Some aspects of the present disclosure provide genetically engineered cells comprising an artificial PAM. In some embodiments, the genetically engineered cells comprising the artificial PAM are stem cells, for example embryonic stem cells (ESCs), induced pluripotent cell (iPSCs), hematopoietic stem cells, skin stem cells, mesenchymal stem cells, neural stem cells, or epithelial stem cells. In some embodiments, the genetically engineered cells comprising the artificial PAM are progenitor cells, e.g., hematopoietic progenitor cells. In some embodiments, the genetically engineered cells comprising the artificial PAM are differentiated cells. In some embodiments, the genetically engineered cells comprising the artificial PAM are hematopoietic cells, e.g., hematopoietic stem cells, hematopoietic progenitor cells, leukocytes, myeloid cells, or lymphoid cells. In some embodiments, the hematopoietic cells are nucleated blood cells. In some embodiments, the myeloid cells neutrophils, eosinophils, mast cells, basophils, or monocytes. In some embodiments, the monocytes are dendritic cells or macrophages. In some embodiments, the lymphocytes are T cells, B-cells, or natural killer cells (NK cells). In some embodiments, the lymphocytes are helper T cells, memory T cells, cytotoxic T cells, plasma cells, or memory B cells.

Some aspects of this disclosure provide strategies, modalities, methods and compositions for therapeutic use in humans. In some embodiments, such therapeutic use comprises administering a population of genetically engineered cells disclosed herein to a subject. In some embodiments, such therapeutic use comprises administering a CRISPR system described herein, e.g., a ribonucleic acid: protein (RNP) complex comprising a CRISPR/Cas nuclease and a suitable gRNA, or a nucleic acid encoding one or all of the components of a CRISPR/Cas system to a cell, e.g., in vivo, ex vivo, or in vitro. In some embodiments, such therapeutic use further comprises administering a therapeutic agent, e.g., an immunotherapeutic agent, a chemotherapeutic agent, a biologic, or a small molecule therapeutic agent, to a subject in need thereof.

### Compositions

Any of the various components of the CRISPR/Cas systems, e.g., base editing technology or HDR-mediated gene editing technology, or genetically engineered cells described herein can be formulated into a composition, such as a pharmaceutical composition.

Accordingly, in some embodiments, the present disclosure provides a pharmaceutical composition comprising any of the various components of the CRISPR/Cas systems, e.g., base editing technology or HDR-mediated gene editing technology, or genetically engineered cells described herein and a pharmaceutically acceptable carrier.

With respect to pharmaceutical compositions, the pharmaceutically acceptable carrier can be any of those conventionally used and is limited only by chemico-physical considerations, such as solubility and lack of reactivity with the active agent(s), and by the route of administration. Pharmaceutically acceptable carriers described herein, for example, vehicles, adjuvants, excipients, and diluents, are well known to those skilled in the art and are readily available to the public. It is preferred that the pharmaceutically acceptable carrier be one which has no detrimental side effects or toxicity under the conditions of use.

The choice of carrier will be determined in part by the particular CRISPR/Cas system(s), genetically engineered cell(s) or related component(s), as well as by the particular methods of administration used. Accordingly, there are a variety of suitable formulations of the pharmaceutical composition of the invention. Methods for preparing administrable compositions are known or apparent to those skilled in the art and are described in more detail in, for example, Remington: The Science and Practice of Pharmacy, Pharmaceutical Press; 22nd ed. (2012).

Methods of introducing any of the various components of the CRISPR/Cas systems, e.g., base editing technology or HDR-mediated gene editing technology into a cell are known in the art. For example, the CRISPR/Cas systems or a component thereof can be transferred into a cell by physical, chemical, or biological means. Physical methods for introducing the CRISPR/Cas systems or a component thereof into a cell include, without limitation, calcium phosphate precipitation, lipofection, particle bombardment, microinjection, transduction (e.g., lentiviral transduction, retroviral transduction), electroporation (e.g., DNA or RNA electroporation), and the like. Methods for producing cells comprising vectors and/or exogenous nucleic acids are well known in the art. See, for example, Sambrook et al., 2012, MOLECULAR CLONING: A LABORATORY MANUAL, volumes 1-4, Cold Spring Harbor Press, NY). Nucleic acids can be introduced into target cells using commercially available methods which include, for example, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg Germany). Nucleic acids can also be introduced into cells using cationic liposome mediated transfection using lipofection, using polymer encapsulation, using peptide mediated transfection, or using biolistic particle delivery systems, such as “gene guns” (see, for example, Nishikawa, et al. Hum Gene Ther., 12 (8): 861-70 (2001).

Regardless of the method used to introduce any of the various components of the CRISPR/Cas systems, e.g., base editing technology or HDR-mediated gene editing technology, described herein into a cell or otherwise expose a cell to the agents described herein, in order to confirm the presence of the nucleic acids (e.g., modified nucleic acids generated using the CRISPR/Cas systems described herein) in the cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (e.g., ELISAs and Western blots) or by assays described herein to identify agents falling within the scope of the invention. In some embodiments, the methods further involve selecting the cells in which the modified nucleic acids have been generated (and, optionally, expressed) from a population of cells.

It is further contemplated that the CRISPR/Cas systems or genetically engineered cells described herein can be used in methods of treating or preventing a disease, disorder, or condition in a subject. In this regard, in some embodiments, the methods of treating or preventing a disease, disorder, or condition in a subject comprising administering to the subject any of the various components of the CRISPR/Cas systems, e.g., base editing technology or HDR-mediated gene editing technology, or genetically engineered cells, and/or the pharmaceutical compositions described herein in an amount effective to treat or prevent the disease, disorder, or condition in a subject in the subject.

The term “administering” is intended to include routes of administration which allow an agent, such as any of the various components of the CRISPR/Cas systems, e.g., base editing technology or HDR-mediated gene editing technology, or genetically engineered cells described herein, to perform its intended function. Examples of routes of administration for treatment of a subject include, without limitation, oral, inhalation, transdermal, and parenteral routes. Systemic modes of administration may include oral and parenteral routes. Parenteral routes may include, without limitation, intravenous, intrarterial, intraosseous, intramuscular, intradermal, subcutaneous, intranasal and intraperitoneal routes. Local modes of administration may include, without limitation, intrathecal, intraspinal, intra-cerebroventricular, and intraparenchymal.

### CRISPR/Cas Systems

In some embodiments, a genetically engineered cell described herein is made using gene editing technology, also referred to herein as DNA-editing technology, e.g., CRISPR/Cas-based DNA editing as described herein. Exemplary CRISPR/Cas systems are described herein, and additional suitable CRISPR/Cas systems will be apparent to the skilled artisan based on the present disclosure and the knowledge in the art. Exemplary suitable CRISPR/Cas systems include, for example, CRISPR/Cas9 systems, and variants thereof. Exemplary suitable nucleases include CRISPR/Cas nucleases (also referred to as CRISPR/Cas nucleases, Cas nuclease, e.g., Cas9), and CRISPR/Cas nuclease variants, e.g., nuclease-dead variant (dCas), nickases (nCas), and fusion proteins, e.g., nuclease-dead or nickase Cas variant fusions with deaminase domains.

An RNA-guided CRISPR/Cas nuclease or nuclease variant is typically used in combination with a guide RNA (gRNA) configured to direct a CRISPR/Cas system to a target domain to provide an mutation, for example, in some embodiments of this disclosure, to generate an artificial protospacer adjacent motif (PAM) in a double-stranded DNA molecule. In some embodiments, this artificial PAM is generated at a position in the genomic DNA molecule that lacks a suitable PAM sequence. In some embodiments, the introduction of a single amino acid substitution may generate an artificial PAM at a position in the genomic DNA molecule that lacks a suitable PAM sequence. In some embodiments, where the artificial PAM is generated within a protein-encoding sequence, the generation of the artificial PAM results in an amino acid substitution in the protein encoded by the sequence, as compared to the original (non-modified) sequence that does not comprise the artificial PAM. In some embodiments, where the artificial PAM is generated within a protein-encoding sequence, the generation of the artificial PAM results in one or more (e.g., 2, 3, 4, 5, or more) amino acid substitutions in the protein encoded by the sequence, as compared to the original (non-modified) sequence that does not comprise the artificial PAM. In some embodiments, one or more (e.g., 2, 3, 4, 5, or more) modifications, e.g., amino acid substitutions, may be introduced to generate one or more artificial PAM at one or more positions in the genomic DNA molecule that lacks a PAM sequence. In some embodiments, a Cas nuclease (e.g., a nCas9 or dCas9) is used in combination with a first guide RNA (gRNA) configured to direct a first CRISPR/Cas system to a first target domain to provide a first mutation to generate an artificial protospacer adjacent motif (PAM), e.g., within proximity to a second or subsequent target domain that lacks a suitable PAM, in a genomic DNA molecule. In some embodiments, a Cas nuclease (e.g., a nCas9 or dCas9)) is used in combination with two or more (e.g., 2, 3, 4, 5, or more) gRNA configured to direct two or more (e.g., 2, 3, 4, 5, or more) CRISPR/Cas systems to two or more target domains to provide two or more (e.g., 2, 3, 4, 5, or more) mutations to generate two or more (e.g., 2, 3, 4, 5, or more) artificial PAMs, e.g., within proximity to one or more additional target domains that lack a suitable PAM, in a genomic DNA molecule.

In some embodiments, a first mutation, generating the artificial PAM may be obtained using a non-CRISPR/Cas nuclease, e.g., a TAL or TALE nuclease (also sometimes referred to as TALEN), a zinc finger nuclease, a meganuclease, or other sequence-specific nuclease, for example, in the context of an HDR-based gene editing approach. Exemplary suitable DNA-editing enzymes are described herein, and additional suitable DNA-editing enzymes will be apparent to the skilled artisan based on the present disclosure and the knowledge in the art.

In some embodiments, a Cas nuclease is used in combination with a second gRNA configured to direct a second CRISPR/Cas system to a second or subsequent target domain in proximity to the artificial PAM to provide a second mutation, e.g., in a target gene. The second mutation may be in a target gene, such as a cell-surface antigen, that encodes a cell-surface protein epitope. The second mutation may result in an amino acid substitution in the cell-surface protein epitope. The amino acid substitution may be a non-conservative amino acid substitution. In certain embodiments, the cell-surface protein epitope is bound by an agent (e.g., a ligand or an antibody), and the amino acid substitution results in a reduction of the binding activity of the agent (e.g., a ligand or an antibody) to the epitope comprising the amino acid substitution as compared to the unmodified epitope. In some embodiments, a Cas nuclease is used in combination with two or more (e.g., 2, 3, 4, 5, or more) gRNA configured to direct two or more (e.g., 2, 3, 4, 5, or more) CRISPR/Cas systems to two or more (e.g., 2, 3, 4, 5, or more) target domains to provide two or more mutations in two or more target genes. In some embodiments, one or more (e.g., 2, 3, 4, 5, or more) target genes may be modified.

In some embodiments, the Cas nuclease that binds the first gRNA may be any Cas nuclease that recognizes a PAM within proximity to a first target domain, for example, according to any method described herein.

In some embodiments, the Cas nuclease that binds the second gRNA may be any Cas nuclease that recognizes an artificial PAM generated in proximity to a second or subsequent target domain, for example, according to any method described herein.

In some embodiments, a Cas nuclease is used in combination with a cell-surface antigen (e.g., CD33, CD20, CLL-1, CD123, CD38, CD19, CD117, EMR2, and/or CD5) gRNA, e.g., as described herein. Some aspects of this disclosure provide compositions and methods for generating the genetically engineered cells described herein, e.g., genetically engineered cells comprising a modification in their genome that results in a loss of expression of a cell-surface antigen (e.g., CD33, CD20, CLL-1, CD123, CD38, CD19, CD117, EMR2, and/or CD5), loss of surface localization of the antigen, or expression of a variant form of the cell-surface antigen that is not recognized by an agent, such as an immunotherapeutic agent, targeting the cell-surface antigen. A variant form of the cell-surface antigen may comprise an amino acid substitution in a cell-surface epitope. Such compositions and methods provided herein include, without limitation, suitable strategies and approaches for genetically engineering cells, e.g., by using nucleases, such as CRISPR/Cas nucleases, and suitable RNAs able to bind such nucleases and target them to a suitable target site within the genome of a cell to effect a genomic modification resulting in a loss of expression of the cell-surface antigen, loss of surface localization of the antigen, or expression of a variant form of the cell-surface antigen that is not recognized by an immunotherapeutic agent targeting the cell-surface antigen (e.g., CD33, CD20, CLL-1, CD123, CD38, CD19, CD117, EMR2, and/or CD5).

In some embodiments, a genetically engineered cell (e.g., a genetically engineered hematopoietic cell, such as, for example, a genetically engineered hematopoietic stem or progenitor cell or a genetically engineered immune effector cell) described herein is generated via genome editing technology, which includes any technology capable of introducing targeted changes, also referred to as “edits,” into the genome of a cell using a nuclease, such as any of the nucleases described herein.

One exemplary suitable genome editing technology is “gene editing,” comprising the use of a CRISPR/Cas nuclease to introduce targeted single- or double-stranded DNA breaks in the genome of a cell, which trigger cellular repair mechanisms, such as, for example, nonhomologous end joining (NHEJ), microhomology-mediated end joining (MMEJ, also sometimes referred to as “alternative NHEJ” or “alt-NHEJ”), or homology-directed repair (HDR) that typically result in an altered nucleic acid sequence (e.g., via nucleotide or nucleotide sequence insertion, deletion, inversion, or substitution) at or immediately proximal to the site of the nuclease cut. See, Yeh et al. Nat. Cell. Biol. (2019) 21:1468-1478; e.g., Hsu et al. Cell (2014) 157:1262-1278; Jasin et al. DNA Repair (2016) 44:6-16; Sfeir et al. Trends Biochem. Sci. (2015) 40:701-714.

Another exemplary suitable genome editing technology is “base editing,” which includes the use of a base editor, e.g., a nuclease-impaired or partially nuclease impaired enzyme (e.g., RNA-guided CRISPR/Cas protein) fused to a deaminase that targets and deaminates a specific nucleobase, e.g., a cytosine or adenosine nucleobase of a C or A nucleotide, which, via cellular mismatch repair mechanisms, results in a change from a C to a T nucleotide, or a change from an A to a G nucleotide. See, e.g., Komor et al. Nature (2016) 533:420-424; Rees et al. Nat. Rev. Genet. (2018) 19 (12): 770-788; Anzalone et al. Nat. Biotechnol. (2020) 38:824-844.

Yet another exemplary suitable genome editing technology includes “prime editing,” which includes the introduction of new genetic information, e.g., an altered nucleotide sequence, into a specifically targeted genomic site using a catalytically impaired or partially catalytically impaired nuclease (e.g., a CRISPR/Cas nuclease), fused to an engineered reverse transcriptase (RT) domain. The Cas/RT fusion is targeted to a target site within the genome by a guide RNA that also comprises a nucleic acid sequence encoding the desired edit, and that can serve as a primer for the RT. See, e.g., Anzalone et al. Nature (2019) 576 (7785): 149-157.

### CRISPR/Cas Nucleases

In some embodiments, use of genome editing technology features the use of a suitable Cas nuclease, which, in some embodiments, e.g., for base editing or prime editing, may be catalytically impaired, or partially catalytically impaired. Examples of suitable Cas nucleases include, but are not limited to, Cas9 or other Cas nucleases, such as Cas12a (Cpf1) or Cas12b.

In some embodiments, a gRNA targeting a gene of interest (e.g. a cell-surface antigen) described herein is complexed with a Cas9 nuclease. Various Cas9 nuclease orthologs or variants can be used. In some embodiments, a Cas9 nuclease is selected that has the desired PAM specificity to target the gRNA/Cas9 nuclease complex to the target domain in the gene of interest (e.g., cell-surface antigen). In some embodiments, genetically engineering a cell also comprises introducing one or more (e.g., 1, 2, 3 or more) Cas9 nucleases into the cell.

In some embodiments, an artificial PAM gRNA described herein is complexed with a Cas9 nuclease. Various Cas9 nucleases can be used. In some embodiments, a Cas9 nuclease is selected that has the desired PAM specificity to target the gRNA/Cas9 nuclease complex to the target domain for generating an artificial PAM. In some embodiments, genetically engineering a cell also comprises introducing one or more (e.g., 1, 2, 3 or more) Cas9 nucleases into the cell.

In exemplary embodiments, a CD33 gRNA described herein is complexed with a Cas9 nuclease. Various Cas9 nucleases can be used. In some embodiments, a Cas9 nuclease is selected that has the desired PAM specificity to target the gRNA/Cas9 nuclease complex to the target domain in CD33. In some embodiments, genetically engineering a cell also comprises introducing one or more (e.g., 1, 2, 3 or more) Cas9 nucleases into the cell.

In exemplary embodiments, a CD20 gRNA described herein is complexed with a Cas9 nuclease. Various Cas9 nucleases can be used. In some embodiments, a Cas9 nuclease is selected that has the desired PAM specificity to target the gRNA/Cas9 nuclease complex to the target domain in CD20. In some embodiments, genetically engineering a cell also comprises introducing one or more (e.g., 1, 2, 3 or more) Cas9 nucleases into the cell.

In exemplary embodiments, a CD38 gRNA described herein is complexed with a Cas9 nuclease. Various Cas9 nucleases can be used. In some embodiments, a Cas9 nuclease is selected that has the desired PAM specificity to target the gRNA/Cas9 nuclease complex to the target domain in CD38. In some embodiments, genetically engineering a cell also comprises introducing one or more (e.g., 1, 2, 3 or more) Cas9 nucleases into the cell.

In exemplary embodiments, a CD123 gRNA described herein is complexed with a Cas9 nuclease. Various Cas9 nucleases can be used. In some embodiments, a Cas9 nuclease is selected that has the desired PAM specificity to target the gRNA/Cas9 nuclease complex to the target domain in CD123. In some embodiments, genetically engineering a cell also comprises introducing one or more (e.g., 1, 2, 3 or more) Cas9 nucleases into the cell.

Cas9 nucleases of a variety of species can be used in the methods and compositions described herein. In various embodiments, the Cas9 nuclease is of, or derived from, Streptococcus pyogenes (SpCas9), Staphylococcus aureus (SaCas9), or Streptococcus thermophilus (StCas9). Additional suitable Cas9 nucleases include those of, or derived from, Staphylococcus aureus, Neisseria meningitidis (NmCas9), Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni (CjCas9), Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In some embodiments, catalytically impaired, or partially impaired, variants of such Cas9 nucleases may be used. Additional suitable Cas9 nucleases, and nuclease variants, will be apparent to those of skill in the art based on the present disclosure. The disclosure is not limited in this respect.

In some embodiments, the Cas9 nuclease is a naturally occurring Cas9 nuclease. In some embodiments, the Cas9 nuclease is an engineered, altered, or modified Cas9 nuclease that differs, e.g., by at least one amino acid residue, from a reference sequence, e.g., the most similar naturally occurring Cas9 nuclease or a sequence of Table 50 of International Publication No. WO 2015/157070, which is herein incorporated by reference in its entirety.

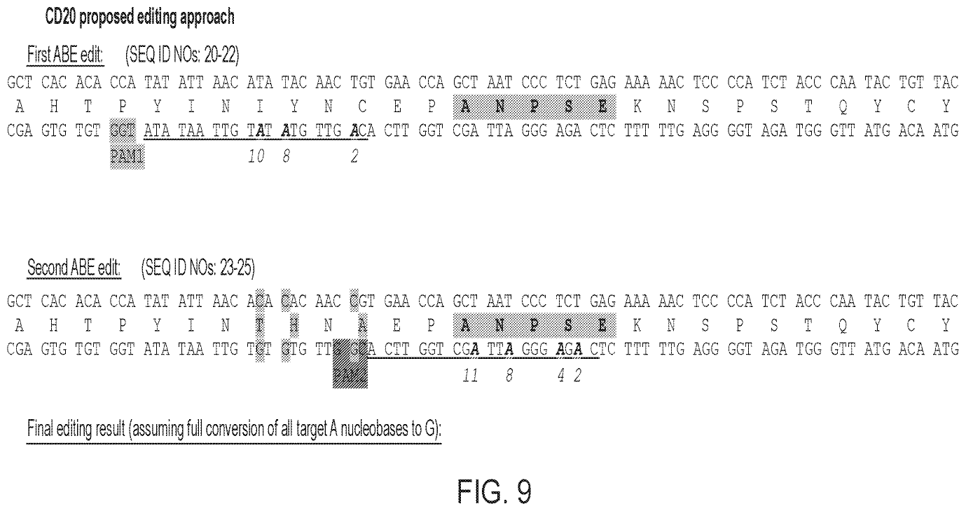

In some embodiments, the Cas nuclease comprises Cpf1 or a fragment or variant thereof. A naturally occurring Cas9 nuclease typically comprises two lobes: a recognition (REC) lobe and a nuclease (NUC) lobe; each of which further comprises domains described, e.g., in International Publication No. WO 2015/157070, e.g., in FIGS. 9A-9B therein (which application is incorporated herein by reference in its entirety).

The REC lobe comprises the arginine-rich bridge helix (BH), the REC1 domain, and the REC2 domain. The REC lobe appears to be a Cas9-specific functional domain. The BH domain is a long alpha helix and arginine rich region and comprises amino acids 60-93 of the sequence of S. pyogenes Cas9. The REC1 domain is involved in recognition of the repeat: anti-repeat duplex, e.g., of a gRNA or a tracrRNA. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat: anti-repeat duplex. The REC2 domain comprises amino acids 180-307 of the sequence of S. pyogenes Cas9.

The NUC lobe comprises the RuvC domain (also referred to herein as RuvC-like domain), the HNH domain (also referred to herein as HNH-like domain), and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule, and comprises amino acids 1099-1368 of the sequence of S. pyogenes Cas9.

Crystal structures have been determined for naturally occurring bacterial Cas9 nucleases (Jinek et al., Science, 343 (6176): 1247997, 2014) and for S. pyogenes Cas9 with a guide RNA (e.g., a synthetic fusion of crRNA and tracrRNA) (Nishimasu et al., Cell, 156:935-949, 2014; and Anders et al., Nature, 2014, doi: 10.1038/naturel3579).

In some embodiments, a Cas nuclease described herein has nuclease activity, e.g., double strand break activity in or directly proximal to a target site. In some embodiments, the Cas9 nuclease has been modified to inactivate one of the catalytic residues of the nuclease. In some embodiments, the Cas9 nuclease is a nickase and produces a single stranded break. See, e.g., Dabrowska et al. Frontiers in Neuroscience (2018) 12 (75). It has been shown that one or more mutations in the RuvC and HNH catalytic domains of the enzyme may improve Cas9 efficiency. See, e.g., Sarai et al. Currently Pharma. Biotechnol. (2017) 18 (13). In some embodiments, the Cas9 nuclease is fused to a second domain, e.g., a domain that modifies DNA or chromatin, e.g., a deaminase or demethylase domain. In some such embodiments, the Cas9 nuclease is modified to eliminate its nuclease activity.

In some embodiments, a Cas nuclease (e.g., a Cas9 nuclease or a Cas9/gRNA complex) described herein is administered together with a template for homology directed repair (HDR). In some embodiments, a Cas nuclease described herein is administered without a HDR template.

In some embodiments, the Cas nuclease is modified to enhance specificity of the enzyme (e.g., reduce off-target effects, maintain robust on-target cleavage). In some embodiments, the Cas9 nuclease is an enhanced specificity Cas9 variant (e.g., eSPCas9). See, e.g., Slaymaker et al. Science (2016) 351 (6268): 84-88. In some embodiments, the Cas9 nuclease is a high fidelity Cas9 variant (e.g., SpCas9-HF1). See, e.g., Kleinstiver et al. Nature (2016) 529:490-495.

Various Cas9 nucleases are known in the art and may be obtained from various sources and/or engineered/modified to modulate one or more activities or specificities of the enzymes. In some embodiments, the Cas9 nuclease has been engineered/modified to recognize one or more PAM sequence. In some embodiments, the Cas9 nuclease has been engineered/modified to recognize one or more PAM sequence that is different than the PAM sequence the Cas9 nuclease recognizes without engineering/modification. In some embodiments, the Cas9 nuclease has been engineered/modified to reduce off-target activity of the enzyme.

In some embodiments, the nucleotide sequence encoding the Cas9 nuclease is modified further to alter the specificity of the nuclease activity (e.g., reduce off-target cleavage, decrease the nuclease activity or lifetime in cells, increase homology-directed recombination and reduce non-homologous end joining). See, e.g., Komor et al. Cell (2017) 168:20-36. In some embodiments, the nucleotide sequence encoding the Cas9 nuclease is modified to alter the PAM recognition of the nuclease. For example, the Cas9 nuclease SpCas9 recognizes PAM sequence NGG, whereas relaxed variants of the SpCas9 comprising one or more modifications of the nuclease (e.g., VQR SpCas9, EQR SpCas9, VRER SpCas9) may recognize the PAM sequences NGA, NGAG, NGCG. PAM recognition of a modified Cas9 nuclease is considered “relaxed” if the Cas9 nuclease recognizes more potential PAM sequences as compared to the Cas9 nuclease that has not been modified. For example, the Cas9 nuclease SaCas9 recognizes PAM sequence NNGRRT, whereas a relaxed variant of the SaCas9 comprising one or more modifications (e.g., KKH SaCas9) may recognize the PAM sequence NNNRRT. In one example, the Cas9 nuclease FnCas9 recognizes PAM sequence NNG, whereas a relaxed variant of the FnCas9 comprising one or more modifications of the nuclease (e.g., RHA FnCas9) may recognize the PAM sequence YG. In one example, the Cas nuclease is a Cpf1 nuclease comprising substitution mutations S542R and K607R and recognize the PAM sequence TYCV. In one example, the Cas nuclease is a Cpf1 nuclease comprising substitution mutations S542R, K607R, and N552R and recognize the PAM sequence TATV. See, e.g., Gao et al. Nat. Biotechnol. (2017) 35 (8): 789-792.

In some embodiments, a Cas nuclease recognizes a PAM and/or an artificial PAM selected from: a SpyCas9 PAM, a SpGCas9 PAM, a SpRYCas9 PAM, a Sth1Cas9 PAM, a SauCas9 PAM, a NmeCas9 PAM, a RspCas9 PAM, a Cca1Cas9 PAM, a PspCas9 PAM, a OrhCas9 PAM, a ScCas9 PAM, a SmacCas9 PAM, a TdeCas9 PAM, a Nme2Cas9 PAM, a CjeCas9 PAM, a SmuCas9 PAM, a Smu2Cas9 PAM, a PmuCas9 PAM, a SpaCas9 PAM, a NciCas9 PAM, a ClaCas9 PAM, a PlaCas9 PAM, a CdCas9 PAM, a IgnaviCas9 PAM, a ThermoCas9 PAM, a GeoCas9 PAM, a G. LC300 Cas9 PAM, a AceCas9 PAM, a Sth3Cas9 PAM, a BlatCas9 PAM, a FnCas9 PAM, a LfeCas9 PAM, a LpnCas9 PAM, a KhuCas9 PAM, a AinCas9 PAM, a CglCas9 PAM, a Esp1Cas9 PAM, a Esp2Cas9 PAM, a FmaCas9 PAM, a LceCas9 PAM, a LrhCas9 PAM, a Lsp1Cas9 PAM, a Lsp2Cas9 PAM, a PacCas9 PAM, a TbaCas9 PAM, a TpuCas9 PAM, a VpaCas9 PAM, a EfaCas9 PAM, a EitCas9 PAM, a LanCas9 PAM, a LmoCas9 PAM, a Sag1Cas9 PAM, a Sag2Cas9 PAM, a SdyCas9 PAM, a Seq1Cas9 PAM, a Seq2Cas9 PAM, a SgaCas9 PAM, a Smu3Cas9 PAM, a SraCas9 PAM, a BniCas9 PAM, a EceCas9 PAM, a EdoCas9 PAM, a FhoCas9 PAM, a MgaCas9 PAM, a MseCas9 PAM, a SgoCas9 PAM, a SmaCas9 PAM, a SsaCas9 PAM, a SsiCas9 PAM, a SsuCas9 PAM, a Sth1ACas9 PAM, a TspCas9 PAM, a BokCas9 PAM, a CcoCas9 PAM, a CpeCas9 PAM, a DdeCas9 PAM, a Ghc2Cas9 PAM, a Ghy3Cas9 PAM, a Ghy4Cas9 PAM, a GspCas9 PAM, a KkiCas9 PAM, a NspCas9 PAM, a TmoCas9 PAM, a NsaCas9 PAM, a JpaCas9 PAM, a BboCas9 PAM, a Cca2Cas9 PAM, a Cme2Cas9 PAM, a Cme3Cas9 PAM, a Cme4Cas9 PAM, a CsaCas9 PAM, a Ghc1Cas9 PAM, a GheCas9 PAM, a Ghh1Cas9 PAM, a Ghh2Cas9 PAM, a Ghy1Cas9 PAM, a MscCas9 PAM, a SdoCas9 PAM, a SpacCas9 PAM, a CgaCas9 PAM, a CmelCas9 PAM, a FfrCas9 PAM, a Ghy2Cas9 PAM, a PhiCas9 PAM, a WviCas9 PAM, a CcCas9 PAM, a FnCas12a PAM, a AsCas12a PAM, a HkCas12a PAM, a PiCas12a PAM, a PdCas12a PAM, a LbCas12a PAM, a Lb2Cas12a or Lb5Cas12a PAM, a CmtCas12a PAM, a MbCas12a PAM, a TsCas12a PAM, a Pb2Cas12a PAM, a MICas12a PAM, a Mb2Cas12a PAM, a Mb3Cas12a PAM, a CmaCas12a PAM, a BsCas12a PAM, a BfCas12a PAM, a BoCas12a PAM, a Adurb193Cas12a PAM, a Adurb336Cas12a PAM, a Fn3Cas12a PAM, a Lb6Cas12a PAM, a EcCas12a PAM, a PsCas12a PAM, a McCas12a PAM, a AacCas12b PAM, a BthCas12b PAM, a AkCas12b PAM, a Ebcas 12b PAM, a bvCas12b PAM, a BhCas12b PAM, a LsCas12b PAM, a BrCas12b PAM, a Cas12c1 PAM, a Cas12c2 PAM, a OspCas12c PAM, a Cas12d.15 PAM, a Cas12d.1 (CasY.1) PAM, a DpbCas12e (DpbCasX) PAM, a PlmCas12e (PlmCasX) PAM, a MilCas12f2 PAM, a Un1Cas12f1 PAM, a Un2Cas12f1 PAM, a Mi2Cas12f2 PAM, a AuCas12f2 PAM, a PtCas12f1 PAM, a AsCas12f1 PAM, a RuCas12f1 PAM, a SpCas12f1 PAM, a CnCas12f1 PAM, a Cas12h1 PAM, a Cas12i1 PAM, a Cas1212 PAM, a Cas12j-1 (CasPhi-1) PAM, a Cas12j-2 (CasPhi-2) PAM, a Cas12j-3 (CasPhi-3) PAM, a ShCas12k PAM, or a AcCas12k PAM.

In some embodiments, a Cas nuclease recognizes a PAM, and/or an artificial PAM comprises, and/or consists of a nucleotide sequence selected from: NGG; NNAGAAW; NNGRRT; NNNNGATT; NVNDCCY; BRTTTTT; NR (A or G) TTTT; NNAAAR (G or A); N (N or A) G; NAAN; NAAAAY; NHDTCCA; NNNVRYM; NNNNRYAC; NAA; GNNNNCNNA; NNGTGA; NNNNGTA; NNGGG; NNNCAT; NNRHHHY; NRRNAT; NNNNCNAA; NNNNCMCA; NNNNCRAA; NNNNGMAA; NNNCC; NGGNG; NNNNCNDD; NYAAA; NRGNN; N (C or D) GGN (T or A or G or C) NN; NRTAW; N (C or K or A) AARC; NAAAG; NV (A or G or C) R (A or G) ACCN; NNGAC; NATGNT; N (T or V) NTAAW (A or T); NNGW (A or T) AY (T or C); NCAA (H (Y or A) B (Y or G); NH (T or C or A) AAAA; NNNATTT; NATAWN (A or T or S); NATARCH; B (T or G or C) GGD (A or T or G) TNN; N (G or T or M) GGAH (T or A or C) N (A or C or K) N; NRG; N (B or A) GG; NGGD (A or K) W (T or A); N (T or C or R) AGAN (A or K or C) NN; NGGD (A or T or G) H (T or M); NGGDT; NGGD (A or T or G) GNN; NNGTAM (A or C) Y; NNGH (W or C) AAA; NTGAR (G or A) N (A or Y or G) N (Y or R); NNGAAAN; NNGAD; NHARMC; NNAAAG; NHGYNAN (A or B); NNAGAAA; NHAAAAA; NH (T or M) AAAAA; NHGYRAA; NNAAACN; NN (H or G) D (A or K) GGDN (A or B); NNNNCTA; NNNNCVGAA; NNNNGYAA; NNNNATN (W or S) ANN; NNWHR (G or A) TA (not G) AA; YHHNGTH; NNNNCDAANN; NNNNCTAA; N (C or D) NNTCCN; NNNNCCAA; NAGRGN (T or V) N (T or C); NNAH (T or M) I; CN (C or W or G) AV (A or S) GAC; NAR (G or A) H (W or C) H (A or T or C) GN (C or T or R); NAGNGC; NATCCTN; NGTGANN; HGCNGCR; NAR (A or G) W (T or A) AC; N (C or D) M (A or C) RN (A or B) AY (C or T); NNNCAC; BGGGTCD; NNRRCC; NRRNTT; KARDAT; BRRTTTW; NARNCCN; NAR (A or G) TC; NAAN (A or T or S) RCN; HHAAATD; NNNNGNA; TTV; TTTV; YYV; KKYV; TTTM; TTYV; TTTN; TTTTA; TTN; BTTV; YTV; YTN; NYTV; DTTD; ATTN; RTTNT; HATT; ATTW; RTTN; TVT; TG; TN; TR; TA; TTCN; TTAT; TTTR; TTR; YTTR; YTTN; CTT; TTC; CCD; RTR; VTTR; TBN; VTTN; NGTT; CGTT; AGG; CGG; GTT, or RGTG, wherein “N” is any nucleotide or base, “W” is adenine (A) or thymine (T), “R” is A or guanine (G), “V” is A, cytosine (C), or G, “Y” is C or T, and “H” is A, C, or T.

In some embodiments, more than one (e.g., 2, 3, or more) Cas nucleases are used. In some embodiments, at least one of the Cas nuclease is a Cas9. In some embodiments, at least one of the Cas nucleases is a Cpf1 enzyme. In some embodiments, at least one of the Cas9 nuclease is derived from Streptococcus pyogenes. In some embodiments, at least one of the Cas9 nuclease is derived from Streptococcus pyogenes and at least one Cas9 nuclease is derived from an organism that is not Streptococcus pyogenes.

In some embodiments, the Cas nuclease comprises a base editor. In some embodiments, a base editor is used to a create a genomic modification resulting in an artificial PAM. In some embodiments, a base editor is used to a create a genomic modification resulting in a loss of expression of a protein antigen of interest (e.g., a cell-surface antigen), or in expression of a protein antigen variant not targeted by an immunotherapy. A base editor nuclease generally comprises a catalytically inactive Cas9 nuclease fused to a functional domain, e.g., a deaminase domain. See, e.g., Eid et al. Biochem. J. (2018) 475 (11): 1955-1964; Rees et al. Nature Reviews Genetics (2018) 19:770-788. In some embodiments, the catalytically inactive Cas9 nuclease is referred to as “dead Cas” or “dCas9.” In some embodiments, the catalytically inactive Cas nuclease has reduced nuclease activity and is, e.g., a nickase (referred to as “nCas”). In some embodiments, the nuclease comprises a dCas9 fused to one or more uracil glycosylase inhibitor (UGI) domains. In some embodiments, the nuclease comprises a dCas9 fused to an adenine base editor (ABE), for example an ABE evolved from the RNA adenine deaminase TadA. In some embodiments, the nuclease comprises a dCas9 fused to cytidine deaminase enzyme (e.g., APOBEC deaminase, pmCDA1, activation-induced cytidine deaminase (AID)). In some embodiments, the catalytically inactive Cas9 nuclease has reduced activity and is nCas9. In some embodiments, the catalytically inactive Cas9 nuclease (dCas9) is fused to one or more uracil glycosylase inhibitor (UGI) domains. In some embodiments, the Cas9 nuclease comprises an inactive Cas9 nuclease (dCas9) fused to an adenine base editor (ABE), for example an ABE evolved from the RNA adenine deaminase TadA. In some embodiments, the Cas9 nuclease comprises a nCas9 fused to an adenine base editor (ABE), for example an ABE evolved from the RNA adenine deaminase TadA. In some embodiments, the Cas9 nuclease comprises a dCas9 fused to cytidine deaminase enzyme (e.g., APOBEC deaminase, pmCDA1, activation-induced cytidine deaminase (AID)). In some embodiments, the Cas9 nuclease comprises a nCas9 fused to cytidine deaminase enzyme (e.g., APOBEC deaminase, pmCDA1, activation-induced cytidine deaminase (AID)).

Examples of base editors include, without limitation, BE1, BE2, BE3, HF-BE3, BE4, BE4max, BE4-Gam, YE1-BE3, EE-BE3, YE2-BE3, YEE-CE3, VQR-BE3, VRER-BE3, SaBE3, SaBE4, SaBE4-Gam, Sa (KKH)-BE3, Target-AID, Target-AID-NG, xBE3, eA3A-BE3, BE-PLUS, TAM, CRISPR-X, ABE7.9, ABE7.10, ABE7.10*, ABE8, ABE8e, ABE8.20-m, xABE, ABESa, VQR-ABE, VRER-ABE, Sa (KKH)-ABE, and CRISPR-SKIP. Additional examples of base editors can be found, for example, in US Publication No. 2018/0312825A1, US Publication No. 2018/0312828A1, International Publication No. WO 2018/165629A1, International Publiation No. WO 2021/030666A1, Internation Publication No. WO 2022/261509A1, Internation Publication No. WO 2021/158921A1, and Gaudelli et al., Nat Biotechnol. 2020; 38 (7): 892-900, which are incorporated by reference herein in their entireties.

In some embodiments, the base editor has been further modified to inhibit base excision repair at the target site and induce cellular mismatch repair. Any of the Cas9 nucleases described herein may be fused to a Gam domain (bacteriophage Mu protein) to protect the Cas9 nuclease from degradation and exonuclease activity. See, e.g., Eid et al. Biochem. J. (2018) 475 (11): 1955-1964.

In some embodiments, the Cas nuclease belongs to class 2 type V of Cas endonuclease. Class 2 type V Cas endonucleases can be further categorized as type V-A, type V-B, type V-C, and type V-U. See, e.g., Stella et al. Nature Structural & Molecular Biology (2017) 24:882-892. In some embodiments, the Cas nuclease is a type V-A Cas endonuclease, such as a Cpf1 (Cas12a) nuclease. In some embodiments, the Cas9 nuclease is a type V-B Cas endonuclease, such as a C2c1 endonuclease. See, e.g., Shmakov et al. Mol Cell (2015) 60:385-397. In some embodiments, the Cas nuclease is MAD7™. Alternatively or in addition, the Cas9 nuclease is a Cpf1 nuclease or a variant thereof. As will be appreciated by one of skill in the art, the Cpf1 nuclease may also be referred to as Cas12a. See, e.g., Strohkendl et al. Mol. Cell (2018) 71:1-9. In some embodiments, a composition or method described herein involves, or a host cell expresses a Cpf1 nuclease derived from Provetella spp. or Francisella spp., Acidaminococcus sp. (AsCpf1), Lachnospiraceae bacterium (LpCpf1), or Eubacterium rectale. In some embodiments, the nucleotide sequence encoding the Cpf1 nuclease may be codon optimized for expression in a host cell. In some embodiments, the nucleotide sequence encoding the Cpf1 endonuclease is further modified to alter the activity of the protein.

Both naturally occurring and modified variants of CRISPR/Cas nucleases are suitable for use according to aspects of this disclosure. For example, dCas or nickase variants, Cas variants having altered PAM specificities, and Cas variants having improved nuclease activities are embraced by some embodiments of this disclosure. In some embodiments, catalytically inactive variants of Cas nucleases (e.g., of Cas9 or Cas12a) are used according to the methods described herein. A catalytically inactive variant of Cpf1 (Cas12a) may be referred to dCas12a. As described herein, catalytically inactive variants of Cpf1 may be fused to a function domain to form a base editor. See, e.g., Rees et al. Nature Reviews Genetics (2018) 19:770-788. In some embodiments, the catalytically inactive Cas9 nuclease is dCas9. In some embodiments, the nuclease comprises a dCas12a fused to one or more uracil glycosylase inhibitor (UGI) domains. In some embodiments, the Cas9 nuclease comprises a dCas12a fused to an adenine base editor (ABE), for example an ABE evolved from the RNA adenine deaminase TadA. In some embodiments, the Cas nuclease comprises a dCas12a fused to cytidine deaminase enzyme (e.g., APOBEC deaminase, pmCDA1, activation-induced cytidine deaminase (AID)).

Alternatively or in addition, the Cas9 nuclease may be a Cas14 nuclease or variant thereof. Cas14 nucleases are derived from archaea and tend to be smaller in size (e.g., 400-700 amino acids). Additionally Cas14 nucleases do not require a PAM sequence. See, e.g., Harrington et al. Science (2018).

Any of the Cas9 nucleases described herein may be modulated to regulate levels of expression and/or activity of the Cas9 nuclease at a desired time. For example, it may be advantageous to increase levels of expression and/or activity of the Cas9 nuclease during particular phase(s) of the cell cycle. It has been demonstrated that levels of homology-directed repair are reduced during the G1 phase of the cell cycle, therefore increasing levels of expression and/or activity of the Cas9 nuclease during the S phase, G2 phase, and/or M phase may increase homology-directed repair following the Cas endonuclease editing. In some embodiments, levels of expression and/or activity of the Cas9 nuclease are increased during the S phase, G2 phase, and/or M phase of the cell cycle. In one example, the Cas9 nuclease fused to the N-terminal region of human Geminin. See, e.g., Gutschner et al. Cell Rep. (2016) 14 (6): 1555-1566. In some embodiments, levels of expression and/or activity of the Cas9 nuclease are reduced during the G1 phase. In one example, the Cas9 nuclease is modified such that it has reduced activity during the G1 phase. See, e.g., Lomova et al. Stem Cells (2018).

Alternatively or in addition, any of the Cas9 nucleases described herein may be fused to an epigenetic modifier (e.g., a chromatin-modifying enzyme, e.g., DNA methylase, histone deacetylase). See, e.g., Kungulovski et al. Trends Genet. (2016) 32 (2): 101-113. Cas9 nuclease fused to an epigenetic modifier may be referred to as “epieffectors” and may allow for temporal and/or transient endonuclease activity. In some embodiments, the Cas9 nuclease is a dCas9 fused to a chromatin-modifying enzyme.

### Base Editors

In some embodiments, a cell or cell population described herein is produced using base editing technology. As described above, base editing includes the use of a base editor, e.g., a nuclease-impaired or partially nuclease impaired enzyme (e.g., RNA-guided CRISPR/Cas protein) fused to a deaminase that targets and deaminates a specific nucleobase, e.g., a cytosine or adenosine nucleobase of a C or A nucleotide, which, via cellular mismatch repair mechanisms, results in a change from a C to a T nucleotide, or a change from an A to a G nucleotide. See, e.g., Komor et al. Nature (2016) 533:420-424; Rees et al. Nat. Rev. Genet. (2018) 19 (12): 770-788; Anzalone et al. Nat. Biotechnol. (2020) 38:824-844.

Base editing technology, as described herein, can be used for editing a double-stranded DNA molecule to generate an artificial protospacer adjacent motif (PAM) in proximity of a target domain. For example, in some embodiments, a method as described herein, may comprise: (i) providing a cell comprising a genomic DNA molecule, and (ii) introducing into the cell (a) a first guide RNA (gRNA) configured to direct a first CRISPR/Cas system to a first target site to provide a first mutation to generate an artificial PAM in a genomic DNA molecule; (b) a second gRNA configured to direct a second CRISPR/Cas system to a second target site to provide a second mutation; (c) a Cas nuclease that binds the first gRNA; and (d) a Cas nuclease that binds the second gRNA, thereby editing the genomic DNA molecule.

In some embodiments, base editing can be used to modify one or more target gene(s) encoding a protein of interest. In exemplary embodiments, base editing can be used to modify a target gene encoding a cell-surface protein epitope (e.g., CD33, CD20, CLL-1, CD123, CD38, CD19, CD117, EMR2, and/or CD5).

In some embodiments, the CRISPR/Cas system comprises a base editor. In some embodiments, a base editor is used to a create a genomic modification resulting in a loss of expression of a protein antigen (e.g., a cell-surface antigen), loss of surface localization of the protein antigen, or in expression of a protein antigen variant not targeted by an immunotherapy. Such a variant may comprise a modification in a protein epitope. In some embodiments, a base editor is used to a create a genomic modification resulting in a loss of expression of CD33, loss of surface localization of CD33, or in expression of a CD33 variant not targeted by an immunotherapy. In some embodiments, a base editor is used to a create a genomic modification resulting in a loss of expression of CD20, loss of surface localization of CD20, or in expression of a CD20 variant not targeted by an immunotherapy. In some embodiments, a base editor is used to a create a genomic modification resulting in a loss of expression of CLL-1, loss of surface localization of CLL-1, or in expression of a CLL-1 variant not targeted by an immunotherapy. In some embodiments, a base editor is used to a create a genomic modification resulting in a loss of expression of CD123, loss of surface localization of CD123, or in expression of a CD123 variant not targeted by an immunotherapy. In some embodiments, a base editor is used to a create a genomic modification resulting in a loss of expression of CD38, loss of surface localization of CD38, or in expression of a CD38 variant not targeted by an immunotherapy. In some embodiments, a base editor is used to a create a genomic modification resulting in a loss of expression of CD19, loss of surface localization of CD19, or in expression of a CD19 variant not targeted by an immunotherapy. In some embodiments, a base editor is used to a create a genomic modification resulting in a loss of expression of CD117, loss of surface localization of CD117, or in expression of a CD117 variant not targeted by an immunotherapy. In some embodiments, a base editor is used to a create a genomic modification resulting in a loss of expression of EMR2, loss of surface localization of EMR2, or in expression of a EMR2 variant not targeted by an immunotherapy. In some embodiments, a base editor is used to a create a genomic modification resulting in a loss of expression of CD5, loss of surface localization of CD5, or in expression of a CD5 variant not targeted by an immunotherapy.

In some embodiments, a base editor is used to create an mutation (e.g., a create a genomic modification) that reduces the activity of a target protein (e.g., cell-surface protein) in a cell. In some embodiments, a base editor is used to create an mutation (e.g., a create a genomic modification) that reduces the expression level of a nucleic acid encoding a cell-surface antigen (e.g., CD33, CD20, CLL-1, CD123, CD38, CD19, CD117, EMR2, and/or CD5) in a cell.

In some embodiments, a base editor is used to create an mutation (e.g., a create a genomic modification) that abolishes the expression of a full-length mRNA for the protein of interest (e.g., CD33, CD20, CLL-1, CD123, CD38, CD19, CD117, EMR2, and/or CD5) in a cell. In some embodiments, a base editor is used to create a mutation (e.g., a genomic modification) that abolishes the expression of a full-length protein of interest (e.g., CD33, CD20, CLL-1, CD123, CD38, CD19, CD117, EMR2, and/or CD5) in a cell.

In some embodiments, the cell expresses a truncated version of the protein of interest (e.g., CD33, CD20, CLL-1, CD123, CD38, CD19, CD117, EMR2, and/or CD5) RNA. In some embodiments, the cell expresses a truncated version of the protein of interest (e.g., CD33, CD20, CLL-1, CD123, CD38, CD19, CD117, EMR2, and/or CD5) protein.

In some embodiments, the truncated version of the protein of interest (e.g., CD33, CD20, CLL-1, CD123, CD38, CD19, CD117, EMR2, and/or CD5) RNA is expressed at a level equal to or greater than a level of a full-length protein of interest (e.g., CD33, CD20, CLL-1, CD123, CD38, CD19, CD117, EMR2, and/or CD5) RNA in a non-modified cell. In some embodiments, the truncated version of the protein of interest (e.g., CD33, CD20, CLL-1, CD123, CD38, CD19, CD117, EMR2, and/or CD5) protein is expressed at a level equal to or greater than a level of a protein of interest (e.g., CD33, CD20, CLL-1, CD123, CD38, CD19, CD117, EMR2, and/or CD5) protein in a non-modified cell.

In some embodiments, wherein a function or an activity of the truncated version of the protein of interest (e.g., CD33, CD20, CLL-1, CD123, CD38, CD19, CD117, EMR2, and/or CD5) RNA is impaired or abolished. In some embodiments, wherein a function or an activity of the truncated version of the protein of interest (e.g., CD33, CD20, CLL-1, CD123, CD38, CD19, CD117, EMR2, and/or CD5) protein is impaired or abolished. In some embodiments, a function or an activity of the truncated version of the protein of interest (e.g., CD33, CD20, CLL-1, CD123, CD38, CD19, CD117, EMR2, and/or CD5) RNA that is impaired or abolished comprises binding to an antibody or a chimeric antigen receptor (CAR).

Base editor nuclease generally comprises a catalytically inactive Cas9 nuclease fused to a functional domain, e.g., a deaminase domain. See, e.g., Eid et al. Biochem. J. (2018) 475 (11): 1955-1964; Rees et al. Nature Reviews Genetics (2018) 19:770-788. In some embodiments, the catalytically inactive Cas9 nuclease is referred to as “dead Cas” or “dCas9.” In some embodiments, the catalytically inactive Cas nuclease has reduced nuclease activity and is, e.g., a nickase (referred to as “nCas”). In some embodiments, the nuclease comprises a dCas9 fused to one or more uracil glycosylase inhibitor (UGI) domains. In some embodiments, the nuclease comprises a dCas9 fused to an adenine base editor (ABE), for example an ABE evolved from the RNA adenine deaminase TadA. In some embodiments, the nuclease comprises a dCas9 fused to cytidine deaminase enzyme (e.g., APOBEC deaminase, pmCDA1, activation-induced cytidine deaminase (AID)). In some embodiments, the catalytically inactive Cas9 nuclease has reduced activity and is nCas9. In some embodiments, the catalytically inactive Cas9 nuclease (dCas9) is fused to one or more uracil glycosylase inhibitor (UGI) domains. In some embodiments, the Cas9 nuclease comprises an inactive Cas9 nuclease (dCas9) fused to an adenine base editor (ABE), for example an ABE evolved from the RNA adenine deaminase TadA. In some embodiments, the Cas9 nuclease comprises a nCas9 fused to an adenine base editor (ABE), for example an ABE evolved from the RNA adenine deaminase TadA. In some embodiments, the Cas9 nuclease comprises a dCas9 fused to cytidine deaminase enzyme (e.g., APOBEC deaminase, pmCDA1, activation-induced cytidine deaminase (AID)). In some embodiments, the Cas9 nuclease comprises a nCas9 fused to cytidine deaminase enzyme (e.g., APOBEC deaminase, pmCDA1, activation-induced cytidine deaminase (AID)).

In some embodiments, the base editor is a cytosine base editor (CBE). In some embodiments, the CBE is CBE1, CBE2, CBE3, or CBE4. In some embodiments, the CBE is nCas9-2xUGI; BE4-rAPOBEC1; BE4-rAPOBEC1 K34A H122A; BE4-PpAPOBEC1; BE4-PpAPOBEC1 R33A; BE4-PpAPOBEC1 H122A; BE4-RrA3F; BE4-AmAPOBEC1; or BE4-SsAPOBEC3B.

In some embodiments, the base editor is an adenine base editor (ABE). In some embodiments, the ABE is ABE1, ABE2, ABE3, ABE4, ABE5, ABE6, ABE7, or ABE8. In some embodiments, the ABE is ABE7.10-m; ABE7.10-d; ABE8e; ABE8.8-m; ABE8.8-d; ABE8.13-m; ABE8.13-d; ABE8.17-m; ABE8.17-d; ABE8.20-m; or ABE8.20-d.

In some embodiments, the base editors includes, without limitation, BE1, BE2, BE3, HF-BE3, BE4, BE4max, BE4-Gam, YE1-BE3, EE-BE3, YE2-BE3, YEE-CE3, VQR-BE3, VRER-BE3, SaBE3, SaBE4, SaBE4-Gam, Sa (KKH)-BE3, Target-AID, Target-AID-NG, xBE3, eA3A-BE3, BE-PLUS, TAM, CRISPR-X, ABE7.9, ABE7.10, ABE7.10*, xABE, ABESa, VQR-ABE, VRER-ABE, Sa (KKH)-ABE, and CRISPR-SKIP. In preferred embodiments, the base editor is ABE8e or ABE8.20-m.

Additional examples of base editors can be found, for example, in US Publication No. 2018/0312825A₁, US Publication No. 2018/0312828A₁, International Publication No. WO 2018/165629A₁, Yu et al. Nat Commun. (2020) 11 (1): 2052, and Gaudelli et al. Nat Biotechnol. (2020) 38 (7): 892-900. which are incorporated by reference herein in their entireties.

An exemplary abe8_20m sequence is provided below:
(SEQ ID NO: 91)
atgtccgaagtcgagttttcccatgagtactggatgagacacgcattgactctcgcaaagag
ggctcgagatgaacgcgaggtgcccgtgggggcagtactcgtgctcaacaatcgcgtaatcg
gcgaaggttggaatagggcaatcggactccacgaccccactgcacatgcggaaatcatggcc
cttcgacagggagggcttgtgatgcagaattatcgactttatgatgcgacgctgtactcgac
gtttgaaccttgcgtaatgtgcgcgggagctatgattcactcccgcattggacgagttgtat
tcggtgttcgcaacgccaagacgggtgccgcaggttcactgatggacgtgctgcatcatcca
ggcatgaaccaccgggtagaaatcacagaaggcatattggcggacgaatgtgcggcgctgtt
gtgtcgtttttttcgcatgcccaggcgggtctttaacgcccagaaaaaagcacaatcctcta
ctgactctggtggttcttctggtggttctagcggcagcgagactcccgggacctcagagtcc
gccacacccgaaagttctggtggttcttctggtggttctgacaagaagtacagcatcggcct
ggccatcggcaccaactctgtgggctgggccgtgatcaccgacgagtacaaggtgcccagca
agaaattcaaggtgctgggcaacaccgaccggcacagcatcaagaagaacctgatcggagcc
ctgctgttcgacagcggcgaaacagccgaggccacccggctgaagagaaccgccagaagaag
atacaccagacggaagaaccggatctgctatctgcaagagatcttcagcaacgagatggcca
aggtggacgacagcttcttccacagactggaagagtccttcctggtggaagaggataagaag
cacgagcggcaccccatcttcggcaacatcgtggacgaggtggcctaccacgagaagtaccc
caccatctaccacctgagaaagaaactggtggacagcaccgacaaggccgacctgcggctga
tctatctggccctggcccacatgatcaagttccggggccacttcctgatcgagggcgacctg
aaccccgacaacagcgacgtggacaagctgttcatccagctggtgcagacctacaaccagct
gttcgaggaaaaccccatcaacgccagcggcgtggacgccaaggccatcctgtctgccagac
tgagcaagagcagacggctggaaaatctgatcgcccagctgcccggcgagaagaagaatggc
ctgttcggaaacctgattgccctgagcctgggcctgacccccaacttcaagagcaacttcga
cctggccgaggatgccaaactgcagctgagcaaggacacctacgacgacgacctggacaacc
tgctggcccagatcggcgaccagtacgccgacctgtttctggccgccaagaacctgtccgac
gccatcctgctgagcgacatcctgagagtgaacaccgagatcaccaaggcccccctgagcgc
ctctatgatcaagagatacgacgagcaccaccaggacctgaccctgctgaaagctctcgtgc
ggcagcagctgcctgagaagtacaaagagattttcttcgaccagagcaagaacggctacgcc
ggctacattgacggcggagccagccaggaagagttctacaagttcatcaagcccatcctgga
aaagatggacggcaccgaggaactgctcgtgaagctgaacagagaggacctgctgcggaagc
agcggaccttcgacaacggcagcatcccccaccagatccacctgggagagctgcacgccatt
ctgcggcggcaggaagatttttacccattcctgaaggacaaccgggaaaagatcgagaagat
cctgaccttccgcatcccctactacgtgggccctctggccaggggaaacagcagattcgcct
ggatgaccagaaagagcgaggaaaccatcaccccctggaacttcgaggaagtggtggacaag
ggcgcttccgcccagagcttcatcgagcggatgaccaacttcgataagaacctgcccaacga
gaaggtgctgcccaagcacagcctgctgtacgagtacttcaccgtgtataacgagctgacca
aagtgaaatacgtgaccgagggaatgagaaagcccgccttcctgagcggcgagcagaaaaag
gccatcgtggacctgctgttcaagaccaaccggaaagtgaccgtgaagcagctgaaagagga
ctacttcaagaaaatcgagtgcttcgactccgtggaaatctccggcgtggaagatcggttca
acgcctccctgggcacataccacgatctgctgaaaattatcaaggacaaggacttcctggac
aatgaggaaaacgaggacattctggaagatatcgtgctgaccctgacactgtttgaggacag
agagatgatcgaggaacggctgaaaacctatgcccacctgttcgacgacaaagtgatgaagc
agctgaagcggcggagatacaccggctggggcaggctgagccggaagctgatcaacggcatc
cgggacaagcagtccggcaagacaatcctggatttcctgaagtccgacggcttcgccaacag
aaacttcatgcagctgatccacgacgacagcctgacctttaaagaggacatccagaaagccc
aggtgtccggccagggcgatagcctgcacgagcacattgccaatctggccggcagccccgcc
attaagaagggcatcctgcagacagtgaaggtggtggacgagctcgtgaaagtgatgggccg
gcacaagcccgagaacatcgtgatcgaaatggccagagagaaccagaccacccagaagggac
agaagaacagccgcgagagaatgaagcggatcgaagagggcatcaaagagctgggcagccag
atcctgaaagaacaccccgtggaaaacacccagctgcagaacgagaagctgtacctgtacta
cctgcagaatgggcgggatatgtacgtggaccaggaactggacatcaaccggctgtccgact
acgatgtggaccatatcgtgcctcagagctttctgaaggacgactccatcgacaacaaggtg
ctgaccagaagcgacaagaaccggggcaagagcgacaacgtgccctccgaagaggtcgtgaa
gaagatgaagaactactggcggcagctgctgaacgccaagctgattacccagagaaagttcg
acaatctgaccaaggccgagagaggcggcctgagcgaactggataaggccggcttcatcaag
agacagctggtggaaacccggcagatcacaaagcacgtggcacagatcctggactcccggat
gaacactaagtacgacgagaatgacaagctgatccgggaagtgaaagtgatcaccctgaagt
ccaagctggtgtccgatttccggaaggatttccagttttacaaagtgcgcgagatcaacaac
taccaccacgcccacgacgcctacctgaacgccgtcgtgggaaccgccctgatcaaaaagta
ccctaagctggaaagcgagttcgtgtacggcgactacaaggtgtacgacgtgcggaagatga
tcgccaagagcgagcaggaaatcggcaaggctaccgccaagtacttcttctacagcaacatc
atgaactttttcaagaccgagattaccctggccaacggcgagatccggaagcggcctctgat
cgagacaaacggcgaaaccggggagatcgtgtgggataagggccgggattttgccaccgtgc
ggaaagtgctgagcatgccccaagtgaatatcgtgaaaaagaccgaggtgcagacaggcggc
ttcagcaaagagtctatcctgcccaagaggaacagcgataagctgatcgccagaaagaagga
ctgggaccctaagaagtacggcggcttcgacagccccaccgtggcctattctgtgctggtgg
tggccaaagtggaaaagggcaagtccaagaaactgaagagtgtgaaagagctgctggggatc
accatcatggaaagaagcagcttcgagaagaatcccatcgactttctggaagccaagggcta
caaagaagtgaaaaaggacctgatcatcaagctgcctaagtactccctgttcgagctggaaa
acggccggaagagaatgctggcctctgccggcgaactgcagaagggaaacgaactggccctg
ccctccaaatatgtgaacttcctgtacctggccagccactatgagaagctgaagggctcccc
cgaggataatgagcagaaacagctgtttgtggaacagcacaagcactacctggacgagatca
tcgagcagatcagcgagttctccaagagagtgatcctggccgacgctaatctggacaaagtg
ctgtccgcctacaacaagcaccgggataagcccatcagagagcaggccgagaatatcatcca
cctgtttaccctgaccaatctgggagcccctgccgccttcaagtactttgacaccaccatcg
accggaagaggtacaccagcaccaaagaggtgctggacgccaccctgatccaccagagcatc
accggcctgtacgagacacggatcgacctgtctcagctgggaggtgacgagggagctgataa
gcgcaccgccgatggttccgagttcgaaagccccaagaagaagaggaaagtc
An exemplary ppabobec1-orf-r33a sequence is provided below:
(SEQ ID NO: 92)
atgacctctgagaagggccctagcacaggcgaccccaccctgcggcggagaatcgagagctg
ggagttcgacgtgttctacgaccctagagaactggccaaggaaacctgcctgctgtacgaga
tcaagtggggcatgagcagaaagatctggcggagctctggcaagaacaccaccaaccacgtg
gaagtgaatttcatcaagaagttcaccagcgagagaaggttccacagcagcatcagctgcag
catcacctggttcctgagctggtccccttgctgggaatgcagccaggccatcagagagttcc
tgagccaacaccccggagtgacactggtgatctacgtggccagactgttctggcacatggac
cagagaaacagacagggcctgagagatctggtcaacagcggcgtgactatccagatcatgcg
ggccagcgagtactaccactgttggcggaacttcgtgaactacccccccggcgatgaggccc
actggcctcagtaccctcctctgtggatgatgctgtacgccctggaactgcactgcatcatc
ctgtctctgcctccatgtctgaagatctctagaagatggcagaaccacctggccttcttcag
actgcacctgcagaattgccactaccagaccatccccccccacatcctgctggctacaggcc
tgatccacccttctgtgacctggagacttaagagcggaggatctagcggcggctctagcgga
tctgagacacctggcacaagcgagtctgccacacctgagagtagcggcggatcttctggtgg
ctctgacaagaagtacagcatcggcctggccatcggcaccaactctgtgggctgggccgtga
tcaccgacgagtacaaggtgcccagcaagaaattcaaggtgctgggcaacaccgaccggcac
agcatcaagaagaacctgatcggagccctgctgttcgacagcggcgaaacagccgaggccac
ccggctgaagagaaccgccagaagaagatacaccagacggaagaaccggatctgctatctgc
aagagatcttcagcaacgagatggccaaggtggacgacagcttcttccacagactggaagag
tccttcctggtggaagaggataagaagcacgagcggcaccccatcttcggcaacatcgtgga
cgaggtggcctaccacgagaagtaccccaccatctaccacctgagaaagaaactggtggaca
gcaccgacaaggccgacctgcggctgatctatctggccctggcccacatgatcaagttccgg
ggccacttcctgatcgagggcgacctgaaccccgacaacagcgacgtggacaagctgttcat
ccagctggtgcagacctacaaccagctgttcgaggaaaaccccatcaacgccagcggcgtgg
acgccaaggccatcctgtctgccagactgagcaagagcagacggctggaaaatctgatcgcc
cagctgcccggcgagaagaagaatggcctgttcggaaacctgattgccctgagcctgggcct
gacccccaacttcaagagcaacttcgacctggccgaggatgccaaactgcagctgagcaagg
acacctacgacgacgacctggacaacctgctggcccagatcggcgaccagtacgccgacctg
tttctggccgccaagaacctgtccgacgccatcctgctgagcgacatcctgagagtgaacac
cgagatcaccaaggcccccctgagcgcctctatgatcaagagatacgacgagcaccaccagg
acctgaccctgctgaaagctctcgtgcggcagcagctgcctgagaagtacaaagagattttc
ttcgaccagagcaagaacggctacgccggctacattgacggcggagccagccaggaagagtt
ctacaagttcatcaagcccatcctggaaaagatggacggcaccgaggaactgctcgtgaagc
tgaacagagaggacctgctgcggaagcagcggaccttcgacaacggcagcatcccccaccag
atccacctgggagagctgcacgccattctgcggcggcaggaagatttttacccattcctgaa
ggacaaccgggaaaagatcgagaagatcctgaccttccgcatcccctactacgtgggccctc
tggccaggggaaacagcagattcgcctggatgaccagaaagagcgaggaaaccatcaccccc
tggaacttcgaggaagtggtggacaagggcgcttccgcccagagcttcatcgagcggatgac
caacttcgataagaacctgcccaacgagaaggtgctgcccaagcacagcctgctgtacgagt
acttcaccgtgtataacgagctgaccaaagtgaaatacgtgaccgagggaatgagaaagccc
gccttcctgagcggcgagcagaaaaaggccatcgtggacctgctgttcaagaccaaccggaa
agtgaccgtgaagcagctgaaagaggactacttcaagaaaatcgagtgcttcgactccgtgg
aaatctccggcgtggaagatcggttcaacgcctccctgggcacataccacgatctgctgaaa
attatcaaggacaaggacttcctggacaatgaggaaaacgaggacattctggaagatatcgt
gctgaccctgacactgtttgaggacagagagatgatcgaggaacggctgaaaacctatgccc
acctgttcgacgacaaagtgatgaagcagctgaagcggcggagatacaccggctggggcagg
ctgagccggaagctgatcaacggcatccgggacaagcagtccggcaagacaatcctggattt
cctgaagtccgacggcttcgccaacagaaacttcatgcagctgatccacgacgacagcctga
cctttaaagaggacatccagaaagcccaggtgtccggccagggcgatagcctgcacgagcac
attgccaatctggccggcagccccgccattaagaagggcatcctgcagacagtgaaggtggt
ggacgagctcgtgaaagtgatgggccggcacaagcccgagaacatcgtgatcgaaatggcca
gagagaaccagaccacccagaagggacagaagaacagccgcgagagaatgaagcggatcgaa
gagggcatcaaagagctgggcagccagatcctgaaagaacaccccgtggaaaacacccagct
gcagaacgagaagctgtacctgtactacctgcagaatgggcgggatatgtacgtggaccagg
aactggacatcaaccggctgtccgactacgatgtggaccatatcgtgcctcagagctttctg
aaggacgactccatcgacaacaaggtgctgaccagaagcgacaagaaccggggcaagagcga
caacgtgccctccgaagaggtcgtgaagaagatgaagaactactggcggcagctgctgaacg
ccaagctgattacccagagaaagttcgacaatctgaccaaggccgagagaggcggcctgagc
gaactggataaggccggcttcatcaagagacagctggtggaaacccggcagatcacaaagca
cgtggcacagatcctggactcccggatgaacactaagtacgacgagaatgacaagctgatcc
gggaagtgaaagtgatcaccctgaagtccaagctggtgtccgatttccggaaggatttccag
ttttacaaagtgcgcgagatcaacaactaccaccacgcccacgacgcctacctgaacgccgt
cgtgggaaccgccctgatcaaaaagtaccctaagctggaaagcgagttcgtgtacggcgact
acaaggtgtacgacgtgcggaagatgatcgccaagagcgagcaggaaatcggcaaggctacc
gccaagtacttcttctacagcaacatcatgaactttttcaagaccgagattaccctggccaa
cggcgagatccggaagcggcctctgatcgagacaaacggcgaaaccggggagatcgtgtggg
ataagggccgggattttgccaccgtgcggaaagtgctgagcatgccccaagtgaatatcgtg
aaaaagaccgaggtgcagacaggcggcttcagcaaagagtctatcctgcccaagaggaacag
cgataagctgatcgccagaaagaaggactgggaccctaagaagtacggcggcttcgacagcc
ccaccgtggcctattctgtgctggtggtggccaaagtggaaaagggcaagtccaagaaactg
aagagtgtgaaagagctgctggggatcaccatcatggaaagaagcagcttcgagaagaatcc
catcgactttctggaagccaagggctacaaagaagtgaaaaaggacctgatcatcaagctgc
ctaagtactccctgttcgagctggaaaacggccggaagagaatgctggcctctgccggcgaa
ctgcagaagggaaacgaactggccctgccctccaaatatgtgaacttcctgtacctggccag
ccactatgagaagctgaagggctcccccgaggataatgagcagaaacagctgtttgtggaac
agcacaagcactacctggacgagatcatcgagcagatcagcgagttctccaagagagtgatc
ctggccgacgctaatctggacaaagtgctgtccgcctacaacaagcaccgggataagcccat
cagagagcaggccgagaatatcatccacctgtttaccctgaccaatctgggagcccctgccg
ccttcaagtactttgacaccaccatcgaccggaagaggtacaccagcaccaaagaggtgctg
gacgccaccctgatccaccagagcatcaccggcctgtacgagacacggatcgacctgtctca
gctgggaggtgactctggtggaagcggaggatctggcggcagcaccaatctgagcgacatca
tcgagaaagagacaggcaagcagctggtcatccaagagtccatcctgatgctgcctgaagag
gtggaagaagtgatcggcaacaagcccgagtccgacatcctggtgcacaccgcctacgatga
gagcaccgacgagaacgtgatgctgctgacctctgacgcccctgagtacaagccttgggctc
tcgtgatccaggacagcaacggcgagaacaagatcaagatgctgagcggcggctctggtggc
tctggcggatctacaaacctgtccgatattattgagaaagaaaccgggaaacagctcgtgat
tcaagagtctattctcatgctcccggaagaagtcgaggaagtcattggaaacaagcctgaga
gcgatattctggtccatacagcctacgacgagtctaccgatgagaatgtcatgctcctcacc
agcgacgctcccgagtataagccatgggcacttgtcattcaggactccaatggggaaaacaa
aatcaaaatgctcccaaagaaaaaacgcaaggtg
An exemplary ppabobec1-orf-wt sequence is provided below:
(SEQ ID NO: 93)
atgacctctgagaagggccctagcacaggcgaccccaccctgcggcggagaatcgagagctg
ggagttcgacgtgttctacgaccctagagaactgagaaaggaaacctgcctgctgtacgaga
tcaagtggggcatgagcagaaagatctggcggagctctggcaagaacaccaccaaccacgtg
gaagtgaatttcatcaagaagttcaccagcgagagaaggttccacagcagcatcagctgcag
catcacctggttcctgagctggtccccttgctgggaatgcagccaggccatcagagagttcc
tgagccaacaccccggagtgacactggtgatctacgtggccagactgttctggcacatggac
cagagaaacagacagggcctgagagatctggtcaacagcggcgtgactatccagatcatgcg
ggccagcgagtactaccactgttggcggaacttcgtgaactacccccccggcgatgaggccc
actggcctcagtaccctcctctgtggatgatgctgtacgccctggaactgcactgcatcatc
ctgtctctgcctccatgtctgaagatctctagaagatggcagaaccacctggccttcttcag
actgcacctgcagaattgccactaccagaccatccccccccacatcctgctggctacaggcc
tgatccacccttctgtgacctggagacttaagagcggaggatctagcggcggctctagcgga
tctgagacacctggcacaagcgagtctgccacacctgagagtagcggcggatcttctggtgg
ctctgacaagaagtacagcatcggcctggccatcggcaccaactctgtgggctgggccgtga
tcaccgacgagtacaaggtgcccagcaagaaattcaaggtgctgggcaacaccgaccggcac
agcatcaagaagaacctgatcggagccctgctgttcgacagcggcgaaacagccgaggccac
ccggctgaagagaaccgccagaagaagatacaccagacggaagaaccggatctgctatctgc
aagagatcttcagcaacgagatggccaaggtggacgacagcttcttccacagactggaagag
tccttcctggtggaagaggataagaagcacgagcggcaccccatcttcggcaacatcgtgga
cgaggtggcctaccacgagaagtaccccaccatctaccacctgagaaagaaactggtggaca
gcaccgacaaggccgacctgcggctgatctatctggccctggcccacatgatcaagttccgg
ggccacttcctgatcgagggcgacctgaaccccgacaacagcgacgtggacaagctgttcat
ccagctggtgcagacctacaaccagctgttcgaggaaaaccccatcaacgccagcggcgtgg
acgccaaggccatcctgtctgccagactgagcaagagcagacggctggaaaatctgatcgcc
cagctgcccggcgagaagaagaatggcctgttcggaaacctgattgccctgagcctgggcct
gacccccaacttcaagagcaacttcgacctggccgaggatgccaaactgcagctgagcaagg
acacctacgacgacgacctggacaacctgctggcccagatcggcgaccagtacgccgacctg
tttctggccgccaagaacctgtccgacgccatcctgctgagcgacatcctgagagtgaacac
cgagatcaccaaggcccccctgagcgcctctatgatcaagagatacgacgagcaccaccagg
acctgaccctgctgaaagctctcgtgcggcagcagctgcctgagaagtacaaagagattttc
ttcgaccagagcaagaacggctacgccggctacattgacggcggagccagccaggaagagtt
ctacaagttcatcaagcccatcctggaaaagatggacggcaccgaggaactgctcgtgaagc
tgaacagagaggacctgctgcggaagcagcggaccttcgacaacggcagcatcccccaccag
atccacctgggagagctgcacgccattctgcggcggcaggaagatttttacccattcctgaa
ggacaaccgggaaaagatcgagaagatcctgaccttccgcatcccctactacgtgggccctc
tggccaggggaaacagcagattcgcctggatgaccagaaagagcgaggaaaccatcaccccc
tggaacttcgaggaagtggtggacaagggcgcttccgcccagagcttcatcgagcggatgac
caacttcgataagaacctgcccaacgagaaggtgctgcccaagcacagcctgctgtacgagt
acttcaccgtgtataacgagctgaccaaagtgaaatacgtgaccgagggaatgagaaagccc
gccttcctgagcggcgagcagaaaaaggccatcgtggacctgctgttcaagaccaaccggaa
agtgaccgtgaagcagctgaaagaggactacttcaagaaaatcgagtgcttcgactccgtgg
aaatctccggcgtggaagatcggttcaacgcctccctgggcacataccacgatctgctgaaa
attatcaaggacaaggacttcctggacaatgaggaaaacgaggacattctggaagatatcgt
gctgaccctgacactgtttgaggacagagagatgatcgaggaacggctgaaaacctatgccc
acctgttcgacgacaaagtgatgaagcagctgaagcggcggagatacaccggctggggcagg
ctgagccggaagctgatcaacggcatccgggacaagcagtccggcaagacaatcctggattt
cctgaagtccgacggcttcgccaacagaaacttcatgcagctgatccacgacgacagcctga
cctttaaagaggacatccagaaagcccaggtgtccggccagggcgatagcctgcacgagcac
attgccaatctggccggcagccccgccattaagaagggcatcctgcagacagtgaaggtggt
ggacgagctcgtgaaagtgatgggccggcacaagcccgagaacatcgtgatcgaaatggcca
gagagaaccagaccacccagaagggacagaagaacagccgcgagagaatgaagcggatcgaa
gagggcatcaaagagctgggcagccagatcctgaaagaacaccccgtggaaaacacccagct
gcagaacgagaagctgtacctgtactacctgcagaatgggcgggatatgtacgtggaccagg
aactggacatcaaccggctgtccgactacgatgtggaccatatcgtgcctcagagctttctg
aaggacgactccatcgacaacaaggtgctgaccagaagcgacaagaaccggggcaagagcga
caacgtgccctccgaagaggtcgtgaagaagatgaagaactactggcggcagctgctgaacg
ccaagctgattacccagagaaagttcgacaatctgaccaaggccgagagaggcggcctgagc
gaactggataaggccggcttcatcaagagacagctggtggaaacccggcagatcacaaagca
cgtggcacagatcctggactcccggatgaacactaagtacgacgagaatgacaagctgatcc
gggaagtgaaagtgatcaccctgaagtccaagctggtgtccgatttccggaaggatttccag
ttttacaaagtgcgcgagatcaacaactaccaccacgcccacgacgcctacctgaacgccgt
cgtgggaaccgccctgatcaaaaagtaccctaagctggaaagcgagttcgtgtacggcgact
acaaggtgtacgacgtgcggaagatgatcgccaagagcgagcaggaaatcggcaaggctacc
gccaagtacttcttctacagcaacatcatgaactttttcaagaccgagattaccctggccaa
cggcgagatccggaagcggcctctgatcgagacaaacggcgaaaccggggagatcgtgtggg
ataagggccgggattttgccaccgtgcggaaagtgctgagcatgccccaagtgaatatcgtg
aaaaagaccgaggtgcagacaggcggcttcagcaaagagtctatcctgcccaagaggaacag
cgataagctgatcgccagaaagaaggactgggaccctaagaagtacggcggcttcgacagcc
ccaccgtggcctattctgtgctggtggtggccaaagtggaaaagggcaagtccaagaaactg
aagagtgtgaaagagctgctggggatcaccatcatggaaagaagcagcttcgagaagaatcc
catcgactttctggaagccaagggctacaaagaagtgaaaaaggacctgatcatcaagctgc
ctaagtactccctgttcgagctggaaaacggccggaagagaatgctggcctctgccggcgaa
ctgcagaagggaaacgaactggccctgccctccaaatatgtgaacttcctgtacctggccag
ccactatgagaagctgaagggctcccccgaggataatgagcagaaacagctgtttgtggaac
agcacaagcactacctggacgagatcatcgagcagatcagcgagttctccaagagagtgatc
ctggccgacgctaatctggacaaagtgctgtccgcctacaacaagcaccgggataagcccat
cagagagcaggccgagaatatcatccacctgtttaccctgaccaatctgggagcccctgccg
ccttcaagtactttgacaccaccatcgaccggaagaggtacaccagcaccaaagaggtgctg
gacgccaccctgatccaccagagcatcaccggcctgtacgagacacggatcgacctgtctca
gctgggaggtgactctggtggaagcggaggatctggcggcagcaccaatctgagcgacatca
tcgagaaagagacaggcaagcagctggtcatccaagagtccatcctgatgctgcctgaagag
gtggaagaagtgatcggcaacaagcccgagtccgacatcctggtgcacaccgcctacgatga
gagcaccgacgagaacgtgatgctgctgacctctgacgcccctgagtacaagccttgggctc
tcgtgatccaggacagcaacggcgagaacaagatcaagatgctgagcggcggctctggtggc
tctggcggatctacaaacctgtccgatattattgagaaagaaaccgggaaacagctcgtgat
tcaagagtctattctcatgctcccggaagaagtcgaggaagtcattggaaacaagcctgaga
gcgatattctggtccatacagcctacgacgagtctaccgatgagaatgtcatgctcctcacc
agcgacgctcccgagtataagccatgggcacttgtcattcaggactccaatggggaaaacaa
aatcaaaatgctcccaaagaaaaaacgcaaggtg
An exemplary prtn_SzW8eqL7-abe8_20m sequence is provided below:
(SEQ ID NO: 94)
MSEVEFSHEYWMRHALTLAKRARDEREVPVGAVLVLNNRVIGEGWNRAIGLHDPTAHAEIMA
LRQGGLVMQNYRLYDATLYSTFEPCVMCAGAMIHSRIGRVVFGVRNAKTGAAGSLMDVLHHP
GMNHRVEITEGILADECAALLCRFFRMPRRVFNAQKKAQSSTDSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFDSPTVAYSVLVVAKVEKGKSKKLKSVKELLGI
TIMERSSFEKNPIDFLEAKGYKEVKKDLIIKLPKYSLFELENGRKRMLASAGELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKRYTSTKEVLDATLIHQSI
TGLYETRIDLSQLGGDEGADKRTADGSEFESPKKKRKV
An exemplary prtn_ZJVPExXY-ppabobec1-r33a-protein sequence is provided below:
(SEQ ID NO: 95)
MTSEKGPSTGDPTLRRRIESWEFDVFYDPRELAKETCLLYEIKWGMSRKIWRSSGKNTTNHV
EVNFIKKFTSERRFHSSISCSITWFLSWSPCWECSQAIREFLSQHPGVTLVIYVARLFWHMD
QRNRQGLRDLVNSGVTIQIMRASEYYHCWRNFVNYPPGDEAHWPQYPPLWMMLYALELHCII
LSLPPCLKISRRWQNHLAFFRLHLQNCHYQTIPPHILLATGLIHPSVTWRLKSGGSSGGSSG
SETPGTSESATPESSGGSSGGSDKKYSIGLAIGTNSVGWAVITDEYKVPSKKFKVLGNTDRH
SIKKNLIGALLFDSGETAEATRLKRTARRRYTRRKNRICYLQEIFSNEMAKVDDSFFHRLEE
SFLVEEDKKHERHPIFGNIVDEVAYHEKYPTIYHLRKKLVDSTDKADLRLIYLALAHMIKFR
GHFLIEGDLNPDNSDVDKLFIQLVQTYNQLFEENPINASGVDAKAILSARLSKSRRLENLIA
QLPGEKKNGLFGNLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRKQRTFDNGSIPHQ
IHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKP
AFLSGEQKKAIVDLLFKTNRKVTVKQLKEDYFKKIECFDSVEISGVEDRFNASLGTYHDLLK
IIKDKDFLDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQGDSLHEH
IANLAGSPAIKKGILQTVKVVDELVKVMGRHKPENIVIEMARENQTTQKGQKNSRERMKRIE
EGIKELGSQILKEHPVENTQLQNEKLYLYYLQNGRDMYVDQELDINRLSDYDVDHIVPQSFL
KDDSIDNKVLTRSDKNRGKSDNVPSEEVVKKMKNYWRQLLNAKLITQRKFDNLTKAERGGLS
ELDKAGFIKRQLVETRQITKHVAQILDSRMNTKYDENDKLIREVKVITLKSKLVSDFRKDFQ
FYKVREINNYHHAHDAYLNAVVGTALIKKYPKLESEFVYGDYKVYDVRKMIAKSEQEIGKAT
AKYFFYSNIMNFFKTEITLANGEIRKRPLIETNGETGEIVWDKGRDFATVRKVLSMPQVNIV
KKTEVQTGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEIIEQISEFSKRVI
LADANLDKVLSAYNKHRDKPIREQAENIIHLFTLTNLGAPAAFKYFDTTIDRKRYTSTKEVL
DATLIHQSITGLYETRIDLSQLGGDSGGSGGSGGSINLSDIIEKETGKQLVIQESILMLPEE
VEEVIGNKPESDILVHTAYDESTDENVMLLTSDAPEYKPWALVIQDSNGENKIKMLSGGSGG
SGGSTNLSDIIEKETGKQLVIQESILMLPEEVEEVIGNKPESDILVHTAYDESTDENVMLLT
SDAPEYKPWALVIQDSNGENKIKMLPKKKRKV
An exemplary prtn_ZyqE8AYc-ppabobec1-wt-protein sequence is provided below:
(SEQ ID NO: 96)
MTSEKGPSTGDPTLRRRIESWEFDVFYDPRELRKETCLLYEIKWGMSRKIWRSSGKNTTNHV
EVNFIKKFTSERRFHSSISCSITWFLSWSPCWECSQAIREFLSQHPGVTLVIYVARLFWHMD
QRNRQGLRDLVNSGVTIQIMRASEYYHCWRNFVNYPPGDEAHWPQYPPLWMMLYALELHCII
LSLPPCLKISRRWQNHLAFFRLHLQNCHYQTIPPHILLATGLIHPSVTWRLKSGGSSGGSSG
SETPGTSESATPESSGGSSGGSDKKYSIGLAIGTNSVGWAVITDEYKVPSKKFKVLGNTDRH
SIKKNLIGALLFDSGETAEATRLKRTARRRYTRRKNRICYLQEIFSNEMAKVDDSFFHRLEE
SFLVEEDKKHERHPIFGNIVDEVAYHEKYPTIYHLRKKLVDSTDKADLRLIYLALAHMIKFR
GHFLIEGDLNPDNSDVDKLFIQLVQTYNQLFEENPINASGVDAKAILSARLSKSRRLENLIA
QLPGEKKNGLFGNLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRKQRTFDNGSIPHQ
IHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKP
AFLSGEQKKAIVDLLFKTNRKVTVKQLKEDYFKKIECFDSVEISGVEDRFNASLGTYHDLLK
IIKDKDFLDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQGDSLHEH
IANLAGSPAIKKGILQTVKVVDELVKVMGRHKPENIVIEMARENQTTQKGQKNSRERMKRIE
EGIKELGSQILKEHPVENTQLQNEKLYLYYLQNGRDMYVDQELDINRLSDYDVDHIVPQSFL
KDDSIDNKVLTRSDKNRGKSDNVPSEEVVKKMKNYWRQLLNAKLITQRKFDNLTKAERGGLS
ELDKAGFIKRQLVETRQITKHVAQILDSRMNTKYDENDKLIREVKVITLKSKLVSDFRKDFQ
FYKVREINNYHHAHDAYLNAVVGTALIKKYPKLESEFVYGDYKVYDVRKMIAKSEQEIGKAT
AKYFFYSNIMNFFKTEITLANGEIRKRPLIETNGETGEIVWDKGRDFATVRKVLSMPQVNIV
KKTEVQTGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEIIEQISEFSKRVI
LADANLDKVLSAYNKHRDKPIREQAENIIHLFTLINLGAPAAFKYFDTTIDRKRYTSTKEVL
DATLIHQSITGLYETRIDLSQLGGDSGGSGGSGGSINLSDIIEKETGKQLVIQESILMLPEE
VEEVIGNKPESDILVHTAYDESTDENVMLLTSDAPEYKPWALVIQDSNGENKIKMLSGGSGG
SGGSTNLSDIIEKETGKQLVIQESILMLPEEVEEVIGNKPESDILVHTAYDESTDENVMLLT
SDAPEYKPWALVIQDSNGENKIKMLPKKKRKV

In some embodiments, the base editor has been further modified to inhibit base excision repair at the target site and induce cellular mismatch repair. Any of the Cas9 nucleases described herein may be fused to a Gam domain (bacteriophage Mu protein) to protect the Cas9 nuclease from degradation and exonuclease activity. See, e.g., Eid et al. Biochem. J. (2018) 475 (11): 1955-1964.

In some embodiments, the Cas9 nuclease belongs to class 2 type V of Cas endonuclease. Class 2 type V Cas endonucleases can be further categorized as type V-A, type V-B, type V-C, and type V-U. See, e.g., Stella et al. Nature Structural & Molecular Biology (2017) 24:882-892. In some embodiments, the Cas nuclease is a type V-A Cas endonuclease, such as a Cpf1 (Cas12a) nuclease. In some embodiments, the Cas9 nuclease is a type V-B Cas endonuclease, such as a C2c1 endonuclease. See, e.g., Shmakov et al. Mol Cell (2015) 60:385-397. In some embodiments, the Cas nuclease is MAD7™. Alternatively or in addition, the Cas9 nuclease is a Cpf1 nuclease or a variant thereof. As will be appreciated by one of skill in the art, the Cpf1 nuclease may also be referred to as Cas12a. See, e.g., Strohkendl et al. Mol. Cell (2018) 71:1-9. In some embodiments, a composition or method described herein involves, or a host cell expresses a Cpf1 nuclease derived from Provetella spp. or Francisella spp., Acidaminococcus sp. (AsCpf1), Lachnospiraceae bacterium (LpCpf1), or Eubacterium rectale. In some embodiments, the nucleotide sequence encoding the Cpf1 nuclease may be codon optimized for expression in a host cell. In some embodiments, the nucleotide sequence encoding the Cpf1 nuclease is further modified to alter the activity of the protein.

Both naturally occurring and modified variants of CRISPR/Cas nucleases are suitable for use according to aspects of this disclosure. For example, dCas or nickase variants, Cas variants having altered PAM specificities, and Cas variants having improved nuclease activities are embraced by some embodiments of this disclosure. In some embodiments, catalytically inactive variants of Cas nucleases (e.g., of Cas9 or Cas12a) are used according to the methods described herein. A catalytically inactive variant of Cpf1 (Cas12a) may be referred to dCas12a. As described herein, catalytically inactive variants of Cpf1 may be fused to a function domain to form a base editor. See, e.g., Rees et al. Nature Reviews Genetics (2018) 19:770-788. In some embodiments, the catalytically inactive Cas9 nuclease is dCas9. In some embodiments, the nuclease comprises a dCas12a fused to one or more uracil glycosylase inhibitor (UGI) domains. In some embodiments, the Cas9 nuclease comprises a dCas12a fused to an adenine base editor (ABE), for example an ABE evolved from the RNA adenine deaminase TadA. In some embodiments, the Cas nuclease comprises a dCas12a fused to cytidine deaminase enzyme (e.g., APOBEC deaminase, pmCDA1, activation-induced cytidine deaminase (AID)).

gRNA Sequences and Configurations
gRNA Configuration Generally

A gRNA can comprise a number of domains. In an embodiment, a unimolecular, sgRNA, or chimeric, gRNA comprises, e.g., from 5′ to 3′:
a targeting domain (which is complementary, or partially complementary, to a target nucleic acid sequence in a target gene, e.g., in the lineage-specific cell-surface antigen (e.g., CD33, CD20, CLL-1, CD123, CD38, CD19, CD117, EMR2, and/or CD5) gene;
a first complementarity domain;
a linking domain;
a second complementarity domain (which is complementary to the first complementarity domain);
a proximal domain; and
optionally, a tail domain.

Each of these domains is now described in more detail.

The targeting domain may comprise a nucleotide sequence that is complementary, e.g., at least 80, 85, 90, or 95% complementary, e.g., fully complementary, to the target sequence on the target nucleic acid. The targeting domain is part of an RNA molecule and will therefore typically comprise the base uracil (U), while any DNA encoding the gRNA molecule will comprise the base thymine (T). While not wishing to be bound by theory, in an embodiment, it is believed that the complementarity of the targeting domain with the target sequence contributes to specificity of the interaction of the gRNA/Cas9 nuclease complex with a target nucleic acid. It is understood that in a targeting domain and target sequence pair, the uracil bases in the targeting domain will pair with the adenine bases in the target sequence. In an embodiment, the target domain itself comprises in the 5′ to 3′ direction, an optional secondary domain, and a core domain. In an embodiment, the core domain is fully complementary with the target sequence. In an embodiment, the targeting domain is 5 to 50 nucleotides in length. The targeting domain may be between 15 and 30 nucleotides, 15-25 nucleotides, 18-22 nucleotides, or 19-21 nucleotides in length. In some embodiments, the targeting domain is 15, 16, 17, 18, 19, 20, 21, 22, 23, 24, or 25 nucleotides in length. In some embodiments, the targeting domain is between 10-30, or between 15-25, nucleotides in length. The targeting domain corresponds fully with the target domain sequence (e.g., without any mismatch nucleotides), or may comprise one or more, but typically not more than 4, mismatches. As the targeting domain is part of an RNA molecule, the gRNA, it will typically comprise ribonucleotides, while the DNA targeting domain will comprise deoxyribonucleotides. In some embodiments, the targeting domain is between 10-30, or between 15-25, nucleotides in length and comprises 5-20 contiguous nucleotides as set forth in any one of SEQ ID NOs: 68-71, 81-87, and 89. In some embodiments, the targeting domain comprises between 10-30 nucleotides in length, wherein 5, 6, 7, 8, 9, 10, 11, 12, 13, 14, 15, 16, 17, 18, 19, or 20 contiguous nucleotides of said targeting domain comprise a sequence set forth in any one of SEQ ID NOs: 68-71, 81-87, and 89. In some embodiments, the targeting domain comprises a sequence set forth in any one of SEQ ID NOs: 68-71, 81-87, and 89. In some embodiments, the targeting domain consists a sequence set forth in any one of SEQ ID NOs: 68-71, 81-87, and 89.

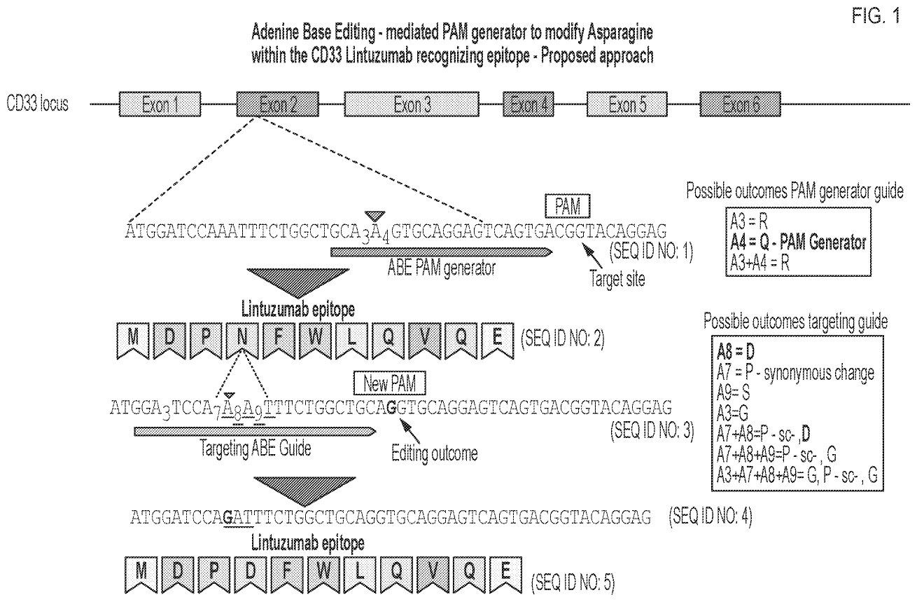

The targeting domain of the gRNA thus base-pairs (in full or partial complementarity) with the sequence of the double-stranded genomic target site that is complementary to the sequence of the target domain, and thus with the strand complementary to the strand that comprises the PAM sequence. It will be understood that the targeting domain of the gRNA typically does not include the PAM sequence. It will further be understood that the location of the PAM may be 5′ or 3′ of the target domain sequence, depending on the nuclease employed. For example, the PAM is typically 3′ of the target domain sequences for Cas9 nucleases, and 5′ of the target domain sequence for Cas12a nucleases. For an illustration of the location of the PAM and the mechanism of gRNA binding a target site, see, e.g., FIG. 1 of Vanegas et al., Fungal Biol Biotechnol. 2019; 6:6, which is incorporated by reference herein. For additional illustration and description of the mechanism of gRNA targeting an CRISPR/Cas nuclease to a target site, see Fu Y et al, Nat Biotechnol 2014 (doi: 10.1038/nbt.2808) and Sternberg S H et al., Nature 2014 (doi: 10.1038/naturel3011), both incorporated herein by reference.

An exemplary illustration of a Cas9 target site, comprising a 22 nucleotide target domain, and an NGG PAM sequence, as well as of a gRNA comprising a targeting domain that fully corresponds to the target domain (and thus base-pairs with full complementarity with the DNA strand complementary to the strand comprising the target domain and PAM) is provided below:

```
      [ target domain (DNA) ] [ PAM ]
5′-N-N-N-N-N-N-N-N-N-N-N-N-N-N-N-N-N-N-N-N-N-N-N-G-G-3′ (DNA)
3′-N-N-N-N-N-N-N-N-N-N-N-N-N-N-N-N-N-N-N-N-N-N-N-C-C-5′ (DNA)
      | | | | | | | | | | | | | | | | | | | | | |
5′-N-N-N-N-N-N-N-N-N-N-N-N-N-N-N-N-N-N-N-N-N-N-[gRNA scaffold]-3′ (RNA)
      [ targeting domain (RNA) ] [binding domain]
```

An exemplary illustration of a Cas12a target site, comprising a 22 nucleotide target domain, and a TTN PAM sequence, as well as of a gRNA comprising a targeting domain that fully corresponds to the target domain (and thus base-pairs with full complementarity with the DNA strand complementary to the strand comprising the target domain and PAM) is provided below:

```
                            [ PAM ] [ target domain (DNA) ]
                    5′-T-T-N-N-N-N-N-N-N-N-N-N-N-N-N-N-N-N-N-N-N-N-N-N-N-3′ (DNA)
                    3′-A-A-N-N-N-N-N-N-N-N-N-N-N-N-N-N-N-N-N-N-N-N-N-N-N-5′ (DNA)
                                      | | | | | | | | | | | | | | | | | | | | | |
5′-[gRNA scaffold]-N-N-N-N-N-N-N-N-N-N-N-N-N-N-N-N-N-N-N-N-N-N-3′ (RNA)
[binding domain][ targeting domain (RNA) ]
```

In some embodiments, the Cas12a PAM sequence is 5′-T-T-T-V-3′.

While not wishing to be bound by theory, at least in some embodiments, it is believed that the length and complementarity of the targeting domain with the target sequence contributes to specificity of the interaction of the gRNA/Cas9 nuclease complex with a target nucleic acid. In some embodiments, the targeting domain of a gRNA provided herein is 5 to 50 nucleotides in length. In some embodiments, the targeting domain is 15 to 25 nucleotides in length. In some embodiments, the targeting domain is 18 to 22 nucleotides in length. In some embodiments, the targeting domain is 19-21 nucleotides in length. In some embodiments, the targeting domain is 15 nucleotides in length. In some embodiments, the targeting domain is 16 nucleotides in length. In some embodiments, the targeting domain is 17 nucleotides in length. In some embodiments, the targeting domain is 18 nucleotides in length. In some embodiments, the targeting domain is 19 nucleotides in length. In some embodiments, the targeting domain is 20 nucleotides in length. In some embodiments, the targeting domain is 21 nucleotides in length. In some embodiments, the targeting domain is 22 nucleotides in length. In some embodiments, the targeting domain is 23 nucleotides in length. In some embodiments, the targeting domain is 24 nucleotides in length. In some embodiments, the targeting domain is 25 nucleotides in length. In some embodiments, the targeting domain fully corresponds, without mismatch, to a target domain sequence provided herein, or a part thereof. In some embodiments, the targeting domain of a gRNA provided herein comprises 1 mismatch relative to a target domain sequence provided herein. In some embodiments, the targeting domain comprises 2 mismatches relative to the target domain sequence. In some embodiments, the target domain comprises 3 mismatches relative to the target domain sequence.

In some embodiments, a targeting domain comprises a core domain and a secondary targeting domain, e.g., as described in International Publication No. WO 2015/157070, which is incorporated by reference in its entirety. In some embodiments, the core domain comprises about 8 to about 13 nucleotides from the 3′ end of the targeting domain (e.g., the most 3′ 8 to 13 nucleotides of the targeting domain). In an embodiment, the secondary domain is positioned 5′ to the core domain. In many embodiments, the core domain has exact complementarity (corresponds fully) with the corresponding region of the target sequence, or part thereof. In other embodiments, the core domain can have 1 or more nucleotides that are not complementary (mismatched) with the corresponding nucleotide of the target domain sequence.

The first complementarity domain is complementary with the second complementarity domain, and in an embodiment, has sufficient complementarity to the second complementarity domain to form a duplexed region under at least some physiological conditions. In an embodiment, the first complementarity domain is 5 to 30 nucleotides in length. In an embodiment, the first complementarity domain comprises 3 subdomains, which, in the 5′ to 3′ direction are: a 5′ subdomain, a central subdomain, and a 3′ subdomain. In an embodiment, the 5′ subdomain is 4 to 9, e.g., 4, 5, 6, 7, 8 or 9 nucleotides in length. In an embodiment, the central subdomain is 1, 2, or 3, e.g., 1, nucleotide in length. In an embodiment, the 3′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. The first complementarity domain can share homology with, or be derived from, a naturally occurring first complementarity domain. In an embodiment, it has at least 50% homology with a S. pyogenes, S. aureus or S. thermophilus, first complementarity domain.

The sequence and placement of the above-mentioned domains are described in more detail in International Publication No. WO2015/157070, which is herein incorporated by reference in its entirety, including p. 88-112 therein.

A linking domain serves to link the first complementarity domain with the second complementarity domain of a unimolecular gRNA. The linking domain can link the first and second complementarity domains covalently or non-covalently. In an embodiment, the linkage is covalent. In an embodiment, the linking domain is, or comprises, a covalent bond interposed between the first complementarity domain and the second complementarity domain. In some embodiments, the linking domain comprises one or more, e.g., 2, 3, 4, 5, 6, 7, 8, 9, or 10 nucleotides. In some embodiments, the linking domain comprises at least one non-nucleotide bond, e.g., as disclosed in International Publication No. WO2018/126176, the entire contents of which are incorporated herein by reference.

The second complementarity domain is complementary, at least in part, with the first complementarity domain, and in an embodiment, has sufficient complementarity to the second complementarity domain to form a duplexed region under at least some physiological conditions. In an embodiment, the second complementarity domain can include a sequence that lacks complementarity with the first complementarity domain, e.g., a sequence that loops out from the duplexed region. In an embodiment, the second complementarity domain is 5 to 27 nucleotides in length. In an embodiment, the second complementarity domain is longer than the first complementarity region. In an embodiment, the complementary domain is 5, 6, 7, 8, 9, 10, 11, 12, 13, 14, 15, 16, 17, 18, 19, 20, 21, 22, 23, 24 or 25 nucleotides in length. In an embodiment, the second complementarity domain comprises 3 subdomains, which, in the 5′ to 3′ direction are: a 5′ subdomain, a central subdomain, and a 3′ subdomain. In an embodiment, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. In an embodiment, the central subdomain is 1, 2, 3, 4 or 5, e.g., 3, nucleotides in length. In an embodiment, the 3′ subdomain is 4 to 9, e.g., 4, 5, 6, 7, 8 or 9 nucleotides in length. In an embodiment, the 5′ subdomain and the 3′ subdomain of the first complementarity domain, are respectively, complementary, e.g., fully complementary, with the 3′ subdomain and the 5′ subdomain of the second complementarity domain.

In an embodiment, the proximal domain is 5 to 20 nucleotides in length. In an embodiment, the proximal domain can share homology with or be derived from a naturally occurring proximal domain. In an embodiment, it has at least 50% homology with an S. pyogenes, S. aureus or S. thermophilus, proximal domain.

A broad spectrum of tail domains are suitable for use in gRNAs. In an embodiment, the tail domain is 0 (absent), 1, 2, 3, 4, 5, 6, 7, 8, 9, or 10 nucleotides in length. In some embodiments, the tail domain nucleotides are from or share homology with a sequence from the 5′ end of a naturally occurring tail domain. In some embodiments, the tail domain includes sequences that are complementary to each other and which, under at least some physiological conditions, form a duplexed region. In some embodiments, the tail domain is absent or is 1 to 50 nucleotides in length. In some embodiments, the tail domain can share homology with or be derived from a naturally occurring proximal tail domain. In some embodiments, it has at least 50% homology with an S. pyogenes, S. aureus or S. thermophilus, tail domain. In an embodiment, the tail domain includes nucleotides at the 3′ end that are related to the method of in vitro or in vivo transcription.

In some embodiments, modular gRNA comprises:
a first strand comprising, e.g., from 5′ to 3′:
   a targeting domain (which is complementary to a target nucleic acid in the lineage-specific cell-surface antigen (e.g., CD33, CD20, CLL-1, CD123, CD38, CD19, CD117, EMR2, and/or CD5) gene) and a first complementarity domain; and
a second strand, comprising, preferably from 5′ to 3′:
   optionally, a 5′ extension domain;
   a second complementarity domain;
   a proximal domain; and
   optionally, a tail domain.

In some embodiments, a targeting domain which is complementary to a target nucleic acid in a lineage-specific cell-surface antigen is complementary to a target nucleic acid encoding human CD33 or a portion thereof, e.g., having the amino acid sequence of SEQ ID NO: 97 or a sequence at least 70%, 75%, 80%, 85%, 90%, 95%, 96%, 97%, 98%, or 99% identical thereto.

(SEQ ID NO: 97)
MPLLLLLPLLWAGALAMDPNFWLQVQESVTVQEGLCVLVPCTFFHPIPY
YDKNSPVHGYWFREGAIISRDSPVATNKLDQEVQEETQGRFRLLGDPSR
NNCSLSIVDARRRDNGSYFFRMERGSTKYSYKSPQLSVHVIDLTHRPKI
LIPGTLEPGHSKNLTCSVSWACEQGTPPIFSWLSAAPTSLGPRTTHSSV
LIITPRPQDHGTNLTCQVKFAGAGVTTERTIQLNVTYVPQNPTTGIFPG
DGSGKQETRAGVVHGAIGGAGVTALLALCLCLIFFIVKTHRRKAARTAV
GRNDTHPTTGSASPKHQKKSKLHGPTETSSCSGAAPTVEMDEELHYASL
NFHGMNPSKDTSTEYSEVRTQ (Uniprot No. P20138)

In some embodiments, a targeting domain which is complementary to a target nucleic acid in a lineage-specific cell-surface antigen is complementary to a target nucleic acid encoding human CD20 or a portion thereof, e.g., having the amino acid sequence of SEQ ID NO: 98 or a sequence at least 70%, 75%, 80%, 85%, 90%, 95%, 96%, 97%, 98%, or 99% identical thereto.

(SEQ ID NO: 98)
MTTPRNSVNGTFPAEPMKGPIAMQSGPKPLFRRMSSLVGPTQSFFMRES
KTLGAVQIMNGLFHIALGGLLMIPAGIYAPICVTVWYPLWGGIMYIISG
SLLAATEKNSRKCLVKGKMIMNSLSLFAAISGMILSIMDILNIKISHFL
KMESLNFIRAHTPYINIYNCEPANPSEKNSPSTQYCYSIQSLFLGILSV
MLIFAFFQELVIAGIVENEWKRTCSRPKSNIVLLSAEEKKEQTIEIKEE
VVGLTETSSQPKNEEDIEIIPIQEEEEEETETNFPEPPQDQESSPIEND
SSPVRTQ (Uniprot No. P11836)

In some embodiments, a targeting domain which is complementary to a target nucleic acid in a lineage-specific cell-surface antigen is complementary to a target nucleic acid encoding human CLL-1 or a portion thereof, e.g., having the amino acid sequence of SEQ ID NO: 99 or a sequence at least 70%, 75%, 80%, 85%, 90%, 95%, 96%, 97%, 98%, or 99% identical thereto.

(SEQ ID NO: 99)
MSEEVTYADLQFQNSSEMEKIPEIGKFGEKAPPAPSHVWRPAALFLTLL
CLLLLIGLGVLASMFHVTLKIEMKKMNKLQNISEELQRNISLQLMSNMN
ISNKIRNLSTTLQTIATKLCRELYSKEQEHKCKPCPRRWIWHKDSCYFL
SDDVQTWQESKMACAAQNASLLKINNKNALEFIKSQSRSYDYWLGLSPE
EDSTRGMRVDNIINSSAWVIRNAPDLNNMYCGYINRLYVQYYHCTYKKR
MICEKMANPVQLGSTYFREA (Uniprot No. Q5QGZ9)

In some embodiments, a targeting domain which is complementary to a target nucleic acid in a lineage-specific cell-surface antigen is complementary to a target nucleic acid encoding human CD123 or a portion thereof, e.g., having the amino acid sequence of SEQ ID NO: 100 or a sequence at least 70%, 75%, 80%, 85%, 90%, 95%, 96%, 97%, 98%, or 99% identical thereto.

(SEQ ID NO: 100)
MVLLWLTLLLIALPCLLQTKEDPNPPITNLRMKAKAQQLTWDLNRNVTD
IECVKDADYSMPAVNNSYCQFGAISLCEVINYTVRVANPPFSTWILFPE
NSGKPWAGAENLTCWIHDVDELSCSWAVGPGAPADVQYDLYLNVANRRQ
QYECLHYKTDAQGTRIGCREDDISRLSSGSQSSHILVRGRSAAFGIPCT
DKFVVFSQIEILTPPNMTAKCNKTHSFMHWKMRSHFNRKFRYELQIQKR
MQPVITEQVRDRTSFQLLNPGTYTVQIRARERVYEFLSAWSTPQRFECD
QEEGANTRAWRTSLLIALGTLLALVCVFVICRRYLVMQRLFPRIPHMKD
PIGDSFQNDKLVVWEAGKAGLEECLVTEVQVVQKT
(Uniport No. P26951)

In some embodiments, a targeting domain which is complementary to a target nucleic acid in a lineage-specific cell-surface antigen is complementary to a target nucleic acid encoding human CD38 or a portion thereof, e.g., having the amino acid sequence of SEQ ID NO: 101 or a sequence at least 70%, 75%, 80%, 85%, 90%, 95%, 96%, 97%, 98%, or 99% identical thereto.

(SEQ ID NO: 101)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCKNIYRPDKFLQCVKNPEDS
SCTSEI (Uniprot No. P28907)

In some embodiments, a targeting domain which is complementary to a target nucleic acid in a lineage-specific cell-surface antigen is complementary to a target nucleic acid encoding human CD19 or a portion thereof, e.g., having the amino acid sequence of SEQ ID NO: 102 or a sequence at least 70%, 75%, 80%, 85%, 90%, 95%, 96%, 97%, 98%, or 99% identical thereto.

(SEQ ID NO: 102)
MPPPRLLFFLLFLTPMEVRPEEPLVVKVEEGDNAVLQCLKGTSDGPTQQ
LTWSRESPLKPFLKLSLGLPGLGIHMRPLAIWLFIFNVSQQMGGFYLCQ
PGPPSEKAWQPGWTVNVEGSGELFRWNVSDLGGLGCGLKNRSSEGPSSP
SGKLMSPKLYVWAKDRPEIWEGEPPCLPPRDSLNQSLSQDLTMAPGSTL
WLSCGVPPDSVSRGPLSWTHVHPKGPKSLLSLELKDDRPARDMWVMETG
LLLPRATAQDAGKYYCHRGNLTMSFHLEITARPVLWHWLLRTGGWKVSA
VTLAYLIFCLCSLVGILHLQRALVLRRKRKRMTDPTRRFFKVTPPPGSG
PQNQYGNVLSLPTPTSGLGRAQRWAAGLGGTAPSYGNPSSDVQADGALG
SRSPPGVGPEEEEGEGYEEPDSEEDSEFYENDSNLGQDQLSQDGSGYEN
PEDEPLGPEDEDSFSNAESYENEDEELTQPVARTMDELSPHGSAWDPSR
EATSLGSQSYEDMRGILYAAPQLRSIRGQPGPNHEEDADSYENMDNPDG
PDPAWGGGGRMGTWSTR (Uniprot No. P15391)

In some embodiments, a targeting domain which is complementary to a target nucleic acid in a lineage-specific cell-surface antigen is complementary to a target nucleic acid encoding human CD117 or a portion thereof, e.g., having the amino acid sequence of SEQ ID NO: 103 or a sequence at least 70%, 75%, 80%, 85%, 90%, 95%, 96%, 97%, 98%, or 99% identical thereto.

(SEQ ID NO: 103)
MRGARGAWDFLCVLLLLLRVQTGSSQPSVSPGEPSPPSIHPGKSDLIVR
VGDEIRLLCTDPGFVKWTFEILDETNENKQNEWITEKAEATNTGKYTCT
NKHGLSNSIYVFVRDPAKLFLVDRSLYGKEDNDTLVRCPLTDPEVINYS
LKGCQGKPLPKDLRFIPDPKAGIMIKSVKRAYHRLCLHCSVDQEGKSVL
SEKFILKVRPAFKAVPVVSVSKASYLLREGEEFTVTCTIKDVSSSVYST
WKRENSQTKLQEKYNSWHHGDFNYERQATLTISSARVNDSGVFMCYANN
TFGSANVTTTLEVVDKGFINIFPMINTTVFVNDGENVDLIVEYEAFPKP
EHQQWIYMNRTFTDKWEDYPKSENESNIRYVSELHLTRLKGTEGGTYTF
LVSNSDVNAAIAFNVYVNTKPEILTYDRLVNGMLQCVAAGFPEPTIDWY
FCPGTEQRCSASVLPVDVQTLNSSGPPFGKLVVQSSIDSSAFKHNGTVE
CKAYNDVGKTSAYFNFAFKGNNKEQIHPHTLFTPLLIGFVIVAGMMCII
VMILTYKYLQKPMYEVQWKVVEEINGNNYVYIDPTQLPYDHKWEFPRNR
LSFGKTLGAGAFGKVVEATAYGLIKSDAAMTVAVKMLKPSAHLTEREAL
MSELKVLSYLGNHMNIVNLLGACTIGGPTLVITEYCCYGDLLNFLRRKR
DSFICSKQEDHAEAALYKNLLHSKESSCSDSTNEYMDMKPGVSYVVPTK
ADKRRSVRIGSYIERDVTPAIMEDDELALDLEDLLSFSYQVAKGMAFLA
SKNCIHRDLAARNILLTHGRITKICDFGLARDIKNDSNYVVKGNARLPV
KWMAPESIFNCVYTFESDVWSYGIFLWELFSLGSSPYPGMPVDSKFYKM
IKEGFRMLSPEHAPAEMYDIMKTCWDADPLKRPTFKQIVQLIEKQISES
TNHIYSNLANCSPNRQKPVVDHSVRINSVGSTASSSQPLLVHDDV
(Uniprot No. P10721)

In some embodiments, a targeting domain which is complementary to a target nucleic acid in a lineage-specific cell-surface antigen is complementary to a target nucleic acid encoding human EMR2 or a portion thereof, e.g., having the amino acid sequence of SEQ ID NO: 104 or a sequence at least 70%, 75%, 80%, 85%, 90%, 95%, 96%, 97%, 98%, or 99% identical thereto.

(SEQ ID NO: 104)
MGGRVFLVFLAFCVWLTLPGAETQDSRGCARWCPQDSSCVNATACRCN
PGESSESEIITTPMETCDDINECATLSKVSCGKFSDCWNTEGSYDCVC
SPGYEPVSGAKTFKNESENTCQDVDECQQNPRLCKSYGTCVNTLGSYT
CQCLPGFKLKPEDPKLCTDVNECTSGQNPCHSSTHCLNNVGSYQCRCR
PGWQPIPGSPNGPNNTVCEDVDECSSGQHQCDSSTVCENTVGSYSCRC
RPGWKPRHGIPNNQKDTVCEDMTFSTWTPPPGVHSQTLSRFFDKVQDL
GRDYKPGLANNTIQSILQALDELLEAPGDLETLPRLQQHCVASHLLDG
LEDVLRGLSKNLSNGLLNFSYPAGTELSLEVQKQVDRSVTLRQNQAVM
QLDWNQAQKSGDPGPSVVGLVSIPGMGKLLAEAPLVLEPEKQMLLHET
HQGLLQDGSPILLSDVISAFLSNNDTQNLSSPVTFTFSHRSVIPRQKV
LCVFWEHGQNGCGHWATTGCSTIGTRDTSTICRCTHLSSFAVLMAHYD
VQEEDPVLIVITYMGLSVSLLCLLLAALTFLLCKAIQNTSTSLHLQLS
LCLFLAHLLFLVAIDQTGHKVLCSIIAGTLHYLYLATLTWMLLEALYL
FLTARNLTVVNYSSINRFMKKLMFPVGYGVPAVTVAISAASRPHLYGT
PSRCWLQPEKGFIWGFLGPVCAIFSVNLVLFLVTLWILKNRLSSLNSE
VSTLRNTRMLAFKATAQLFILGCTWCLGILQVGPAARVMAYLFTIINS
LQGVFIFLVYCLLSQQVREQYGKWSKGIRKLKTESEMHTLSSSAKADT
SKPSTVN (Uniprot No. Q9UHX3)

In some embodiments, a targeting domain which is complementary to a target nucleic acid in a lineage-specific cell-surface antigen is complementary to a target nucleic acid encoding human CD5 or a portion thereof, e.g., having the amino acid sequence of SEQ ID NO: 105 or a sequence at least 70%, 75%, 80%, 85%, 90%, 95%, 96%, 97%, 98%, or 99% identical thereto.

(SEQ ID NO: 105)
MPMGSLQPLATLYLLGMLVASCLGRLSWYDPDFQARLTRSNSKCQGQLE
VYLKDGWHMVCSQSWGRSSKQWEDPSQASKVCQRLNCGVPLSLGPFLVT
YTPQSSIICYGQLGSFSNCSHSRNDMCHSLGLTCLEPQKTTPPTTRPPP
TTTPEPTAPPRLQLVAQSGGQHCAGVVEFYSGSLGGTISYEAQDKTQDL
ENFLCNNLQCGSFLKHLPETEAGRAQDPGEPREHQPLPIQWKIQNSSCT
SLEHCFRKIKPQKSGRVLALLCSGFQPKVQSRLVGGSSICEGTVEVRQG
AQWAALCDSSSARSSLRWEEVCREQQCGSVNSYRVLDAGDPTSRGLFCP
HQKLSQCHELWERNSYCKKVFVTCQDPNPAGLAAGTVASIILALVLLVV
LLVVCGPLAYKKLVKKFRQKKQRQWIGPTGMNQNMSFHRNHTATVRSHA
ENPTASHVDNEYSQPPRNSHLSAYPALEGALHRSSMQPDNSSDSDYDLH
GAQRL (Uniprot No. P06127)

In some embodiments, the gRNA is chemically modified. In some embodiments, any of the gRNAs provided herein comprise one or more nucleotides that are chemically modified. Chemical modifications of gRNAs have previously been described, and suitable chemical modifications include any modifications that are beneficial for gRNA function and do not measurably increase any undesired characteristics, e.g., off-target effects, of a given gRNA. Suitable chemical modifications include, for example, those that make a gRNA less susceptible to endo- or exonuclease catalytic activity, and include, without limitation, that the gRNA may comprise one or more modification chosen from phosphorothioate backbone modification, 2′-O-Me-modified sugars (e.g., at one or both of the 3′ and 5′ termini), 2′F-modified sugar, replacement of the ribose sugar with the bicyclic nucleotide-cEt, 3′thioPACE (MSP), or any combination thereof. Additional suitable gRNA modifications will be apparent to the skilled artisan based on this disclosure, and such suitable gRNA modification include, without limitation, those described, e.g., in Rahdar et al. PNAS Dec. 22, 2015 112 (51) E7110-E7117 and Hendel et al., Nat Biotechnol. 2015 September; 33 (9): 985-989, each of which is incorporated herein by reference in its entirety. In some embodiments, a gRNA described herein comprises one or more 2′-O-methyl-3′-phosphorothioate nucleotides, e.g., at least 2, 3, 4, 5, or 6 2′-O-methyl-3′-phosphorothioate nucleotides. In some embodiments, a gRNA described herein comprises modified nucleotides (e.g., 2′-O-methyl-3′-phosphorothioate nucleotides) at the three terminal positions and the 5′ end and/or at the three terminal positions and the 3′ end. In some embodiments, the gRNA may comprise one or more modified nucleotides, e.g., as described in International Publication Nos. WO2017/214460, WO2016/089433, and WO2016/164356, which are incorporated by reference their entirety.

In some embodiments, a gRNA described herein is chemically modified. For example, the gRNA may comprise one or more 2′-O modified nucleotides, e.g., 2′-O-methyl nucleotides. In some embodiments, the gRNA comprises a 2′-O modified nucleotide, e.g., 2′-O-methyl nucleotide at the 5′ end of the gRNA. In some embodiments, the gRNA comprises a 2′-O modified nucleotide, e.g., 2′-O-methyl nucleotide at the 3′ end of the gRNA. In some embodiments, the gRNA comprises a 2′-O-modified nucleotide, e.g., 2′-O-methyl nucleotide at both the 5′ and 3′ ends of the gRNA. In some embodiments, the gRNA is 2′-O-modified, e.g. 2′-O-methyl-modified at the nucleotide at the 5′ end of the gRNA, the second nucleotide from the 5′ end of the gRNA, and the third nucleotide from the 5′ end of the gRNA. In some embodiments, the gRNA is 2′-O-modified, e.g. 2′-O-methyl-modified at the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the second nucleotide from the 3′ end of the gRNA, the third nucleotide from the 3′ end of the gRNA, and at the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified, e.g. 2′-O-methyl-modified, at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA. In some embodiments, the 2′-O-methyl nucleotide comprises a phosphate linkage to an adjacent nucleotide. In some embodiments, the 2′-O-methyl nucleotide comprises a phosphorothioate linkage to an adjacent nucleotide. In some embodiments, the 2′-O-methyl nucleotide comprises a thioPACE linkage to an adjacent nucleotide.

In some embodiments, the gRNA may comprise one or more 2′-O-modified and 3′phosphorous-modified nucleotide, e.g., a 2′-O-methyl 3′phosphorothioate nucleotide. In some embodiments, the gRNA comprises a 2′-O-modified and 3′phosphorous-modified, e.g., 2′-O-methyl 3′phosphorothioate nucleotide at the 5′ end of the gRNA. In some embodiments, the gRNA comprises a 2′-O-modified and 3′phosphorous-modified, e.g., 2′-O-methyl 3′phosphorothioate nucleotide at the 3′ end of the gRNA. In some embodiments, the gRNA comprises a 2′-O-modified and 3′phosphorous-modified, e.g., 2′-O-methyl 3′phosphorothioate nucleotide at the 5′ and 3′ ends of the gRNA. In some embodiments, the gRNA comprises a backbone in which one or more non-bridging oxygen atoms has been replaced with a sulfur atom. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′phosphorothioate-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, the gRNA may comprise one or more 2′-O-modified and 3′-phosphorous-modified, e.g., 2′-O-methyl 3′thioPACE nucleotide. In some embodiments, the gRNA comprises a 2′-O-modified and 3′phosphorous-modified, e.g., 2′-O-methyl 3′thioPACE nucleotide at the 5′ end of the gRNA. In some embodiments, the gRNA comprises a 2′-O-modified and 3′phosphorous-modified, e.g., 2′-O-methyl 3′thioPACE nucleotide at the 3′ end of the gRNA. In some embodiments, the gRNA comprises a 2′-O-modified and 3′phosphorous-modified, e.g., 2′-O-methyl 3′thioPACE nucleotide at the 5′ and 3′ ends of the gRNA. In some embodiments, the gRNA comprises a backbone in which one or more non-bridging oxygen atoms have been replaced with a sulfur atom and one or more non-bridging oxygen atoms have been replaced with an acetate group. In some embodiments, the gRNA is 2′-O-modified and 3′phosphorous-modified, e.g. 2′-O-methyl 3′ thioPACE-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′thioPACE-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, the gRNA comprises a chemically modified backbone. In some embodiments, the gRNA comprises a phosphorothioate linkage. In some embodiments, one or more non-bridging oxygen atoms have been replaced with a sulfur atom. In some embodiments, the nucleotide at the 5′ end of the gRNA, the second nucleotide from the 5′ end of the gRNA, and the third nucleotide from the 5′ end of the gRNA each comprise a phosphorothioate linkage. In some embodiments, the nucleotide at the 3′ end of the gRNA, the second nucleotide from the 3′ end of the gRNA, and the third nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a phosphorothioate linkage. In some embodiments, the second nucleotide from the 3′ end of the gRNA, the third nucleotide from the 3′ end of the gRNA, and at the fourth nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a phosphorothioate linkage.

In some embodiments, the gRNA comprises a thioPACE linkage. In some embodiments, the gRNA comprises a backbone in which one or more non-bridging oxygen atoms have been replaced with a sulfur atom and one or more non-bridging oxygen atoms have been replaced with an acetate group. In some embodiments, the nucleotide at the 5′ end of the gRNA, the second nucleotide from the 5′ end of the gRNA, and the third nucleotide from the 5′ end of the gRNA each comprise a thioPACE linkage. In some embodiments, the nucleotide at the 3′ end of the gRNA, the second nucleotide from the 3′ end of the gRNA, and the third nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a thioPACE linkage. In some embodiments, the second nucleotide from the 3′ end of the gRNA, the third nucleotide from the 3′ end of the gRNA, and at the fourth nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a thioPACE linkage.

Some exemplary, non-limiting embodiments of modifications, e.g., chemical modifications, suitable for use in connection with the guide RNAs and genetic engineering methods provided herein have been described above. Additional suitable modifications, e.g., chemical modifications, will be apparent to those of skill in the art based on the present disclosure and the knowledge in the art, including, but not limited to those described in Hendel, A. et al., Nature Biotech., 2015, Vol 33, No. 9; in International Publication No. WO2017/214460; WO2016/089433; and in WO2016/164356; each one of which is herein incorporated by reference in its entirety.

The lineage-specific cell-surface antigen (e.g., CD33, CD20, CLL-1, CD123, CD38, CD19, CD117, EMR2, and/or CD5) targeting gRNAs provided herein can be delivered to a cell in any manner suitable. In some embodiments, the gRNA is a gRNA disclosed in any of International Publication Nos. WO2017/066760, WO2020/047164, WO2020/150478, and WO2020/237217, WO2019/046285, WO2018/160768, WO2021/041971, WO2021/041977, or Borot et al. PNAS Jun. 11, 2019 116 (24) 11978-11987, each of which is incorporated herein by reference in its entirety. Various suitable methods for the delivery of CRISPR/Cas systems, e.g., comprising an ribonucleoprotein (RNP) complex including a gRNA bound to a Cas nuclease, have been described, and exemplary suitable methods include, without limitation, electroporation of an RNP into a cell, electroporation of mRNA encoding a Cas nuclease and a gRNA into a cell, various protein or nucleic acid transfection methods, and delivery of encoding RNA or DNA via viral vectors, such as, for example, retroviral (e.g., lentiviral) vectors. Any suitable delivery method is embraced by this disclosure, and the disclosure is not limited in this respect.

gRNAs for Generating Artificial PAMs

The present disclosure provides a number of useful gRNAs that are capable of targeting a Cas nuclease to a first target domain to provide a first mutation to generate an artificial PAM in a double-stranded genomic DNA molecule.

In some embodiments, the generated artificial PAM is a SpyCas9 PAM, a SpGCas9 PAM, a SpRYCas9 PAM, a Sth1Cas9 PAM, a SauCas9 PAM, a NmeCas9 PAM, a RspCas9 PAM, a Cca1Cas9 PAM, a PspCas9 PAM, a OrhCas9 PAM, a ScCas9 PAM, a SmacCas9 PAM, a TdeCas9 PAM, a Nme2Cas9 PAM, a CjeCas9 PAM, a SmuCas9 PAM, a Smu2Cas9 PAM, a PmuCas9 PAM, a SpaCas9 PAM, a NciCas9 PAM, a ClaCas9 PAM, a PlaCas9 PAM, a CdCas9 PAM, a IgnaviCas9 PAM, a ThermoCas9 PAM, a GeoCas9 PAM, a G. LC300 Cas9 PAM, a AceCas9 PAM, a Sth3Cas9 PAM, a BlatCas9 PAM, a FnCas9 PAM, a LfeCas9 PAM, a LpnCas9 PAM, a KhuCas9 PAM, a AinCas9 PAM, a CglCas9 PAM, a Esp1Cas9 PAM, a Esp2Cas9 PAM, a FmaCas9 PAM, a LceCas9 PAM, a LrhCas9 PAM, a Lsp1Cas9 PAM, a Lsp2Cas9 PAM, a PacCas9 PAM, a TbaCas9 PAM, a TpuCas9 PAM, a VpaCas9 PAM, a EfaCas9 PAM, a EitCas9 PAM, a LanCas9 PAM, a LmoCas9 PAM, a Sag1Cas9 PAM, a Sag2Cas9 PAM, a SdyCas9 PAM, a Seq1Cas9 PAM, a Seq2Cas9 PAM, a SgaCas9 PAM, a Smu3Cas9 PAM, a SraCas9 PAM, a BniCas9 PAM, a EceCas9 PAM, a EdoCas9 PAM, a FhoCas9 PAM, a MgaCas9 PAM, a MseCas9 PAM, a SgoCas9 PAM, a SmaCas9 PAM, a SsaCas9 PAM, a SsiCas9 PAM, a SsuCas9 PAM, a Sth1ACas9 PAM, a TspCas9 PAM, a BokCas9 PAM, a CcoCas9 PAM, a CpeCas9 PAM, a DdeCas9 PAM, a Ghc2Cas9 PAM, a Ghy3Cas9 PAM, a Ghy4Cas9 PAM, a GspCas9 PAM, a KkiCas9 PAM, a NspCas9 PAM, a TmoCas9 PAM, a NsaCas9 PAM, a JpaCas9 PAM, a BboCas9 PAM, a Cca2Cas9 PAM, a Cme2Cas9 PAM, a Cme3Cas9 PAM, a Cme4Cas9 PAM, a CsaCas9 PAM, a Ghc1Cas9 PAM, a GheCas9 PAM, a Ghh1Cas9 PAM, a Ghh2Cas9 PAM, a Ghy1Cas9 PAM, a MscCas9 PAM, a SdoCas9 PAM, a SpacCas9 PAM, a CgaCas9 PAM, a Cme1Cas9 PAM, a FfrCas9 PAM, a Ghy2Cas9 PAM, a PhiCas9 PAM, a WviCas9 PAM, a CcCas9 PAM, a FnCas12a PAM, a AsCas12a PAM, a HkCas12a PAM, a PiCas12a PAM, a PdCas12a PAM, a LbCas12a PAM, a Lb2Cas12a or Lb5Cas12a PAM, a CMtCas12a PAM, a MbCas12a PAM, a TsCas12a PAM, a Pb2Cas12a PAM, a MICas12a PAM, a Mb2Cas12a PAM, a Mb3Cas12a PAM, a CMaCas12a PAM, a BsCas12a PAM, a BfCas12a PAM, a BoCas12a PAM, a Adurb193Cas12a PAM, a Adurb336Cas12a PAM, a Fn3Cas12a PAM, a Lb6Cas12a PAM, a EcCas12a PAM, a PsCas12a PAM, a McCas12a PAM, a AacCas12b PAM, a BthCas12b PAM, a AkCas12b PAM, a EbCas12b PAM, a BvCas12b PAM, a BhCas12b PAM, a LsCas12b PAM, a BrCas12b PAM, a Cas12c1 PAM, a Cas12c2 PAM, a OspCas12c PAM, a Cas12d.15 PAM, a Cas12d.1 (CasY.1) PAM, a DpbCas12e (DpbCasX) PAM, a PlmCas12e (PlmCasX) PAM, a MilCas12f2 PAM, a Un1Cas12f1 PAM, a Un2Cas12f1 PAM, a Mi2Cas12f2 PAM, a AuCas12f2 PAM, a PtCas12f1 PAM, a AsCas12f1 PAM, a RuCas12f1 PAM, a SpCas12f1 PAM, a CnCas12f1 PAM, a Cas12h1 PAM, a Cas12i1 PAM, a Cas1212 PAM, a Cas12j-1 (CasPhi-1) PAM, a Cas12j-2 (CasPhi-2) PAM, a Cas12j-3 (CasPhi-3) PAM, a ShCas12k PAM, and/or a AcCas12k PAM.

In some embodiments, the generated artificial PAM may comprises and/or consists of a nucleotide sequence selected from: NGG; NNAGAAW; NNGRRT; NNNNGATT; NVNDCCY; BRTTTTT; NR (A or G) TTTT; NNAAAR (G or A); N (N or A) G; NAAN; NAAAAY; NHDTCCA; NNNVRYM; NNNNRYAC; NAA; GNNNNCNNA; NNGTGA; NNNNGTA; NNGGG; NNNCAT; NNRHHHY; NRRNAT; NNNNCNAA; NNNNCMCA; NNNNCRAA; NNNNGMAA; NNNCC; NGGNG; NNNNCNDD; NYAAA; NRGNN; N (C or D) GGN (T or A or G or C) NN; NRTAW; N (C or K or A) AARC; NAAAG; NV (A or G or C) R (A or G) ACCN; NNGAC; NATGNT; N (T or V) NTAAW (A or T); NNGW (A or T) AY (T or C); NCAA (H (Y or A) B (Y or G); NH (T or C or A) AAAA; NNNATTT; NATAWN (A or T or S); NATARCH; B (T or G or C) GGD (A or T or G) TNN; N (G or T or M) GGAH (T or A or C) N (A or C or K) N; NRG; N (B or A) GG; NGGD (A or K) W (T or A); N (T or C or R) AGAN (A or K or C) NN; NGGD (A or T or G) H (T or M); NGGDT; NGGD (A or T or G) GNN; NNGTAM (A or C) Y; NNGH (W or C) AAA; NTGAR (G or A) N (A or Y or G) N (Y or R); NNGAAAN; NNGAD; NHARMC; NNAAAG; NHGYNAN (A or B); NNAGAAA; NHAAAAA; NH (T or M) AAAAA; NHGYRAA; NNAAACN; NN (H or G) D (A or K) GGDN (A or B); NNNNCTA; NNNNCVGAA; NNNNGYAA; NNNNATN (W or S) ANN; NNWHR (G or A) TA (not G) AA; YHHNGTH; NNNNCDAANN; NNNNCTAA; N (C or D) NNTCCN; NNNNCCAA; NAGRGN (T or V) N (T or C); NNAH (T or M) ACN; CN (C or W or G) AV (A or S) GAC; NAR (G or A) H (W or C) H (A or T or C) GN (C or T or R); NAGNGC; NATCCTN; NGTGANN; HGCNGCR; NAR (A or G) W (T or A) AC; N (C or D) M (A or C) RN (A or B) AY (C or T); NNNCAC; BGGGTCD; NNRRCC; NRRNTT; KARDAT; BRRTTTW; NARNCCN; NAR (A or G) TC; NAAN (A or T or S) RCN; HHAAATD; NNNNGNA; TTV; TTTV; YYV; KKYV; TTTM; TTYV; TTTN; TTTTA; TTN; BTTV; YTV; YTN; NYTV; DTTD; ATTN; RTTNT; HATT; ATTW; RTTN; TVT; TG; TN; TR; TA; TTCN; TTAT; TTTR; TTR; YTTR; YTTN; CTT; TTC; CCD; RTR; VTTR; TBN; VTTN; NGTT; CGTT; AGG; CGG; GTT, or RGTG, wherein “N” is any nucleotide or base, “W” is adenine (A) or thymine (T), “R” is A or guanine (G), “V” is A, cytosine (C), or G, “Y” is C or T, and “H” is A, C, or T.

gRNAs for Modifying Cell-Surface Protein Epitopes

The present disclosure further provides a number of useful gRNAs that is capable of targeting a Cas nuclease to a second or subsequent target domain to provide a second mutation, e.g., in a target gene encoding a cell-surface protein (e.g., CD33, CD20, CLL-1, CD123, CD38, CD19, CD117, EMR2, and/or CD5) epitope, wherein the second mutation results in a modification (e.g., an amino acid substitution or deletion) in the cell-surface protein epitope that reduces the binding activity of a ligand or an antibody to the epitope comprising the modification as compared to the unmodified epitope.

In some embodiments, a second or subsequent target domain to provide a second or subsequent mutation, e.g., in a target gene encoding a cell-surface protein epitope, is complementary to a target nucleic acid encoding an epitope in a CD33 cell-surface epitope or a fragment of the epitope sufficient to bind to an agent (e.g., a ligand or an antibody), e.g., wherein the CD33 comprises the amino acid sequence of SEQ ID NO: 97 or a sequence at least 70%, 75%, 80%, 85%, 90%, 95%, 96%, 97%, 98%, or 99% identical thereto.

(SEQ ID NO: 97)
MPLLLLLPLLWAGALAMDPNFWLQVQESVTVQEGLCVLVPCTFFHPIPY
YDKNSPVHGYWFREGAIISRDSPVATNKLDQEVQEETQGRFRLLGDPSR
NNCSLSIVDARRRDNGSYFFRMERGSTKYSYKSPQLSVHVTDLTHRPKI
LIPGTLEPGHSKNLTCSVSWACEQGTPPIFSWLSAAPTSLGPRTTHSSV
LIITPRPQDHGTNLTCQVKFAGAGVTTERTIQLNVTYVPQNPTTGIFPG
DGSGKQETRAGVVHGAIGGAGVTALLALCLCLIFFIVKTHRRKAARTAV
GRNDTHPTTGSASPKHQKKSKLHGPTETSSCSGAAPTVEMDEELHYASL
NFHGMNPSKDTSTEYSEVRTQ (Uniprot No. P20138)

In some embodiments, a second or subsequent target domain to provide a second or subsequent mutation, e.g., in a target gene encoding a cell-surface protein epitope, is complementary to a target nucleic acid encoding an epitope in a CD20 cell-surface epitope or a fragment of the epitope sufficient to bind to an agent (e.g., a ligand or an antibody), e.g., wherein the CD20 comprises the amino acid sequence of SEQ ID NO: 98 or a sequence at least 70%, 75%, 80%, 85%, 90%, 95%, 96%, 97%, 98%, or 99% identical thereto.

(SEQ ID NO: 98)
MTTPRNSVNGTFPAEPMKGPIAMQSGPKPLFRRMSSLVGPTQSFFMRES
KILGAVQIMNGLFHIALGGLLMIPAGIYAPICVTVWYPLWGGIMYIISG
SLLAATEKNSRKCLVKGKMIMNSLSLFAAISGMILSIMDILNIKISHFL
KMESLNFIRAHTPYINIYNCEPANPSEKNSPSTQYCYSIQSLFLGILSV
MLIFAFFQELVIAGIVENEWKRTCSRPKSNIVLLSAEEKKEQTIEIKEE
VVGLTETSSQPKNEEDIEIIPIQEEEEEETETNFPEPPQDQESSPIEND
SSPVRTQ (Uniprot No. P11836)

In some embodiments, a second or subsequent target domain to provide a second or subsequent mutation, e.g., in a target gene encoding a cell-surface protein epitope, is complementary to a target nucleic acid encoding an epitope in a CLL-1 cell-surface epitope or a fragment of the epitope sufficient to bind to an agent (e.g., a ligand or an antibody), e.g., wherein the CLL-1 comprises the amino acid sequence of SEQ ID NO: 99 or a sequence at least 70%, 75%, 80%, 85%, 90%, 95%, 96%, 97%, 98%, or 99% identical thereto.

(SEQ ID NO: 99)
MSEEVTYADLQFQNSSEMEKIPEIGKFGEKAPPAPSHVWRPAALFLTLL
CLLLLIGLGVLASMFHVTLKIEMKKMNKLQNISEELQRNISLQLMSNMN
ISNKIRNLSTTLQTIATKLCRELYSKEQEHKCKPCPRRWIWHKDSCYFL
SDDVQTWQESKMACAAQNASLLKINNKNALEFIKSQSRSYDYWLGLSPE
EDSTRGMRVDNIINSSAWVIRNAPDLNNMYCGYINRLYVQYYHCTYKKR
MICEKMANPVQLGSTYFREA (Uniprot No. Q5QGZ9)

In some embodiments, a second or subsequent target domain to provide a second or subsequent mutation, e.g., in a target gene encoding a cell-surface protein epitope, is complementary to a target nucleic acid encoding an epitope in a CD123 cell-surface epitope or a fragment of the epitope sufficient to bind to an agent (e.g., a ligand or an antibody), e.g., wherein the CD123 comprises the amino acid sequence of SEQ ID NO: 100 or a sequence at least 70%, 75%, 80%, 85%, 90%, 95%, 96%, 97%, 98%, or 99% identical thereto.

(SEQ ID NO: 100)
MVLLWLTLLLIALPCLLQTKEDPNPPITNLRMKAKAQQLTWDLNRNVTD
IECVKDADYSMPAVNNSYCQFGAISLCEVTNYTVRVANPPFSTWILFPE
NSGKPWAGAENLTCWIHDVDFLSCSWAVGPGAPADVQYDLYLNVANRRQ
QYECLHYKTDAQGTRIGCRFDDISRLSSGSQSSHILVRGRSAAFGIPCT
DKFVVFSQIEILTPPNMTAKCNKTHSFMHWKMRSHFNRKFRYELQIQKR
MQPVITEQVRDRTSFQLLNPGTYTVQIRARERVYEFLSAWSTPQRFECD
QEEGANTRAWRTSLLIALGTLLALVCVFVICRRYLVMQRLFPRIPHMKD
PIGDSFQNDKLVVWEAGKAGLEECLVTEVQVVQKT
(Uniport No. P26951)

In some embodiments, a second or subsequent target domain to provide a second or subsequent mutation, e.g., in a target gene encoding a cell-surface protein epitope, is complementary to a target nucleic acid encoding an epitope in a CD38 cell-surface epitope or a fragment of the epitope sufficient to bind to an agent (e.g., a ligand or an antibody), e.g., wherein the CD38 comprises the amino acid sequence of SEQ ID NO: 101 or a sequence at least 70%, 75%, 80%, 85%, 90%, 95%, 96%, 97%, 98%, or 99% identical thereto.

(SEQ ID NO: 101)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCKNIYRPDKFLQCVKNPEDS
SCTSEI (Uniprot No. P28907)

In some embodiments, a second or subsequent target domain to provide a second or subsequent mutation, e.g., in a target gene encoding a cell-surface protein epitope, is complementary to a target nucleic acid encoding an epitope in a CD19 cell-surface epitope or a fragment of the epitope sufficient to bind to an agent (e.g., a ligand or an antibody), e.g., wherein the CD19 comprises the amino acid sequence of SEQ ID NO: 102 or a sequence at least 70%, 75%, 80%, 85%, 90%, 95%, 96%, 97%, 98%, or 99% identical thereto.

(SEQ ID NO: 102)
MPPPRLLFFLLFLTPMEVRPEEPLVVKVEEGDNAVLQCLKGTSDGPTQQ
LTWSRESPLKPFLKLSLGLPGLGIHMRPLAIWLFIFNVSQQMGGFYLCQ
PGPPSEKAWQPGWTVNVEGSGELFRWNVSDLGGLGCGLKNRSSEGPSSP
SGKLMSPKLYVWAKDRPEIWEGEPPCLPPRDSLNQSLSQDLIMAPGSTL
WLSCGVPPDSVSRGPLSWTHVHPKGPKSLLSLELKDDRPARDMWVMETG
LLLPRATAQDAGKYYCHRGNLTMSFHLEITARPVLWHWLLRTGGWKVSA
VTLAYLIFCLCSLVGILHLQRALVLRRKRKRMTDPTRRFFKVTPPPGSG
PQNQYGNVLSLPTPTSGLGRAQRWAAGLGGTAPSYGNPSSDVQADGALG
SRSPPGVGPEEEEGEGYEEPDSEEDSEFYENDSNLGQDQLSQDGSGYEN
PEDEPLGPEDEDSFSNAESYENEDEELTQPVARTMDFLSPHGSAWDPSR
EATSLGSQSYEDMRGILYAAPQLRSIRGQPGPNHEEDADSYENMDNPDG
PDPAWGGGGRMGTWSTR (Uniprot No. P15391)

In some embodiments, a second or subsequent target domain to provide a second or subsequent mutation, e.g., in a target gene encoding a cell-surface protein epitope, is complementary to a target nucleic acid encoding an epitope in a CD117 cell-surface epitope or a fragment of the epitope sufficient to bind to an agent (e.g., a ligand or an antibody), e.g., wherein the CD117 comprises the amino acid sequence of or a sequence at least 70%, 75%, 80%, 85%, 90%, 95%, 96%, 97%, 98%, or 99% identical thereto.

(SEQ ID NO: 103)
MRGARGAWDFLCVLLLLLRVQTGSSQPSVSPGEPSPPSIHPGKSDLIVR
VGDEIRLLCTDPGFVKWTFEILDETNENKQNEWITEKAEATNTGKYTCT
NKHGLSNSIYVFVRDPAKLFLVDRSLYGKEDNDTLVRCPLTDPEVINYS
LKGCQGKPLPKDLRFIPDPKAGIMIKSVKRAYHRLCLHCSVDQEGKSVL
SEKFILKVRPAFKAVPVVSVSKASYLLREGEEFTVTCTIKDVSSSVYST
WKRENSQTKLQEKYNSWHHGDFNYERQATLTISSARVNDSGVFMCYANN
TFGSANVTTTLEVVDKGFINIFPMINTTVFVNDGENVDLIVEYEAFPKP
EHQQWIYMNRIFTDKWEDYPKSENESNIRYVSELHLTRLKGTEGGTYTF
LVSNSDVNAAIAFNVYVNTKPEILTYDRLVNGMLQCVAAGFPEPTIDWY
FCPGTEQRCSASVLPVDVQTLNSSGPPFGKLVVQSSIDSSAFKHNGTVE
CKAYNDVGKISAYFNFAFKGNNKEQIHPHILFTPLLIGFVIVAGMMCII
VMILTYKYLQKPMYEVQWKVVEEINGNNYVYIDPTQLPYDHKWEFPRNR
LSFGKTLGAGAFGKVVEATAYGLIKSDAAMTVAVKMLKPSAHLTEREAL
MSELKVLSYLGNHMNIVNLLGACTIGGPTLVITEYCCYGDLLNFLRRKR
DSFICSKQEDHAEAALYKNLLHSKESSCSDSTNEYMDMKPGVSYVVPTK
ADKRRSVRIGSYIERDVTPAIMEDDELALDLEDLLSFSYQVAKGMAFLA
SKNCIHRDLAARNILLTHGRITKICDFGLARDIKNDSNYVVKGNARLPV
KWMAPESIFNCVYTFESDVWSYGIFLWELFSLGSSPYPGMPVDSKFYKM
IKEGFRMLSPEHAPAEMYDIMKTCWDADPLKRPTFKQIVQLIEKQISES
TNHIYSNLANCSPNRQKPVVDHSVRINSVGSTASSSQPLLVHDDV
(Uniprot No. P10721)

In some embodiments, a second or subsequent target domain to provide a second or subsequent mutation, e.g., in a target gene encoding a cell-surface protein epitope, is complementary to a target nucleic acid encoding an epitope in a EMR2 cell-surface epitope or a fragment of the epitope sufficient to bind to an agent (e.g., a ligand or an antibody), e.g., wherein the EMR2 comprises the amino acid sequence of SEQ ID NO: 104 or a sequence at least 70%, 75%, 80%, 85%, 90%, 95%, 96%, 97%, 98%, or 99% identical thereto.

(SEQ ID NO: 104)
MGGRVFLVFLAFCVWLTLPGAETQDSRGCARWCPQDSSCVNATACRCNP
GFSSFSEIITTPMETCDDINECATLSKVSCGKFSDCWNTEGSYDCVCSP
GYEPVSGAKTFKNESENTCQDVDECQQNPRLCKSYGTCVNTLGSYTCQC
LPGFKLKPEDPKLCTDVNECTSGQNPCHSSTHCLNNVGSYQCRCRPGWQ
PIPGSPNGPNNTVCEDVDECSSGQHQCDSSTVCFNTVGSYSCRCRPGWK
PRHGIPNNQKDTVCEDMTFSTWTPPPGVHSQTLSRFFDKVQDLGRDYKP
GLANNTIQSILQALDELLEAPGDLETLPRLQQHCVASHLLDGLEDVLRG
LSKNLSNGLLNFSYPAGTELSLEVQKQVDRSVTLRQNQAVMQLDWNQAQ
KSGDPGPSVVGLVSIPGMGKLLAEAPLVLEPEKQMLLHETHQGLLQDGS
PILLSDVISAFLSNNDTQNLSSPVTFTFSHRSVIPRQKVLCVFWEHGQN
GCGHWATTGCSTIGTRDTSTICRCTHLSSFAVLMAHYDVQEEDPVLIVI
TYMGLSVSLLCLLLAALTFLLCKAIQNTSTSLHLQLSLCLFLAHLLFLV
AIDQTGHKVLCSIIAGTLHYLYLATLTWMLLEALYLFLTARNLTVVNYS
SINRFMKKLMFPVGYGVPAVTVAISAASRPHLYGTPSRCWLQPEKGFIW
GFLGPVCAIFSVNLVLFLVTLWILKNRLSSLNSEVSTLRNTRMLAFKAT
AQLFILGCTWCLGILQVGPAARVMAYLFTIINSLQGVFIFLVYCLLSQQ
VREQYGKWSKGIRKLKTESEMHTLSSSAKADTSKPSTVN
(Uniprot No. Q9UHX3)

In some embodiments, a second or subsequent target domain to provide a second or subsequent mutation, e.g., in a target gene encoding a cell-surface protein epitope, is complementary to a target nucleic acid encoding an epitope in a CD5 cell-surface epitope or a fragment of the epitope sufficient to bind to an agent (e.g., a ligand or an antibody), e.g., wherein the CD5 comprises the amino acid sequence of SEQ ID NO: 105 or a sequence at least 70%, 75%, 80%, 85%, 90%, 95%, 96%, 97%, 98%, or 99% identical thereto.

(SEQ ID NQ: 105)
MPMGSLQPLATLYLLGMLVASCLGRLSWYDPDFQARLTRSNSKCQGQLE
VYLKDGWHMVCSQSWGRSSKQWEDPSQASKVCQRLNCGVPLSLGPFLVT
YTPQSSIICYGQLGSFSNCSHSRNDMCHSLGLTCLEPQKITPPTTRPPP
TTTPEPTAPPRLQLVAQSGGQHCAGVVEFYSGSLGGTISYEAQDKTQDL
ENFLCNNLQCGSFLKHLPETEAGRAQDPGEPREHQPLPIQWKIQNSSCT
SLEHCFRKIKPQKSGRVLALLCSGFQPKVQSRLVGGSSICEGTVEVRQG
AQWAALCDSSSARSSLRWEEVCREQQCGSVNSYRVLDAGDPTSRGLFCP
HQKLSQCHELWERNSYCKKVFVTCQDPNPAGLAAGTVASIILALVLLVV
LLVVCGPLAYKKLVKKFRQKKQRQWIGPTGMNQNMSFHRNHTATVRSHA
ENPTASHVDNEYSQPPRNSHLSAYPALEGALHRSSMQPDNSSDSDYDLH
GAQRL (Uniprot No. P06127)

gRNAs Targeting CD33

The present disclosure provides a number of exemplary useful gRNAs that is capable of targeting a Cas nuclease to human CD33. In particular embodiments, the present disclosure provides gRNAs that is capable of targeting a nuclease to a nucleic acid sequence that encodes the amino acid sequence of a CD33 cell-surface epitope or a fragment of the epitope sufficient to bind to an agent (e.g., a ligand or an antibody). In particular embodiments, the present disclosure provides gRNAs that is capable of targeting a nuclease to a nucleic acid sequence that encodes the amino acid sequence of a CD33 cell-surface epitope or a fragment of the epitope sufficient to bind to Lintuzumab (hM195) (FIGS. 1-6).

In some embodiments, the first gRNA comprising a targeting domain which binds a target site in a CD33 gene, wherein the target site comprises the nucleic acid sequence of GCAAGTGCAGGAGTCAGTGA (SEQ ID NO: 68), or a portion thereof.

In some embodiments, the first gRNA comprising a targeting domain which binds a target site in a CD33 gene, wherein the target site comprises the nucleic acid sequence of GCAAGTGCAGGAGTCAGTG (SEQ ID NO: 86), or a portion thereof.

In some embodiments, the second gRNA comprising a targeting domain which binds a target site in a CD33 gene, wherein the target site comprises the nucleic acid sequence of GGATCCAAATTTCTGGCTGC (SEQ ID NO: 70), or a portion thereof.

In some embodiments, the second gRNA comprising a targeting domain which binds a target site in a CD33 gene, wherein the target site comprises the nucleic acid sequence of GGATCCAAATTTCTGGCTG (SEQ ID NO: 87), or a portion thereof.

gRNAs Targeting CD20

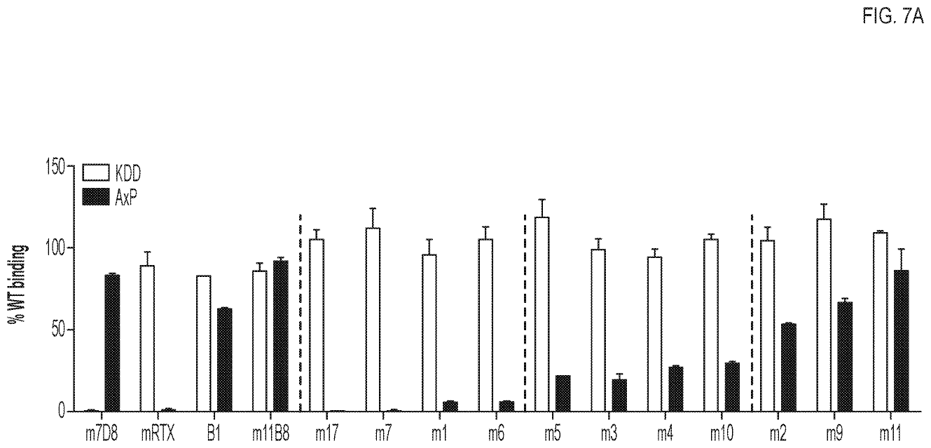

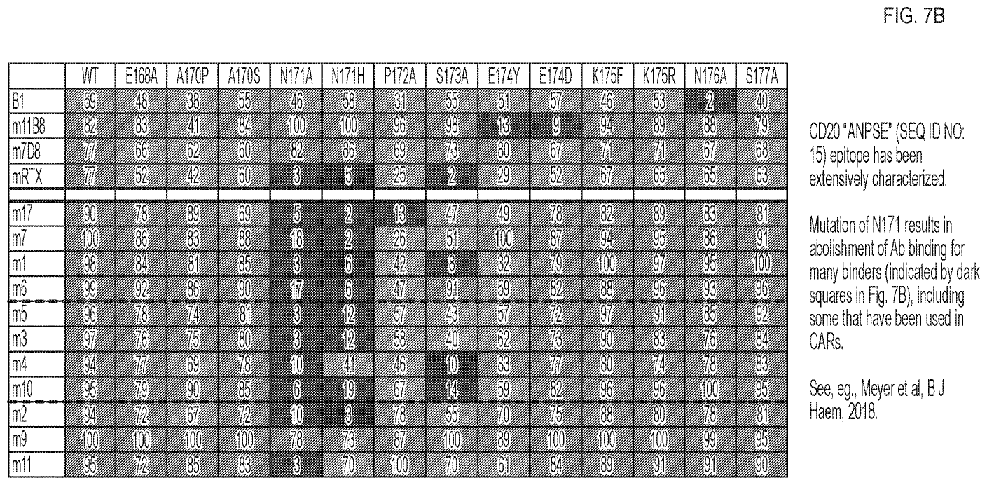

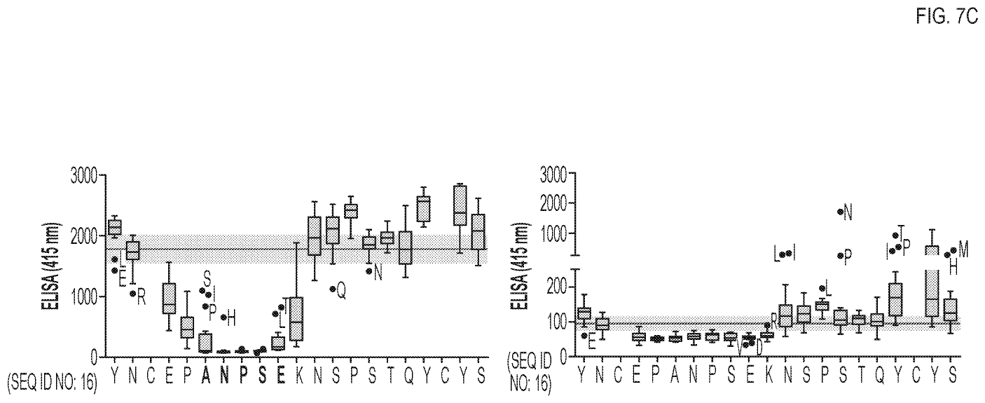

The present disclosure provides a number of exemplary useful gRNAs that is capable of targeting a Cas nuclease to human CD20. In particular embodiments, the present disclosure provides gRNAs that is capable of targeting a nuclease to a nucleic acid sequence that encodes the amino acid sequence of a CD20 cell-surface epitope or a fragment of the epitope sufficient to bind to an agent (e.g., a ligand or an antibody). In particular embodiments, the present disclosure provides gRNAs that is capable of targeting a nuclease to a nucleic acid sequence that encodes the amino acid sequence of a CD20 cell-surface epitope or a fragment of the epitope sufficient to bind to a Type I CD20 mAbs, for example, m708 and mRTX, and/or a Type II mAbs, for example, B1 and m1188 (FIGS. 7-9).

In some embodiments, the first gRNA comprising a targeting domain which binds a target site in a CD20 gene, wherein the target site comprises the nucleic acid sequence of ATATAATTGTATATGTTGAC (SEQ ID NO: 69), or a portion thereof.

In some embodiments, the second gRNA comprising a targeting domain which binds a target site in a CD20 gene, wherein the target site comprises the nucleic acid sequence of ACTTGGTCGATTAGGGAGAC (SEQ ID NO: 71), or a portion thereof.

gRNAs Targeting CD38

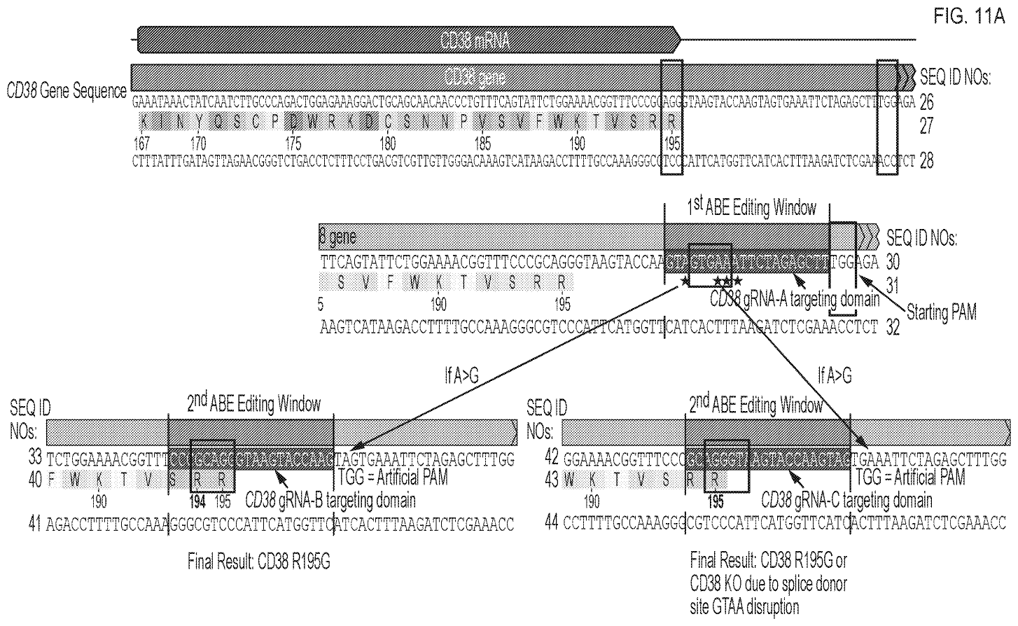

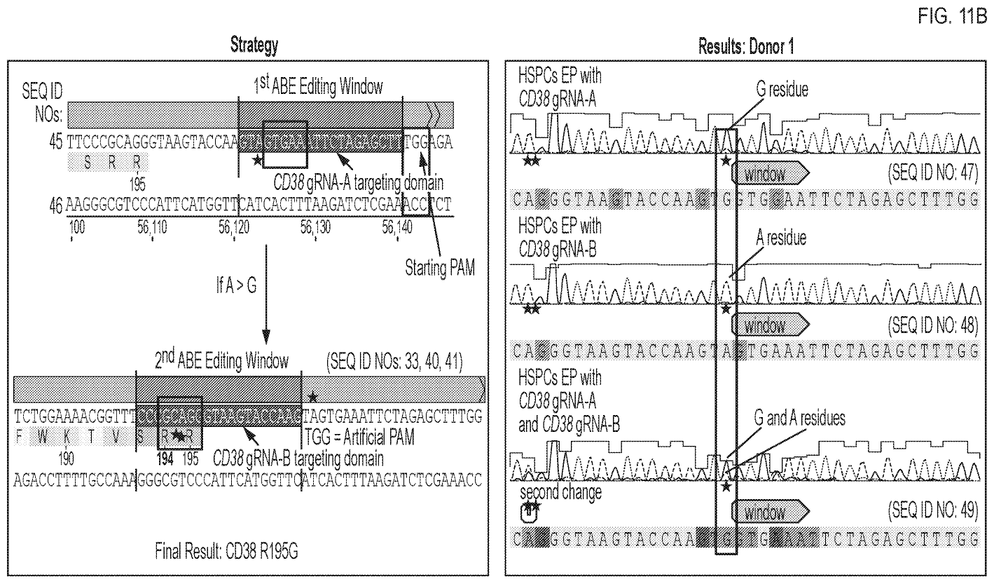

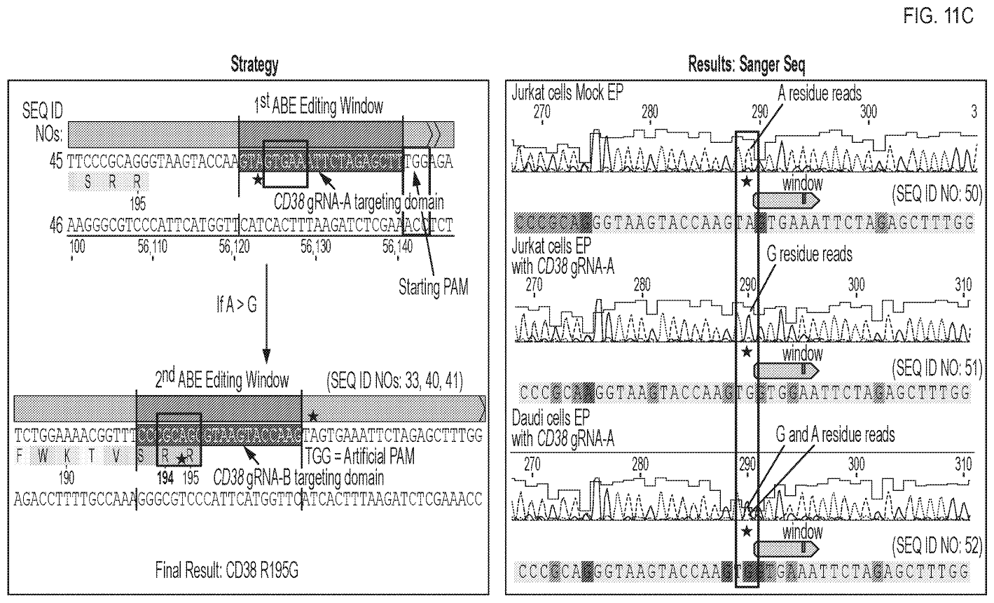

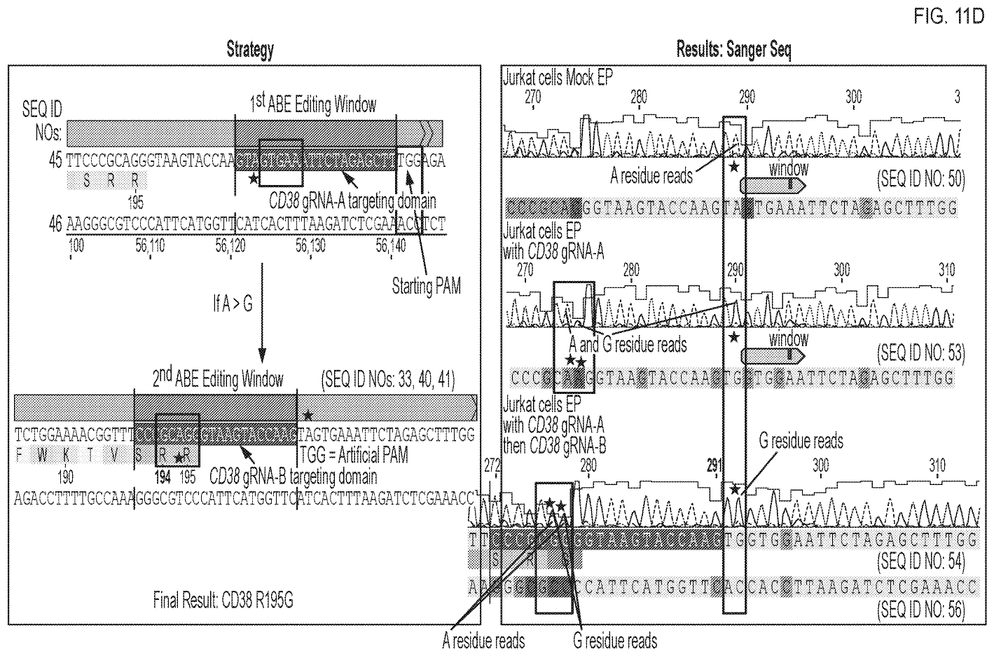

The present disclosure provides a number of exemplary useful gRNAs that is capable of targeting a Cas nuclease to human CD38. In particular embodiments, the present disclosure provides gRNAs that is capable of targeting a nuclease to a nucleic acid sequence that encodes the amino acid sequence of a CD38 cell-surface epitope or a fragment of the epitope sufficient to bind to an agent (e.g., a ligand or an antibody). In particular embodiments, the present disclosure provides gRNAs that is capable of targeting a nuclease to a nucleic acid sequence that encodes the amino acid sequence of a CD38 cell-surface epitope or a fragment of the epitope sufficient to bind to isatuximab (FIG. 11A).

In some embodiments, the first gRNA comprising a targeting domain which binds a target site in a CD38 gene, wherein the target site comprises the nucleic acid sequence of GTAGTGAAATTCTAGAGCTT (SEQ ID NO: 81), or a portion thereof.

In some embodiments, the second gRNA comprising a targeting domain which binds a target site in a CD38 gene, wherein the target site comprises the nucleic acid sequence of CCCGCAGGGTAAGTACCAAG (SEQ ID NO: 83) or GCAGGGTAAGTACCAAGTAG (SEQ ID NO: 84), or a portion thereof.

gRNAs Targeting CD123

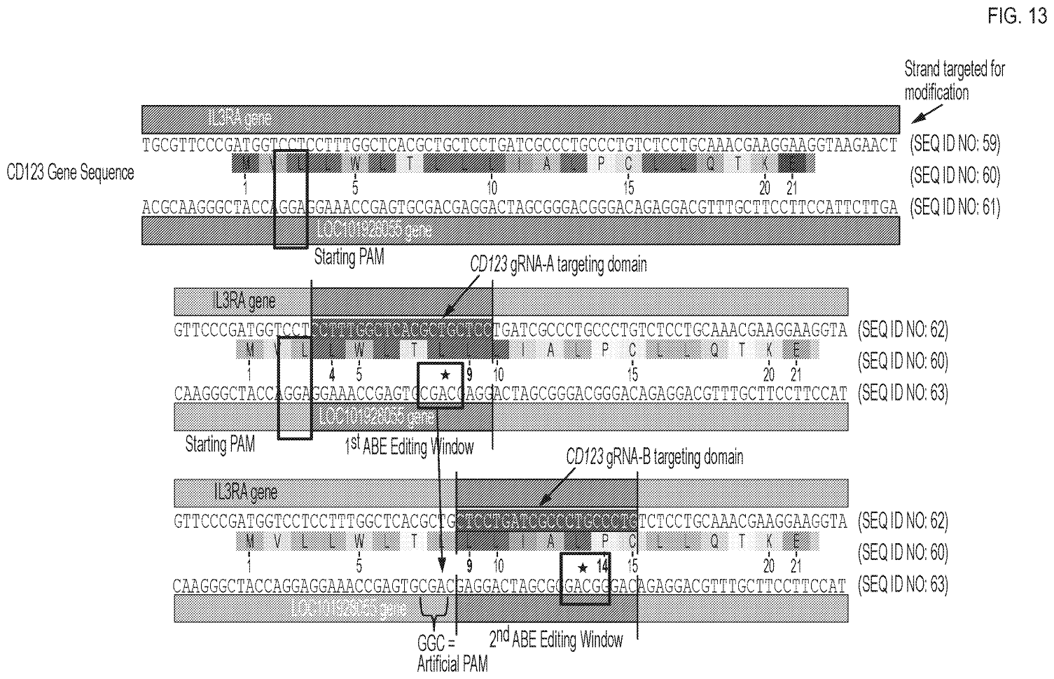

The present disclosure provides a number of exemplary useful gRNAs that is capable of targeting a Cas nuclease to human CD123. In particular embodiments, the present disclosure provides gRNAs that is capable of targeting a nuclease to a nucleic acid sequence that encodes the amino acid sequence of a CD123 cell-surface epitope or a fragment of the epitope sufficient to bind to an agent (e.g., a ligand or an antibody). In particular embodiments, the present disclosure provides gRNAs that is capable of targeting a nuclease to a nucleic acid sequence that encodes the amino acid sequence of a CD123 cell-surface epitope or a fragment of the epitope sufficient to bind to one or more of flotetuzumab, vibecotamab, JNJ-63709178, APVO436, 6H6, 9F5, 7G3 (JNJ-56022473, or a humanized variant thereof (e.g., antibody CSL-362)), and SAR440234 (FIG. 13).

In some embodiments, the first gRNA comprising a targeting domain which binds a target site in a CD123 gene, wherein the target site comprises the nucleic acid sequence of CCTTTGGCTCACGCTGCTCC (SEQ ID NO: 82), or a portion thereof.

In some embodiments, the second gRNA comprising a targeting domain which binds a target site in a CD123 gene, wherein the target site comprises the nucleic acid sequence of CTCCTGATCGCCCTGCCTG (SEQ ID NO: 85), or a portion thereof.

Dual and Multiple gRNA Compositions and Uses Thereof

In some embodiments, a gRNA described herein can be used in combination with one or more gRNA, e.g., for directing nucleases to one or more sites in a genome. In some embodiments, multiple gRNA described herein can be used in combination, e.g., for directing nucleases to multiple sites in a genome. In some embodiments, a gRNA described herein can be used in combination with a second gRNA, e.g., for directing nucleases to two sites in a genome. In some embodiments, a gRNA described herein can be used in combination with a third gRNA, e.g., for directing nucleases to three sites in a genome. In some embodiments, a gRNA described herein can be used in combination with a fourth gRNA, e.g., for directing nucleases to four sites in a genome. In some embodiments, a gRNA described herein can be used in combination with a fifth gRNA, e.g., for directing nucleases to five sites in a genome. In some embodiments, a gRNA described herein can be used in combination with a sixth gRNA, e.g., for directing nucleases to six sites in a genome. In some embodiments, a gRNA described herein can be used in combination with a seventh gRNA, e.g., for directing nucleases to seven sites in a genome. In some embodiments, a gRNA described herein can be used in combination with an eight gRNA, e.g., for directing nucleases to eight sites in a genome. In some embodiments, a gRNA described herein can be used in combination with a ninth gRNA, e.g., for directing nucleases to nine sites in a genome. In some embodiments, a gRNA described herein can be used in combination with a tenth gRNA, e.g., for directing nucleases to ten sites in a genome. In some embodiments, a gRNA described herein can be used in combination with more than tenth gRNA, e.g., for directing nucleases to more than ten sites in a genome.

For instance, in some embodiments, it is desired to produce a hematopoietic cell that includes a modified cell-surface epitope in a first lineage-specific cell-surface antigen (e.g., a lineage-specific cell-surface antigen, e.g., CD33, CD20, CLL-1, CD123, CD38, CD19, CD117, EMR2, and/or CD5) and modified cell-surface epitope in a second lineage-specific cell-surface antigen (e.g., a lineage-specific cell-surface antigen, e.g., CD33, CD20, CLL-1, CD123, CD38, CD19, CD117, EMR2, and/or CD5), e.g., so that the cell can be resistant to two agents: an agent targeting the first lineage-specific cell-surface antigen and an agent targeting the second lineage-specific cell-surface antigen. In some embodiments, it is desirable to contact a cell with two or more different gRNAs that target different gene sequences encoding cell-surface epitopes of a lineage-specific cell-surface antigen (e.g., a lineage-specific cell-surface antigen, e.g., CD33, CD20, CLL-1, CD123, CD38, CD19, CD117, EMR2, and/or CD5), in order to make two or more cuts and create a deletion between the two cut sites.

In some embodiments, it is desired to produce a hematopoietic cell that is deficient for a first lineage-specific cell-surface antigen (e.g., a lineage-specific cell-surface antigen, e.g., CD33, CD20, CLL-1, CD123, CD38, CD19, CD117, EMR2, and/or CD5) and a second lineage-specific cell-surface antigen (e.g., a lineage-specific cell-surface antigen, e.g., CD33, CD20, CLL-1, CD123, CD38, CD19, CD117, EMR2, and/or CD5), e.g., so that the cell can be resistant to two agents: an agent targeting the first lineage-specific cell-surface antigen and an agent targeting the second lineage-specific cell-surface antigen. In some embodiments, it is desirable to contact a cell with two or more different gRNAs that target different regions of a gene encoding a lineage-specific cell-surface antigen (e.g., a lineage-specific cell-surface antigen, e.g., CD33, CD20, CLL-1, CD123, CD38, CD19, CD117, EMR2, and/or CD5), in order to make two or more cuts and create a deletion between the two cut sites.

Accordingly, the disclosure provides various combinations of gRNAs and related CRISPR systems, as well as cells created by genome editing methods using such combinations of gRNAs and related CRISPR systems. In some embodiments, the first lineage-specific cell-surface antigen gRNA binds a different nuclease than the second gRNA. For example, in some embodiments, the first lineage-specific cell-surface antigen gRNA may bind Cas9 and the second gRNA may bind Cas12a, or vice versa.

Accordingly, the disclosure provides various combinations of gRNAs and related base editing systems, as well as cells created by genome editing methods using such combinations of gRNAs and related base editing systems.

In some embodiments, two or more (e.g., 3, 4, or more) gRNAs described herein are admixed. In some embodiments, each gRNA is in a separate container. In some embodiments, a kit described herein (e.g., a kit comprising one or more gRNAs described herein for generating an artificial PAM and/or modifying a cell-surface epitope) also comprises a Cas9 nuclease, or a nucleic acid encoding the Cas9 nuclease.

In some embodiments, it is desirable to contact a cell with two or more different gRNAs that target different sites of CD33, CD20, CLL-1, CD123, CD38, CD19, CD117, EMR2, and/or CD5, e.g., in order to make multiple chemical alteration to a nucleobase(s). In some embodiments, the first and second gRNAs are gRNAs as described herein, or variants thereof.

In some embodiments, it is desirable to contact a cell with two or more different gRNAs that target different sites of CD33, CD38, CD123, and/or CD20, e.g., in order to make multiple chemical alteration to a nucleobase(s). In some embodiments, the first and second gRNAs are gRNAs as described herein, or variants thereof.

In some embodiments, it is desirable to contact a cell with two or more different gRNAs that target different sites of CD33, e.g., in order to make multiple chemical alteration to a nucleobase(s). In some embodiments, the first and second gRNAs are gRNAs described herein, or variants thereof.

In some embodiments, it is desirable to contact a cell with two or more different gRNAs that target different sites of CD20, e.g., in order to make multiple chemical alteration to a nucleobase(s). In some embodiments, the first and second gRNAs are gRNAs described herein, or variants thereof.

In some embodiments, it is desirable to contact a cell with two or more different gRNAs that target different sites of CD38, e.g., in order to make multiple chemical alteration to a nucleobase(s). In some embodiments, the first and second gRNAs are gRNAs described herein, or variants thereof.

In some embodiments, it is desirable to contact a cell with two or more different gRNAs that target different sites of CD123, e.g., in order to make multiple chemical alteration to a nucleobase(s). In some embodiments, the first and second gRNAs are gRNAs described herein, or variants thereof.

In some embodiments, the first gRNA is a CD33, CD20, CLL-1, CD123, CD38, CD19, CD117, EMR2, and/or CD5 gRNA and the second gRNA targets a gene encoding a lineage-specific cell-surface antigen chosen from: CD33, CD20, CLL-1, CD123, CD38, CD19, CD117, EMR2, and/or CD5.

In some embodiments, the first gRNA is a CD33 gRNA, a CD38 gRNA, a CD123 gRNA, or a CD20 gRNA described herein and the second gRNA targets a gene encoding a lineage-specific cell-surface antigen chosen from: CD33, CD20, CLL-1, CD123, CD38, CD19, CD117, EMR2, and/or CD5. In some embodiments, the first gRNA is a CD33 gRNA, a CD38 gRNA, a CD123 gRNA, or a CD20 gRNA described herein and the second gRNA targets a gene encoding a lineage-specific cell-surface antigen chosen from: BCMA, CD19, CD20, CD30, ROR1, B7H6, B7H3, CD23, CD33, CD38, C-type lectin like molecule-1, CS1, IL-5, L1-CAM, PSCA, PSMA, CD138, CD133, CD70, CD7, CD13, NKG2D, NKG2D ligand, CLEC12A, CD11, CD123, CD56, CD30, CD34, CD14, CD66b, CD41, CD61, CD62, CD235a, CD146, CD326, LMP2, CD22, CD52, CD10, CD3/TCR, CD79/BCR, and CD26.

In some embodiments, the first gRNA is a CD33 gRNA, a CD38 gRNA, a CD123 gRNA, or a CD20 gRNA described herein and the second gRNA targets a gene encoding a lineage-specific cell-surface antigen associated with a specific type of cancer, such as, without limitation, CD20, CD22 (Non-Hodgkin's lymphoma, B-cell lymphoma, chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (epithelial and lymphoid malignancies), human leukocyte antigen (HLA)-DR, HLA-DP, and HLA-DQ (lymphoid malignancies), RCAS1 (gynecological carcinomas, biliary adenocarcinomas and ductal adenocarcinomas of the pancreas) as well as prostate specific membrane antigen.

In some embodiments, the first gRNA is a CD33 gRNA, a CD38 gRNA, a CD123 gRNA, or a CD20 gRNA described herein and the second gRNA targets a gene encoding a lineage-specific cell-surface antigen chosen from: CD7, CD13, CD19, CD22, CD20, CD25, CD32, CD33, CD38, CD44, CD45, CD47, CD56, 96, CD117, CD123, CD135, CD174, CLL-1, folate receptor B, IL1RAP, MUC1, NKG2D/NKG2DL, TIM-3, or WT1.

In some embodiments, the first gRNA is a CD33 gRNA, a CD38 gRNA, a CD123 gRNA, or a CD20 gRNA described herein and the second gRNA targets a gene encoding a lineage-specific cell-surface antigen chosen from: CD1a, CD1b, CD1c, CD1d, CDle, CD2, CD3, CD3d, CD3e, CD3g, CD4, CD5, CD6, CD7, CD8a, CD8b, CD9, CD10, CD11a, CD11b, CD11c, CD11d, CDw12, CD13, CD14, CD15, CD16, CD16b, CD17, CD18, CD19, CD20, CD21, CD22, CD23, CD24, CD25, CD26, CD27, CD28, CD29, CD30, CD31, CD32a, CD32b, CD32c, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD61, CD62E, CD62L, CD62P, CD63, CD64a, CD65, CD65s, CD66a, CD66b, CD66c, CD66F, CD68, CD69, CD70, CD71, CD72, CD73, CD74, CD75, CD75S, CD77, CD79a, CD79b, CD80, CD81, CD82, CD83, CD84, CD85A, CD85C, CD85D, CD85E, CD85F, CD85G, CD85H, CD85I, CD85J, CD85K, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD14, CDw145, CD146, CD147, CD148, CD150, CD152, CD152, CD153, CD154, CD155, CD156a, CD156b, CD156c, CD157, CD158b1, CD158b2, CD158d, CD158e1/e2, CD158f, CD158g, CD158h, CD158i, CD158j, CD158k, CD159a, CD159c, CD160, CD161, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203c, CD204, CD205, CD206, CD207, CD208, CD209, CD210a, CDw210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270, CD272, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300e, CD301, CD302, CD303, CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD359, CD360, CD361, CD362, CD363, CD364, CD365, CD366, CD367, CD368, CD369, CD370, or CD371. See also examples of lineage-specific cell-surface antigens from BD Biosciences Human CD Marker Chart, bdbiosciences.com/content/dam/bdb/campaigns/reagent-education/BD_Reagents_CDMarkerHuman_Poster.pdf (incorporated by reference in its entirety).

In some embodiments, the second gRNA is a gRNA disclosed in any of International Publication Nos. WO2017/066760, WO2019/046285, WO/2018/160768, or in Borot et al. PNAS (2019) 116 (24): 11978-11987, each of which is incorporated herein by reference in its entirety.

In some embodiments, the first gRNA is a CD33 gRNA, a CD38 gRNA, a CD123 gRNA, or a CD20 gRNA described herein and the second gRNA targets a gene encoding a lineage-specific cell-surface antigen chosen from: CD19; CD123; CD22; CD30; CD171; CS-1 (also referred to as CD2 subset 1, CRACC, SLAMF7, CD319, and 19A₂₄); C-type lectin-like molecule-1 (CLECL1); epidermal growth factor receptor variant III (EGFRvIII); ganglioside G2 (CD2); ganglioside GD3 (aNeu5Ac (2-8) aNeu5Ac (2-3) bDGalp (1-4) bDGlep (1-1) Cer); TNF receptor family member B cell maturation (BCMA), Tn antigen ((Tn Ag) or (GalNAca-Ser/Thr)); prostate-specific membrane antigen (PSMA); Receptor tyrosine kinase-like orphan receptor 1 (ROR1); Fms-Like tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A₂); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell-surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor I receptor (IGF-I receptor), carbonic anhydrase IX (CAIX), Proteasome (Prosome, Macropain) Subunit, Beta Type 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac (2-3) bDGalp (1-4) bDGlcp (1-1) Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex; locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A₁), ETS translocation-variant gene 6, located on chromosome 12p (ETV6-AML); sperm protein 17 (SPA17); X Antigen Family, member 1A (XAGE1); angiopoietin-binding cell-surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-1AP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxy esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2), lymphocyte antigen 75 (LY75); Glypican-3 (GPC3); Fc receptor-like 5 (FCRL5); and immunoglobulin lambda-like polypeptide 1 (IGLL1).

In some embodiments, the first gRNA is a CD33 gRNA, CD38 gRNA, CD123 gRNA, or a CD20 gRNA described herein and the second gRNA targets a gene encoding a lineage-specific cell-surface antigen chosen from: CD11a, CD18, CD19, CD20, CD31, CD33, CD34, CD44, CD45, CD47, CD51, CD58, CD59, CD63, CD97, CD99, CD100, CD102, CD123, CD127, CD133, CD135, CD157, CD172b, CD217, CD300a, CD305, CD317, CD321, and CLL-1.

In some embodiments, the first gRNA is a CD33 gRNA, CD38 gRNA, CD123 gRNA, or a CD20 gRNA described herein and the second gRNA targets a gene encoding a lineage-specific cell-surface antigen chosen from: CD123, CLL-1, CD38, CD135 (FLT3), CD56 (NCAM1), CD117 (c-KIT), FRβ (FOLR2), CD47, CD82, TNFRSF1B (CD120B), CD191, CD96, PTPRJ (CD148), CD70, LILRB2 (CD85D), CD25 (IL2Ralpha), CD44, CD96, NKG2D Ligand, CD45, CD7, CD15, CD19, CD20, CD22, CD37, and CD82. In some embodiments, the first gRNA is a CD33 gRNA, CD38 gRNA, CD123 gRNA, or a CD20 gRNA described herein and the second gRNA targets a gene encoding a lineage-specific cell-surface antigen chosen from: CD7, CD11a, CD15, CD18, CD19, CD20, CD22, CD25, CD31, CD34, CD37, CD38, CD44, CD45, CD47, CD51, CD56, CD58, CD59, CD63, CD70, CD82, CD85D, CD96, CD97, CD99, CD100, CD102, CD117, CD120B, CD123, CD127, CD133, CD135, CD148, CD157, CD172b, CD191, CD217, CD300a, CD305, CD317, CD321, CLL-1, FRβ (FOLR2), or NKG2D Ligand. In some embodiments, the first gRNA is a CD33 gRNA described herein and the second gRNA targets a gene encoding a lineage-specific cell-surface antigen selected from a group consisting of: CD7, CD11a, CD15, CD18, CD19, CD20, CD22, CD31, CD34, CD37, CD38, CD44, CD45, CD47, CD51, CD58, CD59, CD63, CD70, CD82, CD85D, CD96, CD97, CD99, CD100, CD102, CD120B, CD123, CD127, CD133, CD157, CD172b, CD191, CD217, CD300a, CD305, CD317, CD321, CLL-1, FRβ (FOLR2), CD135 (FLT3), CD56 (NCAM1), CD117 (c-KIT), FRβ (FOLR2), TNFRSF1B (CD120B), CD96, PTPRJ (CD148), CD70, LILRB2 (CD85D), CD25 (IL2Ralpha), and NKG2D Ligand.

In some embodiments, the first gRNA is a CD123 gRNA described herein and the second gRNA targets a gene encoding lineage-specific cell-surface antigen selected from a group consisting of: CD7, CD11a, CD15, CD18, CD19, CD20, CD22, CD31, CD33, CD34, CD37, CD38, CD44, CD45, CD47, CD51, CD58, CD59, CD63, CD70, CD82, CD85D, CD96, CD97, CD99, CD100, CD102, CD120B, CD127, CD133, CD157, CD172b, CD191, CD217, CD300a, CD305, CD317, CD321, CLL-1, FRβ (FOLR2), CD135 (FLT3), CD56 (NCAM1), CD117 (c-KIT), FRβ (FOLR2), TNFRSF1B (CD120B), CD96, PTPRJ (CD148), CD70, LILRB2 (CD85D), CD25 (IL2Ralpha), and NKG2D Ligand.

In some embodiments, the first gRNA is a CD38 gRNA described herein and the second gRNA targets a gene encoding a lineage-specific cell-surface antigen selected from a group consisting of: CD7, CD11a, CD15, CD18, CD19, CD20, CD22, CD31, CD33, CD34, CD37, CD44, CD45, CD47, CD51, CD58, CD59, CD63, CD70, CD82, CD85D, CD96, CD97, CD99, CD100, CD102, CD120B, CD123, CD127, CD133, CD157, CD172b, CD191, CD217, CD300a, CD305, CD317, CD321, CLL-1, FRβ (FOLR2), CD135 (FLT3), CD56 (NCAM1), CD117 (c-KIT), FRβ (FOLR2), TNFRSF1B (CD120B), CD96, PTPRJ (CD148), CD70, LILRB2 (CD85D), CD25 (IL2Ralpha), and NKG2D Ligand.

In some embodiments, the first gRNA is a CD20 gRNA described herein and the second gRNA targets a gene encoding a lineage-specific cell-surface antigen selected from a group consisting of: CD7, CD11a, CD15, CD18, CD19, CD22, CD31, CD33, CD34, CD37, CD38, CD44, CD45, CD47, CD51, CD58, CD59, CD63, CD70, CD82, CD85D, CD96, CD97, CD99, CD100, CD102, CD120B, CD123, CD127, CD133, CD157, CD172b, CD191, CD217, CD300a, CD305, CD317, CD321, CLL-1, FRβ (FOLR2), CD135 (FLT3), CD56 (NCAM1), CD117 (c-KIT), FRβ (FOLR2), TNFRSF1B (CD120B), CD96, PTPRJ (CD148), CD70, LILRB2 (CD85D), CD25 (IL2Ralpha), and NKG2D Ligand.

In some embodiments, the second gRNA is a CD33 gRNA described herein and the first gRNA targets a gene encoding a lineage-specific cell-surface antigen selected from a group consisting of: CD7, CD11a, CD15, CD18, CD19, CD20, CD22, CD31, CD34, CD37, CD38, CD44, CD45, CD47, CD51, CD58, CD59, CD63, CD70, CD82, CD85D, CD96, CD97, CD99, CD100, CD102, CD120B, CD123, CD127, CD133, CD157, CD172b, CD191, CD217, CD300a, CD305, CD317, CD321, CLL-1, FRβ (FOLR2), CD135 (FLT3), CD56 (NCAM1), CD117 (c-KIT), FRβ (FOLR2), TNFRSF1B (CD120B), CD96, PTPRJ (CD148), CD70, LILRB2 (CD85D), CD25 (IL2Ralpha), and NKG2D Ligand.

In some embodiments, the second gRNA is a CD123 gRNA described herein and the first gRNA targets a gene encoding a lineage-specific cell-surface antigen selected from a group consisting of: CD7, CD11a, CD15, CD18, CD19, CD20, CD22, CD31, CD33, CD34, CD37, CD38, CD44, CD45, CD47, CD51, CD58, CD59, CD63, CD70, CD82, CD85D, CD96, CD97, CD99, CD100, CD102, CD120B, CD127, CD133, CD157, CD172b, CD191, CD217, CD300a, CD305, CD317, CD321, CLL-1, FRβ (FOLR2), CD135 (FLT3), CD56 (NCAM1), CD117 (c-KIT), FRβ (FOLR2), TNFRSF1B (CD120B), CD96, PTPRJ (CD148), CD70, LILRB2 (CD85D), CD25 (IL2Ralpha), and NKG2D Ligand.

In some embodiments, the second gRNA is a CD38 gRNA described herein and the first gRNA targets a gene encoding a lineage-specific cell-surface antigen selected from a group consisting of: CD7, CD11a, CD15, CD18, CD19, CD20, CD22, CD31, CD33, CD34, CD37, CD44, CD45, CD47, CD51, CD58, CD59, CD63, CD70, CD82, CD85D, CD96, CD97, CD99, CD100, CD102, CD120B, CD123, CD127, CD133, CD157, CD172b, CD191, CD217, CD300a, CD305, CD317, CD321, CLL-1, FRβ (FOLR2), CD135 (FLT3), CD56 (NCAM1), CD117 (c-KIT), FRβ (FOLR2), TNFRSF1B (CD120B), CD96, PTPRJ (CD148), CD70, LILRB2 (CD85D), CD25 (IL2Ralpha), and NKG2D Ligand.

In some embodiments, the second gRNA is a CD20 gRNA described herein and the first gRNA targets a gene encoding a lineage-specific cell-surface antigen selected from a group consisting of: CD7, CD11a, CD15, CD18, CD19, CD22, CD31, CD33, CD34, CD37, CD38, CD44, CD45, CD47, CD51, CD58, CD59, CD63, CD70, CD82, CD85D, CD96, CD97, CD99, CD100, CD102, CD120B, CD123, CD127, CD133, CD157, CD172b, CD191, CD217, CD300a, CD305, CD317, CD321, CLL-1, FRβ (FOLR2), CD135 (FLT3), CD56 (NCAM1), CD117 (c-KIT), FRβ (FOLR2), TNFRSF1B (CD120B), CD96, PTPRJ (CD148), CD70, LILRB2 (CD85D), CD25 (IL2Ralpha), and NKG2D Ligand.

In some embodiments, the second gRNA is a gRNA disclosed in any of WO2017/066760, WO2019/046285, WO/2018/160768, or Borot et al. PNAS Jun. 11, 2019 116 (24) 11978-11987, each of which is incorporated herein by reference in its entirety.

### Cells

In some embodiments, the cells, e.g., genetically engineered cells, are eukaryotic cells, such as mammalian cells, e.g., human cells.

In some embodiments, the cells are immune cells. As used herein, the term “immune cell,” used interchangeably herein with the term “immune effector cell,” refers to a cell that is involved in an immune response, e.g., promotion of an immune response. Examples of immune cells include, but are not limited to, T-lymphocytes, natural killer (NK) cells, macrophages, monocytes, dendritic cells, neutrophils, eosinophils, mast cells, platelets, large granular lymphocytes, Langerhans' cells, or B-lymphocytes. A source of immune cells (e.g., T lymphocytes, B lymphocytes, NK cells) can be obtained from a subject. In some embodiments, the cells, e.g., genetically engineered cells, provided herein are hematopoietic cells, e.g., hematopoietic stem cells (HSCs), hematopoietic progenitor cells (HPCs), hematopoietic stem and progenitor cells (HSPCs). In some embodiments, HSPCs are CD34+ (e.g., express CD34 on their cell surface). Hematopoietic stem cells (HSCs) are cells characterized by pluripotency, self-renewal properties, and/or the ability to generate and/or reconstitute all lineages of the hematopoietic system, including both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively.

In some aspects, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic, and/or autologous.

In some embodiments, the cells are CD4+ cells and/or CD8+ cells.

In some embodiments, the cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In some embodiments, the cells are natural killer (NK) cells.

In some embodiments, the cells are monocytes or granulocytes, e.g., myeloid cells, macrophages, neutrophils, dendritic cells, mast cells, eosinophils, and/or basophils.

In some embodiments, the cells are Jurkat cells or Daudi cells.

Other exemplary cells include stem cells, such as multipotent and pluripotent stem cells, including induced pluripotent stem cells (iPSCs).

In some embodiments, the methods include isolating cells from the subject, preparing, processing, culturing, and/or engineering them, as described herein, and re-introducing them into the same subject or a different subject, before or after a period of storage.

### Exemplary Modified Cells

Some aspects of this disclosure provide genetically engineered cells comprising a modification in their genome that results in a loss of expression of a lineage-specific protein antigen, e.g., CD33, CD38, CD123, and/or CD20, or expression of a variant form of CD33, CD38, CD123, and/or CD20 that is not recognized by an immunotherapeutic agent targeting the lineage-specific protein antigen, e.g., CD33, CD38, CD123, and/or CD20. Such modifications can be introduced via a base editing event, i.e., a mutation. Such a base editing event, i.e., mutation, may include, without limitation, a chemical alteration to a nucleobase. In particular embodiments, the mutation may comprise the deamination of a cytosine. In some embodiments, the mutation may comprise the deamination of an adenine. In particular embodiments, the mutation may comprise a nucleobase transition. In particular embodiments, the mutation may comprise a nucleobase transversion. In particular embodiments, the mutation may comprise converting a cytosine-guanine (C-G) base pair into a thymine-adenine (T-A) base pair within the target nucleic acid molecule. In particular embodiments, the mutation may comprise converting a thymine-adenine (T-A) base pair into a cytosine-guanine (C-G) base pair within the target nucleic acid molecule. In particular embodiments, the mutation may comprise introducing a premature STOP codon within a target nucleic acid molecule. In particular embodiments, the mutation may comprise introducing a splice site within a target nucleic acid molecule. In particular embodiments, the mutation may comprise disrupting a splice site within a target nucleic acid molecule. Accordingly, in some aspects of this disclosure provide genetically engineered cells comprising a plurality of modifications in their genome that results in a loss of expression of CD33, CD38, CD123, and/or CD20, or expression of a variant form of CD33, CD38, CD123, and/or CD20 that is not recognized by an immunotherapeutic agent targeting CD33 and/or CD20. Such variants may include mutations within a cell-surface protein epitope.

For example, some aspects of this disclosure provide, e.g., novel cells having a modification in the endogenous CD33, CD38, CD123, and/or CD20 gene(s) that results in a mutation of a cell-surface protein epitope. In particular, provided herein are cell populations comprising a plurality of genetically engineered hematopoietic stem or progenitor cells, wherein at least a portion of the cells comprise: (i) an artificial PAM; and (ii) a modified cell-surface protein, such as a modified CD33 gene, CD38 gene, CD123 gene, and/or a modified CD20 gene, having a mutant epitope. In some embodiments, a cell (e.g., an HSC or HPC) having a modification resulting in the generation of an artificial PAM is made using a nuclease and/or a gRNA described herein. In some embodiments, a cell (e.g., an HSC or HPC) having a modification of CD33, CD38, CD123, and/or CD20 is made using a nuclease and/or a gRNA described herein. In some embodiments, a cell (e.g., an HSC or HPC) having a modification of CD33, CD38, CD123, and/or CD20 and a modification of a second lineage-specific cell-surface antigen is made using a nuclease and/or a gRNA described herein. In some embodiments, the modification in the genome of the cell is a mutation in a genomic sequence encoding CD33, CD38, CD123, and/or CD20. In some embodiments, the modification is effected via genome editing, e.g., using a Cas nuclease and a gRNA targeting a CD33, CD38, CD123, and/or CD20 target site provided herein or comprising a targeting domain sequence provided herein. It is understood that the cell can be made by contacting the cell itself with the nuclease and/or a gRNA, or the cell can be the daughter cell of a cell that was contacted with the nuclease and/or a gRNA. In some embodiments, a cell described herein (e.g., an HSC) is capable of reconstituting the hematopoietic system of a subject. In some embodiments, a cell described herein (e.g., an HSC) is capable of one or more of (e.g., all of): engrafting in a human subject, producing myeloid lineage cell, and producing and lymphoid lineage cells.

While the compositions, methods, strategies, and treatment modalities provided herein may be applied to any cell or cell type, some exemplary cells and cell types that are particularly suitable for genomic modification in the CD33, CD38, CD123, and/or CD20 gene according to aspects of this disclosure are described in more detail herein. The skilled artisan will understand, however, that the provision of such examples is for the purpose of illustrating some specific embodiments, and additional suitable cells and cell types will be apparent to the skilled artisan based on the present disclosure, which is not limited in this respect.

In some embodiments, a population of cells described herein comprises hematopoietic stem cells (HSCs), hematopoietic progenitor cells (HPCs), or both (HSPCs). In some embodiments, the cells are CD34+. In some embodiments, the cell is a hematopoietic cell. In some embodiments, the cell is a hematopoietic stem cell. In some embodiments, the cell is a hematopoietic progenitor cell. In some embodiments, the cell is an immune effector cell. In some embodiments, the cell is a lymphocyte. In some embodiments, the cell is a T-lymphocyte. In some embodiments, the cell is a NK cell. In some embodiments, the cell is a stem cell. In some embodiments, the stem cell is an embryonic stem cell (ESC), an induced pluripotent stem cell (iPSC), a mesenchymal stem cell, or a tissue-specific stem cell.

In some embodiments, the cell comprises only one genetic modification. In some embodiments, the genetically engineered cell is only genetically modified to comprise an artificial PAM. In some embodiments, the genetically engineered cell is further genetically modified at the CD33, CD38, CD123, and/or CD20 locus. In some embodiments, the genetically engineered cell is genetically modified at a second locus. In some embodiments, the cell does not comprise a transgenic protein, e.g., does not comprise a CAR.

Some aspects of this disclosure provide genetically engineered hematopoietic cells comprising a modification in their genome that results in a loss of expression of CD33, CD38, CD123, and/or CD20, or expression of a variant form of CD33, CD38, CD123, and/or CD20 that is not recognized by an immunotherapeutic agent targeting CD33, CD38, CD123, and/or CD20. In some embodiments, a genetically engineered cell described herein comprises substantially no CD33, CD38, CD123, and/or CD20 protein. In some embodiments, a genetically engineered cell described herein comprises substantially no wild-type CD33, CD38, CD123, and/or CD20 protein, but comprises mutant CD33, CD38, CD123, and/or CD20 protein, e.g., having a mutant cell-surface epitope. In some embodiments, the mutant CD33, CD38, CD123, and/or CD20 protein is not bound by an agent that targets CD33, CD38, CD123, and/or CD20 for therapeutic purposes. In some embodiments, the genetically engineered cells comprising a modification in their genome results in reduced cell-surface expression of CD33, CD38, CD123, and/or CD20 and/or reduced binding by an immunotherapeutic agent targeting CD33, CD38, CD123, and/or CD20, e.g., as compared to a hematopoietic cell (e.g., HSC) of the same cell type but not comprising a genomic modification.

In some embodiments, the cells are hematopoietic cells, e.g., hematopoietic stem cells, hematopoietic progenitor cell (HPC), hematopoietic stem or progenitor cell. Hematopoietic stem cells (HSCs) are cells characterized by pluripotency, self-renewal properties, and/or the ability to generate and/or reconstitute all lineages of the hematopoietic system, including both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. HSCs are characterized by the expression of one or more cell-surface markers, e.g., CD34 (e.g., CD34+), which can be used for the identification and/or isolation of HSCs, and absence of cell-surface markers associated with commitment to a cell lineage. In some embodiments, a genetically engineered cell (e.g., genetically engineered HSC) described herein does not express one or more cell-surface markers typically associated with HSC identification or isolation, expresses a reduced amount of the cell-surface markers, or expresses a variant cell-surface marker not recognized by an immunotherapeutic agent targeting the cell-surface marker, but nevertheless is capable of self-renewal and can generate and/or reconstitute all lineages of the hematopoietic system.

In some embodiments, a population of cells described herein comprises a plurality of hematopoietic stem cells; in some embodiments, a population of cells described herein comprises a plurality of hematopoietic progenitor cells; and in some embodiments, a population of cells described herein comprises a plurality of hematopoietic stem cells and a plurality of hematopoietic progenitor cells.

In some embodiments, a genetically engineered cell provided herein comprises two or more genomic modifications, e.g., one or more genomic modifications in addition to a genomic modification that results in a loss of expression of CD33, CD38, CD123, and/or CD20, or expression of a variant form of CD33, CD38, CD123, and/or CD20 that is not recognized by an immunotherapeutic agent targeting CD33, CD38, CD123, and/or CD20.

In some embodiments, a genetically engineered cell provided herein comprises a genomic modification that results in a loss of expression of CD33, CD38, CD123, and/or CD20, or expression of a variant form of CD33, CD38, CD123, and/or CD20 that is not recognized by an immunotherapeutic agent targeting CD33, CD38, CD123, and/or CD20, and further comprises an expression construct that encodes a chimeric antigen receptor, e.g., in the form of an expression construct encoding the CAR integrated in the genome of the cell. In some embodiments, the CAR comprises a binding domain, e.g., an antibody fragment, that binds CD33, CD38, CD123, and/or CD20.

Some aspects of this disclosure provide genetically engineered immune effector cells comprising a modification in their genome that results in a loss of expression of CD33, CD38, CD123, and/or CD20, or expression of a variant form of CD33, CD38, CD123, and/or CD20 that is not recognized by an immunotherapeutic agent targeting CD33, CD38, CD123, and/or CD20 In some embodiments, the immune effector cell is a lymphocyte. In some embodiments, the immune effector cell is a T-lymphocyte. In some embodiments, the T-lymphocyte is an alpha/beta T-lymphocyte. In some embodiments, the T-lymphocyte is a gamma/delta T-lymphocyte. In some embodiments, the immune effector cell is a natural killer T (NKT) cell. In some embodiments, the immune effector cell is a natural killer (NK) cell. In some embodiments, the immune effector cell does not express an endogenous transgene, e.g., a transgenic protein. In some embodiments, the immune effector cell expresses a chimeric antigen receptor (CAR). In some embodiments, the immune effector cell expresses a CAR targeting CD33, CD38, CD123, and/or CD20. In some embodiments, the immune effector cell does not express a CAR targeting CD33, CD38, CD123, and/or CD20.

In some embodiments, a genetically engineered cell provided herein expresses substantially no CD33, CD38, CD123, and/or CD20 protein, e.g., expresses no CD33, CD38, CD123, and/or CD20 protein that can be measured by a suitable method, such as an immunostaining method. In some embodiments, a genetically engineered cell provided herein expresses substantially no wild-type CD33, CD38, CD123, and/or CD20 protein, but expresses a mutant CD33, CD38, CD123, and/or CD20 protein variant, e.g., a variant not recognized by an immunotherapeutic agent targeting CD33, CD38, CD123, and/or CD20, e.g., a CAR-T cell therapeutic, or an anti-CD33, CD38, CD123, and/or CD20 antibody, antibody fragment, or antibody-drug conjugate (ADC).

In some embodiments, the HSCs are obtained from a subject, such as a human subject. Methods of obtaining HSCs are described, e.g., in PCT/US2016/057339, which is herein incorporated by reference in its entirety. In some embodiments, the HSCs are peripheral blood HSCs. In some embodiments, the mammalian subject is a non-human primate, a rodent (e.g., mouse or rat), a bovine, a porcine, an equine, or a domestic animal. In some embodiments, the HSCs are obtained from a human subject, such as a human subject having a hematopoietic malignancy. In some embodiments, the HSCs are obtained from a healthy donor. In some embodiments, the HSCs are obtained from the subject to whom the immune cells expressing the chimeric receptors will be subsequently administered. HSCs that are administered to the same subject from which the cells were obtained are referred to as autologous cells, whereas HSCs that are obtained from a subject who is not the subject to whom the cells will be administered are referred to as allogeneic cells.

In some embodiments, a population of genetically engineered cells is a heterogeneous population of cells, e.g., heterogeneous population of genetically engineered cells containing different CD33, CD38, CD123, and/or CD20 mutations. In some embodiments, at least 40%, at least 50%, at least 60%, at least 70%, at least 75%, at least 80%, at least 85%, at least 90%, or at least 95% of copies of CD33, CD38, CD123, and/or CD20 in the population of genetically engineered cells have a mutation. In some embodiments, at least 40%, at least 50%, at least 60%, at least 70%, at least 75%, at least 80%, at least 85%, at least 90%, or at least 95% of copies of CD33, CD38, CD123, and/or CD20 in the population of genetically engineered cells have a mutation effected by a genomic editing approach described herein, e.g., by a CRISPR/Cas system using a gRNA provided herein. By way of example, a population can comprise a plurality of different CD33, CD38, CD123, and/or CD20 mutations and each mutation of the plurality contributes to the percent of copies of CD33, CD38, CD123, and/or CD20 in the population of cells that have a mutation.

In some embodiments, the expression of CD33, CD38, CD123, and/or CD20 on the genetically engineered hematopoietic cell is compared to the expression of CD33, CD38, CD123, and/or CD20 on a naturally occurring hematopoietic cell (e.g., a wild-type counterpart). In some embodiments, the genetic engineering results in a reduction in the expression level of CD33, CD38, CD123, and/or CD20 by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of CD33, CD38, CD123, and/or CD20 on a naturally occurring hematopoietic cell (e.g., a wild-type counterpart). For example, in some embodiments, the genetically engineered hematopoietic cell expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of CD33, CD38, CD123, and/or CD20 as compared to a naturally occurring hematopoietic cell (e.g., a wild-type counterpart).

In some embodiments, the genetic engineering results in a reduction in the expression level of wild-type CD33, CD38, CD123, and/or CD20 by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of the level of wild-type CD33, CD38, CD123, and/or CD20 on a naturally occurring hematopoietic cell (e.g., a wild-type counterpart). That is, in some embodiments, the genetically engineered hematopoietic cell expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of CD33, CD38, CD123, and/or CD20 as compared to a naturally occurring hematopoietic cell (e.g., a wild-type counterpart).

In some embodiments, the genetic engineering results in a reduction in the expression level of wild-type lineage-specific cell-surface antigen (e.g., CD33, CD38, CD123, and/or CD20) by at least 50%, at least 60%, at least 70%, at least 80%, at least 90%, at least 91%, at least 92%, at least 93%, at least 94%, at least 95%, at least 96%, at least 97%, at least 98%, or at least 99% as compared to a suitable control (e.g., a cell or plurality of cells). In some embodiments, the suitable control comprises the level of the wild-type lineage-specific cell-surface antigen measured or expected in a plurality of non-engineered cells from the same subject. In some embodiments, the suitable control comprises the level of the wild-type lineage-specific cell-surface antigen measured or expected in a plurality of cells from a healthy subject. In some embodiments, the suitable control comprises the level of the wild-type lineage-specific cell-surface antigen measured or expected in a population of cells from a pool of healthy individuals (e.g., 10, 20, 50, or 100 individuals). In some embodiments, the suitable control comprises the level of the wild-type lineage-specific cell-surface antigen measured or expected in a subject in need of a treatment described herein, e.g., an anti-CD33, CD38, CD123, and/or CD20 therapy, e.g., wherein the subject has a cancer, wherein cells of the cancer express CD33, CD38, CD123, and/or CD20.

In some embodiments, a method of genetically engineering cells described herein comprises a step of providing a wild-type cell, e.g., a wild-type hematopoietic stem or progenitor cell. In some embodiments, the wild-type cell is an un-modified cell comprising (e.g., expressing) two functional copies of a gene encoding CD33, CD38, CD123, and/or CD20.

In some embodiments, the cell used in the method is a naturally occurring cell or a non-engineered cell.

In some embodiments, the wild-type cell expresses CD33, CD38, CD123, and/or CD20, or gives rise to a more differentiated cell that expresses CD33, CD38, CD123, and/or CD20 at a level comparable to (or within 90%-110%, 80%-120%, 70%-130%, 60-140%, or 50%-150% of) a cell line expressing CD33, CD38, CD123, and/or CD20.

In some embodiments, the wild-type cell binds an antibody that binds CD33, CD38, CD123, and/or CD20 (e.g., an anti-CD33, an anti-CD38, an anti-CD123, and/or an anti-CD20 antibody), or gives rise to a more differentiated cell that binds such an antibody at a level comparable to (or within 90%-110%, 80%-120%, 70%-130%, 60-140%, or 50%-150% of) binding of the antibody to a cell line expressing CD33, CD38, CD123, and/or CD20. Antibody binding may be measured, for example, by flow cytometry or immunohistochemistry.

Cells Comprising One or More Artificial Protospacer Adjacent Motifs (PAMs) and/or Chemical Alterations to a Nucleobase

In some embodiments, an engineered cell described herein comprises one or more (e.g., 2, 3, 4, 5, 6, 7, 8, 9, 10, or more) chemical alterations to a nucleobase. In some embodiments, an engineered cell described herein comprises one or more mutations (e.g., 2, 3, 4, 5, 6, 7, 8, 9, 10, or more). In some embodiments, an engineered cell described herein comprises one or more (e.g., 2, 3, 4, 5, 6, 7, 8, 9, 10, or more) artificial protospacer adjacent motifs (PAMs). In some embodiments, an engineered cell described herein comprises two or more (e.g., 2, 3, 4, 5, 6, 7, 8, 9, 10, or more) chemical alterations to a nucleobase in CD33. In some embodiments, an engineered cell described herein comprises two or more (e.g., 2, 3, 4, 5, 6, 7, 8, 9, 10, or more) chemical alterations to a nucleobase in CD20. In some embodiments, an engineered cell described herein comprises two or more (e.g., 2, 3, 4, 5, 6, 7, 8, 9, 10, or more) chemical alterations to a nucleobase in CD38. In some embodiments, an engineered cell described herein comprises two or more (e.g., 2, 3, 4, 5, 6, 7, 8, 9, 10, or more) chemical alterations to a nucleobase in CD123.

In some embodiments, an engineered cell described herein comprises two or more (e.g., 2, 3, 4, 5, 6, 7, 8, 9, 10, or more) chemical alterations to a nucleobase, the first chemical alteration to a nucleobase resulting in the generation of an artificial PAM and the second chemical alteration to a nucleobase being in a target gene, such as a lineage-specific cell-surface antigen. The chemical alteration may result in an amino acid substitution in a cell-surface protein epitope encoded by the target gene. Such a cell can, in some embodiments, be resistant to an agent targeting the lineage-specific cell-surface antigen. In some embodiments, such a cell can be produced using two or more gRNAs described herein. The disclosure also provides populations comprising cells described herein.

In some embodiments, an engineered cell described herein comprises a first mutation including (i) a base (BE) editing mutation, optionally, wherein the base editing mutation comprises a modification which converts C to T, A to G, T to C, or G to A; (ii) a high-fidelity homology directed repair (HDR)-based mutation, optionally, wherein the HDR-based mutation comprises a single nucleotide change and/or a insertion of a PAM sequence; (iii) a ssODN mutation; (iv) a prime editing (PE) mutation; (v) a nucleobase transition, optionally, wherein nucleobase transition comprises the interchange of purine nucleobases A and G, or the interchange of pyrimidine nucleobases C and T; or (vi) a nucleobase transversion, optionally, wherein the nucleobase transversion comprises the interchange of purine nucleobases A and G and pyrimidine nucleobases C and T.

In some embodiments, an engineered cell described herein comprises a second mutation including (i) a base (BE) editing mutation, optionally, wherein the base editing mutation comprises a modification which converts C to T, A to G, T to C, or G to A; (ii) a high-fidelity homology directed repair (HDR)-based mutation, optionally, wherein the HDR-based mutation comprises a single nucleotide change and/or a insertion of a PAM sequence; (iii) a ssODN mutation; (iv) a prime editing (PE) mutation; (v) a nucleobase transition, optionally, wherein nucleobase transition comprises the interchange of purine nucleobases A and G, or the interchange of pyrimidine nucleobases C and T; (vi) a nucleobase transversion, optionally, wherein the nucleobase transversion comprises the interchange of purine nucleobases A and G and pyrimidine nucleobases C and T; or (vii) a non-homologous end joining (NHEJ)-based mutation, optionally, wherein the NHEJ-based mutation comprises an indel that results in an amino acid deletion, insertion, or frameshift mutation, and/or wherein the NHEJ-based mutation results in a premature stop codon within the open reading frame (ORF) of the target gene.

In some embodiments, an engineered cell described herein comprises three or more (e.g., 3, 4, 5, 6, 7, 8, 9, 10, or more) mutations (e.g., a plurality of mutations), such that the first mutation generates an artificial PAM in the genomic DNA molecule and the last mutation generates a desired modification or effect on a target gene and/or on a protein encoded by the target gene.

In some embodiments, an engineered cell described herein comprises a desired modification or effect on the target gene and/or the protein encoded by the target gene comprises healing a mutation, knocking out gene and/or protein expression, generating a target epitope that is not bound by a specific agent, optionally, wherein the agent is a ligand or an antibody.

In some embodiments, an engineered cell described herein comprises four or more mutations, five or more mutations, six or more mutations, seven or more mutations, eight or more mutations, nine or more mutations, or ten or more mutations.

In some embodiments, the mutation comprises the deamination of a cytosine. In some embodiments, the mutation comprises the deamination of an adenine. In some embodiments, the mutation comprises a nucleobase transition. In some embodiments, the mutation comprises a nucleobase transversion. In some embodiments, the mutation comprises converting a cytosine-guanine (C-G) base pair into a thymine-adenine (T-A) base pair within the target nucleic acid molecule. In some embodiments, the mutation comprises converting a thymine-adenine (T-A) base pair into a cytosine-guanine (C-G) base pair within the target nucleic acid molecule. In some embodiments, the mutation comprises introducing a premature STOP codon within the target nucleic acid molecule. In some embodiments, the mutation comprises introducing a splice site within the target nucleic acid molecule. In some embodiments, the mutation comprises disrupting a splice site within the target nucleic acid molecule. In some embodiments, the mutation comprises disrupting a splice donor. In some embodiments, the mutation comprises disrupting a splice acceptor. In some embodiments, the mutation comprises disrupting a splice enhancer. In some embodiments, the mutation comprises disrupting a exonic splice enhancer (ESE). In some embodiments, the mutation comprises a mutation. In some embodiments, the first mutation comprises a substitution. In some embodiments, the second mutation comprises any edit produced by or producible by a CRISPR system. In some embodiments, the mutation does not comprises an indel produced by or producible by a CRISPR system. In some embodiments, the mutation reduces the activity of and/or expression of a target gene in a cell. In some embodiments, the e target gene encodes a lineage-specific cell-surface antigen. In some embodiments, the editing alters an epitope on a cell-surface protein, optionally, wherein such editing reduces the binding of an active agent to the cell-surface protein.

In some embodiments, the second chemical alteration to a nucleobase is at a gene encoding a lineage-specific cell-surface antigen. Typically, a mutation effected by the methods and compositions provided herein, e.g., a mutation in a target gene or epitope thereof, such as, for example, CD33, CD20, CLL-1, CD123, CD38, CD19, CD117, EMR2, and/or CD5 and/or any other target gene mentioned in this disclosure, results in a loss of function of a gene product encoded by the target gene, e.g., in the case of a mutation in the CD33, CD20, CLL-1, CD123, CD38, CD19, CD117, EMR2, and/or CD5 gene, in a loss of function of a CD33, CD20, CLL-1, CD123, CD38, CD19, CD117, EMR2, and/or CD5 protein.

In some embodiments, the second chemical alteration to a nucleobase is at a gene encoding a lineage-specific cell-surface antigen. Typically, a mutation effected by the methods and compositions provided herein, e.g., a mutation in a target gene or epitope thereof, such as, for example, CD33, CD38, CD123, and/or CD20 and/or any other target gene mentioned in this disclosure, results in a loss of function of a gene product encoded by the target gene, e.g., in the case of a mutation in the CD33, CD38, CD123, and/or CD20 gene, in a loss of function of a CD33, CD38, CD123, and/or CD20 protein. In some embodiments, the loss of function is a reduction in the level of binding of the gene product, e.g., reduction to a lower level of binding, or a complete abolishment of binding of the gene product, to a therapeutic agent. In some embodiments, the loss of function is a reduction in the level of expression of the gene product, e.g., reduction to a lower level of expression, or a complete abolishment of expression of the gene product. In some embodiments, the mutation results in the expression of a non-functional variant of the gene product. For example, in the case of the mutation generating a premature stop codon in the encoding sequence, a truncated gene product, or, in the case of the mutation generating a nonsense or missense mutation, a gene product characterized by an altered amino acid sequence, which renders the gene product non-functional. In some embodiments, the function of a gene product is binding or recognition of a binding partner. In some embodiments, the reduction in expression of the gene product, e.g., of CD33, CD20, CLL-1, CD123, CD38, CD19, CD117, EMR2, and/or CD5, of the second lineage-specific cell-surface antigen, or both, is to less than or equal to 50%, less than or equal to 40%, less than or equal to 30%, less than or equal to 20%, less than or equal to 10%, less than or equal to 5%, less than or equal to 2%, or less than or equal to 1% of the level in a wild-type or non-engineered counterpart cell. In some embodiments, the reduction in expression of the gene product, e.g., of CD33, CD38, CD123, and/or CD20, of the second lineage-specific cell-surface antigen, or both, is to less than or equal to 50%, less than or equal to 40%, less than or equal to 30%, less than or equal to 20%, less than or equal to 10%, less than or equal to 5%, less than or equal to 2%, or less than or equal to 1% of the level in a wild-type or non-engineered counterpart cell.

In some embodiments, at least 40%, at least 50%, at least 60%, at least 70%, at least 75%, at least 80%, at least 85%, at least 90%, or at least 95% of copies of CD33, CD20, CLL-1, CD123, CD38, CD19, CD117, EMR2, and/or CD5 in the population of cells generated by the methods and/or using the compositions provided herein have a mutation, e.g., in a cell-surface protein epitope. In some embodiments, at least at least 40%, at least 50%, at least 60%, at least 70%, at least 75%, at least 80%, at least 85%, at least 90%, or at least 95% of copies of the second lineage-specific cell-surface antigen in the population of cells have a mutation. In some embodiments, at least 40%, at least 50%, at least 60%, at least 70%, at least 75%, at least 80%, at least 85%, at least 90%, or at least 95% of copies of CD33, CD20, CLL-1, CD123, CD38, CD19, CD117, EMR2, and/or CD5 and of the second lineage-specific cell-surface antigen in the population of cells have a mutation. In some embodiments, the population comprises one or more wild-type cells. In some embodiments, the population comprises one or more cells that comprise one wild-type copy of CD33, CD20, CLL-1, CD123, CD38, CD19, CD117, EMR2, and/or CD5. In some embodiments, the population comprises one or more cells that comprise one wild-type copy of the second lineage-specific cell-surface antigen.

In some embodiments, at least 40%, at least 50%, at least 60%, at least 70%, at least 75%, at least 80%, at least 85%, at least 90%, or at least 95% of copies of CD33, CD38, CD123, and/or CD20 in the population of cells generated by the methods and/or using the compositions provided herein have a mutation, e.g., in a cell-surface protein epitope. In some embodiments, at least at least 40%, at least 50%, at least 60%, at least 70%, at least 75%, at least 80%, at least 85%, at least 90%, or at least 95% of copies of the second lineage-specific cell-surface antigen in the population of cells have a mutation. In some embodiments, at least 40%, at least 50%, at least 60%, at least 70%, at least 75%, at least 80%, at least 85%, at least 90%, or at least 95% of copies of CD33, CD38, CD123, and/or CD20 and of the second lineage-specific cell-surface antigen in the population of cells have a mutation. In some embodiments, the population comprises one or more wild-type cells. In some embodiments, the population comprises one or more cells that comprise one wild-type copy of CD33, CD38, CD123, and/or CD20. In some embodiments, the population comprises one or more cells that comprise one wild-type copy of the second lineage-specific cell-surface antigen.

### Exemplary Cells for Immuno-Oncology (I-O)

Some aspects of this disclosure provide genetically engineered cells suitable for use in combination therapies with immuno-oncology (I-O) agents. Immuno-oncology therapy is based, at least in part, on the concept of modulating, e.g., activating, the immune system of a subject to generate an immune response, e.g., an anti-tumor immunity. In the context of immuno-oncology (I-O), immune checkpoint inhibition can act on inhibitory signaling pathways that function to suppress immune cells, such as T cells. Without wishing to be bound by theory, when this suppression is removed by immune checkpoint inhibition, the suppression of anti-tumor T cell activity is released.

Accordingly, in one aspect, methods for treating or preventing a cancer in a subject are provided. In some embodiments, the method comprises administering to the subject a therapeutic amount of an immuno-oncology (I-O) agent as described herein or a pharmaceutical composition comprising an immuno-oncology (I-O) agent as described herein. In some embodiments, the subject is a human, e.g., a human adult or a human child.

In some embodiments, the method comprises administering to the subject a therapeutic amount of a cell, e.g., a modified cell, as described herein in combination with an immuno-oncology (I-O) agent in any amount and by any route of administration effective for treating or lessening the severity of a disease or disorder, such as a cancer, in the subject.

In some embodiments, the cells, e.g., modified cells, and the immuno-oncology (I-O) agent as described herein may be formulated into pharmaceutical compositions, together or separately, as described herein. In some embodiments, the cells, e.g., modified cells, and compositions as described herein can be administered sequentially prior to administration of the immune-oncology agent. In some embodiments, the cells, e.g., modified cells, and compositions as described herein can be administered concurrently with an immuno-oncology agent. In some embodiments, the cells, e.g., modified cells, and compositions as described herein can be administered sequentially after administration of the immune-oncology agent.

As used herein, the term an “immuno-oncology (I-O) agent” or an “immune-oncology (I-O) therapy,” refers to an agent that enhances, stimulates, or upregulates an immune response in a subject, e.g., against a cancer (e.g., stimulates an immune response to inhibit tumor growth). In some embodiments, the immuno-oncology agent is a small molecule, antibody, peptide, protein, cyclic peptide, peptidomimetic, polynucleotide, inhibitory RNA, aptamer, pharmaceutical compound, or other compound.

In some embodiments, an “immune-oncology agent” comprises a therapeutic agent that targets at least one immune checkpoint protein to alter or modulate an immune response (e.g., activate or block an immune response) in a subject. In some embodiments, the immuno-oncology agent comprises (i) an agonist of a stimulating (including co-stimulating) receptor to a T cell, or (ii) an antagonist that inhibits (including co-inhibiting) signaling, both of which result in an expanded antigen-specific T cell response. In some embodiments, the immune checkpoint inhibitor enhances or suppresses the function of one or more targeted immune checkpoint proteins. In some embodiments, the immune-oncology agent comprises an agent effective to enhance, stimulate and/or up-regulate an immune response in a subject.

In some embodiments, administration of the immune-oncology agent and the cells, e.g., modified cells, as described herein has a synergistic effect in treating a cancer.

Immune checkpoint proteins are known in the art and include, but are not limited to, cytotoxic T lymphocyte antigen 4 (CTLA-4), programmed cell death 1 (PD-1), programmed cell death ligand 1 (PD-L1), programmed cell death ligand 2 (PD-L2), T cell activated V domain Ig inhibitor (VISTA), B7-H2, B7-H3, B7-H4, B7-H6, 2B4, ICOS, HVEM, CD160, gp49B, PIR-B, KIR family receptor, TIM-1, TIM-3, TIM-4, LAG-3, BTLA, sirpa (CD47), CD48, 2B4 (CD244), B7.1, B7.2, ILT-2, ILT-4, tig, LAG-3, BTLA, IDO, 40, and A2 aR. In some embodiments, the immune checkpoint protein may be expressed on the surface of immune cells, such as activated T cells. In some embodiments, suitable immune-oncology agents comprise immune checkpoint antibodies, such as agonistic (i.e., activating) antibodies that bind to an activating immune checkpoint, receptor or molecule, such as CD28, OX40, and/or GITR. In some embodiments, suitable immune-oncology agents comprise immune checkpoint antibodies, such as antagonistic (e.g., blocking) antibodies that bind to an immune checkpoint modulator (e.g., an inhibitory or stimulatory receptor or molecule), such as PD-1, PD-L1, CTLA-4, TIM-3, LAG-3, OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, VISTA, BTLA, TIGIT, LAIR1, CD160, 2B4 and/or TGF-β. In some embodiments, suitable immune-oncology agents comprise immune checkpoint therapeutics, such as pembrolizumab, nivolumab, ipilimumab, atezolizumab, avelumab, durvalumab, tremelimumab, and/or cemiplimab.

Treatment of Diseases and/or Disorders

The present disclosure provides, among other things, compositions and methods for treating a subject with a disease or disorder.

Any of the methods described herein can be used to treat a disease or disorder, such as a hematopoietic malignancy, in a subject. As used herein, the term “treating” or “treatment” refers to alleviating or alleviating at least one symptom associated with a disease or disorder, or slowing or reversing the progression of a disease or disorder. Within the meaning of the present disclosure, the term “treating” also means arresting a disease, delaying the onset of a disease (e.g., the period prior to clinical manifestation of a disease) and/or reducing the risk of developing or worsening a disease. For example, with respect to a cancer, the term “treating” may mean eliminating the cancer or reducing the number or replication of cancer cells, and/or preventing, delaying, or inhibiting metastasis, and the like.

Accordingly, in one aspect, the present invention provides a method of treating a disease or disorder in a subject, comprising administering an effective amount of any of the various components of the CRISPR/Cas systems, e.g., base editing technology or HDR-mediated gene editing technology, or cells, e.g., genetically engineered cells, or compositions described herein.

In some embodiments, the method comprises administering an effective number of genetically engineered cells as described herein, e.g., comprising an artificial PAM and a modified cell-surface protein, e.g., having a mutant cell-surface epitope, to a subject.

In some embodiments, the method may further comprise administering an additional therapeutic agent to the subject.

In some embodiments, the disease or disorder is a cancer, auto-immune disease, or genetic disease. In some embodiments, the disease or disorder is associated with expression of a cell-surface protein or a condition associated with cells expressing a cell-surface protein, including, for example, a proliferative disease, such as a cancer or malignancy (e.g., a hematopoietic malignancy), or a precancerous condition, such as a myelodysplasia, a myelodysplastic syndrome or a preleukemia.

In some embodiments, the hematopoietic malignancy or a hematological disorder is associated with CD33, CD38, CD123, and/or CD20 expression. A hematopoietic malignancy has been described as a malignant abnormality involving hematopoietic cells (e.g., blood cells, including progenitor and stem cells). Examples of hematopoietic malignancies include, without limitation, Hodgkin's lymphoma, non-Hodgkin's lymphoma, leukemia, or multiple myeloma. Exemplary leukemia include, without limitation, acute myeloid leukemia, acute lymphoid leukemia, chronic myelogenous leukemia, acute lymphoblastic leukemia or chronic lymphoblastic leukemia, and chronic lymphoid leukemia.

In some embodiments, cells involved in the hematopoietic malignancy are resistant to conventional or standard therapeutics used to treat the malignancy. For example, the cells (e.g., cancer cells) may be resistant to a chemotherapeutic agent and/or CAR T cells used to treat the malignancy.

In some embodiments, the malignancy is a hematologic malignancy, or a cancer of the blood. In some embodiments, the hematopoietic malignancy is acute myeloid leukemia (AML). AML is characterized as a heterogeneous, clonal, neoplastic disease that originates from transformed cells that have progressively acquired critical genetic changes that disrupt key differentiation and growth-regulatory pathways. (Dohner et al., NEJM, (2015) 373:1136). Without wishing to be bound by theory, it is believed in some embodiments, that CD33, CD38, CD123, and/or CD20 is expressed on myeloid leukemia cells as well as on normal myeloid and monocytic precursors and is an attractive target for AML therapy.

In some cases, a subject may initially respond to a therapy (e.g., for a hematopoietic malignancy) and subsequently experience relapse. Any of the methods or populations of genetically engineered cells described herein may be used to reduce or prevent relapse of a malignancy. Alternatively or in addition, any of the methods described herein may involve administering any of the populations of genetically engineered cells described herein and an agent (e.g., cytotoxic agent) that targets cells associated with the malignancy and further administering one or more additional immunotherapeutic agents when the malignancy relapses. In some embodiments, the subject has or is susceptible to relapse of a malignancy (e.g., AML) following administration of one or more previous therapies. In some embodiments, the methods described herein reduce the subject's risk of relapse or the severity of relapse.

In some embodiments, the malignancy or disorder associated with expression of a cell-surface protein is a precancerous condition, such as a myelodysplasia, a myelodysplastic syndrome or a preleukemia. Myelodysplastic syndromes (MDS) are hematological medical conditions characterized by disorderly and ineffective hematopoiesis, or blood production. Thus, the number and quality of blood-forming cells decline irreversibly. Some patients with MDS can develop severe anemia, while others are asymptomatic. The classification scheme for MDS is known in the art, with criteria designating the ratio or frequency of particular blood cell types, e.g., myeloblasts, monocytes, and red cell precursors. MDS includes refractory anemia, refractory anemia with ring sideroblasts, refractory anemia with excess blasts, refractory anemia with excess blasts in transformation, chronic myelomonocytic leukemia (CML). In some embodiments, MDS can progress to a AML.

### Exemplary Methods of Treatment and Administration

In another aspect, the present invention provides a method of treating a cancer in a subject, comprising administering an effective amount of any of the various components of the CRISPR/Cas systems, e.g., base editing technology or HDR-mediated gene editing technology, or cells, e.g., genetically engineered cells, or compositions described herein.

In some embodiments, the method comprises administering an effective number of genetically engineered cells as described herein, e.g., comprising an artificial PAM and a modified cell-surface protein, e.g., having a mutant cell-surface epitope, to a subject.

In some embodiments, the method may further comprise administering a therapeutic agent to the subject. In some embodiments, the therapeutic agent may bind to a cell-surface protein, such as a cell surface lineage specific protein. In some embodiments, therapeutic agent may comprise a cytotoxic agent, and/or an anti-cancer therapy.

In some embodiments, the method may further comprises administering to the subject a therapeutic amount of a cell, e.g., a modified cell, as described herein in combination with an immuno-oncology (I-O) agent in any amount and by any route of administration effective for treating or lessening the severity of a disease or disorder, such as a cancer, in the subject.

In some embodiments, a subject is administered a therapeutic agent that binds to an epitope of a cell surface protein, such as a cell surface lineage specific protein, and a cell that (i) expresses the cell surface protein but has been manipulated such that they do not bind the therapeutic agent, and/or (ii) does not express the cell surface protein. Thus, in such embodiments of any of the methods described herein, the therapeutic agent can recognize (i.e., bind to) target cells expressing epitopes of cell surface proteins for targeted killing. Cells, such as the modified cells described herein, that express the cell surface protein but do not bind the therapeutic agent (e.g., because they lack epitopes) can allow for the repopulation of the cell type targeted by the therapeutic agent.

The efficacy of treatment methods using therapeutic agents, e.g., comprising antigen-binding fragments that bind to cell surface lineage specific proteins, and hematopoietic cell populations lacking cell surface lineage specific proteins can be assessed by any method known in the art and will be apparent to the skilled medical professional. For example, the efficacy of a treatment can be assessed by the survival of the subject or the cancer burden in the subject or a tissue or sample thereof. In some embodiments, the efficacy of a treatment is assessed by quantifying the number of cells belonging to a particular cell population or lineage. In some embodiments, the efficacy of the treatment is assessed by quantifying the number of cells presenting cell surface lineage specific proteins.

In some embodiments, an effective number of CD33, CD38, CD123, and/or CD20-modified cells described herein is administered to a subject in combination with an anti-CD33, an anti-CD38, an anti-CD123, and/or an anti-CD20 therapy, e.g., an anti-CD33, an anti-CD38, an anti-CD123, and/or an anti-CD20 cancer therapy.

In some embodiments, an effective number of cells comprising an artificial PAM and a modified CD33 and, optionally, a modified second lineage-specific cell-surface antigen are administered in combination with an anti-CD33 therapy, e.g., an anti-CD33 cancer therapy. In some embodiments, the anti-CD33 therapy comprises an antibody, a bispecific T cell engager, an ADC, or an immune cell expressing a CAR.

In some embodiments, an effective number of genetically engineered cells comprising an artificial PAM and a modified CD20 and, optionally, a modified second lineage-specific cell-surface antigen are administered in combination with an anti-CD20 therapy, e.g., an anti-CD20 cancer therapy. In some embodiments, the anti-CD20 therapy comprises an antibody, a bispecific T cell engager, an ADC, or an immune cell expressing a CAR.

In some embodiments, an effective number of genetically engineered cells comprising an artificial PAM and a modified CD38 and, optionally, a modified second lineage-specific cell-surface antigen are administered in combination with an anti-CD38 therapy, e.g., an anti-CD38 cancer therapy. In some embodiments, the anti-CD38 therapy comprises an antibody, a bispecific T cell engager, an ADC, or an immune cell expressing a CAR.

In some embodiments, an effective number of genetically engineered cells comprising an artificial PAM and a modified CD123 and, optionally, a modified second lineage-specific cell-surface antigen are administered in combination with an anti-CD123 therapy, e.g., an anti-CD123 cancer therapy. In some embodiments, the anti-CD123 therapy comprises an antibody, a bispecific T cell engager, an ADC, or an immune cell expressing a CAR.

In some embodiments, the number of genetically engineered cells provided herein that are administered to a subject in need thereof, is within the range of 106-1011. However, amounts below or above this exemplary range are also within the scope of the present disclosure. For example, in some embodiments, the number of genetically engineered cells provided herein, e.g., HSCs, HPCs, or immune effector cells that are administered to a subject in need thereof is about 106, about 107, about 108, about 109, about 1010, or about 1011 In some embodiments, the number of genetically engineered cells provided herein that are administered to a subject in need thereof, is within the range of 106-109, within the range of 106-108, within the range of 107-109, within the range of about 107-1010, within the range of 108-1010, or within the range of 109-1011.

It is understood that when agents (e.g., CD33, CD38, CD123, and/or CD20-modified cells and an anti-CD33, CD38, CD123, and/or CD20 therapy) are administered in combination, the agent may be administered at the same time or at different times in temporal proximity. Furthermore, the agents may be admixed or in separate volumes. For example, in some embodiments, administration in combination includes administration in the same course of treatment, e.g., in the course of treating a cancer with an anti-CD33 therapy, the subject may be administered an effective number of CD33-modified cells concurrently or sequentially, e.g., before, during, or after the treatment, with the anti-CD33 therapy. In some embodiments, administration in combination includes administration in the same course of treatment, e.g., in the course of treating a cancer with an anti-CD20 therapy, the subject may be administered an effective number of CD20-modified cells concurrently or sequentially, e.g., before, during, or after the treatment, with the anti-CD20 therapy. In some embodiments, administration in combination includes administration in the same course of treatment, e.g., in the course of treating a cancer with an anti-CD38 therapy, the subject may be administered an effective number of CD38-modified cells concurrently or sequentially, e.g., before, during, or after the treatment, with the anti-CD38 therapy. In some embodiments, administration in combination includes administration in the same course of treatment, e.g., in the course of treating a cancer with an anti-CD123 therapy, the subject may be administered an effective number of CD123-modified cells concurrently or sequentially, e.g., before, during, or after the treatment, with the anti-CD123 therapy.

Non-limiting examples of agents that target CD33 include Lintuzumab, gemtuzumab ozogamicin, P67.6, and antigen-binding fragments thereof.

Non-limiting examples of agents that target CD123 include flotetuzumab, vibecotamab, JNJ-63709178, APVO436, 7G3 (JNJ-56022473), a humanized variant thereof (e.g., antibody CSL-362), 6H6, 9F5, SAR440234, and antigen-binding fragments thereof.

Non-limiting examples of agents that target CD20 include Type I mABs, such as m708 and mRTX, and antigen-binding fragments thereof, and Type II mAbs, such as B1 and m1188, and antigen-binding fragments thereof. See, e.g., Meyer et al. British Journal of Haematology (2018) 180:808-820.

Non-limiting examples of agents that target CD38 include daratumumab, isatuximab, HB7, MIR202, TAK-079, and antigen-binding fragments thereof.

In some embodiments, the agent that targets a CD33, CD38, CD123, and/or CD20 as described herein is an immune cell that expresses a chimeric receptor, which comprises an antigen-binding fragment (e.g., a single-chain antibody) capable of binding to CD33, CD38, CD123, and/or CD20. The immune cell may be, e.g., a T cell (e.g., a CD4+ or CD8+ T cell) or an NK cell.

In some embodiments, treatment of a subject may include the steps of: (1) administering to the patient a therapeutically effective amount of a therapeutic agent, such as a cytotoxic agent, and (2) infusing or reinfusing into the subject autologous or allogeneic cells, such as modified cells as described herein, wherein the cells have been manipulated such that they do not bind the therapeutic agent, e.g., cytotoxic agent. In some embodiments, treatment of a subject may include the steps of: (1) administering to the patient a therapeutically effective amount of an immune cell expressing a chimeric receptor, wherein the immune cell comprises a nucleic acid encoding the chimeric receptor that binds to an epitope of a cell surface lineage specific disease-associated protein; and (2) infusing or reinfusing into the patient autologous or allogeneic hematopoietic cells (e.g., hematopoietic stem cells) that have been manipulated such that they do not bind cytotoxic agents.

A Chimeric Antigen Receptor (CAR) can comprise a recombinant polypeptide comprising at least an extracellular antigen binding domain, a transmembrane domain and a cytoplasmic signaling domain comprising a functional signaling domain, e.g., one derived from a stimulatory molecule. In one some embodiments, the cytoplasmic signaling domain further comprises one or more functional signaling domains derived from at least one costimulatory molecule, such as 4-1BB (i.e., CD137), CD27 and/or CD28 or fragments of those molecules. The extracellular antigen binding domain of the CAR may comprise a CD33, CD38, CD123, and/or CD20-binding antibody fragment. The antibody fragment can comprise one or more CDRs, the variable region (or portions thereof), the constant region (or portions thereof), or combinations of any of the foregoing.

Exemplary CD33 CAR constructs are found, e.g., in International Publication No. WO2019/178382, incorporated herein by reference in its entirety.

Exemplary chimeric receptor component sequences are provided in Table 1 below.

TABLE 1
Exemplary components of a chimeric receptor

| Chimeric receptor component | Amino acid sequence |
|---|---|
| Antigen-binding fragment | Light chain-GSTSSGSGKPGSGEGSTKG |
| | (SEQ ID NO: 34)-Heavy chain |
| 4-1BB costimulatory domain | KRGRKKLLYIFKQPFMRPVQTTQEEDGCSC |
| | RFPEEEEGGCE (SEQ ID NO: 115) |
| CD28 costimulatory domain | IEVMYPPPYLDNEKSNGTIIHVKGKHLCPSP |
| | LFPGPSKPFWVLVVVGGVLACYSLLVTVA |
| | FIIFWVRSKRSRLLHSDYMNMTPRRPGPTR |
| | KHYQPYAPPRDFAAYRS (SEQ ID NO: 35) |
| ICOS costimulatory domain (boldface), | LSIFDPPPFKVTLTGGYLHIYESQLCCQLKF |
| ICOS transmembrane domain (italics) | WLPIGCAAFVVVCILGCILICWLTKKKYSSS |
| and a portion of the extracellular | VHDPNGEYMFMRAVNTAKKSRLTDVTL |
| domain of ICOS (underlined) | (SEQ ID NO: 36) |
| ICOS costimulatory domain | CWLTKKKYSSSVHDPNGEYMFMRAVNTA |
| | KKSRLTDVTL (SEQ ID NO: 37) |
| CD28/ICOS chimera (the ICOS portion | IEVMYPPPYLDNEKSNGTIIHVKGKHLCPSPL |
| shown in underline) including the hinge | FPGPSKPFWVLVVVGGVLACYSLLVTVA |
| domain (italics) and transmembrane | FIIFWVRSKRSRLLHSDYMFMRAVNTAKK |
| domain (bold) from CD28 | SRLTDVTL (SEQ ID NO: 38) |
| CD8α transmembrane domain (italics) | TTTPAPRPPTPAPTIASQPLSLRPEACRPAA |
| and a portion of the extracellular | GGAVHTRGLDFACDIYIWAPLAGTCGVLLLS |
| domain of CD8α (underlined) | LVITLYC (SEQ ID NO: 55) |
| CD3ζ cytoplasmic signaling domain | RVKFSRSADAPAYQQGQNQLYNELNLGRR |
| | EEYDVLDKRRGRDPEMGGKPQRRKNPQE |
| | GLYNELQKDKMAEAYSEIGMKGERRRGK |
| | GHDGLYQGLSTATKDTYDALHMQALPPR |
| | (SEQ ID NO: 39) |

In some embodiments, the CAR comprises a 4-1BB costimulatory domain (e.g., as shown in Table 1), a CD8α transmembrane domain and a portion of the extracellular domain of CD8α (e.g., as shown in Table 1), and a CD3ζ cytoplasmic signaling domain (e.g., as shown in Table 1).

A typical number of cells, e.g., immune cells or hematopoietic cells, administered to a mammal (e.g., a human) can be, for example, in the range of one million to 100 billion cells; however, amounts below or above this exemplary range are also within the scope of the present disclosure.

In some embodiments, the agent that targets CD33, CD38, CD123, and/or CD20 is an antibody-drug conjugate (ADC). The ADC may be a molecule comprising an antibody or antigen-binding fragment thereof conjugated to a toxin or drug molecule. Binding of the antibody or fragment thereof to the corresponding antigen allows for delivery of the toxin or drug molecule to a cell that presents the antigen on its cell-surface (e.g., target cell), thereby resulting in death of the target cell. Toxins or drugs compatible for use in antibody-drug conjugates known in the art and will be evident to one of ordinary skill in the art. See, e.g., Peters et al. Biosci. Rep. (2015) 35 (4): e00225; Beck et al. Nature Reviews Drug Discovery (2017) 16:315-337; Marin-Acevedo et al. J. Hematol. Oncol. (2018) 11:8; Elgundi et al. Advanced Drug Delivery Reviews (2017) 122:2-19.

In some embodiments, the antibody-drug conjugate may further comprise a linker (e.g., a peptide linker, such as a cleavable linker) attaching the antibody and drug molecule.

Examples of antibody-drug conjugates include, without limitation, brentuximab vedotin, glembatumumab vedotin/CDX-011, depatuxizumab mafodotin/ABT-414, PSMA ADC, polatuzumab vedotin/RG7596/DCDS4501A, denintuzumab mafodotin/SGN-CD19A, AGS-16C3F, CDX-014, RG7841/DLYE5953A, RG7882/DMUC406A, RG7986/DCDS0780A, SGN-LIVIA, enfortumab vedotin/ASG-22ME, AG-15ME, AGS67E, telisotuzumab vedotin/ABBV-399, ABBV-221, ABBV-085, GSK-2857916, tisotumab vedotin/HuMax-TF-ADC, HuMax-Axl-ADC, pinatuzumab veodtin/RG7593/DCDT2980S, lifastuzumab vedotin/RG7599/DNIB0600A, indusatumab vedotin/MLN-0264/TAK-264, vandortuzumab vedotin/RG7450/DSTP3086S, sofituzumab vedotin/RG7458/DMUC5754A, RG7600/DMOT4039A, RG7336/DEDN6526A, ME1547, PF-06263507/ADC 5T4, trastuzumab emtansine/T-DM1, mirvetuximab soravtansine/IMGN853, coltuximab ravtansine/SAR3419, naratuximab emtansine/IMGN529, indatuximab ravtansine/BT-062, anetumab ravtansine/BAY 94-9343, SAR408701, SAR428926, AMG 224, PCA062, HKT288, LY3076226, SAR566658, lorvotuzumab mertansine/IMGN901, cantuzumab mertansine/SB-408075, cantuzumab ravtansine/IMGN242, laprituximab emtansine/IMGN289, IMGN388, bivatuzumab mertansine, AVE9633, BIIB015, MLN2704, AMG 172, AMG 595, LOP 628, vadastuximab talirine/SGN-CD123A, SGN-CD70A, SGN-CD19B, SGN-CD123A, SGN-CD352A, rovalpituzumab tesirine/SC16LD6.5, SC-002, SC-003, ADCT-301/HuMax-TAC-PBD, ADCT-402, MEDI3726/ADC-401, IMGN779, IMGN632, gemtuzumab ozogamicin, inotuzumab ozogamicin/CMC-544, PF-06647263, CMD-193, CMB-401, trastuzumab duocarmazine/SYD985, BMS-936561/MDX-1203, sacituzumab govitecan/IMMU-132, labetuzumab govitecan/IMMU-130, DS-8201a, U3-1402, milatuzumab doxorubicin/IMMU-110/hLL1-DOX, BMS-986148, RC48-ADC/hertuzumab-vc-MMAE, PF-06647020, PF-06650808, PF-06664178/RN927C, lupartumab amadotin/BAY1129980, aprutumab ixadotin/BAY1187982, ARX788, AGS62P1, XMT-1522, AbGn-107, MEDI4276, DSTA4637S/RG7861. In one example, the antibody-drug conjugate is gemtuzumab ozogamicin.

In some embodiments, binding of the antibody-drug conjugate to the epitope of the cell-surface lineage-specific protein induces internalization of the antibody-drug conjugate, and the drug (or toxin) may be released intracellularly. In some embodiments, binding of the antibody-drug conjugate to the epitope of a cell-surface lineage-specific protein induces internalization of the toxin or drug, which allows the toxin or drug to kill the cells expressing the lineage-specific protein (target cells). In some embodiments, binding of the antibody-drug conjugate to the epitope of a cell-surface lineage-specific protein induces internalization of the toxin or drug, which may regulate the activity of the cell expressing the lineage-specific protein (target cells). The type of toxin or drug used in the antibody-drug conjugates described herein is not limited to any specific type.

### EXAMPLES

### Example 1: Evaluation of Sequential Editing Using Base Editors to Generate an Artificial PAM and to Disrupt Lintuzumab Binding to CD33 in CD34+ Cells

A sequential base editing strategy was devised to perform sequential delivery of an Adenine Base Editor (A→G) enzyme in CD34+ cells to first generate a suitable PAM and subsequently modify the key asparagine residue in the CD33 locus to disrupt lintuzumab binding.

Preliminary data suggested that the asparagine residue at position 20 (N20) is important for lintuzumab binding, and modification of the asparagine to aspartic acid (N20D) disrupts binding.

Designs of modifying base editing guides to target asparagine N20 within the lintuzumab-targeted CD33 locus epitope using canonical NGG PAM-recognizing adenine base editors are not feasible. Adenine base editors (ABE) mediate a chemical conversion of A-T to G-C base pairing, and it was hypothesized that this property could be used to generate a new (artificial) PAM nearby the desired target (PAM generator guide). A second targeting guide (gRNA) can then be used to target an “A” within the +4-+8 base editing window to target asparagine and convert this residue into aspartic acid, which would be expected to disrupt lintuzumab binding.

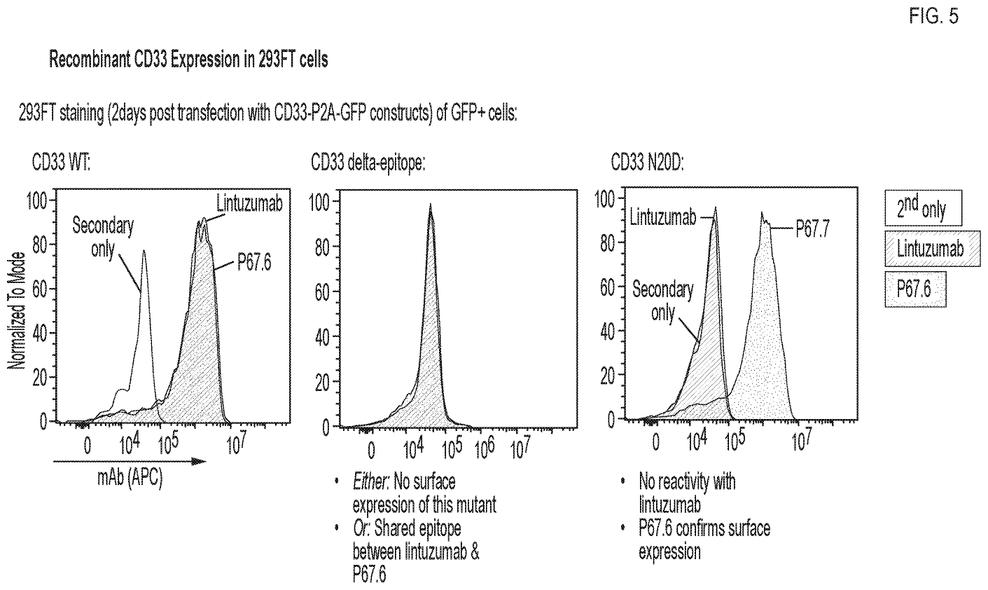

An exemplary approach for adenine base editing within exon 2 of the CD33 gene to generate an artificial protospacer adjacent motif (PAM) is shown in FIG. 1. FIG. 1 shows a first target domain having the nucleic acid sequence GCAAGTGCAGGAGTCAGTGA (SEQ ID NO: 68) (or GCAAGTGCAGGAGTCAGTG (SEQ ID NO: 86)) in proximity to a CGG PAM which is suitable to provide a first mutation to generate an artificial PAM having the nucleic acid sequence of AGG in the genomic DNA molecule in proximity to a second or subsequent target domain having the nucleic acid sequence of GGATCCAAATTTCTGGCTGC (SEQ ID NO: 70) (or GGATCCAAATTTCTGGCTG (SEQ ID NO: 87)) that lacks a PAM suitable to provide a second mutation. The schematic further shows a second mutation that results in the amino acid substitution of N20D in the CD33 epitope recognized by lintuzumab, a humanized monoclonal antibody used in the treatment of cancer. The amino acid substitution results in a reduction of the binding activity of lintuzumab to the epitope as compared to the unmodified epitope, as shown in FIG. 5.

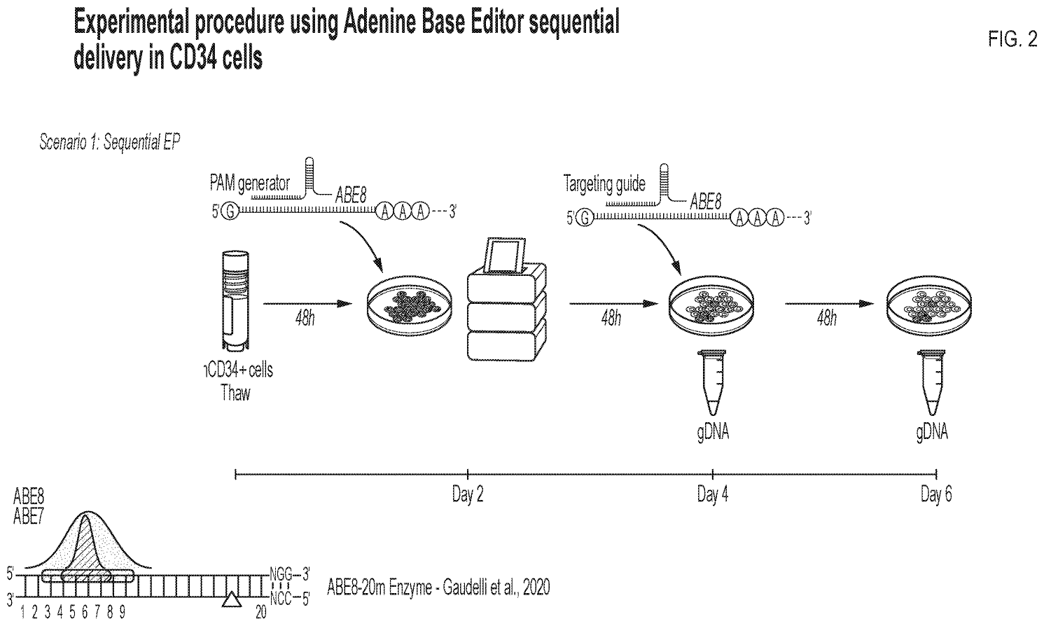

An exemplary experimental procedure using adenine base editor sequential delivery in CD34+ cells to generate an artificial protospacer adjacent motif (PAM) in proximity of a target domain which lacks a suitable PAM is shown in FIG. 2. At Day 0, CD34+ cells were thawed for culture. At Day 2, a first CRISPR/Cas system and a first guide RNA (gRNA), also referred to herein as a “PAM generator,” configured to direct the first CRISPR/Cas system to a first target domain to provide a first mutation to generate an artificial PAM in a genomic DNA molecule were introduced into the CD34+ cells. At Day 4, genomic DNA (gDNA) was obtained from the modified CD34+ cell populations to analyze genomic editing, e.g., via DNA sequencing. Additionally, at Day 4, a second CRISPR/Cas system and a second gRNA, also referred to herein as a “targeting guide,” configured to direct the second CRISPR/Cas system to a second or subsequent target domain in proximity to the artificial PAM was introduced to the CD34+ cells to provide a second mutation. An exemplary CRISPR/Cas system that was used to bind both to the first gRNA and to the second gRNA was the adenine base editor ABE8. At Day 6, gDNA was obtained from the sequentially modified CD34+ cell populations to analyze genomic editing, e.g., via DNA sequencing.

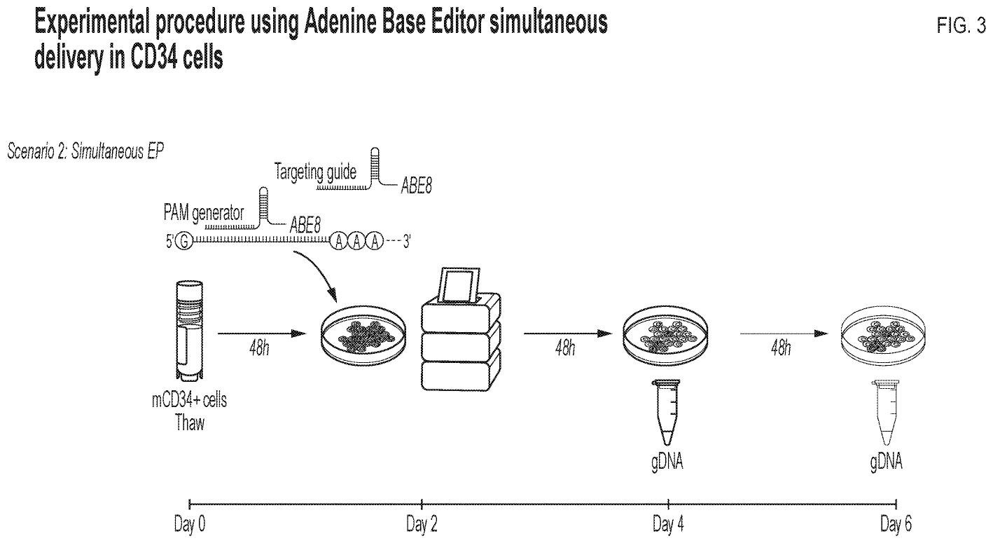

An exemplary experimental procedure using adenine base editor simultaneous delivery in CD34+ cells to generate an artificial protospacer adjacent motif (PAM) in proximity of a target domain which lacks a suitable PAM is shown in FIG. 3. At Day 0, CD34+ cells were thawed for culture. At Day 2, a CRISPR/Cas system; a first guide RNA (gRNA), also referred to herein as a “PAM generator,” configured to direct a first CRISPR/Cas system to a first target domain to provide a first mutation to generate an artificial PAM in a genomic DNA molecule; and a second gRNA, also referred to herein as a “targeting guide,” configured to direct a second CRISPR/Cas system to a second or subsequent target domain in proximity to the artificial PAM to provide a second mutation were introduced into the CD34+ cells. An exemplary CRISPR/Cas system that was used to bind both to the first gRNA and to the second gRNA was the adenine base editor ABE8. At Day 6, gDNA was obtained from the simultaneously modified CD34+ cell populations to analyze genomic editing, e.g., via DNA sequencing.

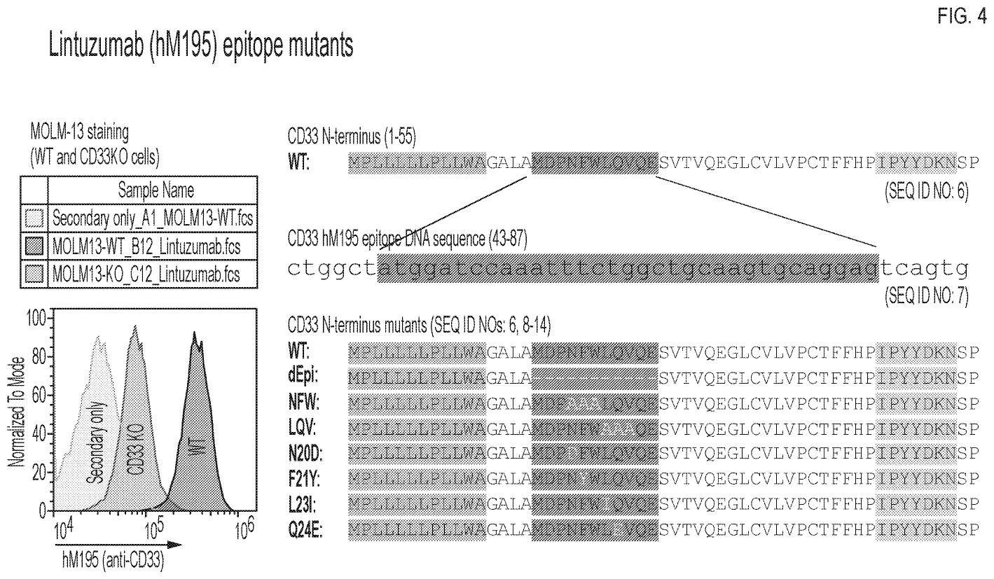

Histograms of MOLM-13 wildtype (WT) and CD33 knockout (CD33KO) cells stained with lintuzumab (hM195), and sequence alignments of various CD33 N-terminus mutants found to have one or more amino acid substitutions in the lintuzumab epitope (MDPNFWLQVQE (SEQ ID NO: 2)) are shown in FIG. 4. The CD33 N-terminus mutants tested included:
dEpi, characterized by the deletion of MDPNFWLQVQE (SEQ ID NO: 2) at residue positions 17-27;
NFW, characterized by the substitution of NEW with AAA at residue positions 20-22;
LQV, characterized by the substitution of LQV with AAA at residue positions 23-25;
N20D, characterized by the substitution of N with D at residue position 20;
F21Y, characterized by the substitution of F with Y at residue position 21;
L23I, characterized by the substitution of L with I at residue position 23; and
Q24E, characterized by the substitution of Q with E at residue position 24.

Table 2 below shows the full-length wildtype CD33 amino acid sequence which has been annotated to show the amino acid residue positions that are modified in the CD33 N-terminus mutants. The full-length amino acid sequences of the CD33 N-terminus mutants are also shown in Table 2.

TABLE 2
Full-length amino acid sequences of the CD33 N-terminus mutants

| | Amino Acid Sequence (N′ terminus | SEQ |
|---|---|---|
| Name | to C′ terminus) | ID NO: |
| CD33 Wildtype | MPLLLLLPLLWAGALA [symbol] SVTVQE | 97 |
| (Uniprot No. P20138) | GLCVLVPCTFFHPIPYYDKNSPVHGYWFREGAI | |
| Key: | ISRDSPVATNKLDQEVQEETQGRFRLLGDPSRN | |
| [symbol] (SEQ ID NO: 2) | NCSLSIVDARRRDNGSYFFRMERGSTKYSYKSP | |
| residues (positions 17-27) = deleted from | QLSVHVTDLTHRPKILIPGTLEPGHSKNLTCSV | |
| dEpi mutant | SWACEQGTPPIFSWLSAAPTSLGPRTTHSSVLI | |
| NFW residues (positions 20-22) = | ITPRPQDHGTNLTCQVKFAGAGVTTERTIQLNV | |
| substituted with AAA residues in NFW | TYVPQNPTTGIFPGDGSGKQETRAGVVHGAIGG | |
| mutant | AGVTALLALCLCLIFFIVKTHRRKAARTAVGRN | |
| [symbol] residues (positions 23-25) = | DTHPTTGSASPKHQKKSKLHGPTETSSCSGAAP | |
| substituted with AAA residues in LQV | Q | |
| mutant | | |
| N residue (position 20) = substituted for | | |
| D residue in N20D mutant | | |
| F residue (position 21) = substituted with | | |
| Y residue in F21Y mutant | | |
| L residue (position 23) = substituted with I | | |
| residue in L23I mutant | | |
| [symbol] residue (position 24) = substituted with | | |
| E residue in Q24E mutant | | |
| CD33 delta-epitope (dEpi) | MPLLLLLPLLWAGALASVTVQEGLCVLVPCTFF | 106 |
| | HPIPYYDKNSPVHGYWFREGAIISRDSPVATNK | |
| | LDQEVQEETQGRFRLLGDPSRNNCSLSIVDARR | |
| | RDNGSYFFRMERGSTKYSYKSPQLSVHVTDLTH | |
| | RPKILIPGTLEPGHSKNLTCSVSWACEQGTPPI | |
| | FSWLSAAPTSLGPRTTHSSVLIITPRPQDHGTN | |
| | LTCQVKFAGAGVTTERTIQLNVTYVPQNPTTGI | |
| | FPGDGSGKQETRAGVVHGAIGGAGVTALLALCL | |
| | CLIFFIVKTHRRKAARTAVGRNDTHPTTGSASP | |
| | KHQKKSKLHGPTETSSCSGAAPTVEMDEELHYA | |
| | SLNFHGMNPSKDTSTEYSEVRTQ | |
| CD33 NFW | MPLLLLLPLLWAGALAMDPAAALQVQESVTVQE | 107 |
| | GLCVLVPCTFFHPIPYYDKNSPVHGYWFREGAI | |
| | ISRDSPVATNKLDQEVQEETQGRFRLLGDPSRN | |
| | NCSLSIVDARRRDNGSYFFRMERGSTKYSYKSP | |
| | QLSVHVTDLTHRPKILIPGTLEPGHSKNLTCSV | |
| | SWACEQGTPPIFSWLSAAPTSLGPRTTHSSVLI | |
| | ITPRPQDHGTNLTCQVKFAGAGVTTERTIQLNV | |
| | TYVPQNPTTGIFPGDGSGKQETRAGVVHGAIGG | |
| | AGVTALLALCLCLIFFIVKTHRRKAARTAVGRN | |
| | DTHPTTGSASPKHQKKSKLHGPTETSSCSGAAP | |
| | TVEMDEELHYASLNFHGMNPSKDTSTEYSEVRT | |
| | Q | |
| CD33 LQV | MPLLLLLPLLWAGALAMDPNFWAAAQESVTVQE | 108 |
| | GLCVLVPCTFFHPIPYYDKNSPVHGYWFREGAI | |
| | ISRDSPVATNKLDQEVQEETQGRFRLLGDPSRN | |
| | NCSLSIVDARRRDNGSYFFRMERGSTKYSYKSP | |
| | QLSVHVTDLTHRPKILIPGTLEPGHSKNLTCSV | |
| | SWACEQGTPPIFSWLSAAPTSLGPRTTHSSVLI | |
| | ITPRPQDHGTNLTCQVKFAGAGVTTERTIQLNV | |
| | TYVPQNPTTGIFPGDGSGKQETRAGVVHGAIGG | |
| | AGVTALLALCLCLIFFIVKTHRRKAARTAVGRN | |
| | DTHPTTGSASPKHQKKSKLHGPTETSSCSGAAP | |
| | TVEMDEELHYASLNFHGMNPSKDTSTEYSEVRT | |
| | Q | |
| CD33 N20D | MPLLLLLPLLWAGALAMDPDFWLQVQESVTVQE | 109 |
| | GLCVLVPCTFFHPIPYYDKNSPVHGYWFREGAI | |
| | ISRDSPVATNKLDQEVQEETQGRFRLLGDPSRN | |
| | NCSLSIVDARRRDNGSYFFRMERGSTKYSYKSP | |
| | QLSVHVTDLTHRPKILIPGTLEPGHSKNLTCSV | |
| | SWACEQGTPPIFSWLSAAPTSLGPRTTHSSVLI | |
| | ITPRPQDHGTNLTCQVKFAGAGVTTERTIQLNV | |
| | TYVPQNPTTGIFPGDGSGKQETRAGVVHGAIGG | |
| | AGVTALLALCLCLIFFIVKTHRRKAARTAVGRN | |
| | DTHPTTGSASPKHQKKSKLHGPTETSSCSGAAP | |
| | TVEMDEELHYASLNFHGMNPSKDTSTEYSEVRT | |
| | Q | |
| CD33 F21Y | MPLLLLLPLLWAGALAMDPNYWLQVQESVTVQE | 110 |
| | GLCVLVPCTFFHPIPYYDKNSPVHGYWFREGAI | |
| | ISRDSPVATNKLDQEVQEETQGRFRLLGDPSRN | |
| | NCSLSIVDARRRDNGSYFFRMERGSTKYSYKSP | |
| | QLSVHVTDLTHRPKILIPGTLEPGHSKNLTCSV | |
| | SWACEQGTPPIFSWLSAAPTSLGPRTTHSSVLI | |
| | ITPRPQDHGTNLTCQVKFAGAGVTTERTIQLNV | |
| | TYVPQNPTTGIFPGDGSGKQETRAGVVHGAIGG | |
| | AGVTALLALCLCLIFFIVKTHRRKAARTAVGRN | |
| | DTHPTTGSASPKHQKKSKLHGPTETSSCSGAAP | |
| | TVEMDEELHYASLNFHGMNPSKDTSTEYSEVRT | |
| | Q | |
| CD33 L23I | MPLLLLLPLLWAGALAMDPNFWIQVQESVTVQE | 111 |
| | GLCVLVPCTFFHPIPYYDKNSPVHGYWFREGAI | |
| | ISRDSPVATNKLDQEVQEETQGRFRLLGDPSRN | |
| | NCSLSIVDARRRDNGSYFFRMERGSTKYSYKSP | |
| | QLSVHVTDLTHRPKILIPGTLEPGHSKNLTCSV | |
| | SWACEQGTPPIFSWLSAAPTSLGPRTTHSSVLI | |
| | ITPRPQDHGTNLTCQVKFAGAGVTTERTIQLNV | |
| | TYVPQNPTTGIFPGDGSGKQETRAGVVHGAIGG | |
| | AGVTALLALCLCLIFFIVKTHRRKAARTAVGRN | |
| | DTHPTTGSASPKHQKKSKLHGPTETSSCSGAAP | |
| | TVEMDEELHYASLNFHGMNPSKDTSTEYSEVRT | |
| | Q | |
| CD33 Q24E | MPLLLLLPLLWAGALAMDPNFWLEVQESVTVQE | 112 |
| | GLCVLVPCTFFHPIPYYDKNSPVHGYWFREGAI | |
| | ISRDSPVATNKLDQEVQEETQGRFRLLGDPSRN | |
| | NCSLSIVDARRRDNGSYFFRMERGSTKYSYKSP | |
| | QLSVHVTDLTHRPKILIPGTLEPGHSKNLTCSV | |
| | SWACEQGTPPIFSWLSAAPTSLGPRTTHSSVLI | |
| | ITPRPQDHGTNLTCQVKFAGAGVTTERTIQLNV | |
| | TYVPQNPTTGIFPGDGSGKQETRAGVVHGAIGG | |
| | AGVTALLALCLCLIFFIVKTHRRKAARTAVGRN | |
| | DTHPTTGSASPKHQKKSKLHGPTETSSCSGAAP | |
| | TVEMDEELHYASLNFHGMNPSKDTSTEYSEVRT | |
| | Q | |

FIG. 5 shows histograms comparing recombinant CD33 expression in 293 FT cells stained with a secondary antibody (negative control), P67.6 (positive control), or lintuzumab. Expression of CD33 WT, CD33 delta-epitope (dEpi), and CD33 N20D was evaluated. CD33 WT was bound by both P67.6 and lintuzumab; CD33 delta-epitope (dEpi) displayed no surface expression or lacked a an epitope shared between P67.6 and lintuzumab; and CD33 N20D displayed no reactivity with lintuzumab, while P67.6 binding confirmed surface expression.

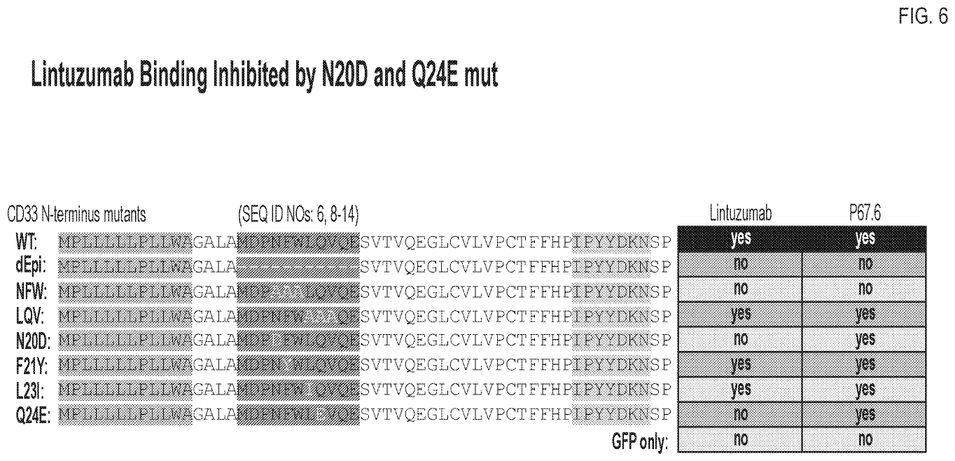

FIG. 6 shows lintuzumab binding activity to the various CD33 N-terminus mutants tested as shown in FIG. 4. Lintuzumab binding was disrupted by the N20D and Q24E mutations.

### Example 2: Evaluation of an Editing Strategy to Generate an Artificial PAM and to Disrupt CD20 ANPSE (SEQ ID NO: 15) Epitope

The CD20 “ANPSE” (SEQ ID NO: 15) epitope has been extensively characterized, and mutation of N171 results in abolishment of Ab binding for many binders, including some that have been used in CARs. See, e.g., Meyer et al, B J Haem, (2018) 180 (6): 808-820. The binding site on CD20 is different among recently developed CD20 mAbs (FIGS. 7A-7C). FIG. 7A shows epitope mapping with CD20 wild type (WT) and CD20 mutants (XDO=T159K/N163D/N1660, AxP=A170S/P1725). HDC293F cells were transfected with plasmids, and binding of monoclonal antibodies (mAbs; 5 μg/ml) to CD20 was measured by fluorescence-activated cell sorting (FACS). Binding was compared to Type I CD20 mAbs (m708 and mRTX) and Type II mAbs (B1 and m1188).

FIG. 7B shows determination of residues important for CD20 mAb binding. To identify crucial amino acids required for binding within the larger extracellular loop, binding of CD20 mAbs (5 μg/ml) to CD20 mutants was evaluated using a single mutant library spanning the ¹⁶⁸EPANPSEKNS¹⁷⁷ sequence (SEQ ID NO: 113) of the CD20 protein transiently expressed on HEK293 cells was measured by FACS.

FIG. 7C shows epitope mapping using variants of the CD20 amino acid sequence of YNCEPANPSEKNSPSTQYCYS (SEQ ID NO: 16) which resulted in identification of the epitope of antibody clone m1 but only marginally of antibody clone m2. Binding of 1 μg/ml mAB to the peptide and a panel of mutants with each amino acid replaced with all other available (positional scan; excluding cysteine) was determined by enzyme-linked immunosorbent assay.

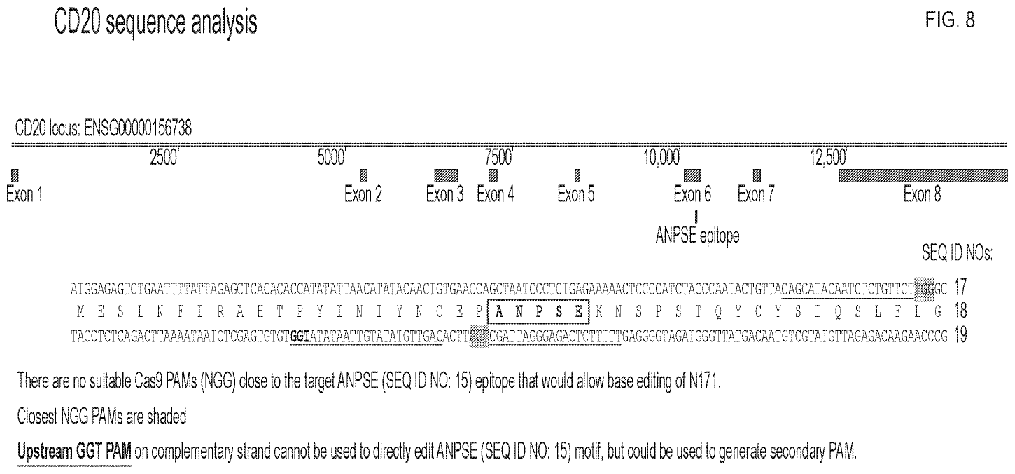

FIG. 8 shows CD20 sequence analysis. There are no suitable Cas9 PAMs (NGG) close to the target ANPSE (SEQ ID NO: 15) epitope that would allow base editing of N171. The closest NGG PAMs are the two right most three nucleotide-long sequences (second GGT from left on lower strand and TGG of last five nucleotides on top strand) which are shaded in grey. The upstream GGT PAM on the complementary strand cannot be used to directly edit the ANPSE (SEQ ID NO: 15) motif but could be used to generate secondary PAM.

FIG. 9 shows an exemplary approach for editing the CD20 ANPSE (SEQ ID NO: 15) epitope. The schematic shows a first target domain having the nucleic acid sequence of ATATAATTGTATATGTTGAC (SEQ ID NO: 69) in proximity to a GGT PAM, which is suitable to provide a first mutation to generate an artificial PAM having the nucleic acid sequence of GGC in the genomic DNA molecule in proximity to a second or subsequent target domain having the nucleic acid sequence of ACTTGGTCGATTAGGGAGAC (SEQ ID NO: 71) that lacks a naturally occurring PAM suitable to provide a second mutation. The schematic further shows a second mutation that results in the modification of the CD20 epitope ANPSE (SEQ ID NO: 15) to ALPPE (SEQ ID NO: 90). The amino acid substitutions are expected to result in a reduction of the binding activity of an anti-CD20 antibody to the epitope as compared to the unmodified epitope.

### Example 3: Evaluation of an Exemplary Editing Strategy to Generate an Artificial PAM and to Disrupt the CD38 R195 Epitope

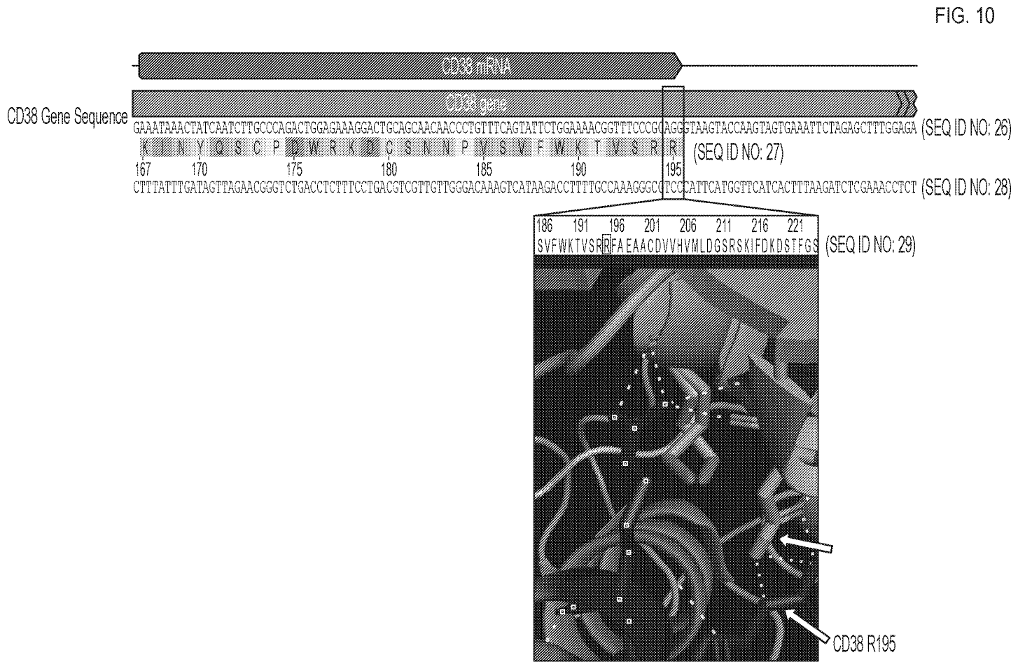

FIG. 10 shows a non-limiting example of a CD38 gene sequence, and its corresponding amino acid sequence, encoding an epitope comprising an arginine at amino acid residue position 195 of CD38 (R195). As shown in the structural model of CD38, R195 is predicted to interact with the anti-CD38 antibody isatuximab via non-covalent, polar interactions (dashed lines). By generating an artificial PAM occurring in an intron region of CD38, editing will not expected to disrupt any protein coding region. Additionally, only one target A nucleotide is present in the boxed nucleic acid sequence in FIG. 10 which reduces bystander modifications (e.g., off-target editing).

FIGS. 11A-11D show a non-limiting example of a strategy for modification of a CD38 epitope comprising arginine 195 (R195) resulting in production of a CD38 variant comprising the amino acid sequence KINYQSCPDWRKDCSNNPVSVFWKTVSRG (SEQ ID NO: 76) (corresponding to residue positions 167-195 of CD38) in various cell line models.

FIG. 11A shows two CD38 modification strategies comprising editing of an endogenous CD38 gene. The first guide RNA comprised a targeting domain sequence of GTAGTGAAATTCTAGAGCTT (SEQ ID NO: 81) (CD38 gRNA-A) which induced an ABE-catalyzed edit in the first target domain, thereby generating an edited endogenous CD38 gene comprising the sequence CGCAGGGTAAGTACCAAGTGGTGAAATTCTAGAGCTTTGGAGA (SEQ ID NO: 72), wherein the artificial PAM comprised the sequence TGG. The artificial PAM was subsequently used by a second gRNA comprising a targeting domain sequence of CCCGCAGGGTAAGTACCAAG (SEQ ID NO: 83) (CD38 gRNA-B) to induce an ABE-catalyzed edit in a second target domain, thereby producing an edited endogenous CD38 gene comprising the sequence CGCGGGGTAAGTACCAAGTGGTGAAATTCTAGAGCTTTGGAGA (SEQ ID NO: 74) encoding a modified CD38 epitope comprising a glycine substituted for arginine at amino acid residue position 195 in CD38 (R195G). Additionally, CD38 gRNA-A was used to induce an ABE-catalyzed edit in the first target domain, thereby generating an edited endogenous CD38 gene comprising the sequence CGCAGGGTAAGTACCAAGTAGTGGAATTCTAGAGCTTTGGAGA (SEQ ID NO: 114), wherein the artificial PAM comprised the sequence TGG adjacent to a spacer sequence. The artificial PAM was subsequently used by a second gRNA (CD38 gRNA-C) comprising a targeting domain sequence of GCAGGGTAAGTACCAAGTAG (SEQ ID NO: 84) to induce an ABE-catalyzed edit in a second target domain, thereby producing an edited endogenous CD38 gene encoding a modified epitope comprising R195G or CD38 knockout.

FIG. 11B shows representative DNA sequencing analysis of genomic DNA obtained from hematopoietic stem cells (HSPCs) that were electroporated with mRNA encoding ABE and CD38 gRNA-A, CD38 gRNA-B, or both CD38 gRNA-A and CD38 gRNA-B simultaneously. Electroporation of HSPCs with CD38 gRNA-A led to A>G conversion at the first A nucleotide target that was detected in more than 80% of sequencing reads. Electroporation of HSPCs with CD38 gRNA-A followed by CD38 gRNA-B resulted in high detection of A>G conversion at the first and second A nucleotide targets in sequencing reads. Following sequencing analysis of editing outcomes, CD38 gRNA-A and CD38 gRNA-B were further pursued in Jurkat and Daudi cell line models.

FIG. 11C shows representative DNA sequencing analysis of genomic DNA obtained from Jurkat cells and Daudi cells that were electroporated with mRNA encoding ABE or mRNA encoding ABE and CD38 gRNA-A. Sequencing results indicated that both Jurkat cells and Daudi cells exhibited A>G conversion at the first A nucleotide target. Higher editing efficiency was observed in Jurkat cells compared to Daudi cells.

FIG. 11D shows representative DNA sequencing analysis of genomic DNA obtained from Jurkat cells that were electroporated with mRNA encoding ABE or mRNA encoding ABE and CD38 gRNA-A or CD38 gRNA-A followed by CD38 gRNA-B. Sequencing results indicated that electroporation of Jurkat cells with CD38 gRNA-A followed by CD38 gRNA-B yielded A>G conversion at the first and second A nucleotide targets. Subsequently, single cell clones of Jurkat cells that were expected to comprise 100% editing (cells comprising an endogenous CD38 gene encoding CD38 R195G) were prepared and further analyzed.

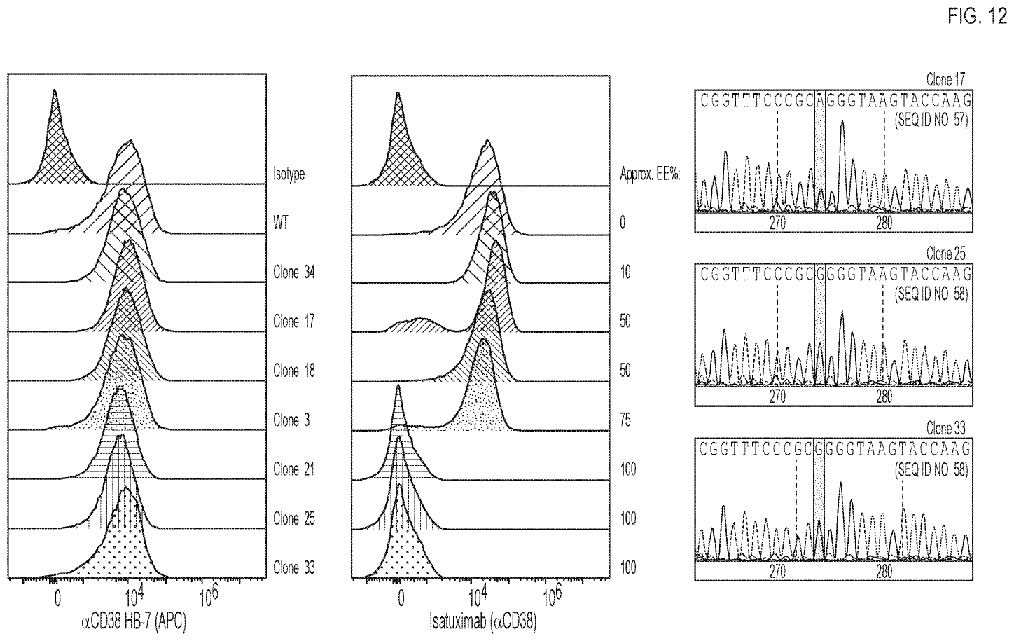

FIG. 12 shows representative flow cytometry analyses of Jurkat cell clones expressing CD38 comprising the R195G mutation. Intact Jurkat cells that underwent electroporation with mRNA encoding ABE and CD38 gRNA-A followed by CD38 gRNA-B as described above were contacted with primary anti-CD38 antibody clone (HB-7) or Isatuximab followed by a fluorescent secondary antibody comprising binding affinity to HB-7 and Isatuximab. The results indicated Jurkat cell clones exhibiting 100% editing efficiency were fully recognized by HB-7 which binds to an epitope not effected by CD38 editing to encode the modified epitope comprising R195G. Jurkat cell clones exhibiting 100% editing and expressing CD38 comprising R195G were not recognized by Isatuximab which binds R195.

### Example 4: Evaluation of an Exemplary Editing Strategy to Generate an Artificial PAM and to Disrupt the CD123 Signal Peptide Region

The signal peptide region of CD123 comprises an amino acid sequence which is recognized by intracellular trafficking machinery. Thus, the signal peptide region plays a role in localizing CD123 protein to the cell membrane. Upon insertion into the cell membrane, CD123 protein may be recognized by antibodies, or antigen-binding fragments thereof, comprising binding affinity to CD123 amino acids that are on the extracellular side of the plasma membrane. The signal peptide region of CD123 naturally comprises leucine residues at amino acid positions 8 and 13 of CD123 (L8 and L13, respectively) which are important for cell surface localization of CD123.

FIG. 13 shows a non-limiting example of a CD123 modification strategy comprising editing of an endogenous CD123 gene sequence encoding the signal peptide region. To modify the signal peptide region of CD123, the first gRNA comprised a targeting domain sequence of CCTTTGGCTCACGCTGCTCC (SEQ ID NO: 82) (CD123 gRNA-A) which induced an ABE-catalyzed edit in a first target domain, thereby generating an edited CD123 endogenous gene comprising the sequence TACCAGGAGGAAACCGAGTGCGGCGAGGACTAGCGGGACGGGACAGAGGACGTTTGCTTCCT T (SEQ ID NO: 77), wherein the artificial PAM comprised the sequence GGC and encodes a proline at amino acid residue position 8 of CD123. The artificial PAM was subsequently used by a second gRNA comprising a targeting domain sequence of CTCCTGATCGCCCTGCCTG (SEQ ID NO: 85) (CD123 gRNA-B) to induce an ABE-catalyzed edit in a second target, thereby producing an edited endogenous CD123 gene comprising the sequence TACCAGGAGGAAACCGAGTGCGGCGAGGACTAGCGGGGGGGGACAGAGGACGTTTGCTTCCT T (SEQ ID NO: 78) which encoded a modified signal peptide region comprising the amino acid sequence MVLLWLTPLLIAPPCLLOTKE (SEQ ID NO: 80) (corresponding to amino acid positions 1-21 of CD123) with prolines at amino acid positions 8 and 13 (L8P and L13P).

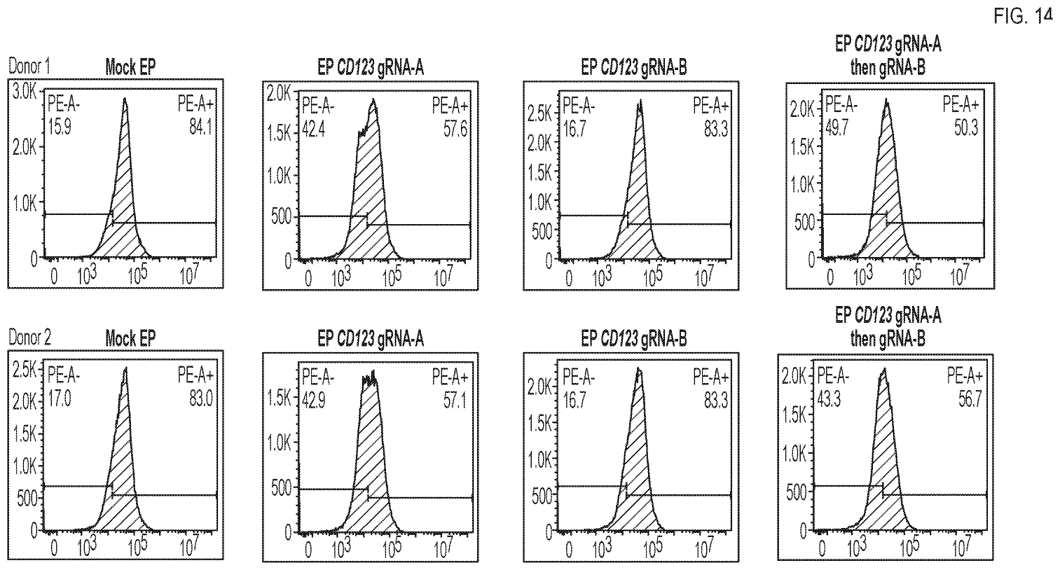

FIG. 14 shows representative flow cytometry analyses of CD34+ hematopoietic stem or progenitor cells (HSPCs) (clone ID numbers: Donor 1 and Donor 2) expressing CD123 comprising the L8P and L13P modification. Intact HSPCs that underwent electroporation with mRNA encoding ABE alone or mRNA encoding ABE and CD123 gRNA-A, CD123 gRNA-B, or both CD123 gRNA-A and CD123 gRNA-B simultaneously as described above were contacted with a primary anti-CD123 antibody (clone 6H6) followed by a fluorescent secondary antibody comprising binding affinity to said primary antibody. Approximately 83-84% of HSPCs that were subjected to mock electroporation (mRNA encoding ABE) were positive for CD123 on the cell surface. Electroporation of HSPCs with CD123 gRNA-B resulted in no signal peptide region modification relative to mock-treated controls, and approximately 83% of said HSPCs were positive for CD123 on the cell surface. This result indicated that editing at the site targeted by CD123 gRNA-B was not achieved without prior electroporation with CD123 gRNA-A. Electroporation with CD123 gRNA-A resulted in signal peptide region disruption and reduced localization of CD123 to the cell surface of HSPCs, wherein approximately 57% of the population was positive for CD123 on the cell surface. Electroporation with CD123 gRNA-A followed by CD123 gRNA-B resulted in further decreases in CD123 cell surface localization, wherein approximately 50-57% of the population were positive for CD123 on the cell surface.

### EQUIVALENTS AND SCOPE

Those skilled in the art will recognize, or be able to ascertain using no more than routine experimentation, many equivalents of the exemplary embodiments described herein. The scope of the present disclosure is not intended to be limited to the above description.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, it is to be understood that every possible individual element or subgroup of the elements is also disclosed, and that any element or subgroup of elements can be removed from the group. It is also noted that the term “comprising” is intended to be open and permits the inclusion of additional elements, features, or steps. It should be understood that, in general, where an embodiment, is referred to as comprising particular elements, features, or steps, embodiments, that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Aug. 28, 2019. When one gene or protein references a plurality of sequence accession numbers, all of the sequence variants are encompassed.

In addition, it is to be understood that any particular embodiment of the present disclosure may be explicitly excluded from any one or more of the claims. Where ranges are given, any value within the range may explicitly be excluded from any one or more of the claims. For purposes of brevity, all of the embodiments in which one or more elements, features, purposes, or aspects is excluded are not set forth explicitly herein.

1. A genetically engineered cell, comprising:
a modified genomic DNA molecule,
wherein the modified genomic DNA molecule comprises a mutation in a nucleotide sequence at a first target domain,
wherein the modified genomic DNA molecule comprises an artificial protospacer adjacent motif (PAM) comprising the mutation at the first target domain, or a part of it, and
wherein an unmodified genomic DNA molecule of the same type does not comprise a PAM sequence at the first target domain.

2. The genetically engineered cell of claim 1, wherein the mutation at the first target domain comprises a nucleotide substitution, insertion, or deletion, as compared to the nucleotide sequence of an unmodified genomic DNA molecule of the same type.

3. The genetically engineered cell of claim 1 or 2, wherein the mutation at the first target domain comprises a nucleotide substitution as compared to the nucleotide sequence of an unmodified genomic DNA molecule of the same type.

4. The genetically engineered cell of any one of claims 1-3, wherein the mutation at the first target domain comprises a nucleotide substitution of 1, 2, 3, 4, 5, 6, 7, 8, 9, or 10 nucleotides as compared to the nucleotide sequence of an unmodified genomic DNA molecule of the same type.

5. The genetically engineered cell of any one of claims 1-4, wherein the first target domain is within proximity of a PAM which is capable of targeting a Clustered Regularly Interspaced Short Palindromic Repeats (CRISPR)-CRISPR associated (Cas) (CRISPR/Cas) system comprising a Cas nuclease and a guide RNA (gRNA) to the first target domain.

6. The genetically engineered cell of any one of claims 1-5, wherein the first target domain is within about 1, 2, 3, 4, 5, 6, 7, 8, 9, 10, 11, 12, 13, 14, 15, 16, 17, 18, 19, 20, 21, 22, 23, 24, or 25 nucleotides of a naturally occurring PAM which is capable of targeting a CRISPR/Cas system to the first target domain.

7. The genetically engineered cell of any one of claims 1-6, wherein the unmodified genomic DNA molecule lacks a PAM in proximity of a second or subsequent target domain which is capable of targeting a CRISPR/Cas system to the second or subsequent target domain.

8. The genetically engineered cell of any one of claims 1-7, wherein the unmodified genomic DNA molecule lacks a PAM within about 1, 2, 3, 4, 5, 6, 7, 8, 9, 10, 11, 12, 13, 14, 15, 16, 17, 18, 19, 20, 21, 22, 23, 24, or 25 nucleotides of a second or subsequent target domain which is capable of targeting a CRISPR/Cas system to the second or subsequent target domain.

9. The genetically engineered cell of any one of claims 1-8, wherein the PAM which is capable of targeting a CRISPR/Cas system to the first target domain and the artificial PAM are separated by about 1, 2, 3, 4, 5, 6, 7, 8, 9, 10, 11, 12, 13, 14, 15, 16, 17, 18, 19, 20, 21, 22, 23, 24, or 25 nucleotides.

10. The genetically engineered cell of any one of claims 1-9, wherein the modified genomic DNA molecule further comprises a mutation at a second or subsequent target domain as compared to the nucleotide sequence of an unmodified genomic DNA molecule of the same type, wherein the second or subsequent target domain is in proximity to the artificial PAM.

11. The genetically engineered cell of claim 10, wherein the mutation at the second or subsequent target domain comprises a mutation within 8, 9, 10, 11, 12, 13, 14, 15, 16, 17, 18, 19, 20, 21, or 22 nucleotides from the artificial PAM.

12. The genetically engineered cell of claim 10, wherein the mutation at the second or subsequent target domain comprises a mutation within 8, 9, 10, 11, 12, 13, 14, 15, 16, 17, 18, 19, 20, 21, or 22 nucleotides upstream of the 5′-terminal nucleotide of the artificial PAM.

13. The genetically engineered cell of claim 10, wherein the mutation at the second or subsequent target domain comprises a mutation within 16-18 nucleotides upstream of the 5′-terminal nucleotide of the artificial PAM.

14. The genetically engineered cell of claim 10, wherein the mutation at the second or subsequent target domain comprises a mutation at least 8, 9, 10, 11, or 12 nucleotides upstream of the 5′-terminal nucleotide of the artificial PAM.

15. The genetically engineered cell of any one of claims 10-14, wherein the mutation at the first target domain and/or the mutation at the second or subsequent target domain comprises a C-to-T substitution and/or an A-to-G nucleotide substitution.

16. The genetically engineered cell of any one of claims 10-15, wherein the mutation at the first target domain comprises an A-to-G nucleotide substitution, and the mutation at the second or subsequent target domain comprises a C-to-T and/or an A-to-G nucleotide substitution.

17. The genetically engineered cell of any one of claims 10-16, wherein the mutation at the first target domain comprises a C-to-T nucleotide substitution, and the mutation at the second or subsequent target domain comprises a C-to-T and/or an A-to-G nucleotide substitution.

18. The genetically engineered cell of any one of claims 10-17, wherein the mutation in the nucleotide sequence at the second or subsequent target domain is in a target gene.

19. The genetically engineered cell of any one of claims 1-18, wherein the target gene encodes a cell-surface protein.

20. The genetically engineered cell of any one of claims 1-19, wherein the target gene encodes a lineage-specific cell-surface protein.

21. The genetically engineered cell of any one of claims 1-20, wherein the target gene encodes a cell-surface protein epitope.

22. The genetically engineered cell of any one of claims 10-21, wherein the mutation in the nucleotide sequence at the second or subsequent target domain alters the amino acid sequence of an epitope in the cell-surface protein epitope, resulting in a modified epitope.

23. The genetically engineered cell of claim 22, wherein the mutation in the nucleotide sequence at the second or subsequent target domain results in a deletion, a substitution, an insertion, or an inversion of one or more amino acid residues, or a combination thereof.

24. The genetically engineered cell of any one of claim 22 or 23, wherein the mutation in the nucleotide sequence at the second or subsequent target domain results in an amino acid substitution in the cell-surface protein epitope.

25. The genetically engineered cell of any one of claim 23 or 24, wherein the amino acid substitution is a non-conservative amino acid substitution.

26. The genetically engineered cell of any one of claims 22-25, wherein the cell-surface protein epitope is bound by an agent, and wherein the modified epitope is characterized by a reduction or an abolishment of binding activity of the agent.

27. The genetically engineered cell of claim 26, wherein the binding activity of the agent to the modified epitope is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, or more, as compared to an unmodified epitope.

28. The genetically engineered cell of claim 26 or 27, wherein the reduction in binding activity comprises an increase in K_{D}, IC₅₀, and/or EC₅₀.

29. The genetically engineered cell of claim 27, wherein:
(i) the K_{D}, IC₅₀, and/or EC₅₀ is increased by the amino acid substitution by at least 5%, 10%, 15%, 20%, 25%, 30%, 35%, 40%, 45%, 50%, 55%, 60%, 65%, 70%, 75%, 80%, 85%, 90%, 95%, or 99% or more as compared to the unmodified epitope; or
(ii) the K_{D}, IC₅₀, and/or EC₅₀ is increased by the amino acid substitution by at least 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold as compared to the unmodified epitope.

30. The genetically engineered cell of anyone of claims 20-29, wherein the lineage-specific cell-surface protein is CD19; CD123; C-type lectin-like molecule-1 (CLL-1) CD38; KIT (CD117); CD20; or EGF-like module-containing mucin-like hormone receptor-like 2 (EMR2).

31. The genetically engineered cell of anyone of claims 20-29, wherein the lineage-specific cell-surface protein is CD5, CD19, CD20, CD38, CD117, or CD123.

32. The genetically engineered cell of anyone of claims 20-29, wherein the lineage-specific cell-surface protein is CD19, CD20, CD38, C-type lectin-like molecule-1 (CLL-1) CD123, or CD33.

33. The genetically engineered cell of anyone of claims 20-29, wherein the cell-surface lineage-specific protein is CD33.

34. The genetically engineered cell of anyone of claims 20-29, wherein the cell-surface lineage-specific protein is CD20.

35. The genetically engineered cell of anyone of claims 20-29, wherein the cell-surface lineage-specific protein is CLL-1.

36. The genetically engineered cell of anyone of claims 20-29, wherein the cell-surface lineage-specific protein is CD123.

37. The genetically engineered cell of anyone of claims 20-29, wherein the cell-surface lineage-specific protein is CD38.

38. The genetically engineered cell of anyone of claims 20-29, wherein the cell-surface lineage-specific protein is CD19.

39. The genetically engineered cell of anyone of claims 20-29, wherein the cell-surface lineage-specific protein is CD117.

40. The genetically engineered cell of anyone of claims 20-29, wherein the cell-surface lineage-specific protein is EMR2.

41. The genetically engineered cell of anyone of claims 20-29, wherein the cell-surface lineage-specific protein is CD5.

42. The genetically engineered cell of any one of claims 1-41, wherein the target gene, the cell-surface protein, and/or the cell-surface lineage-specific protein expression is associated with a cancer.

43. The genetically engineered cell of any one of claims 1-42, wherein the target gene, the cell-surface protein, and/or the cell-surface lineage-specific protein expression is associated with a hematopoietic malignancy.

44. The method of claim 43, wherein the hematopoietic malignancy is Hodgkin's lymphoma, non-Hodgkin's lymphoma, leukemia, multiple myeloma (MM), myelodysplastic syndrome (MDS), or blastic plasmacytoid dendritic cell neoplasm (BPDCN).

45. The method of claim 43 or 44, wherein the hematopoietic malignancy is acute myeloid leukemia, B-cell acute lymphoblastic leukemia (B-ALL), chronic myelogenous leukemia, acute lymphoblastic leukemia, or chronic lymphoblastic leukemia.

46. The genetically engineered cell of any one of claims 1-45, wherein the PAM and/or the artificial PAM is a Cas nuclease PAM.

47. The genetically engineered cell of any one of claims 1-46, wherein the PAM and/or the artificial PAM is a Cas9 PAM.

48. The genetically engineered cell of any one of claims 1-47, wherein the PAM and/or the artificial PAM is a Cas12a PAM

49. The genetically engineered cell of any one of claims 1-48 wherein the PAM and/or the artificial PAM is a SpyCas9 PAM, a SpGCas9 PAM, a SpRYCas9 PAM, a Sth1Cas9 PAM, a SauCas9 PAM, a NmeCas9 PAM, a RspCas9 PAM, a Cca1Cas9 PAM, a PspCas9 PAM, a OrhCas9 PAM, a ScCas9 PAM, a SmacCas9 PAM, a TdeCas9 PAM, a Nme2Cas9 PAM, a CjeCas9 PAM, a SmuCas9 PAM, a Smu2Cas9 PAM, a PmuCas9 PAM, a SpaCas9 PAM, a NciCas9 PAM, a ClaCas9 PAM, a PlaCas9 PAM, a CdCas9 PAM, a IgnaviCas9 PAM, a ThermoCas9 PAM, a GeoCas9 PAM, a G. LC300 Cas9 PAM, a AceCas9 PAM, a Sth3Cas9 PAM, a BlatCas9 PAM, a FnCas9 PAM, a LfeCas9 PAM, a LpnCas9 PAM, a KhuCas9 PAM, a AinCas9 PAM, a CglCas9 PAM, a Esp1Cas9 PAM, a Esp2Cas9 PAM, a FmaCas9 PAM, a LceCas9 PAM, a LrhCas9 PAM, a Lsp1Cas9 PAM, a Lsp2Cas9 PAM, a PacCas9 PAM, a TbaCas9 PAM, a TpuCas9 PAM, a VpaCas9 PAM, a EfaCas9 PAM, a EitCas9 PAM, a LanCas9 PAM, a LmoCas9 PAM, a Sag1Cas9 PAM, a Sag2Cas9 PAM, a SdyCas9 PAM, a Seq1Cas9 PAM, a Seq2Cas9 PAM, a SgaCas9 PAM, a Smu3Cas9 PAM, a SraCas9 PAM, a BniCas9 PAM, a EceCas9 PAM, a EdoCas9 PAM, a FhoCas9 PAM, a MgaCas9 PAM, a MseCas9 PAM, a SgoCas9 PAM, a SmaCas9 PAM, a SsaCas9 PAM, a SsiCas9 PAM, a SsuCas9 PAM, a Sth1ACas9 PAM, a TspCas9 PAM, a BokCas9 PAM, a CcoCas9 PAM, a CpeCas9 PAM, a DdeCas9 PAM, a Ghc2Cas9 PAM, a Ghy3Cas9 PAM, a Ghy4Cas9 PAM, a GspCas9 PAM, a KkiCas9 PAM, a NspCas9 PAM, a TmoCas9 PAM, a NsaCas9 PAM, a JpaCas9 PAM, a BboCas9 PAM, a Cca2Cas9 PAM, a Cme2Cas9 PAM, a Cme3Cas9 PAM, a Cme4Cas9 PAM, a CsaCas9 PAM, a Ghc1Cas9 PAM, a GheCas9 PAM, a Ghh1Cas9 PAM, a Ghh2Cas9 PAM, a Ghy1Cas9 PAM, a MscCas9 PAM, a SdoCas9 PAM, a SpacCas9 PAM, a CgaCas9 PAM, a CmelCas9 PAM, a FfrCas9 PAM, a Ghy2Cas9 PAM, a PhiCas9 PAM, a WviCas9 PAM, a CcCas9 PAM, a FnCas12a PAM, a AsCas12a PAM, a HkCas12a PAM, a PiCas12a PAM, a PdCas12a PAM, a LbCas12a PAM, a Lb2Cas12a or Lb5Cas12a PAM, a CMtCas12a PAM, a MbCas12a PAM, a TsCas12a PAM, a Pb2Cas12a PAM, a MICas12a PAM, a Mb2Cas12a PAM, a Mb3Cas12a PAM, a CMaCas12a PAM, a BsCas12a PAM, a BfCas12a PAM, a BoCas12a PAM, a Adurb193Cas12a PAM, a Adurb336Cas12a PAM, a Fn3Cas12a PAM, a Lb6Cas12a PAM, a EcCas12a PAM, a PsCas12a PAM, a McCas12a PAM, a AacCas12b PAM, a BthCas12b PAM, a AkCas12b PAM, a EbCas12b PAM, a BvCas12b PAM, a BhCas12b PAM, a LsCas12b PAM, a BrCas12b PAM, a Cas12c1 PAM, a Cas12c2 PAM, a OspCas12c PAM, a Cas12d.15 PAM, a Cas12d.1 (CasY.1) PAM, a DpbCas12e (DpbCasX) PAM, a PlmCas12e (PlmCasX) PAM, a MilCas12f2 PAM, a Un1Cas12f1 PAM, a Un2Cas12f1 PAM, a Mi2Cas12f2 PAM, a AuCas12f2 PAM, a PtCas12f1 PAM, a AsCas12f1 PAM, a RuCas12f1 PAM, a SpCas12f1 PAM, a CnCas12f1 PAM, a Cas12h1 PAM, a Cas12i1 PAM, a Cas1212 PAM, a Cas12j-1 (CasPhi-1) PAM, a Cas12j-2 (CasPhi-2) PAM, a Cas12j-3 (CasPhi-3) PAM, a ShCas12k PAM, or a AcCas12k PAM.

50. The genetically engineered cell of any one of claims 1-49, wherein the PAM, and/or the artificial PAM comprises, and/or consists of a nucleotide sequence of NGG; NNAGAAW; NNGRRT; NNNNGATT; NVNDCCY; BRTTTTT; NR (A or G) TTTT; NNAAAR (G or A); N (N or A) G; NAAN; NAAAAY; NHDTCCA; NNNVRYM; NNNNRYAC; NAA; GNNNNCNNA; NNGTGA; NNNNGTA; NNGGG; NNNCAT; NNRHHHY; NRRNAT; NNNNCNAA; NNNNCMCA; NNNNCRAA; NNNNGMAA; NNNCC; NGGNG; NNNNCNDD; NYAAA; NRGNN; N (C or D) GGN (T or A or G or C) NN; NRTAW; N (C or K or A) AARC; NAAAG; NV (A or G or C) R (A or G) ACCN; NNGAC; NATGNT; N (T or V) NTAAW (A or T); NNGW (A or T) AY (T or C); NCAA (H (Y or A) B (Y or G); NH (T or C or A) AAAA; NNNATTT; NATAWN (A or T or S); NATARCH; B (T or G or C) GGD (A or T or G) TNN; N (G or T or M) GGAH (T or A or C) N (A or C or K) N; NRG; N (B or A) GG; NGGD (A or K) W (T or A); N (T or C or R) AGAN (A or K or C) NN; NGGD (A or T or G) H (T or M); NGGDT; NGGD (A or T or G) GNN; NNGTAM (A or C) Y; NNGH (W or C) AAA; NTGAR (G or A) N (A or Y or G) N (Y or R); NNGAAAN; NNGAD; NHARMC; NNAAAG; NHGYNAN (A or B); NNAGAAA; NHAAAAA; NH (T or M) AAAAA; NHGYRAA; NNAAACN; NN (H or G) D (A or K) GGDN (A or B); NNNNCTA; NNNNCVGAA; NNNNGYAA; NNNNATN (W or S) ANN; NNWHR (G or A) TA (not G) AA; YHHNGTH; NNNNCDAANN; NNNNCTAA; N (C or D) NNTCCN; NNNNCCAA; NAGRGN (T or V) N (T or C); NNAH (T or M) ACN; CN (C or W or G) AV (A or S) GAC; NAR (G or A) H (W or C) H (A or T or C) GN (C or T or R); NAGNGC; NATCCTN; NGTGANN; HGCNGCR; NAR (A or G) W (T or A) AC; N (C or D) M (A or C) RN (A or B) AY (C or T); NNNCAC; BGGGTCD; NNRRCC; NRRNTT; KARDAT; BRRTTTW; NARNCCN; NAR (A or G) TC; NAAN (A or T or S) RCN; HHAAATD; NNNNGNA; TTV; TTTV; YYV; KKYV; TTTM; TTYV; TTTN; TTTTA; TTN; BTTV; YTV; YTN; NYTV; DTTD; ATTN; RTTNT; HATT; ATTW; RTTN; TVT; TG; TN; TR; TA; TTCN; TTAT; TTTR; TTR; YTTR; YTTN; CTT; TTC; CCD; RTR; VTTR; TBN; VTTN; NGTT; CGTT; AGG; CGG; GTT, or RGTG,
wherein “N” is any nucleotide or base, “W” is adenine (A) or thymine (T), “R” is A or guanine (G), “V” is A, cytosine (C), or G, “Y” is C or T, and “H” is A, C, or T.

51. The genetically engineered cell of any one of claims 1-50, wherein the PAM, and/or the artificial PAM comprises, and/or consists of a nucleotide sequence of NGG.

52. The genetically engineered cell of any one of claims 1-51, wherein the PAM, and/or the artificial PAM comprises, and/or consists of a nucleotide sequence of TTN.

53. The genetically engineered cell of any one of claims 1-52, wherein the PAM, and/or the artificial PAM comprises, and/or consists of a nucleotide sequence of NGG, wherein “N” is any nucleotide or base.

54. The genetically engineered cell of any one of claims 1-53, wherein the PAM, and/or the artificial PAM comprises, and/or consists of a nucleotide sequence of AGG.

55. The genetically engineered cell of any one of claims 1-54, wherein the PAM, and/or the artificial PAM comprises, and/or consists of a nucleotide sequence of CGG.

56. The genetically engineered cell of any one of claims 1-55, wherein the PAM, and/or the artificial PAM, comprises and/or consists of a nucleotide sequence of GTT.

57. The genetically engineered cell of any one of claims 1-56, wherein the modified genomic DNA molecule encodes CD33, wherein the modified genomic DNA molecule comprises a nucleotide sequence comprising
(SEQ ID NO: 1)
ATGGATCCAAATTTCTGGCTGCA₃A₄GTGCAGGAGTCAGTGACGGTACAG
GAG;
(SEQ ID NO: 3)
ATGGA₃TCCA₇A₈A₉TTTCTGGCTGCAGGTGCAGGAGTCAGTGACGGTAC
AGGAG;
or
(SEQ ID NO: 4)
ATGGATCCAGATTTCTGGCTGCAGGTGCAGGAGTCAGTGACGGTACAGG
AG,
wherein each of A₃, A₄, A₇, A₈, and A₉ independently comprise either A or G.

58. The genetically engineered cell of any one of claims 1-57, wherein the modified genomic DNA molecule encodes a modified CD33 protein, wherein the modified CD33 protein comprises, e.g., a mutation
dEpi, characterized by the deletion of MDPNFWLQVQE (SEQ ID NO: 2) at amino acid residue positions 17-27; NFW, characterized by the substitution of NEW with AAA at residue positions 20-22;
LQV, characterized by the substitution of LQV with AAA at residue positions 23-25;
N20D, characterized by the substitution of N with D at residue position 20;
F21Y, characterized by the substitution of F with Y at residue position 21;
L23I, characterized by the substitution of L with I at residue position 23; and/or
Q24E, characterized by the substitution of Q with E at residue position 24.

59. The genetically engineered cell of any one of claims 1-58, wherein the modified genomic DNA molecule encodes a modified CD33 protein, wherein the modified CD33 protein comprises an amino acid sequence comprising
(SEQ ID NO: 8)
MPLLLLLPLLWAGALA-----------SVTVQEGLCVLVPCTFFHPIPY
YDKNSP (dEpi);
(SEQ ID NO: 9)
MPLLLLLPLLWA9GALAMDPAAALQVQESVTVQEGLCVLVPCTFFHPIP
YYDKNSP (NFW);
(SEQ ID NO: 10)
MPLLLLLPLLWAGALAMDPNFWAAAQESVTVQEGLCVLVPCTFFHPIPY
YDKNSP (LQV);
(SEQ ID NO: 11)
MPLLLLLPLLWAGALAMDPDFWLQVQESVTVQEGLCVLVPCTFFHPIPY
YDKNSP (N20D);
(SEQ ID NO: 12)
MPLLLLLPLLWAGALAMDPNYWLQVQESVTVQEGLCVLVPCTFFHPIPY
YDKNSP (F21Y);
(SEQ ID NO: 13)
MPLLLLLPLLWAGALAMDPNFWIQVQESVTVQEGLCVLVPCTFFHPIPY
YDKNSP (L23I);
or
(SEQ ID NO: 14)
MPLLLLLPLLWAGALAMDPNEWLEVQESVTVQEGLCVLVPCTFFHPIPY
YDKNSP (Q24E).

60. The genetically engineered cell of any one of claims 1-59, wherein the modified genomic DNA molecule encodes CD20, wherein the modified genomic DNA molecule comprises a nucleotide sequence comprising
(SEQ ID NO: 64)
CGAGTGTGTGGTATATAATTGTA₁₀TA₈TGTTGA₂CACTTGGTCGATTAG
GGAGACTCTTTTTGAGGGGTAGATGGGTTATGACAATG;
(SEQ ID NO: 65)
CGAGTGTGTGGTATATAATTGTGTGTGTTGGCACTTGGTCGA₁₁TTA₈G
GGA₄GA₂CTCTTTTTGAGGGGTAGATGGGTTATGACAATG;
or
(SEQ ID NO: 66)
CGAGTGTGTGGTATATAATTGTGTGTGTTGGCACTTGGTCGGTTGGGG
GGGCTCTTTTTGAGGGGTAGATGGGTTATGACAATG,
wherein each of A₂, A₄, A₈, A₁₀, A₁₁ independently comprise either A or G.

61. The genetically engineered cell of any one of claims 1-60, wherein the modified genomic DNA molecule encodes a modified CD20 protein, wherein the modified CD20 protein comprises, e.g., a mutation
(i) characterized by the substitution of I with T at residue position 8;
(ii) characterized by the substitution of Y with H at residue position 9;
(iii) characterized by the substitution of C with A at residue position 11;
(iv) characterized by the substitution of N with L at residue position 15; and/or
(v) characterized by the substitution of S with P at residue position 17.

62. The genetically engineered cell of any one of claims 1-61, wherein the modified genomic DNA molecule encodes a modified CD20 protein, wherein the modified CD20 protein comprises an amino acid sequence comprising
(SEQ ID NO: 24)
AHTPYINTHNAEPANPSEKNSPSTQYCY;
or
(SEQ ID NO: 67)
AHTPYINTHNAEPALPPEKNSPSTQYCY.

63. The genetically engineered cell of any one of claims 1-62, wherein:
(i) the first gRNA comprises a targeting domain which binds a site in a CD33 gene, optionally, wherein the site comprises the nucleic acid sequence of GCAAGTGCAGGAGTCAGTGA (SEQ ID NO: 68), or a portion thereof; or
(ii) the first gRNA comprises a targeting domain which binds a site in a CD20 gene, optionally, wherein the site comprises the nucleic acid sequence of ATATAATTGTATATGTTGAC (SEQ ID NO: 69), or a portion thereof

64. The genetically engineered cell of any one of claims 1-63, wherein:
(i) the second gRNA comprises a targeting domain which binds a site in a CD33 gene, optionally, wherein the site comprises the nucleic acid sequence of GGATCCAAATTTCTGGCTGC (SEQ ID NO: 70), or a portion thereof; or
(ii) the second gRNA comprises a targeting domain which binds a site in a CD20 gene, optionally, wherein the site comprises the nucleic acid sequence of ACTTGGTCGATTAGGGAGAC (SEQ ID NO: 71), or a portion thereof.

65. The genetically engineered cell of any one of claims 1-64, wherein the modified genomic DNA molecule encodes CD38, wherein the modified genomic DNA molecule comprises a nucleotide sequence comprising
(SEQ ID NO: 72)
CGCAGGGTAAGTACCAAGTGGTGAAATTCTAGAGCTTTGGAGA;
(SEQ ID NO: 73)
CGCAGGGTAAGTACCAAGTAGTGA₁A₂A₃TTCTAGAGCTTTGGAGA;
(SEQ ID NO: 74)
CGCGGGGTAAGTACCAAGTGGTGAAATTCTAGAGCTTTGGAGA;
or
(SEQ ID NO: 75)
CGCAGGGTA₄A₅GTACCAAGTAGTGA₁A₂A₃TTCTAGAGCTTTGGAGA,
wherein each of A₁, A₂, A₃, A₄, and A₅ independently comprise either A or G, wherein at least one of A₁, A₂, A₃, A₄, and A₅ comprise a G.

66. The genetically engineered cell of any one of claims 1-65, wherein the modified genomic DNA molecule encodes a modified CD38 protein, wherein the modified CD38 protein comprises R195G, characterized by the substitution of R with G at residue position 195.

67. The genetically engineered cell of any one of claims 1-66, wherein the modified genomic DNA molecule encodes a modified CD38 protein, wherein the modified CD38 protein comprises an amino acid sequence KINYQSCPDWRKDCSNNPVSVFWKTVSRG (SEQ ID NO: 76) corresponding to residue positions 167-195 of CD38.

68. The genetically engineered cell of any one of claims 1-67, wherein the modified genomic DNA molecule encodes CD123, wherein the modified genomic DNA molecule comprises a nucleotide sequence comprising
(SEQ ID NO: 77)
TACCAGGAGGAAACCGAGTGCGGCGAGGACTAGCGGGACGGGACAGAGG
ACGTTTGCTTCCTT;
or
(SEQ ID NO: 78)
TACCAGGAGGAAACCGAGTGCGGCGAGGACTAGCGGGGGGGGACAGAGG
ACGTTTGCTTCCTT.

69. The genetically engineered cell of any one of claims 1-68, wherein the modified genomic DNA molecule encodes a modified CD123 protein, wherein the modified CD123 protein comprises
L8P, characterized by the substitution of L with P at residue position 8; or
L8P and L13P, characterized by the substitutions of L with P at residue position 8 and L with P at residue position 13.

70. The genetically engineered cell of any one of claims 1-69, wherein the modified genomic DNA molecule encodes a modified CD123 protein, wherein the modified CD123 protein comprises an amino acid sequence comprising
(SEQ ID NO: 79)
MVLLWLTPLLIALPCLLQTKE;
or
(SEQ ID NO: 80)
MVLLWLTPLLIAPPCLLQTKE,
corresponding to residue positions 1-21 of CD123.

71. The genetically engineered cell of any one of claims 1-70, wherein:
(i) the first gRNA comprises a targeting domain which binds a site in a CD33 gene, optionally, wherein the site comprises the nucleic acid sequence of GCAAGTGCAGGAGTCAGTGA (SEQ ID NO: 68), or a portion thereof;
(ii) the first gRNA comprises a targeting domain which binds a site in a CD20 gene, optionally, wherein the site comprises the nucleic acid sequence of ATATAATTGTATATGTTGAC (SEQ ID NO: 69), or a portion thereof;
(iii) the first gRNA comprises a targeting domain which binds a site in a CD38 gene, optionally, wherein the site comprises the nucleic acid sequence of GTAGTGAAATTCTAGAGCTT (SEQ ID NO: 81), or a portion thereof; or
(iv) the first gRNA comprises a targeting domain which binds a site in a CD123 gene, optionally, wherein the site comprises the nucleic acid sequence of CCTTTGGCTCACGCTGCTCC (SEQ ID NO: 82), or a portion thereof.

72. The genetically engineered cell of any one of claims 1-71, wherein:
(i) the second gRNA comprises a targeting domain which binds a site in a CD33 gene, optionally, wherein the site comprises the nucleic acid sequence of GGATCCAAATTTCTGGCTGC (SEQ ID NO: 70), or a portion thereof;
(ii) the second gRNA comprises a targeting domain which binds a site in a CD20 gene, optionally, wherein the site comprises the nucleic acid sequence of ACTTGGTCGATTAGGGAGAC (SEQ ID NO: 71), or a portion thereof;
(iii) the second gRNA comprises a targeting domain which binds a site in a CD38 gene, optionally, wherein the site comprises the nucleic acid sequence of CCCGCAGGGTAAGTACCAAG (SEQ ID NO: 83) or GCAGGGTAAGTACCAAGTAG (SEQ ID NO: 84), or a portion thereof; or
(iv) the second gRNA comprises a targeting domain which binds a site in a CD123 gene, optionally, wherein the site comprises the nucleic acid sequence of CTCCTGATCGCCCTGCCTG (SEQ ID NO: 85), or a portion thereof.

73. The genetically engineered cell of any one of claims 1-72, wherein the Cas nuclease is Cas9, Cas12a or Cas12b.

74. The genetically engineered cell of any one of claims 1-73, wherein the Cas nuclease comprises a catalytically inactive Cas molecule.

75. The genetically engineered cell of any one of claims 1-74, wherein the Cas nuclease comprises a dead Cas (dCas).

76. The genetically engineered cell of any one of claims 1-75, wherein the Cas nuclease comprises a dead Cas9 (dCas9).

77. The genetically engineered cell of any one of claims 1-76, wherein the Cas nuclease comprises a nickase (nCas).

78. The genetically engineered cell of any one of claims 1-77, wherein the Cas nuclease comprises a nCas9.

79. The genetically engineered cell of any one of claims 1-78, wherein the Cas nuclease comprises a dCas or a nCas fused to one or more uracil glycosylase inhibitor (UGI) domains.

80. The genetically engineered cell of any one of claims 1-79, wherein the Cas nuclease comprises a dCas or a nCas fused to an adenine base editor (ABE).

81. The genetically engineered cell of claim 80, wherein the ABE comprises an adenine deaminase enzyme.

82. The genetically engineered cell of any one of claims 1-79, wherein the Cas nuclease comprises a dCas or a nCas fused to a cytosine base editor (CBE).

83. The genetically engineered cell of claim 82, wherein the CBE comprises a cytidine deaminase enzyme.

84. The genetically engineered cell of any one of claims 1-83, wherein the cell is a hematopoietic cell, a progenitor cell, or a descendant thereof.

85. The genetically engineered cell of claim 84, wherein the hematopoietic cell is a hematopoietic stem cell (HSC).

86. The genetically engineered cell of any one of claim 84 or 85, wherein the hematopoietic cell is a CD34+ cell.

87. The genetically engineered cell of any one of claims 84-86, wherein the hematopoietic cell is obtained from bone marrow, blood, umbilical cord, or peripheral blood mononuclear cells (PBMCs).

88. The genetically engineered cell of any one of claims 84-87, wherein the hematopoietic cell is a human cell.

89. A cell population, comprising a plurality of the genetically engineered cells of any one of claims 1-88.

90. A cell population comprising a plurality of genetically engineered cells, wherein at least a portion of the cells comprise:
(a) a mutation within a first target domain in a genomic DNA molecule which generates an artificial PAM;
(b) an artificial PAM in proximity of a second or subsequent target domain; and
(c) an edit within the second or subsequent target domain.

91. A method, comprising administering to a subject in need thereof:
(i) a population of the genetically engineered cells of any one of claims 1-90.

92. The method of claim 91, further comprising administering (ii) an effective amount of the agent that specifically binds the cell-surface protein epitope.

93. The method of claim 91 or 92, wherein the subject has a hematopoietic malignancy.

94. The method of claim 92 or 93, wherein the agent is a single-chain antibody fragment (scFv).

95. The method of any one of claim 92 or 93, wherein the agent is an antibody or an antibody-drug conjugate (ADC).

96. The method of claim 92, wherein the agent is an immune cell expressing a chimeric antigen receptor that comprises the antigen-binding fragment.

97. The method of claim 96, wherein the immune cells are T cells.

98. The method of claim 97, wherein the T cells express CD3, CD4, and/or CD8.

99. The method of any one of claims 96-98, wherein the chimeric antigen receptor further comprises:
(a) a hinge domain
(b) a transmembrane domain,
(c) at least one co-stimulatory domain,
(d) a cytoplasmic signaling domain, or
(e) a combination thereof.

100. The method of any one of claims 96-99, wherein the agent comprises: a CD33 antibody, a CD20 antibody, a CLL-1 antibody, a CD123 antibody, a CD38 antibody, a CD19 antibody, CD117 antibody, EMR2 antibody, or a CD5 antibody.

101. The method of any one of claims 93-100, wherein the hematopoietic malignancy is Hodgkin's lymphoma, non-Hodgkin's lymphoma, leukemia, multiple myeloma (MM), myelodysplastic syndrome (MDS), or blastic plasmacytoid dendritic cell neoplasm (BPDCN).

102. The method of any one of claims 93-101, wherein the hematopoietic malignancy is acute myeloid leukemia, B-cell acute lymphoblastic leukemia (B-ALL), chronic myelogenous leukemia, acute lymphoblastic leukemia, or chronic lymphoblastic leukemia.

103. The method of any one of claims 93-102, wherein the hematopoietic malignancy is B-cell acute lymphoblastic leukemia (B-ALL).

104. The method of any one of claims 93-102, wherein the hematopoietic malignancy is acute myeloid leukemia (AML).

105. The method of any one of claims 93-102, wherein the hematopoietic malignancy is multiple myeloma (MM).

106. The method of any one of claims 93-102, wherein the hematopoietic malignancy is myelodysplastic syndrome (MDS).

107. A method comprising:
genetically modifying a cell to generate an artificial PAM in proximity of a target domain, comprising contacting a genomic DNA molecule with:
(a) a first CRISPR/Cas system comprising a Cas nuclease and a first gRNA configured to direct the first CRISPR/Cas system to a first target domain resulting in the generation of an artificial PAM comprising introducing a mutation at the first target domain of the genomic DNA molecule;
(b) a second CRISPR/Cas system comprising a Cas nuclease and a second gRNA configured to direct the second CRISPR/Cas system to a second or subsequent target domain comprising introducing a mutation at the second or subsequent target domain, or a part of it;
wherein the second CRISPR/Cas system recognizes the artificial PAM, and wherein the second or subsequent target domain is in proximity to the artificial PAM.

108. The method of claim 107, wherein the genomic DNA molecule is in a cell.

109. The method of claim 107 or 108, wherein the cell is a genetically engineered cell of any one of claims 1-68.

110. The method of any one claims 107-109, wherein the second or subsequent target domain is in a target gene.

111. The method of claim 110, wherein the target gene encodes a cell-surface protein.

112. The method of claim 110 or 111, wherein the target gene encodes a cell-surface protein epitope.

113. The method of claim 112, wherein the mutation at the second or subsequent target domain results in an amino acid substitution in the cell-surface protein epitope.

114. The method of claim 113, wherein the amino acid substitution is a non-conservative amino acid substitution.

115. The method of claim 113 or 114, wherein the cell-surface protein epitope is bound by an agent, and wherein the amino acid substitution results in a reduction of the binding activity of the agent to the epitope comprising the amino acid substitution as compared to the unmodified epitope.

116. The method of claim 115, wherein:
(i) the binding activity of the agent to the epitope is reduced by the amino acid substitution by at least about 5%, 10%, 15%, 20%, 25%, 30%, 35%, 40%, 45%, 50%, 55%, 60%, 65%, 70%, 75%, 80%, 85%, 90%, 95%, or 99% or more as compared to the unmodified epitope; and/or
(ii) the binding activity of the agent to the epitope is reduced by the amino acid substitution by at least about or at least about 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold as compared to the unmodified epitope.

117. The method of claim 115 or 116, wherein the reduction in binding activity comprises an increase in K_{D}, IC₅₀, and/or EC₅₀.

118. The method of claim 117, wherein:
(i) the K_{D}, IC₅₀, and/or EC₅₀ is increased by the amino acid substitution by at least about 5%, 10%, 15%, 20%, 25%, 30%, 35%, 40%, 45%, 50%, 55%, 60%, 65%, 70%, 75%, 80%, 85%, 90%, 95%, or 99% or more as compared to the unmodified epitope; or
(ii) the K_{D}, IC₅₀, and/or EC₅₀ is increased by the amino acid substitution by at least about or at least about 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold as compared to the unmodified epitope.

119. The method of any one of claims 107-118, wherein the target gene encodes a cell-surface lineage-specific protein.

120. The method of claim 119, wherein the cell-surface lineage-specific protein is CD19;
CD123; CD38; KIT (CD117); CD20; or EGF-like module-containing mucin-like hormone receptor-like 2 (EMR2).

121. The method of claim 119, wherein the cell-surface lineage-specific protein is CD5, CD19, CD20, CD38, CD117, or CD123.

122. The method of claim 119, wherein the cell-surface lineage-specific protein is CD19, CD20, CD38, C-type lectin-like molecule-1 (CLL-1), CD123, or CD33.

123. The method of claim 119, wherein the cell-surface lineage-specific protein is CD33.

124. The method of claim 119, wherein the cell-surface lineage-specific protein is CD20.

125. The method of claim 119, wherein the cell-surface lineage-specific protein is CLL-1.

126. The method of claim 119, wherein the cell-surface lineage-specific protein is CD123.

127. The method of claim 119, wherein the cell-surface lineage-specific protein is CD38.

128. The method of claim 119, wherein the cell-surface lineage-specific protein is CD19.

129. The method of claim 119, wherein the cell-surface lineage-specific protein is CD117.

130. The method of claim 119, wherein the cell-surface lineage-specific protein is EMR2.

131. The method of claim 119, wherein the cell-surface lineage-specific protein is CD5.

132. The method of any one of claims 107-131, wherein the target gene, the cell-surface protein, and/or the cell-surface lineage-specific protein expression is associated with a cancer.

133. The method of any one of claims 107-131, wherein the target gene, the cell-surface protein, and/or the cell-surface lineage-specific protein expression is associated with a hematopoietic malignancy.

134. The method of claim 133, wherein the hematopoietic malignancy is Hodgkin's lymphoma, non-Hodgkin's lymphoma, leukemia, multiple myeloma (MM), myelodysplastic syndrome (MDS), or blastic plasmacytoid dendritic cell neoplasm (BPDCN).

135. The method of claim 133 or 134, wherein the hematopoietic malignancy is acute myeloid leukemia, B-cell acute lymphoblastic leukemia (B-ALL), chronic myelogenous leukemia, acute lymphoblastic leukemia, or chronic lymphoblastic leukemia.

136. The method of any one of claims 107-135, wherein the PAM and/or the artificial PAM is a Cas nuclease PAM.

137. The method of any one of claims 107-135, wherein the PAM and/or the artificial PAM is a Cas9 PAM.

138. The method of any one of claims 107-135, wherein the PAM and/or the artificial PAM is a Cas12a PAM.

139. The method of any one of claims 107-135, wherein the PAM and/or the artificial PAM is a SpyCas9 PAM, a SpGCas9 PAM, a SpRYCas9 PAM, a Sth1Cas9 PAM, a SauCas9 PAM, a NmeCas9 PAM, a RspCas9 PAM, a Cca1Cas9 PAM, a PspCas9 PAM, a OrhCas9 PAM, a ScCas9 PAM, a SmacCas9 PAM, a TdeCas9 PAM, a Nme2Cas9 PAM, a CjeCas9 PAM, a SmuCas9 PAM, a Smu2Cas9 PAM, a PmuCas9 PAM, a SpaCas9 PAM, a NciCas9 PAM, a ClaCas9 PAM, a PlaCas9 PAM, a CdCas9 PAM, a IgnaviCas9 PAM, a ThermoCas9 PAM, a GeoCas9 PAM, a G. LC300 Cas9 PAM, a AceCas9 PAM, a Sth3Cas9 PAM, a BlatCas9 PAM, a FnCas9 PAM, a LfeCas9 PAM, a LpnCas9 PAM, a KhuCas9 PAM, a AinCas9 PAM, a CglCas9 PAM, a Esp1Cas9 PAM, a Esp2Cas9 PAM, a FmaCas9 PAM, a LceCas9 PAM, a LrhCas9 PAM, a Lsp1Cas9 PAM, a Lsp2Cas9 PAM, a PacCas9 PAM, a TbaCas9 PAM, a TpuCas9 PAM, a VpaCas9 PAM, a EfaCas9 PAM, a EitCas9 PAM, a LanCas9 PAM, a LmoCas9 PAM, a Sag1Cas9 PAM, a Sag2Cas9 PAM, a SdyCas9 PAM, a Seq1Cas9 PAM, a Seq2Cas9 PAM, a SgaCas9 PAM, a Smu3Cas9 PAM, a SraCas9 PAM, a BniCas9 PAM, a EceCas9 PAM, a EdoCas9 PAM, a FhoCas9 PAM, a MgaCas9 PAM, a MseCas9 PAM, a SgoCas9 PAM, a SmaCas9 PAM, a SsaCas9 PAM, a SsiCas9 PAM, a SsuCas9 PAM, a Sth1ACas9 PAM, a TspCas9 PAM, a BokCas9 PAM, a CcoCas9 PAM, a CpeCas9 PAM, a DdeCas9 PAM, a Ghc2Cas9 PAM, a Ghy3Cas9 PAM, a Ghy4Cas9 PAM, a GspCas9 PAM, a KkiCas9 PAM, a NspCas9 PAM, a TmoCas9 PAM, a NsaCas9 PAM, a JpaCas9 PAM, a BboCas9 PAM, a Cca2Cas9 PAM, a Cme2Cas9 PAM, a Cme3Cas9 PAM, a Cme4Cas9 PAM, a CsaCas9 PAM, a Ghc1Cas9 PAM, a GheCas9 PAM, a Ghh1Cas9 PAM, a Ghh2Cas9 PAM, a Ghy1Cas9 PAM, a MscCas9 PAM, a SdoCas9 PAM, a SpacCas9 PAM, a CgaCas9 PAM, a CmelCas9 PAM, a FfrCas9 PAM, a Ghy2Cas9 PAM, a PhiCas9 PAM, a WviCas9 PAM, a CcCas9 PAM, a FnCas12a PAM, a AsCas12a PAM, a HkCas12a PAM, a PiCas12a PAM, a PdCas12a PAM, a LbCas12a PAM, a Lb2Cas12a or Lb5Cas12a PAm, a CMtCas12a PAM, a MbCas12a PAM, a TsCas12a PAM, a Pb2Cas12a PAM, a MICas12a PAM, a Mb2Cas12a PAM, a Mb3Cas12a PAm, a CMaCas12a PAM, a BsCas12a PAM, a BfCas12a PAM, a BoCas12a PAM, a Adurb193Cas12a PAM, a Adurb336Cas12a PAM, a Fn3Cas12a PAM, a Lb6Cas12a PAM, a EcCas12a PAM, a PsCas12a PAM, a McCas12a PAM, a AacCas12b PAM, a BthCas12b PAM, a AkCas12b PAM, a EbCas12b PaM, a BvCas12b PAM, a BhCas12b PAM, a LsCas12b PAM, a BrCas12b PAM, a Cas12c1 PAM, a Cas12c2 PAM, a OspCas12c PAM, a Cas12d.15 PAM, a Cas12d.1 (CasY.1) PAM, a DpbCas12e (DpbCasX) PAM, a PlmCas12e (PlmCasX) PAM, a MilCas12f2 PAM, a Un1Cas12f1 PAM, a Un2Cas12f1 PAM, a Mi2Cas12f2 PAM, a AuCas12f2 PAM, a PtCas12f1 PAM, a AsCas12f1 PAM, a RuCas12f1 PAM, a SpCas12f1 PAM, a CnCas12f1 PAM, a Cas12h1 PAM, a Cas12i1 PAM, a Cas1212 PAM, a Cas12j-1 (CasPhi-1) PAM, a Cas12j-2 (CasPhi-2) PAM, a Cas12j-3 (CasPhi-3) PAM, a ShCas12k PAM, or a AcCas12k PAM.

140. The method of any one of claims 107-139, wherein the PAM, and/or the artificial PAM comprises, and/or consists of a nucleotide sequence selected from: NGG; NNAGAAW; NNGRRT; NNNNGATT; NVNDCCY; BRTTTTT; NR (A or G) TTTT; NNAAAR (G or A); N (N or A) G; NAAN; NAAAAY; NHDTCCA; NNNVRYM; NNNNRYAC; NAA; GNNNNCNNA; NNGTGA; NNNNGTA; NNGGG; NNNCAT; NNRHHHY; NRRNAT; NNNNCNAA; NNNNCMCA; NNNNCRAA; NNNNGMAA; NNNCC; NGGNG; NNNNCNDD; NYAAA; NRGNN; N (C or D) GGN (T or A or G or C) NN; NRTAW; N (C or K or A) AARC; NAAAG; NV (A or G or C) R (A or G) ACCN; NNGAC; NATGNT; N (T or V) NTAAW (A or T); NNGW (A or T) AY (T or C); NCAA (H (Y or A) B (Y or G); NH (T or C or A) AAAA; NNNATTT; NATAWN (A or T or S); NATARCH; B (T or G or C) GGD (A or T or G) TNN; N (G or T or M) GGAH (T or A or C) N (A or C or K) N; NRG; N (B or A) GG; NGGD (A or K) W (T or A); N (T or C or R) AGAN (A or K or C) NN; NGGD (A or T or G) H (T or M); NGGDT; NGGD (A or T or G) GNN; NNGTAM (A or C) Y; NNGH (W or C) AAA; NTGAR (G or A) N (A or Y or G) N (Y or R); NNGAAAN; NNGAD; NHARMC; NNAAAG; NHGYNAN (A or B); NNAGAAA; NHAAAAA; NH (T or M) AAAAA; NHGYRAA; NNAAACN; NN (H or G) D (A or K) GGDN (A or B); NNNNCTA; NNNNCVGAA; NNNNGYAA; NNNNATN (W or S) ANN; NNWHR (G or A) TA (not G) AA; YHHNGTH; NNNNCDAANN; NNNNCTAA; N (C or D) NNTCCN; NNNNCCAA; NAGRGN (T or V) N (T or C); NNAH (T or M) ACN; CN (C or W or G) AV (A or S) GAC; NAR (G or A) H (W or C) H (A or T or C) GN (C or T or R); NAGNGC; NATCCTN; NGTGANN; HGCNGCR; NAR (A or G) W (T or A) AC; N (C or D) M (A or C) RN (A or B) AY (C or T); NNNCAC; BGGGTCD; NNRRCC; NRRNTT; KARDAT; BRRTTTW; NARNCCN; NAR (A or G) TC; NAAN (A or T or S) RCN; HHAAATD; NNNNGNA; TTV; TTTV; YYV; KKYV; TTTM; TTYV; TTTN; TTTTA; TTN; BTTV; YTV; YTN; NYTV; DTTD; ATTN; RTTNT; HATT; ATTW; RTTN; TVT; TG; TN; TR; TA; TTCN; TTAT; TTTR; TTR; YTTR; YTTN; CTT; TTC; CCD; RTR; VTTR; TBN; VTTN; NGTT; CGTT; AGG; CGG; GTT, or RGTG,
wherein “N” is any nucleotide or base, “W” is adenine (A) or thymine (T), “R” is A or guanine (G), “V” is A, cytosine (C), or G, “Y” is C or T, and “H” is A, C, or T.

141. The method of any one of claims 107-140, wherein the PAM, and/or the artificial PAM comprises, and/or consists of a nucleotide sequence of NGG, wherein “N” is any nucleotide or base.

142. The method of any one of claims 107-140, wherein the PAM, and/or the artificial PAM comprises, and/or consists of a nucleotide sequence of AGG.

143. The method of any one of claims 107-140, wherein the PAM, and/or the artificial PAM comprises, and/or consists of a nucleotide sequence of CGG.

144. The method of any one of claims 107-140, wherein the PAM, and/or the artificial PAM comprises, and/or consists of a nucleotide sequence of GTT.

145. The method of any one of claims 107-144, wherein the first target domain is within about 1, 2, 3, 4, 5, 6, 7, 8, 9, 10, 11, 12, 13, 14, 15, 16, 17, 18, 19, 20, 21, 22, 23, 24, or 25 nucleotides of a PAM which is capable of targeting a CRISPR/Cas system to the first target domain.

146. The method of any one of claims 107-145, wherein the genomic DNA molecule lacks a PAM in proximity of the second or subsequent target domain which is capable of targeting a CRISPR/Cas system to the second or subsequent target domain in order to provide a mutation within the second or subsequent target domain.

147. The method of claim 146, wherein the genomic DNA molecule lacks a suitable PAM within about 1, 2, 3, 4, 5, 6, 7, 8, 9, 10, 11, 12, 13, 14, 15, 16, 17, 18, 19, 20, 21, 22, 23, 24, or 25 nucleotides of the second or subsequent target domain which is capable of targeting a CRISPR/Cas system to the second or subsequent target domain in order to provide a mutation within the second or subsequent target domain.

148. The method of any one of claims 107-147, wherein the PAM which is capable of targeting a CRISPR/Cas system to the first target domain and the artificial PAM are separated by a predefined number of nucleotides.

149. The method of claim 148, wherein the PAM which is capable of targeting a CRISPR/Cas system to the first target domain and the artificial PAM are separated by about 1, 2, 3, 4, 5, 6, 7, 8, 9, 10, 11, 12, 13, 14, 15, 16, 17, 18, 19, 20, 21, 22, 23, 24, or 25 nucleotides.

150. The method of claim 149, wherein the artificial PAM is within about 1, 2, 3, 4, 5, 6, 7, 8, 9, 10, 11, 12, 13, 14, 15, 16, 17, 18, 19, 20, 21, 22, 23, 24, or 25 nucleotides of the second or subsequent target domain.

151. The method of any one of claims 107-150, wherein the mutation within the first target domain is within a predefined number of nucleotides of the PAM which is capable of targeting a CRISPR/Cas system to the first target domain.

152. The method of claim 151, wherein the mutation within the first target domain is within about 1, 2, 3, 4, 5, 6, 7, 8, 9, 10, 11, 12, 13, 14, 15, 16, 17, 18, 19, 20, 21, 22, 23, 24, or 25 nucleotides of the PAM which is capable of targeting a CRISPR/Cas system to the first target domain.

153. The method of claim 152, wherein the mutation within the second or subsequent target domain is within about 1, 2, 3, 4, 5, 6, 7, 8, 9, 10, 11, 12, 13, 14, 15, 16, 17, 18, 19, 20, 21, 22, 23, 24, or 25 nucleotides of the artificial PAM.

154. The method of any one of claims 148-153, wherein the predefined number of nucleotides comprises between about 1 to about 25 nucleotides, optionally, about 1, 2, 3, 4, 5, 6, 7, 8, 9, 10, 11, 12, 13, 14, 15, 16, 17, 18, 19, 20, 21, 22, 23, 24, or 25 nucleotides.

155. The method of any one of claims 107-154, wherein:
(i) the first gRNA comprising a targeting domain which binds a site in a CD33 gene, optionally, wherein the site comprises the nucleic acid sequence of GCAAGTGCAGGAGTCAGTGA (SEQ ID NO: 68), or a portion thereof; or
(ii) the first gRNA comprising a targeting domain which binds a site in a CD20 gene, optionally, wherein the site comprises the nucleic acid sequence of ATATAATTGTATATGTTGAC (SEQ ID NO: 69), or a portion thereof.

156. The method of any one of claims 107-155, wherein:
(i) the second gRNA comprising a targeting domain which binds a site in a CD33 gene, optionally, wherein the site comprises the nucleic acid sequence of GGATCCAAATTTCTGGCTGC (SEQ ID NO: 70), or a portion thereof; or
(ii) the second gRNA comprising a targeting domain which binds a site in a CD20 gene, optionally, wherein the site comprises the nucleic acid sequence of ACTTGGTCGATTAGGGAGAC (SEQ ID NO: 71), or a portion thereof.

157. The method of any one claims 107-155, wherein:
(i) the first gRNA comprising a targeting domain which binds a site in a CD33 gene, optionally, wherein the site comprises the nucleic acid sequence of GCAAGTGCAGGAGTCAGTGA (SEQ ID NO: 68), or a portion thereof;
(ii) the first gRNA comprising a targeting domain which binds a site in a CD20 gene, optionally, wherein the site comprises the nucleic acid sequence of ATATAATTGTATATGTTGAC (SEQ ID NO: 69), or a portion thereof;
(iii) the first gRNA comprising a targeting domain which binds a site in a CD38 gene, optionally, wherein the site comprises the nucleic acid sequence of GTAGTGAAATTCTAGAGCTT (SEQ ID NO: 81), or a portion thereof; or
(iv) the first gRNA comprising a targeting domain which binds a site in a CD123 gene, optionally, wherein the site comprises the nucleic acid sequence of CCTTTGGCTCACGCTGCTCC (SEQ ID NO: 82), or a portion thereof.

158. The method of any one of claims 107-157, wherein:
(i) the second gRNA comprising a targeting domain which binds a site in a CD33 gene, optionally, wherein the site comprises the nucleic acid sequence of GGATCCAAATTTCTGGCTGC (SEQ ID NO: 70), or a portion thereof;
(ii) the second gRNA comprising a targeting domain which binds a site in a CD20 gene, optionally, wherein the site comprises the nucleic acid sequence of ACTTGGTCGATTAGGGAGAC (SEQ ID NO: 71), or a portion thereof;
(iii) the second gRNA comprising a targeting domain which binds a site in a CD38 gene, optionally, wherein the site comprises the nucleic acid sequence of CCCGCAGGGTAAGTACCAAG (SEQ ID NO: 83) or GCAGGGTAAGTACCAAGTAG (SEQ ID NO: 84), or a portion thereof; or
(iv) the second gRNA comprising a targeting domain which binds a site in a CD123 gene, optionally, wherein the site comprises the nucleic acid sequence of CTCCTGATCGCCCTGCCTG (SEQ ID NO: 85), or a portion thereof.

159. The method of any one of claims 107-158, wherein the Cas nuclease is Cas9, Cas12a, or Cas12b.

160. The method of any one of claims 107-159, wherein the Cas nuclease comprises a catalytically inactive Cas molecule.

161. The method of any one of claims 107-160, wherein the Cas nuclease comprises a dead Cas (dCas).

162. The method of any one of claims 107-161, wherein the Cas nuclease comprises a dead Cas9 (dCas9).

163. The method of any one of claims 107-162, wherein the Cas nuclease comprises a nickase (nCas).

164. The method of any one of claims 107-163, wherein the Cas nuclease comprises a nCas9.

165. The method of any one of claims 107-164, wherein the Cas nuclease comprises a dCas or a nCas fused to one or more uracil glycosylase inhibitor (UGI) domains.

166. The method of any one of claims 107-165, wherein the Cas nuclease comprises a dCas or a nCas fused to an adenine base editor (ABE).

167. The method of claim 166, wherein the ABE comprises an adenine deaminase enzyme.

168. The method of any one of claims 107-167, wherein the Cas nuclease comprises a dCas or a nCas fused to a cytosine base editor (CBE).

169. The method of claim 168, wherein the CBE comprises a cytidine deaminase enzyme.

170. The method of any one of claims 107-169, wherein the contacting comprises introducing the CRISPR/Cas system into the cell in the form of a pre-formed ribonucleoprotein (RNP) complex.

171. The method of any one of claims 107-170, wherein the ribonucleoprotein complex is introduced into the cell via electroporation.

172. The method of any one of claims 107-171, in which (a) and (b) are introduced into the cell at the simultaneously.

173. The method of any one of claims 107-171, in which in which (a) and (b) are introduced into the cell sequentially.

174. The method of any one of claims 107-173, wherein the mutation at the first target domain comprises:
(i) a base (BE) editing mutation, optionally, wherein the base editing mutation comprises a modification which converts C to T, A to G, T to C, or G to A;
(ii) a high-fidelity homology directed repair (HDR)-based mutation, optionally, wherein the HDR-based mutation comprises a single nucleotide change and/or a insertion of a PAM sequence;
(iii) a ssODN mutation;
(iv) a prime editing (PE) mutation;
(v) a nucleobase transition, optionally, wherein nucleobase transition comprises the interchange of purine nucleobases A and G, or the interchange of pyrimidine nucleobases C and T; or
(vi) a nucleobase transversion, optionally, wherein the nucleobase transversion comprises the interchange of purine nucleobases A and G and pyrimidine nucleobases C and T.

175. The method of any one of claims 107-174, wherein the mutation at the second or subsequent target domain comprises:
(i) a base (BE) editing mutation, optionally, wherein the base editing mutation comprises a modification which converts C to T, A to G, T to C, or G to A;
(ii) a high-fidelity homology directed repair (HDR)-based mutation,
optionally, wherein the HDR-based mutation comprises a single nucleotide change and/or a insertion of a PAM sequence;
(iii) a ssODN mutation;
(iv) a prime editing (PE) mutation;
(v) a nucleobase transition, optionally, wherein nucleobase transition comprises the interchange of purine nucleobases A and G, or the interchange of pyrimidine nucleobases C and T;
(vi) a nucleobase transversion, optionally, wherein the nucleobase transversion comprises the interchange of purine nucleobases A and G and pyrimidine nucleobases C and T; or
(vii) a non-homologous end joining (NHEJ)-based mutation, optionally, wherein the NHEJ-based mutation comprises an indel that results in an amino acid deletion, insertion, or frameshift mutation, and/or wherein the NHEJ-based mutation results in a premature stop codon within the open reading frame (ORF) of the target gene.

176. The method of any one of claims 107-175, wherein the method further comprises introducing into the cell:
(a) two or more gRNA configured to direct two or more CRISPR/Cas systems to two or more sites to provide two or more mutations to generate two or more artificial PAMs in the genomic DNA molecule; and, optionally,
(b) two or more CRISPR/Cas systems that binds the two or more gRNA of (a).

177. The method of any one of claims 107-176, wherein the method further comprises introducing into the cell:
(a) two or more gRNA configured to direct two or more CRISPR/Cas systems to two or more sites to provide two or more mutations in two or more target genes; and, optionally,
(b) two or more CRISPR/Cas systems that binds the two or more gRNA of (a).

178. The method of any one of claims 107-177, wherein the method results in three or more mutations, optionally, wherein the method results in a plurality of mutations such that the mutation generates an artificial PAM in the genomic DNA molecule and the last mutation generates a desired modification or effect on a target gene and/or on a protein encoded by the target gene.

179. The method of claim 178, wherein the desired modification or effect on the target gene and/or the protein encoded by the target gene comprises healing a mutation, knocking out gene and/or protein expression, generating a target epitope that is not bound by a specific binding agent, optionally, wherein the binding agent is a ligand or an antibody.

180. The method of any one of claims 107-179, wherein the method results in four or more mutations, five or more mutations, six or more mutations, seven or more mutations, eight or more mutations, nine or more mutations, or ten or more mutations.

181. The method of any one of claims 178-180, wherein the mutation comprises the deamination of a cytosine.

182. The method of claim 181, wherein the mutation comprises the deamination of an adenine.

183. The method of claim 182, wherein the mutation comprises a nucleobase transition.

184. The method of claim 182, wherein the mutation comprises a nucleobase transversion.

185. The method of claim 184, wherein the mutation comprises converting a cytosine-guanine (C-G) base pair into a thymine-adenine (T-A) base pair within the target nucleic acid molecule.

186. The method of claim 182, wherein the mutation comprises converting a thymine-adenine (T-A) base pair into a cytosine-guanine (C-G) base pair within the target nucleic acid molecule.

187. The method of claim 182, wherein the mutation comprises introducing a premature STOP codon within the target nucleic acid molecule.

188. The method of claim 182, wherein the mutation comprises introducing a splice site within the target nucleic acid molecule.

189. The method of claim 182, wherein the mutation comprises disrupting a splice site within the target nucleic acid molecule.

190. The method of claim 189, wherein the mutation comprises disrupting a splice donor.

191. The method of claim 189, wherein the mutation comprises disrupting a splice acceptor.

192. The method of claim 189, wherein the mutation comprises disrupting a splice enhancer.

193. The method of claim 192, wherein the mutation comprises disrupting a exonic splice enhancer (ESE).

194. The method of any one of claims 107-193, wherein the mutation reduces the activity of and/or expression of a target gene in a cell.

195. A mixture comprising an mRNA encoding one, two, three, or all of:
(a) a first guide RNA (gRNA) configured to direct a first CRISPR/Cas system to a first target domain to provide an edit to generate an artificial PAM in a genomic DNA molecule;
(b) a second gRNA configured to direct a second CRISPR/Cas system to a second or subsequent target domain to provide an edit within a target gene;
(c) a CRISPR/Cas system that binds the first gRNA; and
(d) a CRISPR/Cas system that binds the second gRNA.

196. A mixture of claim 195, wherein (a)-(b) are encoding by the same mRNA or separate mRNA.

197. A kit or composition comprising:
(a) a first guide RNA (gRNA) configured to direct a first CRISPR/Cas system to a first target domain to provide an edit to generate an artificial PAM in a genomic DNA molecule; and
(b) a second gRNA configured to direct a second CRISPR/Cas system to a second or subsequent target domain to provide an edit within a target gene.

198. A kit or composition comprising:
(a) a first guide RNA (gRNA) configured to direct a first CRISPR/Cas system to a first target domain to provide an edit to generate an artificial PAM in a genomic DNA molecule;
(b) a second gRNA configured to direct a second CRISPR/Cas system to a second or subsequent target domain to provide an edit within a target gene;
(c) a CRISPR/Cas system that binds the first gRNA; and
(d) a CRISPR/Cas system that binds the second gRNA.


SEQUENCE LISTING (115 sequences)

SEQ ID NO: 1 - DNA, 51 bp, synthetic construct
  source 1..51: mol_type = other DNA
atggatccaaatttctggctgcrrgtgcaggagtcagtgacggtacaggag

SEQ ID NO: 2 - protein, 11 aa, synthetic construct
MDPNFWLQVQE

SEQ ID NO: 3 - DNA, 51 bp, synthetic construct
  source 1..51: mol_type = other DNA
atggrtccrrrtttctggctgcaggtgcaggagtcagtgacggtacaggag

SEQ ID NO: 4 - DNA, 51 bp, synthetic construct
  source 1..51: mol_type = other DNA
atggatccagatttctggctgcaggtgcaggagtcagtgacggtacaggag

SEQ ID NO: 5 - protein, 11 aa, synthetic construct
MDPDFWLQVQE

SEQ ID NO: 6 - protein, 55 aa, Homo sapiens
MPLLLLLPLLWAGALAMDPNFWLQVQESVTVQEGLCVLVPCTFFHPIPYYDKNSP

SEQ ID NO: 7 - DNA, 45 bp, Homo sapiens
  source 1..45: mol_type = genomic DNA
ctggctatggatccaaatttctggctgcaagtgcaggagtcagtg

SEQ ID NO: 8 - protein, 44 aa, synthetic construct
MPLLLLLPLLWAGALASVTVQEGLCVLVPCTFFHPIPYYDKNSP

SEQ ID NO: 9 - protein, 55 aa, synthetic construct
MPLLLLLPLLWAGALAMDPAAALQVQESVTVQEGLCVLVPCTFFHPIPYYDKNSP

SEQ ID NO: 10 - protein, 55 aa, synthetic construct
MPLLLLLPLLWAGALAMDPNFWAAAQESVTVQEGLCVLVPCTFFHPIPYYDKNSP

SEQ ID NO: 11 - protein, 55 aa, synthetic construct
MPLLLLLPLLWAGALAMDPDFWLQVQESVTVQEGLCVLVPCTFFHPIPYYDKNSP

SEQ ID NO: 12 - protein, 55 aa, synthetic construct
MPLLLLLPLLWAGALAMDPNYWLQVQESVTVQEGLCVLVPCTFFHPIPYYDKNSP

SEQ ID NO: 13 - protein, 55 aa, synthetic construct
MPLLLLLPLLWAGALAMDPNFWIQVQESVTVQEGLCVLVPCTFFHPIPYYDKNSP

SEQ ID NO: 14 - protein, 55 aa, synthetic construct
MPLLLLLPLLWAGALAMDPNFWLEVQESVTVQEGLCVLVPCTFFHPIPYYDKNSP

SEQ ID NO: 15 - protein, 5 aa, synthetic construct
ANPSE

SEQ ID NO: 16 - protein, 21 aa, Homo sapiens
YNCEPANPSEKNSPSTQYCYS

SEQ ID NO: 17 - DNA, 132 bp, synthetic construct
  source 1..132: mol_type = other DNA
atggagagtctgaattttattagagctcacacaccatatattaacatatacaactgtgaaccagctaatccctctgagaaaaactccccatctacccaatactgttacagcatacaatctctgttcttgggc

SEQ ID NO: 18 - protein, 44 aa, synthetic construct
MESLNFIRAHTPYINIYNCEPANPSEKNSPSTQYCYSIQSLFLG

SEQ ID NO: 19 - DNA, 132 bp, synthetic construct
  source 1..132: mol_type = other DNA
tacctctcagacttaaaataatctcgagtgtgtggtatataattgtatatgttgacacttggtcgattagggagactctttttgaggggtagatgggttatgacaatgtcgtatgttagagacaagaacccg

SEQ ID NO: 20 - DNA, 84 bp, synthetic construct
  source 1..84: mol_type = other DNA
gctcacacaccatatattaacatatacaactgtgaaccagctaatccctctgagaaaaactccccatctacccaatactgttac

SEQ ID NO: 21 - protein, 28 aa, synthetic construct
AHTPYINIYNCEPANPSEKNSPSTQYCY

SEQ ID NO: 22 - DNA, 84 bp, synthetic construct
  source 1..84: mol_type = other DNA
cgagtgtgtggtatataattgtatatgttgacacttggtcgattagggagactctttttgaggggtagatgggttatgacaatg

SEQ ID NO: 23 - DNA, 84 bp, synthetic construct
  source 1..84: mol_type = other DNA
gctcacacaccatatattaacacacacaaccgtgaaccagctaatccctctgagaaaaactccccatctacccaatactgttac

SEQ ID NO: 24 - protein, 28 aa, synthetic construct
AHTPYINTHNAEPANPSEKNSPSTQYCY

SEQ ID NO: 25 - DNA, 84 bp, synthetic construct
  source 1..84: mol_type = other DNA
cgagtgtgtggtatataattgtgtgtgttggcacttggtcgattagggagactctttttgaggggtagatgggttatgacaatg

SEQ ID NO: 26 - DNA, 124 bp, synthetic construct
  source 1..124: mol_type = other DNA
gaaataaactatcaatcttgcccagactggagaaaggactgcagcaacaaccctgtttcagtattctggaaaacggtttcccgcagggtaagtaccaagtagtgaaattctagagctttggaga

SEQ ID NO: 27 - protein, 29 aa, synthetic construct
KINYQSCPDWRKDCSNNPVSVFWKTVSRR

SEQ ID NO: 28 - DNA, 124 bp, synthetic construct
  source 1..124: mol_type = other DNA
tctccaaagctctagaatttcactacttggtacttaccctgcgggaaaccgttttccagaatactgaaacagggttgttgctgcagtcctttctccagtctgggcaagattgatagtttatttc

SEQ ID NO: 29 - protein, 39 aa, synthetic construct
SVFWKTVSRRFAEAACDVVHVMLDGSRSKIFDKDSTFGS

SEQ ID NO: 30 - DNA, 68 bp, synthetic construct
  source 1..68: mol_type = other DNA
ttcagtattctggaaaacggtttcccgcagggtaagtaccaagtagtgaaattctagagctttggaga

SEQ ID NO: 31 - protein, 10 aa, synthetic construct
SVFWKTVSRR

SEQ ID NO: 32 - DNA, 68 bp, synthetic construct
  source 1..68: mol_type = other DNA
tctccaaagctctagaatttcactacttggtacttaccctgcgggaaaccgttttccagaatactgaa

SEQ ID NO: 33 - DNA, 57 bp, synthetic construct
  source 1..57: mol_type = other DNA
tctggaaaacggtttcccgcagggtaagtaccaagtagtgaaattctagagctttgg

SEQ ID NO: 34 - protein, 19 aa, synthetic construct
GSTSSGSGKPGSGEGSTKG

SEQ ID NO: 35 - protein, 107 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 36 - protein, 89 aa, synthetic construct
LSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 37 - protein, 38 aa, synthetic construct
CWLTKKKYSSSVHDPNGEYMFMRAVNTAKKSRLTDVTL

SEQ ID NO: 38 - protein, 97 aa, synthetic construct
IEVMYPPPYLDNEKSNGTIIHVKGKHLCPSPLFPGPSKPFWVLVVVGGVLACYSLLVTVAFIIFWVRSKRSRLLHSDYMFMRAVNTAKKSRLTDVTL

SEQ ID NO: 39 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 40 - protein, 8 aa, synthetic construct
FWKTVSRR

SEQ ID NO: 41 - DNA, 57 bp, synthetic construct
  source 1..57: mol_type = other DNA
ccaaagctctagaatttcactacttggtacttaccctgcgggaaaccgttttccaga

SEQ ID NO: 42 - DNA, 54 bp, synthetic construct
  source 1..54: mol_type = other DNA
ggaaaacggtttcccgcagggtaagtaccaagtagtgaaattctagagctttgg

SEQ ID NO: 43 - protein, 7 aa, synthetic construct
WKTVSRR

SEQ ID NO: 44 - DNA, 54 bp, synthetic construct
  source 1..54: mol_type = other DNA
ccaaagctctagaatttcactacttggtacttaccctgcgggaaaccgttttcc

SEQ ID NO: 45 - DNA, 47 bp, synthetic construct
  source 1..47: mol_type = other DNA
ttcccgcagggtaagtaccaagtagtgaaattctagagctttggaga

SEQ ID NO: 46 - DNA, 47 bp, synthetic construct
  source 1..47: mol_type = other DNA
tctccaaagctctagaatttcactacttggtacttaccctgcgggaa

SEQ ID NO: 47 - DNA, 38 bp, synthetic construct
  source 1..38: mol_type = other DNA
cagggtaagtaccaagtggtggaattctagagctttgg

SEQ ID NO: 48 - DNA, 38 bp, synthetic construct
  source 1..38: mol_type = other DNA
cagggtaagtaccaagtagtgaaattctagagctttgg

SEQ ID NO: 49 - DNA, 38 bp, synthetic construct
  source 1..38: mol_type = other DNA
cagggtaagtaccaagtggtgaaattctagagctttgg

SEQ ID NO: 50 - DNA, 42 bp, synthetic construct
  source 1..42: mol_type = other DNA
cccgcagggtaagtaccaagtagtgaaattctagagctttgg

SEQ ID NO: 51 - DNA, 42 bp, synthetic construct
  source 1..42: mol_type = other DNA
cccgcaaggtaagtaccaagtggtgaaattctagagctttgg

SEQ ID NO: 52 - DNA, 42 bp, synthetic construct
  source 1..42: mol_type = other DNA
cccgcagggtaagtaccaagtggtgaaattctagagctttgg

SEQ ID NO: 53 - DNA, 42 bp, synthetic construct
  source 1..42: mol_type = other DNA
cccgcaaggtaagtaccaagtggtggaattctagagctttgg

SEQ ID NO: 54 - DNA, 44 bp, synthetic construct
  source 1..44: mol_type = other DNA
ttcccgcggggtaagtaccaagtggtggaattctagagctttgg

SEQ ID NO: 55 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 56 - DNA, 44 bp, synthetic construct
  source 1..44: mol_type = other DNA
ccaaagctctagaattccaccacttggtacttaccccgcgggaa

SEQ ID NO: 57 - DNA, 26 bp, synthetic construct
  source 1..26: mol_type = other DNA
cggtttcccgcagggtaagtaccaag

SEQ ID NO: 58 - DNA, 26 bp, synthetic construct
  source 1..26: mol_type = other DNA
cggtttcccgcggggtaagtaccaag

SEQ ID NO: 59 - DNA, 83 bp, synthetic construct
  source 1..83: mol_type = other DNA
tgcgttcccgatggtcctcctttggctcacgctgctcctgatcgccctgccctgtctcctgcaaacgaaggaaggtaagaact

SEQ ID NO: 60 - protein, 21 aa, synthetic construct
MVLLWLTLLLIALPCLLQTKE

SEQ ID NO: 61 - DNA, 83 bp, synthetic construct
  source 1..83: mol_type = other DNA
agttcttaccttccttcgtttgcaggagacagggcagggcgatcaggagcagcgtgagccaaaggaggaccatcgggaacgca

SEQ ID NO: 62 - DNA, 74 bp, synthetic construct
  source 1..74: mol_type = other DNA
gttcccgatggtcctcctttggctcacgctgctcctgatcgccctgccctgtctcctgcaaacgaaggaaggta

SEQ ID NO: 63 - DNA, 74 bp, synthetic construct
  source 1..74: mol_type = other DNA
taccttccttcgtttgcaggagacagggcagggcgatcaggagcagcgtgagccaaaggaggaccatcgggaac

SEQ ID NO: 64 - DNA, 84 bp, synthetic construct
  source 1..84: mol_type = other DNA
cgagtgtgtggtatataattgtrtrtgttgrcacttggtcgattagggagactctttttgaggggtagatgggttatgacaatg

SEQ ID NO: 65 - DNA, 84 bp, synthetic construct
  source 1..84: mol_type = other DNA
cgagtgtgtggtatataattgtgtgtgttggcacttggtcgrttrgggrgrctctttttgaggggtagatgggttatgacaatg

SEQ ID NO: 66 - DNA, 84 bp, synthetic construct
  source 1..84: mol_type = other DNA
cgagtgtgtggtatataattgtgtgtgttggcacttggtcggttgggggggctctttttgaggggtagatgggttatgacaatg

SEQ ID NO: 67 - protein, 28 aa, synthetic construct
AHTPYINTHNAEPALPPEKNSPSTQYCY

SEQ ID NO: 68 - DNA, 20 bp, synthetic construct
  source 1..20: mol_type = other DNA
gcaagtgcaggagtcagtga

SEQ ID NO: 69 - DNA, 20 bp, synthetic construct
  source 1..20: mol_type = other DNA
atataattgtatatgttgac

SEQ ID NO: 70 - DNA, 20 bp, synthetic construct
  source 1..20: mol_type = other DNA
ggatccaaatttctggctgc

SEQ ID NO: 71 - DNA, 20 bp, synthetic construct
  source 1..20: mol_type = other DNA
acttggtcgattagggagac

SEQ ID NO: 72 - DNA, 43 bp, synthetic construct
  source 1..43: mol_type = other DNA
cgcagggtaagtaccaagtggtgaaattctagagctttggaga

SEQ ID NO: 73 - DNA, 43 bp, synthetic construct
  source 1..43: mol_type = other DNA
cgcagggtaagtaccaagtagtgrrrttctagagctttggaga

SEQ ID NO: 74 - DNA, 43 bp, synthetic construct
  source 1..43: mol_type = other DNA
cgcggggtaagtaccaagtggtgaaattctagagctttggaga

SEQ ID NO: 75 - DNA, 43 bp, synthetic construct
  source 1..43: mol_type = other DNA
cgcagggtrrgtaccaagtagtgrrrttctagagctttggaga

SEQ ID NO: 76 - protein, 29 aa, synthetic construct
KINYQSCPDWRKDCSNNPVSVFWKTVSRG

SEQ ID NO: 77 - DNA, 63 bp, synthetic construct
  source 1..63: mol_type = other DNA
taccaggaggaaaccgagtgcggcgaggactagcgggacgggacagaggacgtttgcttcctt

SEQ ID NO: 78 - DNA, 63 bp, synthetic construct
  source 1..63: mol_type = other DNA
taccaggaggaaaccgagtgcggcgaggactagcggggcgggacagaggacgtttgcttcctt

SEQ ID NO: 79 - protein, 21 aa, synthetic construct
MVLLWLTPLLIALPCLLQTKE

SEQ ID NO: 80 - protein, 21 aa, synthetic construct
MVLLWLTPLLIAPPCLLQTKE

SEQ ID NO: 81 - DNA, 20 bp, synthetic construct
  source 1..20: mol_type = other DNA
gtagtgaaattctagagctt

SEQ ID NO: 82 - DNA, 20 bp, synthetic construct
  source 1..20: mol_type = other DNA
cctttggctcacgctgctcc

SEQ ID NO: 83 - DNA, 20 bp, synthetic construct
  source 1..20: mol_type = other DNA
cccgcagggtaagtaccaag

SEQ ID NO: 84 - DNA, 20 bp, synthetic construct
  source 1..20: mol_type = other DNA
gcagggtaagtaccaagtag

SEQ ID NO: 85 - DNA, 19 bp, synthetic construct
  source 1..19: mol_type = other DNA
ctcctgatcgccctgcctg

SEQ ID NO: 86 - DNA, 19 bp, synthetic construct
  source 1..19: mol_type = other DNA
gcaagtgcaggagtcagtg

SEQ ID NO: 87 - DNA, 19 bp, synthetic construct
  source 1..19: mol_type = other DNA
ggatccaaatttctggctg

SEQ ID NO: 88 - protein, 9 aa, synthetic construct
EPANPSEKN

SEQ ID NO: 89 - DNA, 21 bp, synthetic construct
  source 1..21: mol_type = other DNA
atataattgtatatgttgaca

SEQ ID NO: 90 - protein, 5 aa, synthetic construct
ALPPE

SEQ ID NO: 91 - DNA, 4764 bp, synthetic construct
  source 1..4764: mol_type = other DNA
atgtccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgactttatgatgcgacgctgtactcgacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgactctggtggttcttctggtggttctagcggcagcgagactcccgggacctcagagtccgccacacccgaaagttctggtggttcttctggtggttctgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacgagggagctgataagcgcaccgccgatggttccgagttcgaaagccccaagaagaagaggaaagtc

SEQ ID NO: 92 - DNA, 5490 bp, synthetic construct
  source 1..5490: mol_type = other DNA
atgacctctgagaagggccctagcacaggcgaccccaccctgcggcggagaatcgagagctgggagttcgacgtgttctacgaccctagagaactggccaaggaaacctgcctgctgtacgagatcaagtggggcatgagcagaaagatctggcggagctctggcaagaacaccaccaaccacgtggaagtgaatttcatcaagaagttcaccagcgagagaaggttccacagcagcatcagctgcagcatcacctggttcctgagctggtccccttgctgggaatgcagccaggccatcagagagttcctgagccaacaccccggagtgacactggtgatctacgtggccagactgttctggcacatggaccagagaaacagacagggcctgagagatctggtcaacagcggcgtgactatccagatcatgcgggccagcgagtactaccactgttggcggaacttcgtgaactacccccccggcgatgaggcccactggcctcagtaccctcctctgtggatgatgctgtacgccctggaactgcactgcatcatcctgtctctgcctccatgtctgaagatctctagaagatggcagaaccacctggccttcttcagactgcacctgcagaattgccactaccagaccatccccccccacatcctgctggctacaggcctgatccacccttctgtgacctggagacttaagagcggaggatctagcggcggctctagcggatctgagacacctggcacaagcgagtctgccacacctgagagtagcggcggatcttctggtggctctgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggtggaagcggaggatctggcggcagcaccaatctgagcgacatcatcgagaaagagacaggcaagcagctggtcatccaagagtccatcctgatgctgcctgaagaggtggaagaagtgatcggcaacaagcccgagtccgacatcctggtgcacaccgcctacgatgagagcaccgacgagaacgtgatgctgctgacctctgacgcccctgagtacaagccttgggctctcgtgatccaggacagcaacggcgagaacaagatcaagatgctgagcggcggctctggtggctctggcggatctacaaacctgtccgatattattgagaaagaaaccgggaaacagctcgtgattcaagagtctattctcatgctcccggaagaagtcgaggaagtcattggaaacaagcctgagagcgatattctggtccatacagcctacgacgagtctaccgatgagaatgtcatgctcctcaccagcgacgctcccgagtataagccatgggcacttgtcattcaggactccaatggggaaaacaaaatcaaaatgctcccaaagaaaaaacgcaaggtg

SEQ ID NO: 93 - DNA, 5490 bp, synthetic construct
  source 1..5490: mol_type = other DNA
atgacctctgagaagggccctagcacaggcgaccccaccctgcggcggagaatcgagagctgggagttcgacgtgttctacgaccctagagaactgagaaaggaaacctgcctgctgtacgagatcaagtggggcatgagcagaaagatctggcggagctctggcaagaacaccaccaaccacgtggaagtgaatttcatcaagaagttcaccagcgagagaaggttccacagcagcatcagctgcagcatcacctggttcctgagctggtccccttgctgggaatgcagccaggccatcagagagttcctgagccaacaccccggagtgacactggtgatctacgtggccagactgttctggcacatggaccagagaaacagacagggcctgagagatctggtcaacagcggcgtgactatccagatcatgcgggccagcgagtactaccactgttggcggaacttcgtgaactacccccccggcgatgaggcccactggcctcagtaccctcctctgtggatgatgctgtacgccctggaactgcactgcatcatcctgtctctgcctccatgtctgaagatctctagaagatggcagaaccacctggccttcttcagactgcacctgcagaattgccactaccagaccatccccccccacatcctgctggctacaggcctgatccacccttctgtgacctggagacttaagagcggaggatctagcggcggctctagcggatctgagacacctggcacaagcgagtctgccacacctgagagtagcggcggatcttctggtggctctgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggtggaagcggaggatctggcggcagcaccaatctgagcgacatcatcgagaaagagacaggcaagcagctggtcatccaagagtccatcctgatgctgcctgaagaggtggaagaagtgatcggcaacaagcccgagtccgacatcctggtgcacaccgcctacgatgagagcaccgacgagaacgtgatgctgctgacctctgacgcccctgagtacaagccttgggctctcgtgatccaggacagcaacggcgagaacaagatcaagatgctgagcggcggctctggtggctctggcggatctacaaacctgtccgatattattgagaaagaaaccgggaaacagctcgtgattcaagagtctattctcatgctcccggaagaagtcgaggaagtcattggaaacaagcctgagagcgatattctggtccatacagcctacgacgagtctaccgatgagaatgtcatgctcctcaccagcgacgctcccgagtataagccatgggcacttgtcattcaggactccaatggggaaaacaaaatcaaaatgctcccaaagaaaaaacgcaaggtg

SEQ ID NO: 94 - protein, 1588 aa, synthetic construct
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGADKRTADGSEFESPKKKRKV

SEQ ID NO: 95 - protein, 1830 aa, synthetic construct
MTSEKGPSTGDPTLRRRIESWEFDVFYDPRELAKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWRLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLPKKKRKV

SEQ ID NO: 96 - protein, 1830 aa, synthetic construct
MTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWRLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLPKKKRKV

SEQ ID NO: 97 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 98 - protein, 301 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSPVRTQ

SEQ ID NO: 99 - protein, 265 aa, Homo sapiens
MSEEVTYADLQFQNSSEMEKIPEIGKFGEKAPPAPSHVWRPAALFLTLLCLLLLIGLGVLASMFHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAWVIRNAPDLNNMYCGYINRLYVQYYHCTYKKRMICEKMANPVQLGSTYFREA

SEQ ID NO: 100 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 101 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 102 - protein, 556 aa, Homo sapiens [sequence omitted]

SEQ ID NO: 103 - protein, 976 aa, Homo sapiens [sequence omitted]

SEQ ID NO: 104 - protein, 823 aa, Homo sapiens
MGGRVFLVFLAFCVWLTLPGAETQDSRGCARWCPQDSSCVNATACRCNPGFSSFSEIITTPMETCDDINECATLSKVSCGKFSDCWNTEGSYDCVCSPGYEPVSGAKTFKNESENTCQDVDECQQNPRLCKSYGTCVNTLGSYTCQCLPGFKLKPEDPKLCTDVNECTSGQNPCHSSTHCLNNVGSYQCRCRPGWQPIPGSPNGPNNTVCEDVDECSSGQHQCDSSTVCFNTVGSYSCRCRPGWKPRHGIPNNQKDTVCEDMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGPSVVGLVSIPGMGKLLAEAPLVLEPEKQMLLHETHQGLLQDGSPILLSDVISAFLSNNDTQNLSSPVTFTFSHRSVIPRQKVLCVFWEHGQNGCGHWATTGCSTIGTRDTSTICRCTHLSSFAVLMAHYDVQEEDPVLTVITYMGLSVSLLCLLLAALTFLLCKAIQNTSTSLHLQLSLCLFLAHLLFLVAIDQTGHKVLCSIIAGTLHYLYLATLTWMLLEALYLFLTARNLTVVNYSSINRFMKKLMFPVGYGVPAVTVAISAASRPHLYGTPSRCWLQPEKGFIWGFLGPVCAIFSVNLVLFLVTLWILKNRLSSLNSEVSTLRNTRMLAFKATAQLFILGCTWCLGILQVGPAARVMAYLFTIINSLQGVFIFLVYCLLSQQVREQYGKWSKGIRKLKTESEMHTLSSSAKADTSKPSTVN

SEQ ID NO: 105 - protein, 495 aa, Homo sapiens
MPMGSLQPLATLYLLGMLVASCLGRLSWYDPDFQARLTRSNSKCQGQLEVYLKDGWHMVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNPAGLAAGTVASIILALVLLVVLLVVCGPLAYKKLVKKFRQKKQRQWIGPTGMNQNMSFHRNHTATVRSHAENPTASHVDNEYSQPPRNSHLSAYPALEGALHRSSMQPDNSSDSDYDLHGAQRL

SEQ ID NO: 106 - protein, 353 aa, synthetic construct
MPLLLLLPLLWAGALASVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 107 - protein, 364 aa, synthetic construct
MPLLLLLPLLWAGALAMDPAAALQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 108 - protein, 364 aa, synthetic construct
MPLLLLLPLLWAGALAMDPNFWAAAQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 109 - protein, 364 aa, synthetic construct
MPLLLLLPLLWAGALAMDPDFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 110 - protein, 364 aa, synthetic construct
MPLLLLLPLLWAGALAMDPNYWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 111 - protein, 364 aa, synthetic construct
MPLLLLLPLLWAGALAMDPNFWIQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 112 - protein, 364 aa, synthetic construct
MPLLLLLPLLWAGALAMDPNFWLEVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 113 - protein, 10 aa, synthetic construct
EPANPSEKNS

SEQ ID NO: 114 - DNA, 43 bp, synthetic construct
  source 1..43: mol_type = other DNA
cgcagggtaagtaccaagtagtggaattctagagctttggaga

SEQ ID NO: 115 - protein, 41 aa, synthetic construct
KRGRKKLLYIFKQPFMRPVQTTQEEDGCSCRFPEEEEGGCE